(19) United States
(12) Patent Application Publication
(10) Pub. No.: US 2025/0170111 A1
(43) Pub. Date: May 29, 2025

(54) PYRIDINE BASED CHALCONE ANALOGS AS THERAPEUTIC AGENTS AND USE THEREOF

(71) Applicant: QATAR UNIVERSITY, Doha (QA)

(72) Inventors: Ashraf KHALIL, Doha (QA); Ala-Eddin AL MOUSTAFA, Doha (QA); Ola Jihad HUSSEIN, Doha (QA); Arij Fouzat HASSAN, Doha (QA); Feras ALALI, Doha (QA)

(21) Appl. No.: 18/907,427

(22) Filed: Oct. 4, 2024

Related U.S. Application Data
(60) Provisional application No. 63/542,713, filed on Oct. 5, 2023.

(51) Int. Cl. A61K 31/4436; C07D 409/04

(52) U.S. Cl. CPC A61K 31/4436; C07D 409/04

(57) ABSTRACT

Novel pyridine based chalcone analogs compounds, compositions comprising the compounds and methods of treating diseases with the compounds and compositions.

### CROSS-REFERENCE TO RELATED APPLICATION

This application claims the benefit of U.S. Provisional Application No. 63/542,713, filed Oct. 5, 2023. The entire contents of the above-identified application are hereby fully incorporated herein by reference.

### TECHNICAL FIELD

The subject matter disclosed herein is generally directed to novel compounds for treating cancers.

### BACKGROUND

Breast cancer is the most frequently diagnosed malignancy among women worldwide, of which about 20% are triple-negative breast cancer (TNBC) which is the most metastatic and highest mortality disease form. So far, a limited number of known molecular targets have significantly reduced available TNBC's treatment options. Similarly, prostate cancer (PCa) is the second most frequently diagnosed malignancy, as well as a leading cause of cancer-related mortality in men globally. Despite the initial response to hormone-targeted therapy, most patients ultimately progress to a lethal form of the disease, castration-resistant prostate cancer (CRPC), which currently lacks curative therapeutic options and is associated with a poor prognosis.

Among the attractive scaffolds being investigated for their antitumor activities are chalcone derivatives. Chalcones, also known as benzylideneacetophenone or 1,3-diphenyl-2-propen-1-ones, are open-chain flavonoids that are widely distributed in various plant species. Chalcone is a chemical scaffold with multifarious biological activities, including antidiabetic, anti-inflammatory, antioxidant and anticancer. The variety in biological activity stems from the unique features of the chalcone skeleton. Chalcones are composed of numerous replaceable hydrogens that allow many derivatives to be generated that differ in their specificity and reactivity with biological targets. Due to their biological properties, chalcones-rich plants were historically used in traditional medicine. Several pure chalcones were isolated from plants and were approved for use in humans. However, so far, chalcone analogs have not been studied thoroughly enough for the management of cancer and none has entered subsequent development steps as a potential targeted treatment for cancer. This could be partially attributed to their poor pharmacokinetic profile, lack of selectivity and/or modest potency.

Therefore, there is a need for the development of new alternatives or improved therapeutic options to combat TNBC, PCa, CRPC and other forms of cancer.

### SUMMARY

In one aspect, the present disclosure provides a compound according to Formula (I):

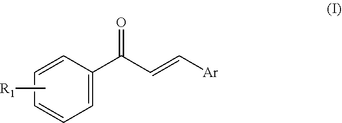

or a pharmaceutically acceptable salt thereof, wherein Ar is

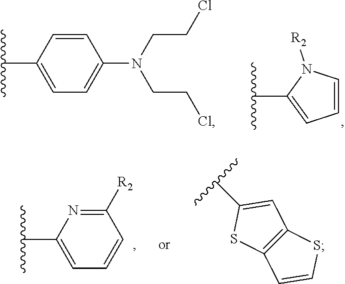

R₁ is —OCH₃, —OH, —CF₃, —SO₂CH₃, or —F; and R₂ is absent, —H, cyclopropyl, —OCH₃, —CF₃, or thienyl.

In some embodiments, the compound has the structure of Formula (II):

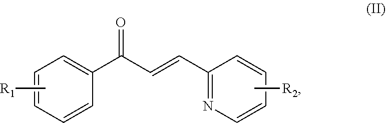

wherein R₂ is OCH₃, CF₃, or thienyl. In some embodiments, the R1 is —OCH₃. In some embodiments, the R1 is-CF₃. In some embodiments, the R₂ is thienyl. In some embodiments, the R₂ is 3-thienyl.

In some embodiments, the compound has the structure of Formula (III)

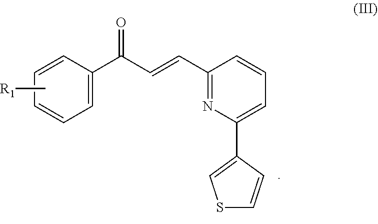

In some embodiments, the compound is any one of compounds 13-26 in FIG. 15. In some embodiments, the compound has the structure of the following formula:

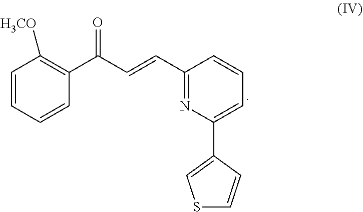 [SMILES: COc1ccccc1C(=O)/C=C/c1cccc(-c2ccsc2)n1]

In some embodiments, the compound has the structure of the following formula:

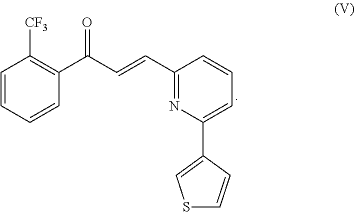 [SMILES: O=C(/C=C/c1cccc(-c2ccsc2)n1)c1ccccc1C(F)(F)F]

In another aspect, the present disclosure provides a pharmaceutical composition comprising the compound herein and a pharmaceutically acceptable carrier.

In another aspect, the present disclosure provides a method of treating a disease, the method comprising administering the compound herein or the pharmaceutical composition herein to a subject in need thereof. In some embodiments, the disease is a cancer. In some embodiments, the cancer is a breast cancer. In some embodiments, the breast cancer is TNBC. In some embodiments, wherein the cancer is a prostate cancer.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1: Chemical structures of example compounds (OH17 and OH 25).

FIG. 2: Methods of identifying and testing exemplary compounds.

FIG. 3: Chemical Structure of chalcone scaffold.

FIG. 4: Structures of clinically approved chalcone-based drugs.

FIG. 5: Molecular hybridization of chalcones.

FIG. 6: Examples of naturally occurring chalcones with anticancer activities.

FIG. 7: Example of chalcone analogs with tubulin assembly inhibitory activities.

FIG. 8: Synthetic scheme of tetralone-based chalcones and non-cyclic chalcone analogs.

FIG. 9: Illustration of the used purification techniques.

FIGS. 10A-10B: Illustration of Alamar Blue Assay. FIG. 10A shows the reaction used in the assay. FIG. 10B shows an exemplary setting of the assay.

FIG. 11: Timeline of CAM Angiogenesis Assay.

FIG. 12: In Silico ADMET Screening.

FIGS. 13A1-13B: Design of nitrogen mustard tetralone-based chalcones. FIG. 13A shows an exemplary cyclic (tetralone)-based chalcone. FIG. 13B shows an exemplary cyclopropyl containing chalcone.

FIG. 14A: Examples of clinically approved drugs containing the heteroaromatic rings (pyrrole, pyridine, and thiophene). FIG. 14B: The design of pyridine-chalcone hybrids.

FIG. 15: Chemical structures of the synthesized chalcone analogs.

FIGS. 16A-16B: Characteristic coupling constants of (FIG. 16A) pyrrole ring (compounds 6-9) and (FIG. 16B) thienyl pyridine rings (compounds 13-26).

FIG. 17: Key ¹H-¹H COSY, 1H-13C HMBC and ¹H-¹⁵N HMBC correlations of compounds 13 and 16.

FIG. 18A: Structure of compound 17. FIG. 18B: Predicted drug-likeness properties of compound 17 by SwissADME web tool (www.swissadme.ch/).

FIG. 19: ADMET Risk scores of the synthesized chalcone analogs (1-26), presented as heat map predicted by ADMET Predictor®. Abbreviations Absn: Absorption; Tox: Toxicity; MUT: Mutagenicity; Sw: Water Solubility; CL: Clearance; Kow n-Octanol/Water Partition Coefficient. Recommended Parameters Range by AMDET Predictor: overall ADMET risk (<6.5), Absn_Risk (<3.5), Tox_Risk (<2), MUT (<1), CYP_Risk (<2.5).

FIGS. 20A-20B: Effect of compounds 1-16 on the cell viability of PC3 (FIG. 20A) and DU145 (FIG. 20B) cell lines.

FIG. 21: Effect of compounds 1-16 on the cell morphology of PC3 cell line.

FIG. 22: Effect of compounds 1-16 on the cell morphology of DU145 cell line.

FIGS. 23A-23C: The effect of compounds 13, 15 and 16 on cell viability of AR-negative prostate cancer cell lines PC3 (FIG. 23A) and DU145 (FIG. 23B), and normal dental cells (FIG. 23C) as determined by Alamar Blue assay, 48 hours post-treatment.

FIG. 24: Effect of compound 13 on the cell morphology of AR-positive (C4-2 and LNCaP) and AR-negative (PC3 and DU145) prostate cancer cell lines.

FIG. 25: Effect of compound 16 on the cell morphology of AR-negative (PC3 and DU145) prostate cancer cell lines.

FIGS. 26A-26B: Induction of apoptosis by compounds 13, 15 and 16 in PC3 cells, as determined by Annexin V-FITC (FIG. 26A) and 7-AAD apoptosis assay (FIG. 26B).

FIGS. 27A-27B: Induction of apoptosis by compounds 13, 15 and 16 in DU145 cells, as determined by Annexin V-FITC (FIG. 27A) and 7-AAD apoptosis assay (FIG. 27B).

FIGS. 28A-28B: Effect of compounds 13, 15 and 16 on the expression of apoptosis-related proteins in PC3 cells. FIG. 28A shows the Western blot. FIG. 28B shows the quantification of the Western blot results.

FIGS. 29A-29B: Effect of compounds 13, 15 and 16 on the expression of apoptosis-related proteins in DU145 cells. FIG. 29A shows the Western blot. FIG. 29B shows the quantification of the Western blot results.

FIGS. 30A-30B: Effect of compounds 13, 15 and 16 on cell cycle progression of PC3 cells. FIG. 30A shows cell counts. FIG. 30B shows cell cycle distribution quantifications.

FIGS. 31A-31B: Effect of compounds 13, 15 and 16 on cell cycle progression of DU145 cells. FIG. 31A shows cell counts. FIG. 31B shows cell cycle distribution quantifications.

FIGS. 32A-32D: Effect of compounds 13 and 16 on colony formation of PC3 cell line. FIG. 32A shows the images of colonies. FIG. 32B shows number of colonies. FIG. 32C shows colony sizes. FIG. 32D shows colony size distribution.

FIGS. 33A-33D: Effect of compounds 13 and 16 on colony formation of DU145 cell line. FIG. 33A shows images of colonies. FIG. 33B shows number of colonies. FIG. 33C shows colony sizes. FIG. 33D shows colony size distribution.

FIGS. 34A-34B: Effect of compounds 13, 15 and 16 on Transwell migration of PC3 cells. FIG. 34A shows the images of cell migration. FIG. 34B shows the quantification of migrated cells.

FIGS. 35A-35B: Effect of compounds 13, 15 and 16 on Transwell migration of DU145 cells. FIG. 35A shows the images of cell migration. FIG. 35B shows the quantification of migrated cells.

FIGS. 36A-36B: Effect of compounds 13, 15 and 16 on the migration of PC3 cells in wound healing assay. FIG. 36A shows the images of the healing assay. FIG. 36B shows the quantification of the result.

FIGS. 37A-37B: Effect of compounds 15 and 16 on the migration of DU145 cells in wound healing assay. FIG. 37A shows the images of the healing assay. FIG. 37B shows the quantification of the result.

FIGS. 38A-38B: Protein expression of EMT-related biomarkers on PC3 cell line. FIG. 38A: Representative outcomes of molecules 13, 15 and 16 (5 and 10 μM) on the expression of E-cadherin, p-β-catenin, β-catenin, and fascin proteins at 48 hours posttreatment. FIG. 38B shows the quantification of the Western blot results.

FIGS. 39A-39B: Protein expression of EMT-related biomarkers on DU145 cell line. FIG. 39A shows the Western blot. FIG. 39B shows the quantification of the Western blot results.

FIGS. 40A-40B: Protein expression of JNK, ERK, and AKT-related biomarkers on PC3 cell line. FIG. 40A shows the Western blot. FIG. 40B shows the quantification of the Western blot results.

FIGS. 41A-41B: Protein expression of JNK, ERK, and AKT-related biomarkers on DU145 cell line. FIG. 41A shows the Western blot. FIG. 41B shows the quantification of the Western blot results.

FIGS. 42A-42C: Effect of Compound 16 on Angiogenesis of the CAM of chicken embryos. FIG. 42A shows the images of angiogenesis. FIG. 42B shows further details of angiogenesis. FIG. 42C shows the angiogenesis parameters obtained from the assay.

FIGS. 43A-42B: Effect of thienyl pyridine chalcone hybrids (13-26) on the cell viability of (FIG. 43A) PC3 and (FIG. 43B) DU145 cell lines.

FIG. 44: Key structure-activity relationship (SAR) findings.

FIGS. 45A-45B: Effect of thienyl pyridine chalcone hybrids (13-26) on the cell viability of MDA-MB-231 (FIG. 45A) and MDA-MB-436 (FIG. 45B) cell lines.

FIGS. 46A-46D: Effect of compound OH17 on the cell viability of MDA-MB231 and MDA-MB436 cell lines. FIG. 46A: MDA-MB231, 24 h; FIG. 46B: MDA-MB436, 24 h; FIG. 46C: MDA-MB231, 48 h; FIG. 46D: MDA-MB436, 48 h.

FIGS. 47A-47D: Effect of compound OH25 on the cell viability of MDA-MB231 and MDA-MB436 cell lines. FIG. 47A: MDA-MB231, 24 h; FIG. 47B: MDA-MB436, 24 h; FIG. 47C: MDA-MB231, 48 h; FIG. 47D: MDA-MB436, 48 h.

FIGS. 48A-48D: Effect of docetaxel (DTX) on the cell viability of MDA-MB231 and MDA-MB436 cell lines. FIG. 48A: MDA-MB231, 24 h; FIG. 48B: MDA-MB436, 24 h; FIG. 48C: MDA-MB231, 48 h; FIG. 48D: MDA-MB436, 48 h.

FIG. 49: Effect of OH25 on the cell viability of MCF 10A cell lines.

FIG. 50: Effect of compounds OH17, OH25 and docetaxel (DTX) on the cell morphology of TNBC cell line MDA-MB-231, 24 hours post treatment.

FIG. 51: Effect of compounds OH17, OH25 and docetaxel (DTX) on the cell morphology of TNBC cell line MDA-MB-231, 48 hours post treatment.

FIG. 52: Effect of compounds OH17, OH25 and docetaxel (DTX) on the cell morphology of TNBC cell line MDA-MB-436, 24 hours post treatment.

FIG. 53: Effect of compounds OH17, OH25 and docetaxel (DTX) on the cell morphology.

FIGS. 54A-54B: Effect of compounds 17 and 25 on the migration of MDA-MB-231 cells in wound healing assay. FIG. 54A shows the images of the assay. FIG. 54B shows the quantification of the results.

FIGS. 55A-55D: Effect of compounds 17 and 25 on colony formation of MDA-MB-231 cell line. Cells were allowed to grow in soft agar for 28 days in the presence of the tested compounds. DMSO was used a negative control and docetaxel (DTX) as a positive control. FIG. 55A: Representative images of the soft agar colony formation on day 28. FIG. 55B: Quantification of the number of colonies (>325 μm²=size of single cells quantified at day 0) expressed as a percentage of treatment relative to the control (Mean±SEM). FIG. 55C: Average size (μm²) of the colonies formed upon exposure to different treatments expressed as Mean±SEM. FIG. 55D: Distribution of colonies into different size categories: Small (<500 μm²), Medium (500-1000 μm²), and Large (>1000 μm²). Colonies' size and number were quantified by ImageJ software in at least 10 random fields per treatment. Results were analyzed using “One-way ANOVA” followed by Dunnett's posthoc test. Results were considered statistically significant when p<0.05.*p<0.05.

FIG. 56: Summary of the known heteroaromatic-based chalcone derivatives with reported activity against PCa in comparison to the novel lead molecule (compound 17).

FIG. 57: (+)-ESI Mass Spectrum of compound 1.

FIG. 58: FT-IR spectrum of compound 1.

FIG. 59: ¹H-NMR spectrum (CDCl3 with 0.05% v/v of TMS, 600 MHz) of compound 1.

FIG. 60: ¹³C-NMR spectrum (CDCl3, 150 MHZ) of compound 1.

FIG. 61: ¹H-¹H COSY NMR of compound 1.

FIG. 62: (+)-ESI Mass spectrum of compound 2.

FIG. 63: FT-IR spectrum of compound 2.

FIG. 64: ¹H-NMR spectrum (CDCl3 with 0.05% v/v of TMS, 600 MHz) of compound 2.

FIG. 65: ¹H-¹H COSY NMR of compound 2.

FIG. 66: ¹³C-NMR spectrum (CDCl3, 150 MHz) of compound 2.

FIG. 67: (+)-ESI Mass spectrum of compound 3.

FIG. 68: FT-IR spectrum of compound 3.

FIG. 69: ¹H-NMR spectrum (CDCl3 with 0.05% v/v of TMS, 600 MHz) of compound 3.

FIG. 70: ¹H-¹H COSY NMR of compound 3.

FIG. 71: ¹³C-NMR spectrum (CDCl3, 150 MHZ) of compound 3.

FIG. 72: (+)-ESI Mass spectrum of compound 4.

FIG. 73: FT-IR spectrum of compound 4.

FIG. 74: ¹H-NMR spectrum (CDCl3 with 0.05% v/v of TMS, 600 MHz) of compound 4.

FIG. 75: ¹H-¹H COSY NMR of compound 4.

FIG. 76: ¹³C-NMR spectrum (CDCl3, 150 MHz) of compound 4.

FIG. 77: (+)-ESI Mass spectrum of compound 5.

FIG. 78: FT-IR spectrum of compound 5.

FIG. 79: ¹H-NMR spectrum (CDCl3 with 0.05% v/v of TMS, 600 MHz) of compound 5.

FIG. 80: ¹H-¹H COSY NMR of compound 5.

FIG. 81: ¹³C-NMR spectrum (CDCl3, 150 MHz) of compound 5.

FIG. 82: (+)-ESI Mass spectrum of compound 6.

FIG. 83: FT-IR spectrum of compound 6.

FIG. 84: ¹H-NMR spectrum (CDCl3 with 0.05% v/v of TMS, 600 MHz) of compound 6.

FIG. 85: ¹H-¹H COSY NMR of compound 6.

FIG. 86: ¹³C-NMR spectrum (CDCl3, 150 MHz) of compound 6.

FIG. 87: (+)-ESI Mass spectrum of compound 7.

FIG. 88: FT-IR spectrum of compound 7.

FIG. 89: ¹H-NMR spectrum (CDCl3 with 0.05% v/v of TMS, 600 MHz) of compound 7.

FIG. 90: ¹H-¹H COSY NMR of compound 7.

FIG. 91: ¹³C-NMR spectrum (CDCl3, 150 MHz) of compound 7.

FIG. 92: (+)-ESI Mass spectrum of compound 8.

FIG. 93: FT-IR spectrum of compound 8.

FIG. 94: ¹H-NMR spectrum (CDCl3 with 0.05% v/v of TMS, 600 MHz) of compound 8.

FIG. 95: ¹H-¹H COSY NMR of compound 8.

FIG. 96: ¹H-¹H NOESY NMR of compound 8.

FIG. 97: Expanded region of ¹H-¹H NOESY NMR of compound 8 showing the correlation between H3 and the Olefinic proton.

FIG. 98: ¹³C-NMR spectrum (CDCl3, 150 MHz) of compound 8.

FIG. 99: (+)-ESI Mass spectrum of compound 9.

FIG. 100: FT-IR spectrum of compound 9.

FIG. 101: ¹H-NMR spectrum (CDCl3 with 0.05% v/v of TMS, 600 MHz) of compound 9.

FIG. 102: ¹H-¹H COSY NMR of compound 9.

FIG. 103: ¹³C-NMR spectrum (CDCl3, 150 MHz) of compound 9.

FIG. 104: (+)-ESI Mass spectrum of compound 10.

FIG. 105: FT-IR spectrum of compound 10.

FIG. 106: ¹H-NMR spectrum (CDCl3 with 0.05% v/v of TMS, 600 MHz) of compound 10.

FIG. 107: ¹³C-NMR spectrum (CDCl3, 150 MHz) of compound 10.

FIG. 108: (+)-ESI Mass spectrum of compound 11.

FIG. 109: FT-IR spectrum of compound 11.

FIG. 110: ¹H-NMR spectrum (CDCl3 with 0.05% v/v of TMS, 600 MHz) of compound 11.

FIG. 111: ¹H-¹H COSY NMR of compound 11.

FIG. 112: ¹³C-NMR spectrum (CDCl3, 150 MHz) of compound 11.

FIG. 113: (+)-ESI Mass spectrum of compound 12.

FIG. 114: FT-IR spectrum of compound 12.

FIG. 115: ¹H-NMR spectrum (CDCl3, 600 MHz) of compound 12.

FIG. 116: ¹³C-NMR spectrum (CDCl3, 150 MHz) of compound 12.

FIG. 117: (+)-ESI Mass spectrum of compound 13.

FIG. 118: FT-IR spectrum of compound 13.

FIG. 119: ¹H-NMR spectrum (CDCl3 with 0.05% v/v of TMS, 600 MHz) of compound 13.

FIG. 120: ¹H-¹H COSY NMR of compound 13.

FIG. 121: ¹H-¹H NOESY NMR of compound 13.

FIG. 122: ¹³C-NMR spectrum (CDCl3, 150 MHZ) of compound 13.

FIG. 123: ¹H-¹³C HMQC NMR of compound 13.

FIG. 124: ¹H-¹³C HMBC NMR of compound 13.

FIG. 125: ¹H-¹⁵N HMBC NMR of compound 13.

FIG. 126: (+)-ESI Mass spectrum of compound 14.

FIG. 127: FT-IR spectrum of compound 14.

FIG. 128: ¹H-NMR spectrum (CDCl3 with 0.05% v/v of TMS, 600 MHz) of compound 14.

FIG. 129: ¹H-¹H COSY NMR of compound 14.

FIG. 130: ¹³C-NMR spectrum (CDCl3, 150 MHz) of compound 14.

FIG. 131: (+)-ESI Mass spectrum of compound 15.

FIG. 132: FT-IR spectrum of compound 15.

FIG. 133: ¹H-NMR spectrum (CDCl3 with 0.05% v/v of TMS, 600 MHz) of compound 15.

FIG. 134: ¹H-¹H COSY NMR of compound 15.

FIG. 135: ¹³C-NMR spectrum (CDCl3, 150 MHz) of compound 15.

FIG. 136: (+)-ESI Mass spectrum of compound 16.

FIG. 137: FT-IR spectrum of compound 16.

FIG. 138: ¹H-NMR spectrum (CDCl3 with 0.05% v/v of TMS, 600 MHz) of compound 16.

FIG. 139: ¹H-¹H COSY NMR of compound 16.

FIG. 140: ¹³C-NMR spectrum (CDCl3, 150 MHz) of compound 16.

FIG. 141: ¹H-¹³C HMQC NMR of compound 16.

FIG. 142: ¹H-¹³C HMBC NMR of compound 16

FIG. 143: (+)-ESI Mass spectrum of compound 17.

FIG. 144: ¹H-NMR spectrum (CDCl3 with 0.05% v/v of TMS, 600 MHz) of compound 17.

FIG. 145: ¹H-¹H COSY NMR of compound 17.

FIG. 146: ¹³C-NMR spectrum (CDCl3, 150 MHz) of compound 17.

FIG. 147: (+)-ESI Mass spectrum of compound 18.

FIG. 148: FT-IR spectrum of compound 18.

FIG. 149: ¹H-NMR spectrum (CDCl3 with 0.05% v/v of TMS, 600 MHz) of compound 18.

FIG. 150: ¹H-¹H COSY NMR of compound 18.

FIG. 151: ¹³C-NMR spectrum (CDCl3, 150 MHz) of compound 18.

FIG. 152: (+)-ESI Mass spectrum of compound 19.

FIG. 153: FT-IR spectrum of compound 19.

FIG. 154: ¹H-NMR spectrum (CDCl3 with 0.05% v/v of TMS, 600 MHz) of compound 19.

FIG. 155: ¹H-¹H COSY NMR of compound 19.

FIG. 156: ¹³C-NMR spectrum (CDCl3, 150 MHz) of compound 19.

FIG. 157: (+)-ESI Mass spectrum of compound 20.

FIG. 158: FT-IR spectrum of compound 20.

FIG. 159: ¹H-NMR spectrum (CDCl3 with 0.05% v/v of TMS, 600 MHz) of compound 20.

FIG. 160: ¹H-¹H COSY NMR of compound 20.

FIG. 161: ¹³C-NMR spectrum (CDCl3, 150 MHz) of compound 20.

FIG. 162: (+)-ESI Mass spectrum of compound 21.

FIG. 163: FT-IR spectrum of compound 21.

FIG. 164: ¹H-NMR spectrum (CDCl3, 600 MHz) of compound 21.

FIG. 165: ¹H-¹H COSY NMR of compound 21.

FIG. 166: ¹³C-NMR spectrum (CDCl3, 150 MHz) of compound 21.

FIG. 167: (+)-ESI Mass spectrum of compound 22.

FIG. 168: ¹H-NMR spectrum (CDCl3 with 0.05% v/v of TMS, 600 MHz) of compound 22.

FIG. 169: ¹H-¹H COSY NMR of compound 22.

FIG. 170: ¹³C-NMR spectrum (CDCl3, 150 MHz) of compound 22.

FIG. 171: (+)-ESI Mass spectrum of compound 23.

FIG. 172: FT-IR spectrum of compound 23.

FIG. 173: ¹H-NMR spectrum (CDCl3 with 0.05% v/v of TMS, 600 MHz) of compound 23.

FIG. 174: ¹H-¹H COSY NNMR of compound 23.

FIG. 175: ¹³C-NMR spectrum (CDCl3, 150 MHz) of compound 23.

FIG. 176: (+)-ESI Mass spectrum of compound 24.

FIG. 177: FT-IR spectrum of compound 24.

FIG. 178: ¹H-NMR spectrum (CDCl3 with 0.05% v/v of TMS, 600 MHz) of compound 24.

FIG. 179: ¹H-¹H COSY NMR of compound 24.

FIG. 180: ¹³C-NMR spectrum (CDCl3, 150 MHz) of compound 24.

FIG. 181: (+)-ESI Mass spectrum of compound 25.

FIG. 182: FT-IR spectrum of compound 25.

FIG. 183: ¹H-NMR spectrum (CDCl3 with 0.05% v/v of TMS, 600 MHz) of compound 25.

FIG. 184: ¹H-¹H COSY NMR of compound 25.

FIG. 185: 19F-NMR spectrum (CDCl3, 600 MHz) of compound 25.

FIG. 186: ¹³C-NMR spectrum (CDCl3, 150 MHz) of compound 25.

FIG. 187: ¹H-NMR spectrum (CDCl3 with 0.05% v/v of TMS, 600 MHz) of compound 227.

The figures herein are for illustrative purposes only and are not necessarily drawn to scale.

### DETAILED DESCRIPTION OF THE EXAMPLE EMBODIMENTS

### Definitions

As used herein, the singular forms “a”, “an”, and “the” include both singular and plural referents unless the context clearly dictates otherwise.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

The term “cyclopropyl” as used herein refers to a radical, substituent, or molecular fragment having a chemical structure derived from cyclopropane and having the chemical formula C₃H₅. A cyclopropyl has the structure of

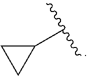 [SMILES: *C1CC1]

The term “thienyl” refers to either two of univalent isomeric radicals C4H3S derived from thiophene by removal of a hydrogen atom from either the alpha (or 2-position) or the beta (3-position). A thienyl group can be a 2-thienyl or 3-thienyl group.

The term “pharmaceutical composition” refers to a composition that comprises a compound disclosed herein and a pharmaceutically acceptable carrier that is conventional in the art and that is suitable for administration to cells or to a subject. The term “pharmaceutically acceptable” as used throughout this specification is consistent with the art and means compatible with the other ingredients of a pharmaceutical composition and not deleterious to the recipient thereof. A pharmaceutical composition is a preparation that is in such form as to permit the biological activity of the active ingredient to be effective, and that contain no additional components that are unacceptably toxic to an individual to which the formulation or composition would be administered. Such compositions may be sterile.

The term “carrier” or “excipient” includes any and all solvents, diluents, buffers (e.g., neutral buffered saline or phosphate buffered saline), solubilizes, colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizes, thickeners, agents for achieving a depot effect, coatings, antifungal agents, preservatives, stabilizers, antioxidants, tonicity controlling agents, absorption delaying agents, and the like. The use of such media and agents for pharmaceutical active components is well known in the art. Such materials should be non-toxic and should not interfere with the activity of the cells or active components.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like.

The terms “subject,” “individual,” and “patient” are used interchangeably herein to refer to a vertebrate, such as a mammal, e.g., a human. Examples of mammals include murines, simians, humans, farm animals, sport animals, and pets. Tissues, cells and their progeny of a biological entity obtained in vivo or cultured in vitro are also encompassed.

The term “therapeutically effective amount” refers to an amount (e.g., of a compound) that is sufficient to provide a therapeutic benefit to a patient in the treatment or management of a disease or disorder, or to delay or minimize one or more symptoms associated with the disease or disorder.

The term “treating” or “treatment” of any disease or disorder refers, in certain embodiments, to ameliorating a disease or disorder, either physically (e.g., stabilization of a discernable symptom) or physiologically (e.g., stabilization of a physical parameter) or both. In yet another embodiment, “treating” or “treatment” of a disease refer to executing a protocol, which may include administering one or more therapeutic agents to an individual (human or otherwise), in an effort to obtain beneficial or desired results in an individual, including clinical and cosmetic results. Beneficial or desired clinical results include, but are not limited to, alleviation or amelioration of one or more symptoms, diminishment of extent of disease(s), stabilized (i.e., not worsening) state of disease, preventing spread or increase in severity of disease, delay or slowing of disease progression, amelioration or palliation of the disease state, and remission (whether partial or total). “Treatment” can also mean prolonging survival as compared to expected survival of an individual not receiving treatment. Further, “treating” and “treatment” may occur by administration of one dose of a therapeutic agent or therapeutic agents, may occur by administration of one dose of a therapeutic agent or therapeutic agents, or may occur upon administration of a series of doses of a therapeutic agent or therapeutic agents. “Treating” or “treatment” does not require complete alleviation of signs or symptoms, and does not require a cure. “Treatment” can also refer to clinical intervention, such as administering one or more therapeutic agents to an individual, designed to alter the natural course of the individual being treated (i.e., to alter the course of the individual that would occur in absence of the clinical intervention).

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides compounds, compositions, and methods for treating cancers, e.g., breast cancer and prostate cancer, e.g., breast cancer such as Triple-negative breast cancer (TNBC) and prostate cancer such as Castration-resistant prostate cancer (CRPC).

In one aspect, the disclosure provides thienyl-pyridine-based chalcone analogs. Optimization of chalcones' activity through hybridization with heteroaromatic rings that are known to possess anticancer activity, such as pyridine and thiophene, can improve chalcones' efficacy and selectivity. Moreover, incorporating in-silico ADMET prediction early in the design process can facilitate the identification of molecules with favorable physicochemical properties that are more likely to move along the drug development process. Combining both strategies simultaneously can lead to overcoming current chalcones' limitations. In some embodiments, in silico-guided development of new heteroaromatic-based chalcones results in promising lead molecules with improved efficacy, safety and pharmacokinetic profiles for the treatment of the widespread deadly tumors (mCRPC and TNBC).

### Compounds

In one aspect, the present disclosure provides compounds for treating diseases such as cancers. In some embodiments, the compound has a structure according to Formula (I):

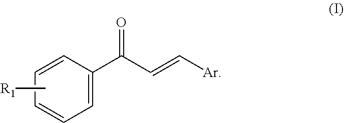

In some embodiments, the compounds disclosed herein can be a pharmaceutically acceptable salt of a compound according to Formula (I).
In some embodiments, Ar is

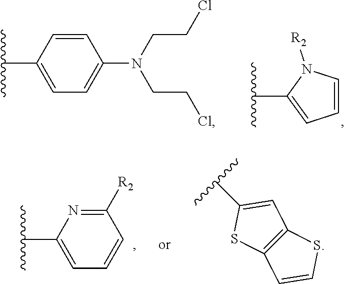

In some examples, Ar is

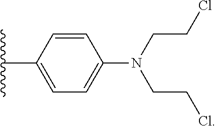 [SMILES: *c1ccc(N(CCCl)CCCl)cc1]

In some examples, Ar is

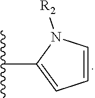

In some examples, Ar is

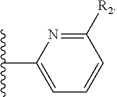

In some examples, Ar is

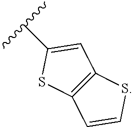 [SMILES: *c1cc2sccc2s1]

In some embodiments, the compounds have a structure according to Formula (II)

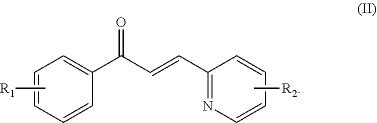

In some embodiments, the compounds have a structure according to

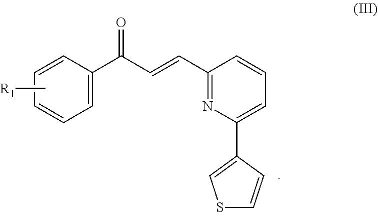

In the formulas herein, R₁ may be −OCH₃, —OH, —CF₃, —SO₂CH₃, or —F. In some examples, R₁ is —OCH₃. In some examples, R₁ is-OH. In some examples, R₁ is-CF₃. In some examples, R₁ is-SO₂CH₃. In some examples, R₁ is-F.

In the formulas herein, R₂ may be absent, —H, cyclopropyl, —OCH₃, —CF₃, or thienyl. In some examples, R₂ is absent. In some examples, R₂ is —H. In some examples, R₂ is cyclopropyl. In some examples, R₂ is —OCH₃. In some examples, R₂ is-CF₃. In some examples, R₂ is thienyl. In some examples, R₂ is 3-thienyl. In some examples, R₂ is 2-thienyl.

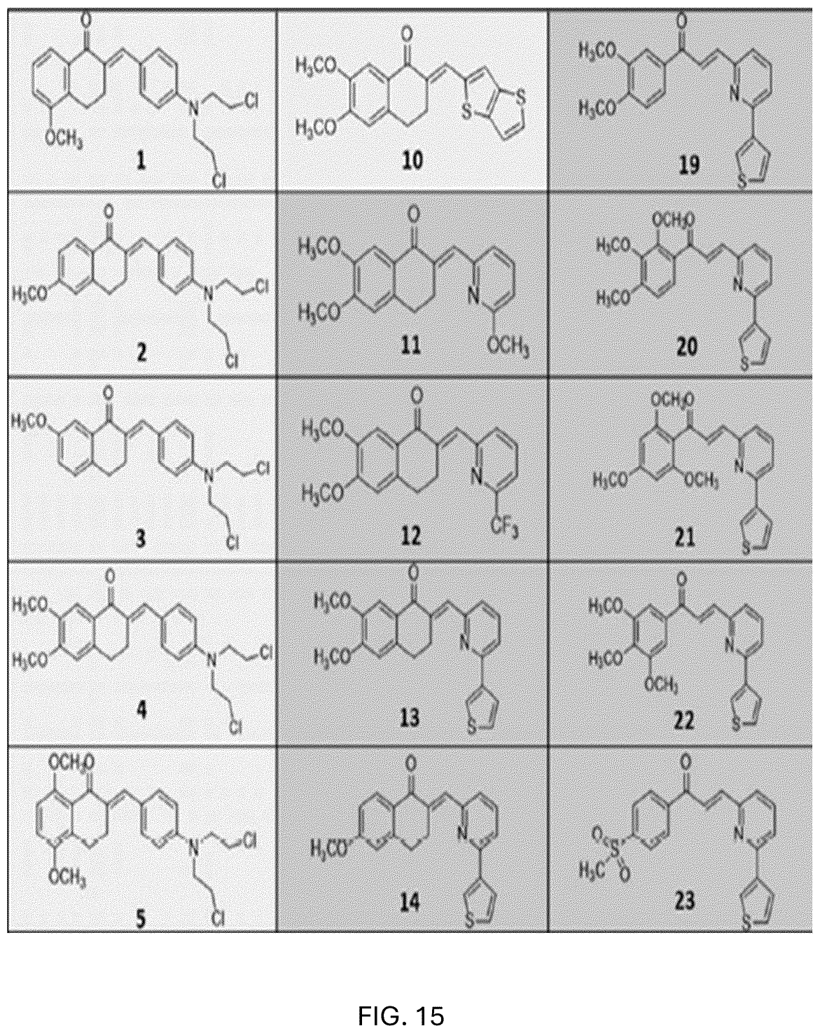

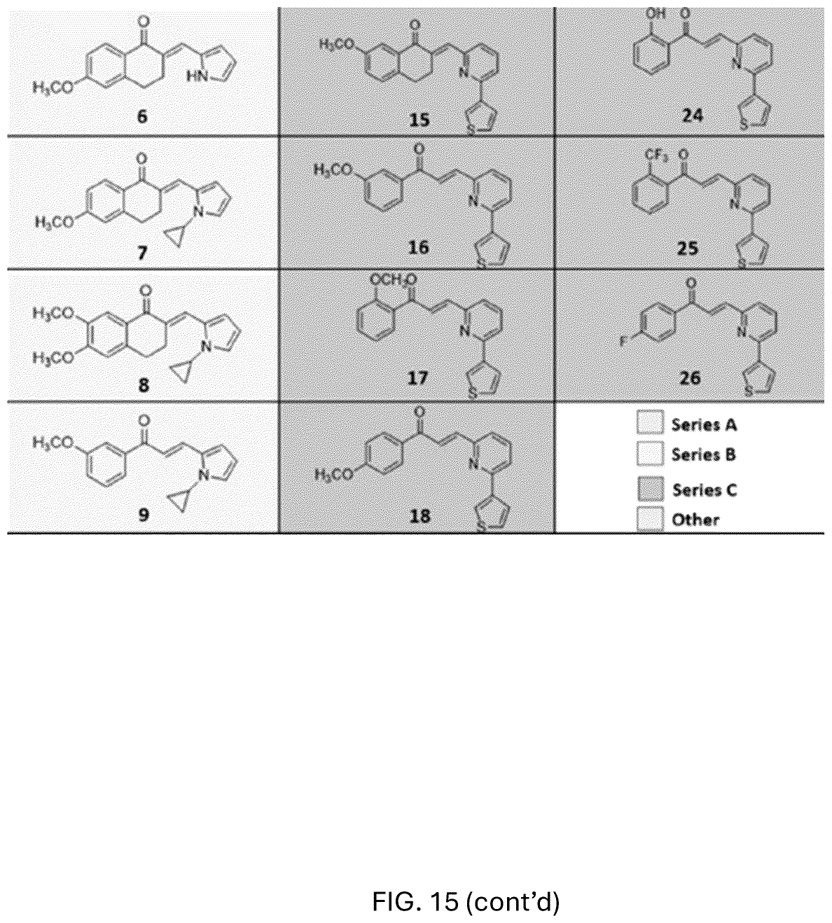

Examples of the compounds include Compounds 1-26 in Table 3 or FIG. 15. In some examples, the compound may be one of Compounds 13-26. In one example, the compound has a structure of Formula (IV).

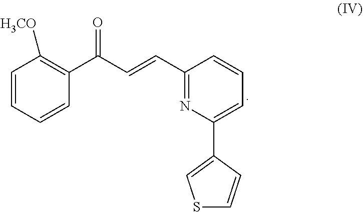 [SMILES: COc1ccccc1C(=O)/C=C/c1cccc(-c2ccsc2)n1]

### In One Example, the Compound has a Structure of Formula (V)

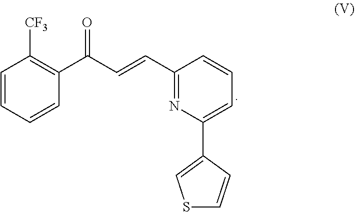 [SMILES: O=C(/C=C/c1cccc(-c2ccsc2)n1)c1ccccc1C(F)(F)F]

Any compound used in or formed by the processes described herein may be modified to make an inorganic or organic acid or base addition salt thereof to form a salt, if appropriate and desired. The salts of the present compounds can be prepared from a parent compound that contains a basic or acidic moiety by chemical processes. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Examples of salts include mineral or organic acid salts of basic residues such as amines; alkali or organic salts of acidic residues such as carboxylic acids; and the like. The salts include the salts and the quaternary ammonium salts of the parent compound formed, for example, from inorganic or organic acids that are not unduly toxic. For example, acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like, or using a different acid that produces the same counterion.

### PHARMACEUTICAL COMPOSITIONS AND METHODS OF TREATMENT

In some aspect, the present disclosure provides pharmaceutical compositions comprising a therapeutically effective amount of a compound disclosed herein and one or more pharmaceutically acceptable carriers. Compositions comprising the compound can be administered in the form of salts provided the salts are pharmaceutically acceptable.

In some embodiments, the pharmaceutical composition is provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

In some embodiments, the pharmaceutical compositions comprise one or more solvents. Examples of solvents include water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

In some embodiments, the pharmaceutical compositions comprise one or more buffering agents. Examples of buffering agents include citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Further disclosed herein are methods of treating a disease (e.g., a cancer) by administering the compound or pharmaceutical composition to a subject. The diseases that can be treated by the compounds, compositions, and methods herein include cancers. In some embodiments, the cancer is breast cancer. Examples of breast cancers include triple negative breast cancer, triple-positive breast cancer, ductal carcinoma, lobular carcinoma, inflammatory breast cancer, metastatic breast cancer, recurrent breast cancer, male breast cancer, micrometastases, and paget disease. In some embodiments, the cancer is prostate cancer. Examples of prostate cancers include Castration-resistant prostate cancer, prostatic acinar adenocarcinoma, ductal adenocarcinoma, small cell prostate cancer, squamous cell carcinoma of the prostate, urothelial carcinoma, and neuroendocrine prostate cancer.

Additional examples of cancers include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), ovarian carcinoma (e.g., ovarian epithelial carcinoma or surface epithelial-stromal tumour including serous tumor, endometrioid tumor and mucinous cystadenocarcinoma, sex-cord-stromal tumor), liver and bile duct carcinoma (e.g., hepatocellular carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medulloblastoma, astrocytoma, medulloblastoma, craniopharyngioma, ependymoma, pinealoma, hemangioblastoma, acoustic neuroma, oligodendroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids.

Methods of administrating the pharmacological compositions include intradermal, intrathecal, intramuscular, intraperitoneal, intravenous, subcutaneous, intranasal, epidural, by inhalation, and oral routes. Administration can be systemic or local.

### Example 1

This example shows the discovery and development of novel pyridine-based chalcone compounds for treating TNBC and CRPC. Applicant designed and synthesized a library of 26 novel chalcone analogs cyclic (tetralone-based) and acyclic chalcone analogs, in which ring B was either substituted with nitrogen mustard or replaced by pyrrole- or pyridine-heterocyclic rings. The design was guided by in silico ADMET prediction in which analogs with favorable drug-likeness properties were prioritized. The new compounds were synthesized by Claisen-Schmidt condensation reaction, purified and characterized by extensive structural elucidation studies (LC-MS, FT-IR, 1D NMR, 2D NMR, and elemental analysis). They were then evaluated for their pharmacological anticancer activities to investigate their efficacy and safety. In vitro studies were conducted against a panel of four cancer cell lines: Androgen receptor-negative prostate cancer cell lines (PC3 and DU145) and invasive TNBCs (MDA-MB-231 and MDA-MB-436), as well as primary human dental bulb cells and normal breast epithelial cell line (MCF10a) to determine their activities on cell proliferation, colony formation, apoptosis, cell cycle, migration and cancer-related pathways. Moreover, in ovo experiment was conducted on the chorioallantoic membrane (CAM) of the chicken embryos to determine the effects on angiogenesis, which is an important event in cancer progression and metastasis.

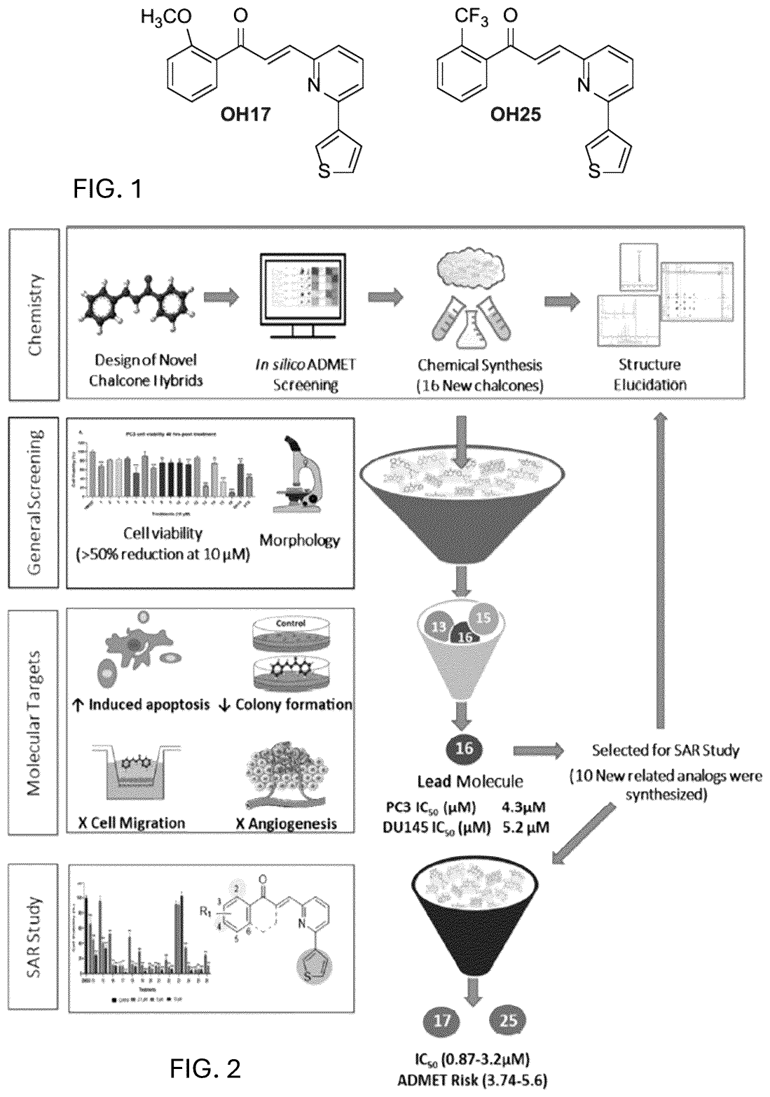

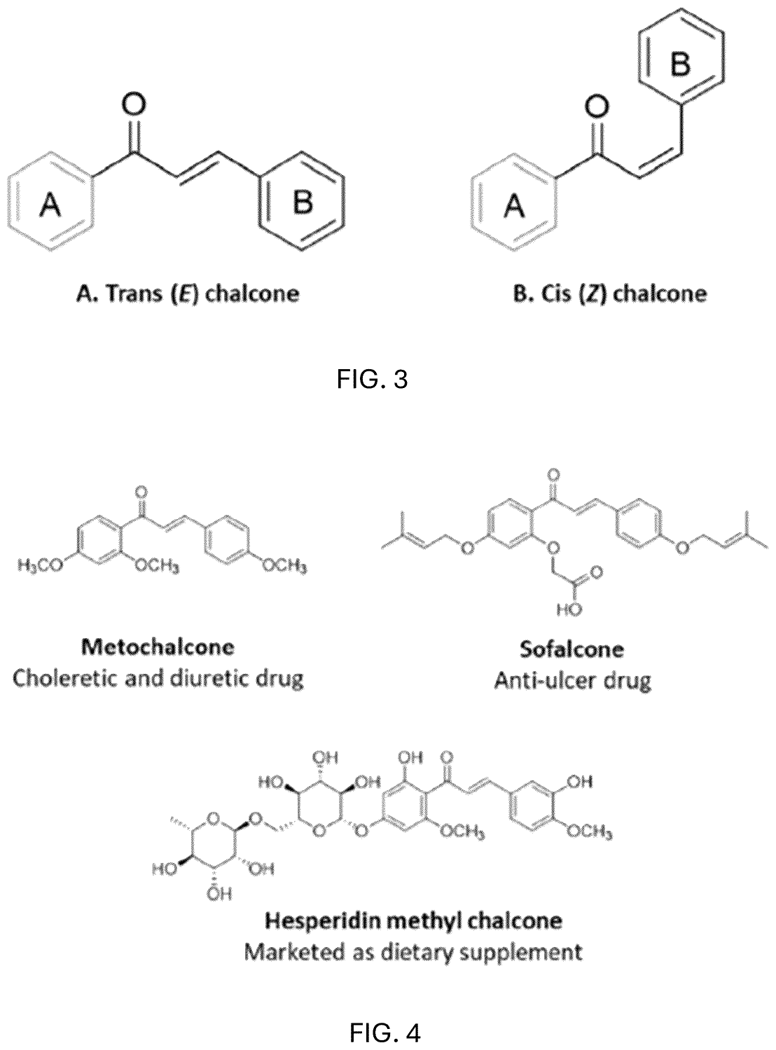

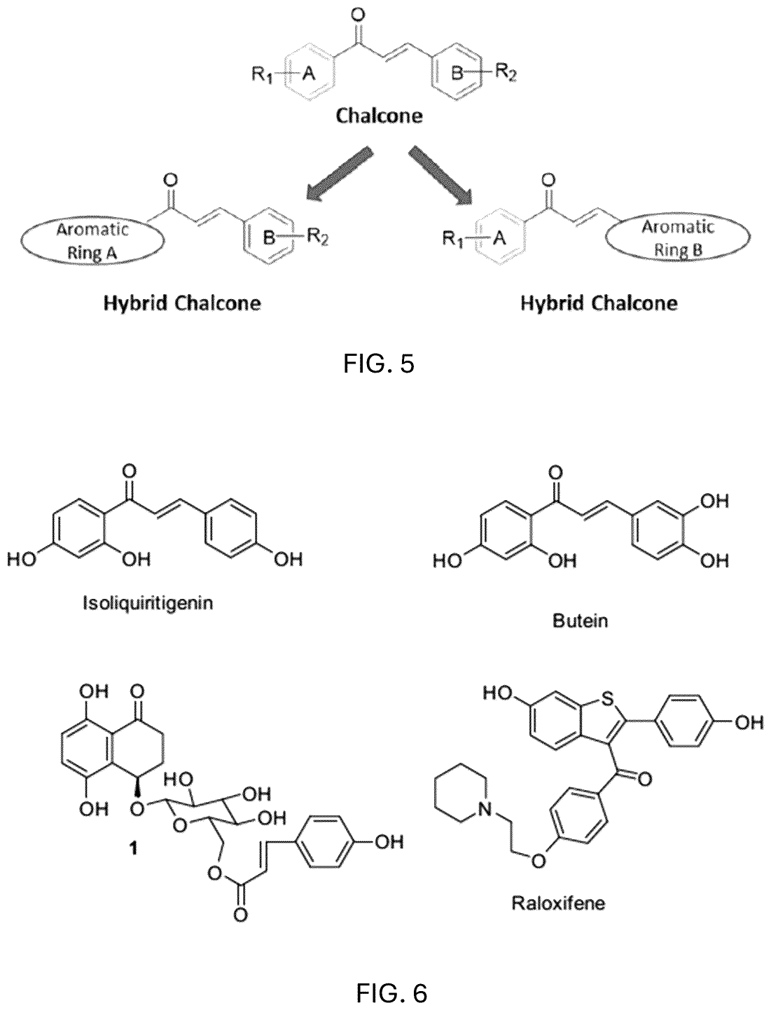

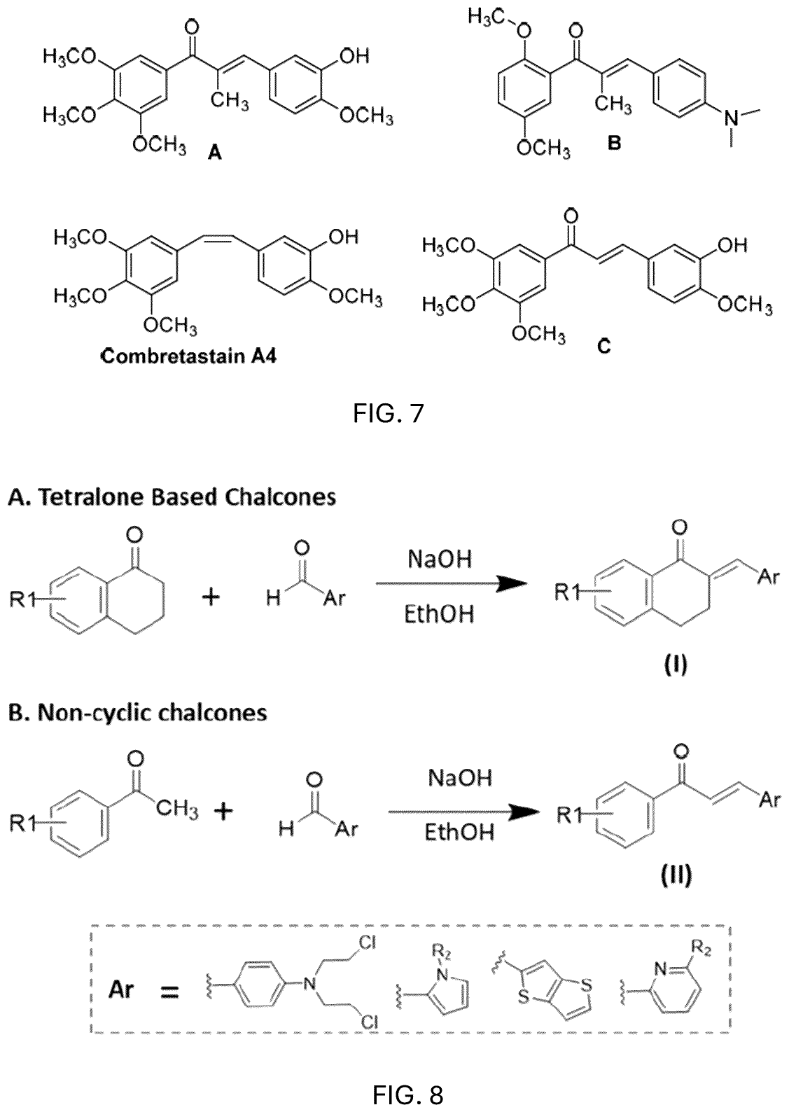

Pharmacological and biological studies identified thienyl-pyridine-based chalcone analogs as the most promising hits against both TNBC and androgen-negative prostate cancer cells. Compounds OH17 and OH25 possessed the most potent anticancer activities against TNBC cell lines with an IC50 ranging between 0.87-3.2 μM against MDA-MB-231 and MDA-MB-436 cell lines. Detailed biological investigations showed that thienyl pyridine-based chalcones possessed promising anticancer activities against recognizable cancer molecular processes with acceptable selectivity. Remarkably, they had dramatically induced apoptosis through upregulation of Bax and downregulation of Bcl-2, inhibited colony formation, reduced cell migration, blocked ERK1/2 and Akt activities. Additionally, in ovo testing revealed statistically significant inhibition of blood vessels development in CAM model. Comparison of thienyl-pyridine-based chalcone with a first-line chemotherapeutic agent in mCRPC and TNBC, docetaxel, in cell proliferation, cell apoptosis and colony formation assays revealed that several analogs of this series are superior to a commercially available therapeutic option. Taken together, the developed series of thienyl pyridine chalcone compounds, particularly OH17 and OH25 (FIG. 1), were identified as molecules for targeted treatment of mCRPC and TNBC patients.

The compounds can be used to treat patients with aggressive types of cancer, e.g., TNBC and castration-resistant prostate cancer, which so far lack effective targeted therapies. Besides, the compounds can also be used for treating patients with other types of aggressive tumors (e.g., HER+ breast cancer and colorectal cancer). Today, patients with these types of tumors have limited treatment options with marginal or no survival benefits. Thus, the identified compounds can aid in meeting the urgent need for effective and safe therapies for patients in need. The new efficient drugs against are not found to have any disadvantage against the tested types of cancer (TNBC and AR negative PCa).

Notably, the compounds showed superior efficacy as compared to one of the first line chemotherapies used in clinics, docetaxel. The compounds are distinguished by their markedly enhanced potency, selectivity, and ADMET risk in comparison to other potential candidates. Besides, Applicant incorporated for the first time thienyl-pyridine moiety in a chalcone scaffold, which was not previously developed for any sort of applications, and proved that it remarkably enhanced the anti-cancer activity of chalcones.

In brief, a library of 26 cyclic (tetralone-based) and acyclic chalcone analogs, in which ring B was either substituted with nitrogen mustard or replaced by pyrrole or pyridine heterocyclic rings, were designed, synthesized and evaluated as potential therapies for CRPC. The design was guided by in-silico ADMET prediction in which analogs with favorable drug-likeness properties were prioritized. The new compounds were synthesized by Claisen-Schmidt condensation reaction, purified and characterized by extensive structural elucidation studies. Compounds 1-16 cytotoxic activity were initially evaluated against two androgen receptor (AR)-negative prostate cancer cell lines (PC3 and DU145). Among the tested compounds, thienyl pyridine-containing analogs (13, 15 and 16) showed potent antiproliferative activities at low micromolar levels with IC50 values ranging between 4.32-6.47 μM against PC3 and DU145 cell lines. Detailed biological studies of the lead molecule 16 revealed that it could significantly induce apoptosis through upregulation of Bax and downregulation of Bcl-2. In addition, compound 16 potently inhibited colony formation and reduced cell migration of AR-negative PCa cell lines (PC3 and DU145). The molecular pathway analysis showed that the anticancer activity of compound 16 is associated with the blocking of ERK1/2 and Akt activities. Furthermore, compound 16 inhibited angiogenesis in the chick chorioallantoic membrane (CAM) model as compared to control. Therefore, the thienyl pyridine-based chalcone series was expanded and ten additional analogs were synthesized and tested against both TNBC and AR negative PCa cell lines. Structure-activity relationship study on thienyl pyridine-based chalcones revealed that the cytotoxicity could dramatically improve via changing the methoxylation pattern by more than 2-folds. Among this series, compounds 17 and 25 showed the most potent activity with an IC50 of 0.87-3.2 μM against MDA-MB-231 and MDA-MB-436 cell lines. Notably, comparison of thienyl-pyridine-based chalcone with a first-line chemotherapeutic agent in mCRPC and TNBC, docetaxel, in cell proliferation, cell apoptosis and colony formation assays revealed that several analogs of this series are superior to a commercially available therapeutic option. These results indicate that thienyl pyridine-based chalcones are promising molecules for the treatment of CRPC and TNBC.

This example demonstrates exemplary methods of designing novel chalcone hybrids with potentially promising anticancer activities; in-silico ADMET screening to eliminate analogs with low drug-likeness properties; synthesizing and elucidating the structures of the proposed chalcone analogs; evaluating the anticancer activity of the synthesized chalcones in AR-negative prostate cancer cells and TNBC cells; investigating the antiangiogenetic activity of chalcone analogs in ovo using the chorioallantoic membrane (CAM) of the chicken embryo model.

### Methodology and Research Design

All chemicals and reagents were obtained from Sigma-Aldrich (USA) unless stated otherwise and used without further purification. Solvents used in the synthesis and purification were of analytical or HPLC grade with purity >98% and were purchased from Sigma-Aldrich (USA) or Merck (Germany). The main used fine chemicals and solvents are listed in Table 1. Doubled distilled H₂O was prepared with a Milli-Q (Bedford, MA, USA) H₂O purification system. Thin-layer chromatography (TLC) was done on pre-coated silica gel aluminum plates from Sigma-Aldrich (USA). Prepacked Biotage® SNAP KP-SIL cartridges (Biotage AB, Sweden) were used for column chromatography.

TABLE 1
List of fine chemicals and solvents used in this study

| | | Molecular |
|---|---|---|
| | | Weight |
| | | (density |
| | Chemical Name | g/ml) |
| Tetralones | 5,8-Dimethoxy-1-tetralone | 206.24 |
| | 5-Methoxy-1-tetralone | 176.21 |
| | 6,7-Dimethoxy-1-tetralone | 206.24 |
| | 6-Methoxy-1-tetralone | 176.21 |
| | 7-Methoxy-1-tetralone | 176.21 |
| Acetophenones | 2-Methoxyacetophenone | 150.17 |
| | 3-Methoxyacetophenone | 150.17 (1.094) |
| | 4-Methoxyacetophenone | 150.17 |
| | 3,4-Dimethoxyacetophenone | 180.20 |
| | 2,3,4-Trimethoxyacetophenone | 210.23 (1.155) |
| | 2,4,6-Trimethoxyacetophenone | 210.23 |
| | 3,4,5-Trimethoxyacetophenone | 210.23 |
| | 4′-(Methylsulfonyl)acetophenone | 198.24 |
| | 2-Hydroxyacetophenone | 136.15 (1.131) |
| | 2-(Trifluoromethyl)acetophenone | 188.15 (1.255) |
| | 4-Fluoroacetophenone | 138.14 (1.138) |
| Carboxy- | 4-[Bis-(2- | 246.13 |
| aldehydes | chloroethyl)amino]benzaldehyde | |
| | Pyrrole-2-carboxaldehyde | 95.10 |
| | 1-Cyclopropyl-1H-pyrrole-2- | 135.16 |
| | carbaldehyde | |
| | 6-Methoxy-2-pyridinecarboxaldehyde | 137.14 |
| | 6-(Trifluoromethyl)pyridine-2- | 175.11 |
| | carboxaldehyde | |
| | 6-(3-Thienyl)pyridine-2-carboxaldehyde | 189.23 |
| | Thieno[3,2-b]thiophene-2- | 168.24 |
| | carboxaldehyde | |
| Reagents | Sodium hydroxide (NaOH) | 40.00 |
| Solvents | Acetonitrile, chloroform, chloroform-d, | |
| | dichloromethane, ethanol, ethyl acetate, | |
| | hexane, isopropanol, methanol, toluene | |
| | and diethyl ether | |

### Chemical Synthesis

Chalcones were synthesized by Claisen-Schmidt condensation reaction (FIG. 8). For each analog, appropriate aromatic carboxaldehyde (1 mmol, 1 eq.) was added to a solution of methoxy substituted-tetralone or -acetophenone (1 mmol, 1 eq.) and stirred until completely dissolved. The mixture was then cooled on Ice to 0° C. and 0.5 ml of aqueous sodium hydroxide (3 mmol, 3 eq.) was added drop wisely. Reaction mixtures were stirred at room temperature, for 1-72 hours, until completion of the reaction or an increase in the ratio of byproducts. Reactions progress was monitored by TLC using appropriate solvent mixture and visualized under UV light (254 nm and 365 nm).

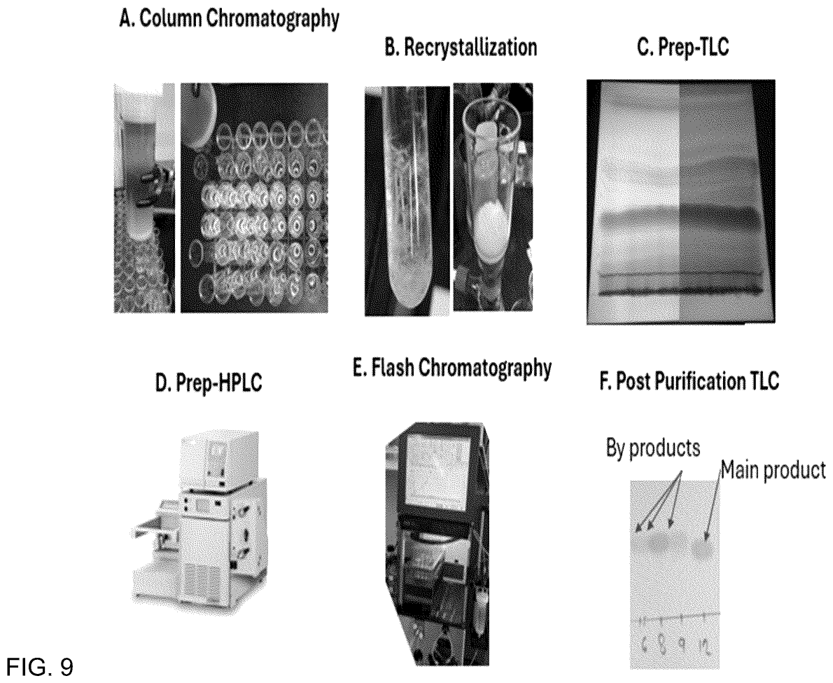

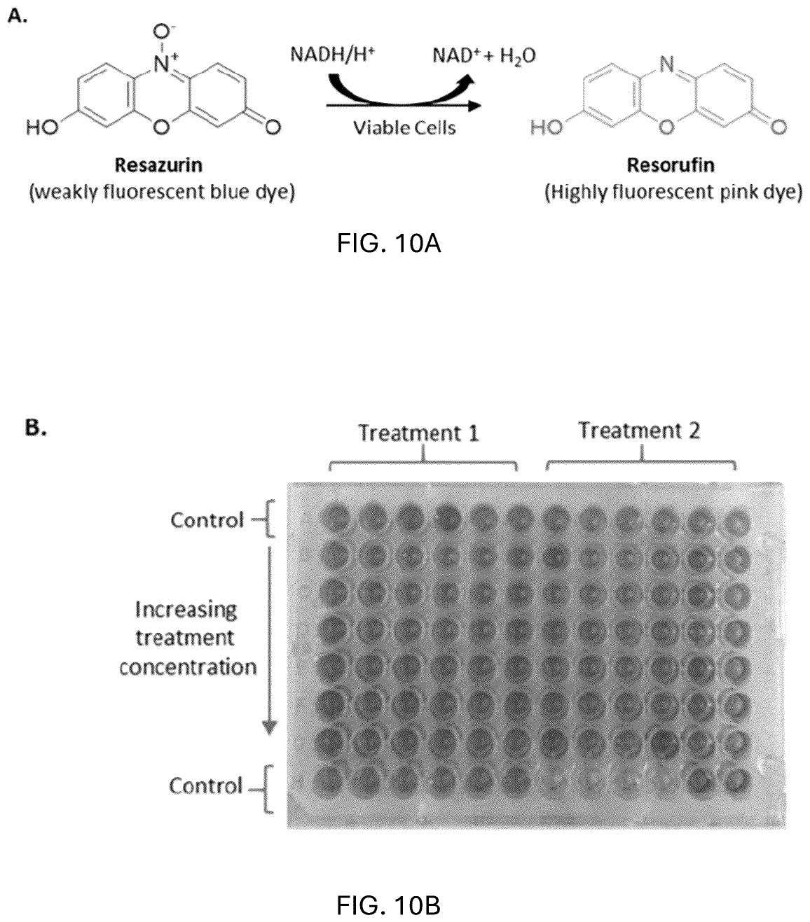

After completion, the formed precipitate was filtered, washed with cold water and ethanol and dried under vacuum. For compounds that do not form a precipitate, the solvent was evaporated under reduced pressure using the rotary evaporator, and the residue was neutralized with dilute HCl. Then, the product was extracted from the aqueous layer using ethyl acetate, followed by evaporation of the organic layer to isolate the product. Crude products were purified by recrystallization, column chromatography, Preparative-HPLC, flash chromatography, preparative-TLC or Sephadex L-H20 using different solvent systems (FIG. 9).

### Fourier Transform-Infrared Analysis (FT-IR)

Infrared (IR) spectra were recorded using Perkin Elmer Spotlight 400 FTIR. All spectra were recorded at room temperature over the mid-infrared range (4000-400 cm⁻¹). FT-IR spectra were used to confirm the presence of the main functional groups in the synthesized analogs.

### Mass Spectroscopy

Mass spectra were recorded on Agilent 6460 Triple Quadrupole Liquid chromatography-mass spectrometry (LC/MS) system combined with electrospray ionization (ESI) source. The analyses were performed using positive ionization mode (ESI+) with the capillary voltage set at 80 v and mass/charge (m/z) ratio acquisition in the range 40-610 m/z. Samples were dissolved in acetonitrile and analyzed by direct infusion using 50% ACN: 50% of 0.1% Formic acid mobile phase at a flow of 0.5 ml/min. The molecular weight of the synthesized compounds was confirmed by the presence of the molecular ion peaks at [M+1]⁺.

### Nuclear Magnetic Resonance (NMR)

¹H NMR and 13C NMR spectra were recorded on JEOL 600 MHz spectrometer at a frequency of 600 and 150 MHz, respectively, using chloroform-d or chloroform-d with/without 0.05% v/v of tetramethylsilane (TMS) as a solvent. Chemical shifts (8) are expressed as parts per million (ppm) relative to the solvent peak (7.24 ppm for 1H NMR and 77 ppm for 13C NMR). ¹H-¹H coupling constant (J) values are reported in Hz. Additional 2-dimensional (2D) ¹H-¹H correlation spectroscopy (COSY) and 1H-13C heteronuclear single quantum coherence (HSQC), ¹H-¹³C—Heteronuclear Multiple Bond Correlation (HMBC), ¹H-¹⁵N HMBC, ¹H-¹H-nuclear overhauser effect spectroscopy (NOESY) NMR and 1D-NMR for ¹⁵N and ¹⁹F nucleus were conducted for selected samples to confirm the assignment of protons and carbons chemical shifts. NMR data were processed using Delta NMR Software, Version 5.1.3. (JEOL, USA). Multiplicities were described using the following abbreviations: s=singlet, d=doublet, t=triplet, q=quartet, dd=doublet of doublets, dt=doublet of triplets, td=triplet of doublets, m=multiplet, and b=broad.

### Elemental Analysis

Elemental analyses were carried out using a Thermo Scientific FLASH 2000 CHN analyzer to aid in confirming molecular structures by calculating the percentage of C, H and N in each of the synthesized compounds.

### Cell Culture

The synthesized compounds were screened against triple negative human breast cancer cell lines (MDA-MB-231 and MDA-MB-436) and AR negative PCa cell lines (PC3 and DU145) as well as primary human dental bulb cells and normal breast epithelial cell line (MCF 10a). Cell lines were obtained from the American Type Tissue Culture (ATCC, Manassas, VA, USA. DU-145 cell line was a kind gift from Dr. Lotfi Chouchane (Weill Cornell Medical College in Qatar), which were originally purchased from ATCC. PCa cell lines were cultured in Gibco® RPMI 1640 with L-glutamine (Thermo Fisher Scientific, USA) whereas TNBCs were cultured in Gibco DMEM medium, supplemented with 10% Fetal bovine serum (FBS), 100 IU/mL Penicillin, and 100 μg/mL streptomycin (Thermo Fisher Scientific, USA), and maintained at 37° C. in a humidified incubator with 5% CO2. Cells were grown in T-75 tissue culture flasks and were routinely passaged when reaching confluency of 80-90% with a sub-culture ratio of 1:3 to 1:6. Cells were harvested using 0.25% trypsin EDTA and cryopreserved in growth media containing 5% dimethyl sulfoxide (DMSO) and supplemented with 35% FBS. The selectivity of the most potent compounds was tested against primary human dental bulb cells (ND) and normal breast epithelial cell line (MCF10a).

### Cell Viability

The in vitro cytotoxicity of the compounds was assessed by Alamar Blue assay (Invitrogen™, ThermoFisher Scientific, USA). Alamar Blue (resazurin) is a cell-permeable, weakly fluorescent blue dye that is reduced by mitochondrial enzymes in viable cells to resorufin, a highly fluorescent red molecule (FIG. 9). Thus, it was used as a cell health indicator by measuring the reducing power of living cells, which quantitatively reflects cell viability.

Briefly, MDA-MB-231 and MDA-MB-436, PC3 and DU145 cells were seeded into 96-well plates at a density of 5000-7500 per well in RPMI media supplemented with 10% FBS and incubated overnight. Once cells were attached, the culture media was aspirated and fresh media containing increasing concentrations of the synthesized compounds (1-40 M), docetaxel (0.001 μM-100 μM), or vehicle (DMSO) was added and incubated for 48 hours. The final concentration of DMSO was ≤0.1%. After 48 hours, treatment was removed and 100 μL of 1:10 Alamar blue reagent in media was added to each well and incubated for 2-4 hours (optimized according to the metabolic activity of each cell line). Then, fluorescence intensity was measured using Infinite 200 PRO Microplate Reader (Tecan, Switzerland) at 560 nm excitation wavelength and 590 emission wavelengths. Relative cell viability was calculated according to the following formula:

$$\% Viability = \frac{Fluorescence of treated wells - Blank}{Fluorescence of control wells - Blank} \times 100 \%$$

For most potent compounds, a dose-response curve was created and IC50, the concentration the induced 50% reduction in viability compared to control, was computed by non-linear regression (curve fit) analysis using GraphPad Prism Software and were expressed as mean +/−SEM. Treatments were tested in 6 technical replicates, and each experiment was repeated independently three times.

### Morphological Examination

Cells were seeded in 6 wells plate at a density of 150,000-200,000 cells/well and were incubated overnight. Then, cells were treated with the synthesized compounds at 5-10 μM concentration for 48 hours. Morphological changes were monitored using a DMi8 inverted microscope (Leica, Germany) and images were captured with a Leica MC170 HD camera.

### Annexin V Apoptosis Assay

Cellular apoptosis was assessed by PE Annexin V apoptosis Detection Kit (BDPharmingen, USA) as per the manufacturer's protocol. Briefly, cells were seeded in 100 mm Petri dishes at a density of 1.5 million cells/dish and incubated overnight. On the next day, cells were treated with one of the three most potent compounds (13, 15, and 16), docetaxel or vehicle alone (DMSO) for 48 hours. Adherent and floating cells were then harvested by trypsin, washed twice by PBS, resuspended in binding buffer and stained with Phycoerythrin (PE) conjugated Annexin-V, 7-AAD or both stains for 15 minutes. After staining, samples were analyzed by BD FACSAria™ II Flow Cytometer and FlowJo software. First, the cell population, excluding debris, was gated with forward scatter (FSC-A) and side scatter (SSC-A). Then, doublets were excluded using FSC-height and FSC-width plots. Singlet cells were then presented as dot plots of FITC-A (annexinV) against PerCP-Cy5.5-A (7-AAD) channels. Quad Gates were used to calculate the percentage of viable cells (annexin V low, 7-AAD low), early pro-apoptotic cells (annexin V high, 7-AAD low) and late apoptosis/dead cells (annexin V high, 7-AAD high).

### Cell Cycle Analysis

The compounds' effect on cell cycle distribution of PC3 and DU145 was assessed by flow cytometry using propidium iodide (PI). Cells were seeded in 100 mm Petri dishes at a density of 1.5 million cells/dish and incubated in RPMI supplemented with 10% FBS overnight at 37° C. Complete culture media was then removed, and cells were starved in serum serum-free for 16 hours to synchronize cells at the G0/G1 phase of the cell cycle. Subsequently, cells were treated with the compounds 13, 15, and 16 or docetaxel for 48 hours. After treatment, floating and adherent cells were collected, washed with ice-cold PBS and centrifuged at 500 g for 10 minutes at 4° C. Next, cells were fixed with ice-cold 70% ethanol added drop wisely while vertexing and stored at −20° C. for at least 24 hours. On the day of analysis, cells were pelleted (centrifuged at 800 g for 10 minutes at 4° C.), washed twice with ice-cold PBS and counted. Around one million cells of each sample were resuspended in 500 μl of FxCycle PI/RNase Staining Solution® (Thermo Fisher Scientific, USA) supplemented with 200 μg/ml RNase and incubated for 50 minutes at 37° C. in a shaking water bath, protected from light. Following incubation, cells were immediately analyzed by BD FACSAria™ II Flow Cytometer, at least 50,000 events were acquired for each sample. The proportion of cells in each cell cycle phase: G1, S and G2-M was determined using FlowJo software based on cells' DNA content as represented by histograms of PI signal intensity.

### Colony Formation Assay

Anchorage-independent growth, a characteristic of transformed cells that correlate with in vivo tumorigenic potential, was assessed by soft agar assay. First, a 2% noble agar (Sigma-Aldrich) stock solution was prepared in deionized and autoclaved water. Next, 10,000 cells of MDA-MB-231, MDA-MB-436, PC3 or DU145 were seeded in duplicate in 1 ml of 0.2% (w/v) agar in a complete RPMI medium containing 5-10 μM of the compounds on top of a pre-solidified 0.4% agar layer in 6-well plates. Colony formation was monitored for 14-21 days, and images were captured from different fields every seven days using a DMi8 inverted microscope equipped with a Leica MC170 HD camera. After 14 days, the average number of colonies in each well was counted manually under the microscope and confirmed by ImageJ; only colonies with an area larger than 100 jim² were counted to avoid the inclusion of non-proliferating dead cells. Besides, the size of colonies in each treatment condition was measured by ImageJ from at least six random fields per well. Colonies were then categorized according to their size into small (100-500 μm²), medium (500-1000 μm²) and large (>1000 μm²).

### Wound Healing Assay

MDA-MB-231, MDA-MB-436, PC3 and DU145 cells were seeded in 6-well plates at a concentration of 400,000 cells/well and were grown to 80-90% confluence in a complete RPMI culture medium. Following, Cells were washed with PBS and incubated in serum-free RPMI for 4 hours. Then, the cell layer was scratched with a 20 μl pipette tip and washed with PBS to remove floating cells. Next, cells were treated with an increasing concentration of the compounds prepared in 0.5% FBS containing medium for 48 hours. Wound areas were imaged in six different locations for each well at 0, 24, 48 hours by DMi8 inverted microscope equipped with a Leica MC170 HD camera and quantified using ImageJ software. The average extent of wound closure was calculated by the following equation:

$$\% Wound closure = \frac{Would area at 0 h - Wound area at 48 h}{Would area at 0 h} \times 100 \%$$

### Trans-Well Migration Assay

Trans-well migration assay was carried out in a multi-well permeable support system with 8.0 μm-pore Polyethylene terephthalate (PET) membrane (BD Falcon™, USA). Briefly, 50,000 PC3 or DU-145 cells were suspended in 500 μL of serum-free RPMI medium, containing 5-10 μM of the compounds or vehicle, and loaded in the upper chamber. Lower chambers were filled with 600 μL of complete growth medium (RPMI with 10% FBS) as a chemoattractant. Cells were allowed to migrate for 48 hours, after which non-migrating cells on the upper side of the chamber were removed with a cotton swab. Migrating cells were then fixed in 3.7% formaldehyde for 10 minutes, permeabilized with methanol for 5 minutes and stained with 0.5% crystal violet prepared in 2% ethanol for 15 minutes. Images were taken with an inverted microscope (10× objective) equipped with a digital camera. The average number of migrated cells from at least four random fields/well was quantified by ImageJ software.

TABLE 2
List of used primary and secondary antibodies

| | | | | MW of | | |
|---|---|---|---|---|---|---|
| | | | | protein | | |
| No. | Antibody | Type | Source | target | | Manufacturer |
| 1 | Anti-Mouse | Polyclonal and | Goat | NA | | Cell Signaling |
| | | monoclonal | | | | Technology, Inc., |
| | | | | | | USA |
| 2 | Anti-Rabbit | Polyclonal and | Goat | NA | | Cell Signaling |
| | | monoclonal | | | | Technology, Inc., |
| | | | | | | USA |
| 3 | GAPDH | | Rabbit | 37 | kDa | Abcam, USA |
| 4 | E-cadherin | Monoclonal | Mouse | 135 | kDa | Abcam, USA |
| 5 | β-Catenin | Polyclonal | | 92 | kDa | Cell Signaling |
| | | | | | | Technology, Inc., |
| | | | | | | USA |
| 6 | Phospho-β- | Monoclonal | | 92 | kDa | Cell Signaling |
| | Catenin | | | | | Technology, Inc., |
| | | | | | | USA |
| 7 | Fascin | polyclonal | | 54 | kDa | Abcam, USA |
| 8 | Cleaved Caspase- | Polyclonal | Rabbit | 17 | kDa | Abcam, USA |
| | 3 | | | | | |
| 9 | Bcl-2 | Monoclonal | Mouse | 26 | kDa | Abcam, USA |
| 10 | Bax | Monoclonal | Mouse | 23 | kDa | Invitrogen, USA |
| 11 | ERK 1/2 | Polyclonal | Rabbit | 44, 42 | kDa | Abcam, USA |
| 12 | P-ERK1/2 | Polyclonal | Rabbit | 44, 42 | kDa | Invitrogen, USA |
| | (Thr202, Tyr204) | | | | | |
| 13 | Total Akt | Polyclonal | Rabbit | 60 | kDa | Cell Signaling |
| | | | | | | Technology, Inc., |
| | | | | | | USA |
| 14 | Phospho-Akt | Polyclonal | Rabbit | 60 | kDa | Cell Signaling |
| | | | | | | Technology, Inc., |
| | | | | | | USA |
| 15 | JNK1/2/3 | Polyclonal | Rabbit | 54 | kDa | Abcam, USA |

NA: Not Applicable

### Chorioallantoic Membrane (CAM) Angiogenesis Assay

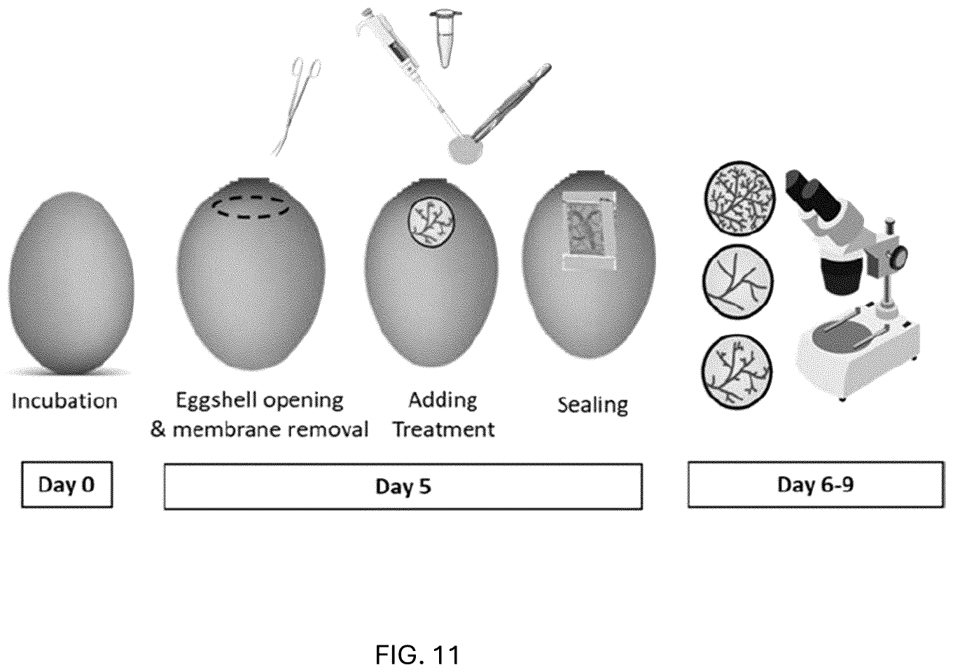

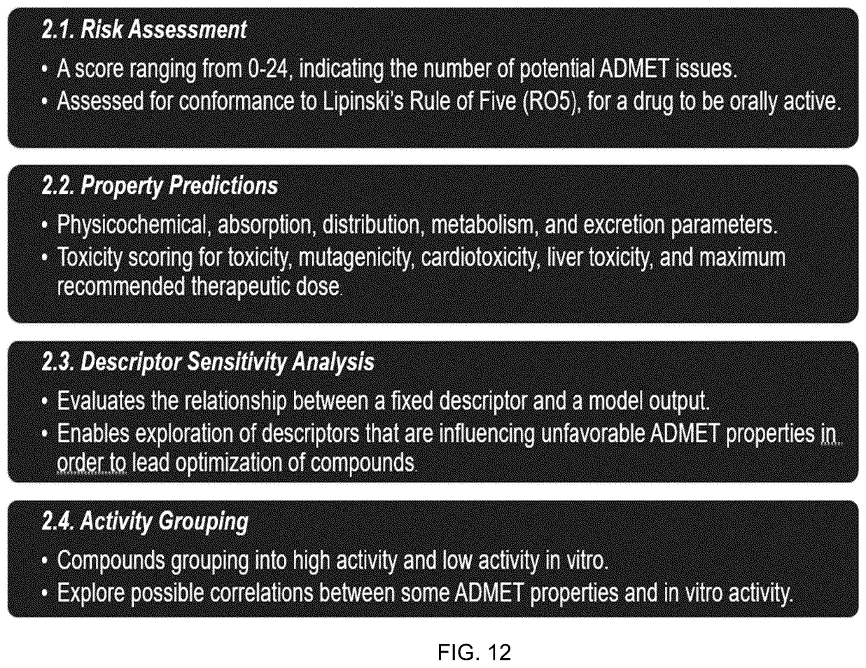

Fertilized white leghorn chicken eggs were obtained from Arab Qatari for Poultry Production, Doha, Qatar. Eggs were incubated in a rotary humidified MultiQuip Incubator at 37° C. with 60% humidity. Ethics approval was obtained from Institutional Animal Care & Use Committee and Institutional Bio-safety committee at Qatar University. After 5 days of incubation, chicken embryos were treated with compound 16 or vehicle (DMSO). Briefly, eggshells were disinfected with ethanol and a small opening was made in the shell over the air sac. Next, 200 μl of 1×PBS was added and the shell membrane was carefully removed. Then, 2 μl of 10 mM (3.21 μg/ml) compound 16 stock solution or 2 μL of DMSO were loaded on a round coverslip (0.5 cm²) and directly placed over the CAM. Subsequently, opened windows were sealed and the eggs were returned to a stationary incubator (FIG. 11).

The effect of compounds on angiogenesis was monitored for 48 hours post-treatment and microscopic images of exposed (under coverslip) and unexposed area in each embryo were captured at equivalent magnification. Images were then analyzed using AngioTool software using the following parameters: vessel diameter thresholds at [10,255], vessel thickness at 7 and 10, removed small particles at 60, and filled holes at 183. The difference in blood vessel formation between treated and untreated embryos was assessed based on average vessel length, vessel percentage area and the total number of blood vessel junctions.

To reduce the negative impact of variabilities between embryos, the exposed area in each embryo was first corrected to the unexposed area within the same embryo before being compared to untreated embryos. Besides, at least 15 embryos were treated in each group.

### Statistical Analysis

All data were analyzed by GraphPad Prism9 software. Data are presented as the mean±standard error of the mean (SEM) of three biologically independent experiments unless otherwise stated in figure legends. Two-group datasets were analyzed by Student's unpaired t-test. One-way analysis of variance (ANOVA) followed by Tukey's or Dunnett's posthoc tests were used to compare three or more groups. Tukey's posthoc test was used to compare treatment groups to each other, while Dunnett's test was used to compare treatment groups with the control only. Differences were considered statistically significant when P-values were <0.05. For all the statistical analysis, *=p<0.05, **=p<0.01, and ***=p<0.001 unless otherwise stated in the figure caption.

### In-Silico Physicochemical, Biopharmaceutical, and Pharmacokinetic Characterization

State-of-the-art computer modeling software programs and open-access platforms were utilized to perform in silico assays on synthesized tetralone-based chalcone analogs. ADMET Predictor™ (Simulations Plus, Lancaster, California, USA) was used for modeling the biopharmaceutical, physicochemical, and pharmacokinetic parameters of synthesized chalcone analogs. The program can accurately predict over 140 properties including solubility, log P, pKa, sites of CYP metabolism, and Ames mutagenicity.

The ADMET Modeler™ module in ADMET Predictor was used to rapidly create high quality QSAR/QSPR models. The structure-based predictions generated were applied to predict the physicochemical properties of real and virtual compounds structurally-related to chalcone analogs, metabolite and toxicity predictions, and determination of dose required to achieve a specific plasma concentration. The Metabolism Module in ADMET Predictor allows to predict Cytochrome P450 metabolites for nine CYP isoforms (1A2, 2C9, 2C19, 2D6, 3A4, 2A6, 2B6, 2C8, 2E1), CYP kinetic parameters (Km, Vmax, CLint) for the five major drug metabolizing CYPs (1A2, 2C9, 2C19, 2D6, 3A4), CYP inhibition for the five major drug metabolizing CYPs (1A2, 2C9, 2C19, 2D6, 3A4), and UGT substrate for nine UGT isoforms (1A1, 1A3, 1A4, 1A6, 1A8, 1A9, 1A10, 2B7, 2B15). CYP kinetic parameters, Km, Vmax, and CLint, were predicted from ANNE regression models. The Toxicity Module was utilized to model the toxicity profile of each synthesized chalcone analog generating predictions of estrogen and androgen receptor toxicities, maximum recommended therapeutic dose, carcinogenicity in rats and mice as TD50, human liver adverse effects, acute lethal toxicity in rats as LD50, and allergenic skin and respiratory sensitizations. Chemical structures of synthesized chalcone analogs were fed into the software program in the format of SDF, RDF, MOL, or SMILES; or modified using MedChem Designer™ to other structurally related compounds.

After the analogs were uploaded and given their respective descriptor, all available models (physicochemical, metabolism, toxicity, and modeling descriptors) were applied to calculate around 500 descriptors. ADMET values were calculated at a pH of 7.4. Data was presented in a spreadsheet, but also in heat maps, which allowed for visualization of attribute progression (e.g. the largest values of each attribute are red colored). In addition, the program automatically generated star plots which resemble pie charts with colored wedges whose length corresponds to the magnitude of the value that it represents.

### Results

### Design of Novel Chalcone Derivatives

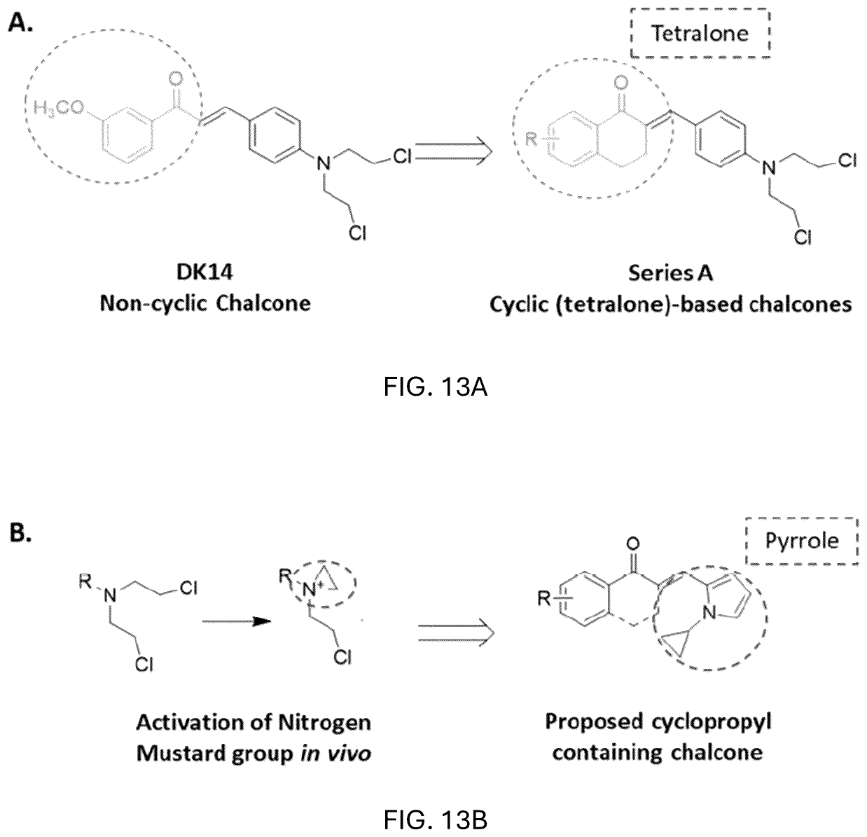

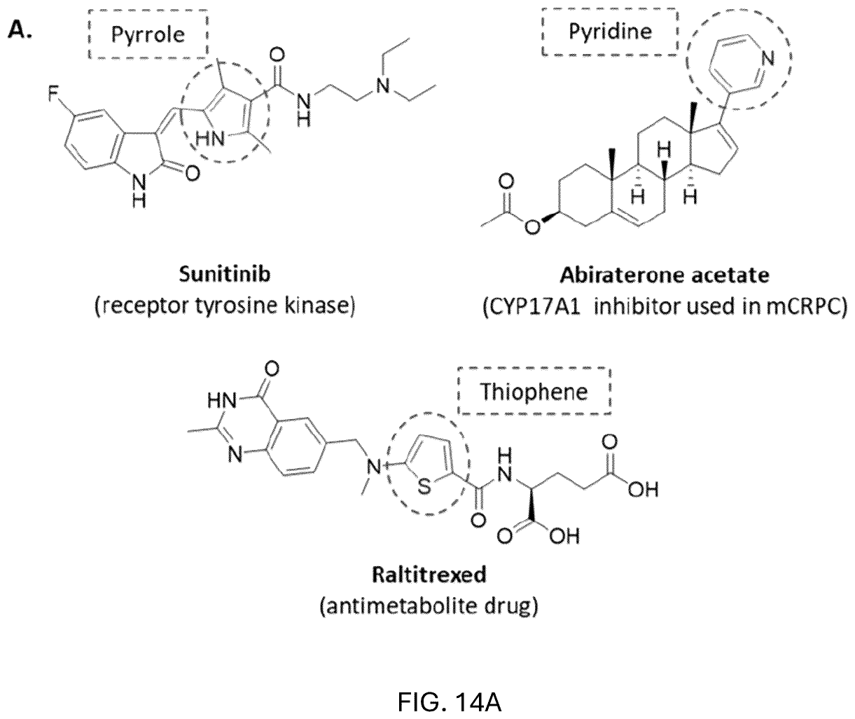

In this study, a new library of chalcone analogs was designed, aiming to improve the potency and the pharmacokinetic profile of the reported analogs. In the first series, Applicant explored the effect of substituting the non-cyclic ketone in (Bis-(2-chloroethyl)amine) based chalcone (DK14) with a closed ring (tetralone), resulting in α-conformationally restricted (cyclic) chalcone analogs (FIG. 13A). Next, a second series was designed to investigate the effect of replacing the nitrogen mustard group in DK14 with cyclopropyl pyrrole. This functional group might potentially act as a bio-isostere that retains similar or enhanced potency while improving the pharmacokinetic profile (FIG. 13B).

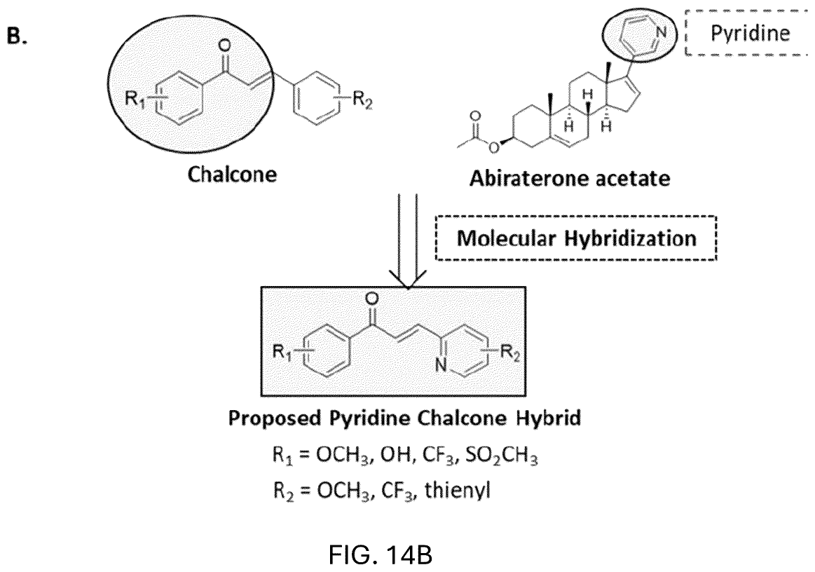

Additionally, a new line of chalcone derivatives was designed through molecular hybridization of chalcones with pharmacologically active heterocycle rings, including pyrrole, pyridine, and thiophene (FIG. 14). Similar to the first series, non-cyclic chalcones were compared to their corresponding cyclic chalcones by incorporating either aryl or tetralone rings, respectively.

The design process was guided by both in silico ADMET study findings and biological screening data. Hits showing promising ADMET profiles or biological activity were selected for expanded investigation in the subsequent phases of the project. The novelty of the proposed analogs was checked by both structure-based search as well as chemical name-based search. Analogs that were previously reported or synthesized were excluded from the study.

### Chemical Synthesis

The designed chalcone analogs were synthesized through base-catalyzed Claisen-Schmidt condensation reaction of substituted tetralone or acetophenone with substituted carboxaldehyde (FIG. 8). The products were primarily collected by filtration and purified by appropriate chromatographic method (i.e., column chromatography, flash-chromatography, Prep-TLC, Prep-HPLC).

Twenty-six (1-26) novel chalcone analogs containing nitrogen mustard, pyridine, pyrrole, or thieno-thiophene in ring B and various electron-donating or withdrawing groups on ring A were successfully synthesized (Table 3 and FIG. 14B).

The time required for reactions to complete and the yield varied significantly between analogs. Tetralone-based chalcones took a significantly longer time to complete (>72 hours) and resulted in relatively low yields, mostly between 31-43%. On the other hand, the synthesis of non-cyclic chalcones took less time to complete and resulted in higher yields. Specifically, the synthesis of thienyl pyridine bearing chalcones completed in few hours and generated a yield of 65-87%.

TABLE 3
General structures and numbering of the synthesized chalcones

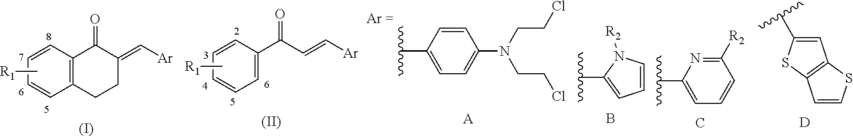

| # | Scheme | Ar | R1 | R2 |
|---|---|---|---|---|
| 1 | I | A | 5-OCH3 | — |
| 2 | I | A | 6-OCH3 | — |
| 3 | I | A | 7-OCH3 | — |
| 4 | I | A | 6,7-OCH3 | — |
| 5 | I | A | 5,8-OCH3 | — |
| DK14 | II | A | 3-OCH3 | — |
| 6 | I | B | 6-OCH3 | H |
| 7 | I | B | 6-OCH3 | cyclopropyl |
| 8 | I | B | 6,7-OCH3 | cyclopropyl |
| 9 | II | B | 3-OCH3 | cyclopropyl |
| 10 | I | D | 6,7-OCH3 | — |
| 11 | I | C | 6,7-OCH3 | OCH3 |
| 12 | I | C | 6,7-OCH3 | CF3 |
| 13 | I | C | 6,7-OCH3 | thiophen-3yl |
| 14 | I | C | 6-OCH3 | thiophen-3yl |
| 15 | I | C | 7-OCH3 | thiophen-3yl |
| 16 | II | C | 3-OCH3 | thiophen-3y1 |
| 17 | II | C | 2-OCH3 | thiophen-3yl |
| 18 | II | C | 4-OCH3 | thiophen-3yl |
| 19 | II | C | 3,4-OCH3 | thiophen-3yl |
| 20 | II | C | 2,3,4-OCH3 | thiophen-3yl |
| 21 | II | C | 2,4,6-OCH3 | thiophen-3yl |
| 22 | II | C | 3,4,5-OCH3 | thiophen-3yl |
| 23 | II | C | 2-CH3SO2 | thiophen-3yl |
| 24 | II | C | 2-OH | thiophen-3yl |
| 25 | II | C | 2-CF3 | thiophen-3yl |
| 26 | II | C | 4-F | thiophen-3yl |

### Structure Elucidation

The structures of the synthesized compounds (1-26) were elucidated by FT-IR, 1H-NMR, ¹³C-NMR, ESI-MS and elemental analysis. The FT-IR spectra confirmed the presence of the major functional groups in the synthesized analogs. All chalcones exhibited a characteristic C═O stretching band at 1644-1662 cm⁻¹. The appearance of the C═O band at a lower wavelength than what is typically observed with ketones (>1700 cm⁻¹), confirms the conjugation of the carbonyl with C═C in chalcone structure. Another characteristic band shown by different chalcone analogs is the C═C stretching vibration between 1514-1610 cm⁻¹. Applicant also noted C—N stretching bands at 1176-1178 in nitrogen mustard-containing analogs (15). The mass spectra (+ESI) of all compounds showed an [M+1]⁺ peak equivalent to their calculated molecular weights, suggesting a correct chemical composition. In addition, elemental analysis results (C, H, N) were relatively similar to the calculated values.

The configuration of the synthesized analogs was confirmed through 1H-NMR. Typically coupling constant between α and β olefinic protons in α, β unsaturated ketones were used to differentiate between F and Z configuration where E isomer present with a larger coupling (J=15 Hz) than Z isomer (J=12 Hz). Proton NMR spectra of the synthesized α-unsubstituted (non-cyclic) chalcones (9 and 17-26) exhibited two doublets with a large coupling constant (J=15.1-15.8 Hz), confirming (I) configuration. However, the tetralone-based chalcones (1-8, 10 and 13-16) lack the α-olefinic proton, thus assignment based on the coupling is not possible. Therefore, alternative methods were used to confirm the configuration. Inspection of the compound's (8) NOESY spectrum revealed correlations between β-olefinic proton and H-3 of the tetralone, which suggests (I) configuration. Besides, x-ray crystallography analysis of previously reported related tetralone-based chalcones confirmed A-configuration (39, 40). Due to steric interactions between the carbonyl and ring B in chalcones, the formation of E-isomer was favored over Z-isomer in the majority of the reported chalcones.

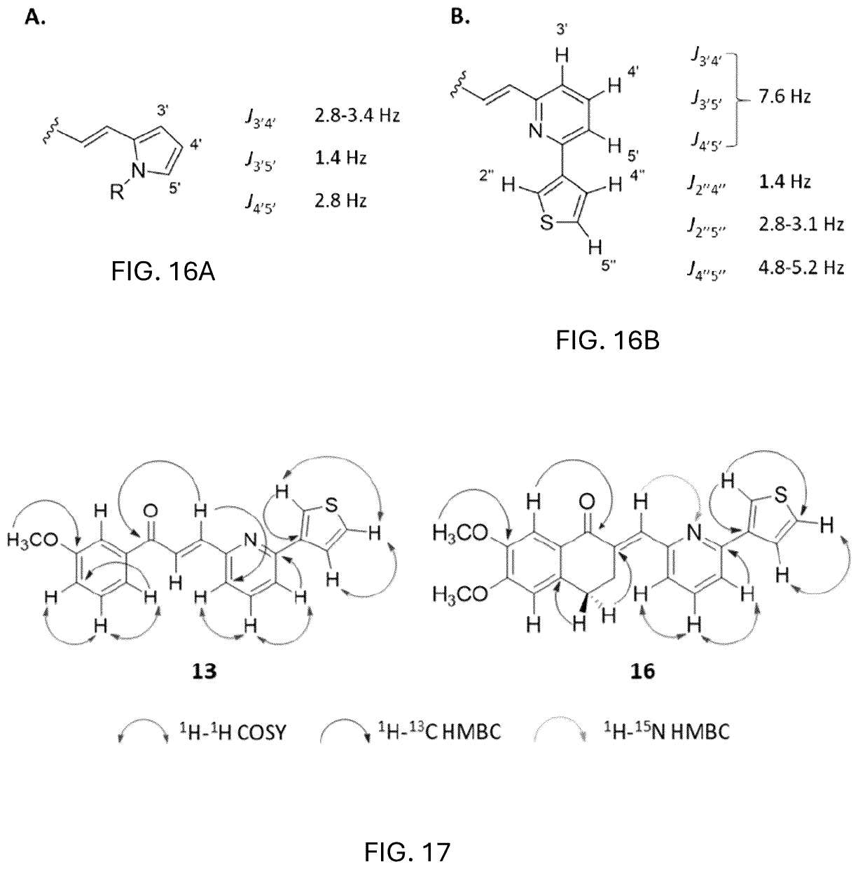

Due to the presence of 2-3 distinct aromatic rings in the synthesized analogs and thus several overlapping aromatic proton peaks in the region of 7.0-8.0 ppm, 2D ¹H-¹H COSY NMR in addition to 1D ¹H and ¹³C NMR were run for all analogs to aid in peak assignment. Synthesis of multiple related analogs within each series enabled assignment with high confidence by comparing spectra of different analogs. Aromatic rings were distinguished by their characteristic J-coupling values and expected splitting pattern (FIGS. 16A-16B).

Additionally, 2D NMR experiments (COSY, HSQC, ¹H-¹³C—HMBC, ¹H-¹⁵N-HMBC and NOESY) were conducted for two representative analogs (13 and 16) for full assignment of carbons, protons and their connectivity (FIG. 17). Notably, ¹H-¹⁵N-HMBC NMR of compound 13 showed a correlation between the nitrogen in the pyridine ring and β-olefinic proton, confirming its location within 2-3 bonds. Another NMR characteristic observation was noted in ¹³C-NMR of compound (25). The presence of the CF₃ functional group was confirmed by the ¹³C-NMR splitting of CF₃ and adjacent aryl carbons to quartets with large J-couplings of 274.5, 32.3 and 4.8, respectively. This observation was further confirmed by ¹⁹F NMR, where a fluorine peak appeared at −57.7 ppm consistent with the expected region for CF₃ (−50 to −70 ppm).

The results of the characterization experiments for compounds 1-26 are summarized below.

### 1. (E)-2-(4-(bis(2-chloroethyl)amino)benzylidene)-5-methoxy-3,4-dihydronaphthalen-1 (2H)-one (1)

Orange-brown semi-solid oil; yield: 31.0%; 1H-NMR (CDCl3 with 0.05% v/v of TMS, 600 MHZ) δ 7.79 (s, 1H, β-olefinic), 7.74 (d, J=6.9 Hz, 1H, H-8), 7.44 (d, J=9.0 Hz, 2H, H2′ and H-6′), 7.31 (t, J=7.9 Hz, 1H, H-7), 7.03 (d, J=7.6 Hz, 1H, H-7), 6.72 (d, J=9.0 Hz, 2H, H-3′ and H-5′), 3.87 (s, 3H, OCH₃-5), 3.79 (t, J=6.9 Hz, 4H, N—CH₂), 3.67 (t, J=7.2 Hz, 4H, Cl—CH₂), 3.12 (td, J=6.5, 1.4 Hz, 2H, H-3), 2.92 (t, J=6.5 Hz, 2H, H-4); ¹³C-NMR (CDCl3, 150 MHz) δ 187.9, 156.1, 146.3, 136.6, 134.7, 132.3, 132.2, 131.9, 127.0, 125.2, 119.8, 114.0, 111.5, 55.7, 53.3, 40.2, 26.6, 21.2; FT-IR (KBr, cm⁻¹): 3072 (C═C—H), 2956, 2837 (C═C—H), 1659 (C═O), 1596, 575, 1514 (C═C), 1176 (C—N); Anal. calcd. for C22H23C12NO2: C, 65.35; H, 5.73; N, 3.46; Found: C, 64.53; H, 5.85; N, 3.44; LC-MS (+)-ESI (m/z): calculated 403.11, observed 404.0 [M+1]⁺.

### 2. (E)-2-(4-(bis(2-chloroethyl)amino) benzylidene)-6-methoxy-3,4-dihydronaphthalen-1 (2H)-one (2)

Dark yellow solid; yield: 40.1%; 1H-NMR (CDCl3 with 0.05% v/v of TMS, 600 MHz) δ 8.09 (d, J=9.0 Hz, 1H, H-8), 7.79 (s, 1H, β-olefinic), 7.42 (d, J=9.0 Hz, 2H, H-2′ and H6′), 6.87 (dd, J=9.0, 2.8 Hz, 1H, H-7), 6.71 (d, J=9.0 Hz, 2H, H-3′ and H-5′), 6.70 (d, J=2.1 Hz, 1H, H-5), 3.87 (s, 3H, OCH₃-6), 3.79 (t, J=7.2 Hz, 4H, N—CH₂), 3.66 (t, J=7.2 Hz, 4H, Cl—CH₂), 3.14 (td, J=6.5, 2.1 Hz, 2H, H-3), 2.91 (t, J=6.5 Hz, 2H, H-4); ¹³C-NMR (CDCl3, 150 MHz) δ 186.7, 163.3, 146.2, 145.4, 136.2, 132.4, 132.2, 130.6, 127.3, 125.3, 113.1, 112.2, 111.5, 55.4, 53.3, 40.3, 29.2, 27.3; FT-IR (KBr, cm⁻¹): 3013 (C═C—H), 2938, 2838 (C═C—H), 1658 (C═O), 1600, 1580, 1515 (C═C), 1185 (C—N); Anal. calcd. for C22H23C12NO2: C, 65.35; H, 5.73; N, 3.46; Found: C, 64.82; H, 6.07; N, 3.41; LC-MS (+)-ESI (m/z): calculated 403.11, observed 404.0 [M+1]⁺.

### 3. (E)-2-(4-(bis(2-chloroethyl)amino) benzylidene)-7-methoxy-3,4-dihydronaphthalen-1 (2H)-one (3)

Yellow-orange solid; yield: 43.2%; 1H-NMR (CDCl3 with 0.05% v/v of TMS, 600 MHz) δ 7.81 (s, 1H, β-olefinic), 7.61 (d, J=2.8 Hz, 1H, H-8), 7.44 (d, J=9.0 Hz, 2H, H-2′ and H-6′), 7.15 (d, J=8.3 Hz, 1H, H-5), 7.05 (dd, J=8.3, 2.8 Hz, 1H, H-6), 6.72 (d, J=9.0 Hz, 2H, H-3′ and H-5′), 3.87 (s, 3H, OCH₃-7), 3.79 (t, J=6.9 Hz, 4H, N—CH₂), 3.67 (t, J=7.2 Hz, 4H, Cl—CH₂), 3.13 (td, J=6.5, 1.4 Hz, 2H, H-3), 2.88 (t, J=6.5 Hz, 2H, H-4); ¹³C-NMR (CDCl3, 150 MHz) δ 187.6, 158.6, 146.4 137.0, 135.6, 134.6, 132.3, 132.2, 129.2, 125.1, 121.0, 111.5, 110.3, 55.5, 53.3, 40.2, 27.9, 27.5; FT-IR (KBr, cm⁻¹): 3071 (C═C—H), 2959, 2835 (CC—H), 1652 (C—O), 1603, 1571, 1516 (C═C), 1177 (C—N); Anal. calcd. for C22H23Cl2NO2: C, 65.35; H, 5.73; N, 3.46; Found: C, 64.96; H, 5.77; N, 3.59; LC-MS (+)-ESI (m/z): calculated 403.11, observed 404.1 [M+1]⁺.

### 4. (E)-2-(4-(bis(2-chloroethyl)amino) benzylidene)-6,7-dimethoxy-3,4-dihydronaphthalen-1 (2H)-one (4)

Yellow crystalline solid; yield: 36.2%; 1H-NMR (CDCl3 with 0.05% v/v of TMS, 600 MHz) δ 7.77 (s, 1H, β-olefinic), 7.62 (s, 1H, H-8), 7.42 (d, J=9.0 Hz, 2H H-2′ and H-6′), 6.71 (d, J=9.0 Hz, 2H, H-3′ and H-5′), 6.67 (s, 1H, H-5), 3.95 (2s, 6H, OCH₃-6 and OCH₃-7), 3.79 (t, J=6.9 Hz, 4H, N—CH₂), 3.66 (t, J=7.2 Hz, 4H, Cl—CH₂), 3.15 (td, J=6.5, 1.4 Hz, 2H, H-3), 2.89 (t, J=6.5 Hz, 2H, H-4); ¹³C-NMR (CDCl3, 150 MHz) δ 186.7, 153.2, 148.1, 146.2, 137.8, 136.2, 132.2, 126.9, 125.3, 111.5, 109.7, 109.6, 56.1, 56.0, 53.3, 40.3, 28.5, 27.6; FT-IR (KBr, cm⁻¹): 3071 (C═C—H), 2934, 2835 (C═C—H), 1647 (C═O), 1599, 1567, 1507 (C═C), 1179 (C—N); Anal. calcd. for C23H25C12NO3: C, 63.60; H, 5.80; N, 3.22; Found: C, 63.50; H, 6.09; N, 3.20; LC-MS (+)-ESI (m/z): calculated 433.12, observed 434.0 [M+1]⁺.

### 5. (E)-2-(4-(bis(2-chloroethyl)amino) benzylidene)-5,8-dimethoxy-3,4-dihydronaphthalen-1 (2H)-one (5)

Orange-brown solid; yield: 80.8%; 1H-NMR (CDCl3 with 0.05% v/v of TMS, 600 MHz) δ 7.78 (s, 1H, β-olefinic), 7.43 (d, J=9.0 Hz, 2H, H-2′ and H-6′), 6.98 (d, J=9.0 Hz, 1H, H-7), 6.83 (d, J=9.0 Hz, 1H, H-6), 6.70 (d, J=9.0 Hz, 2H, H-3′ and H-5′), 3.88 (s, 3H, OCH₃-5), 3.82 (s, 3H, OCH₃-8), 3.78 (t, J=6.9 Hz, 4H, N—CH₂), 3.66 (t, J=6.9 Hz, 4H, Cl—CH₂), 2.99 (td, J=6.7, 2.3 Hz, 2H, H-3), 2.85 (t, J=6.5 Hz, 2H, H-4); ¹³C-NMR (CDCl3, 150 MHz) δ 187.0, 153.9, 149.4, 146.2, 135.8, 133.6, 133.4, 132.2, 125.4, 124.5, 115.3, 111.5, 110.4, 56.4, 56.2, 53.3, 40.2, 26.5, 21.3; FT-IR (KBr, cm⁻¹): 2994 (C═C—H), 2901, 2837 (C═CH), 1661 (C—O), 1584, 1575, 1515 (C═C), 1178 (C—N); Anal. calcd. for C23H25C12NO3: C, 63.60; H, 5.80; N, 3.22; Found: C, 62.91; H, 6.04; N, 3.31; LC-MS (+)-ESI (m/z): calculated 433.12, observed 434.1 [M+1]⁺.

### 6. (E)-2-((1H-pyrrol-2-yl)methylene)-6-methoxy-3,4-dihydronaphthalen-1 (2H)-one (6)

Dark green crystalline solid; yield: 39.2%; 1H-NMR (CDCl3, 600 MHZ) δ 9.23 (s, 1H, NH), 8.08 (d, J=9.0 Hz, 1H, H-8), 7.86 (s, 1H, β-olefinic), 7.01 (td, J=2.8, 1.4 Hz, 1H, H5′), 6.88 (dd, J=8.6, 2.4 Hz, 1H, H-7), 6.73 (d, J=2.1 Hz, 1H, H-5), 6.67 (br, 1H H-3′), 6.38 (m, 1H, H-4′), 3.88 (s, 3H, OCH₃-6), 3.14 (td, J=6.7, 1.8 Hz, 2H, H-3), 2.97 (t, J=6.5 Hz, 2H, H-4); ¹³C-NMR (CDCl3, 150 MHZ) δ 186.3, 163.3, 145.5, 130.3, 129.3, 129.0, 127.4, 126.5, 121.5, 113.3, 113.1, 112.3, 111.2, 55.4, 28.5, 27.0; FT-IR (KBr, cm⁻¹): 3247 (N—H), 3100 (C═C—H), 2919, 2849 (C═C—H), 1644 (C—O), 1598, 1580, 1547 (C═C); Anal. calcd. for C16H15NO2: C, 75.87; H, 5.97; N, 5.53; Found: C, 75.71; H, 6.17; N, 5.65; LC-MS (+)-ESI (m/z): calculated 253.11, observed 254.2 [M+1]⁺.

### 7. (E)-2-((1-cyclopropyl-1H-pyrrol-2-yl)methylene)-6-methoxy-3,4-dihydronaphthalen-1 (2H)-one (7)

Pale yellow solid; yield: 70.3%; 1H-NMR (CDCl3 with 0.05% v/v of TMS, 600 MHZ) δ 8.17 (s, 1H, β-olefinic), 8.09 (d, J=9.0 Hz, 1H, H-8), 6.88-6.87 (overlapped, 1H, H-5′), 6.87 (dd, J=9.0, 2.8 Hz, 2H, H-7), 6.72 (d, J=2.8 Hz, 1H, H-5), 6.56 (d, J=2.8 Hz, 1H, H-3′), 6.21 (t, J=3.4 Hz, 1H, H-4′), 3.87 (s, 3H, OCH₃-6), 3.39-3.29 (m, 1H, H-1″), 3.12 (td, J=6.7, 1.8 Hz, 2H, H-3), 2.95 (t, J=6.5 Hz, 2H, H-4), 1.12 (td, J=7.1, 5.3 Hz, 2H, H-2a″ and H-3a″), 1.04-0.95 (m, 2H, H-2b″ and H-3b″); ¹³C-NMR (CDCl3, 150 MHz) δ 186.1, 163.2, 145.3, 131.1, 130.4, 129.8, 127.5, 125.1, 124.7, 113.9, 113.0, 112.2, 108.8, 55.4, 28.5, 28.3, 27.3, 7.1; FT-IR (KBr, cm⁻¹): 3133,3009 (C═C—H), 2939, 2835 (C═C—H), 1649 (C—O), 1599, 1582, 1570, 1494 (C═C); Anal. calcd. for C19H19NO2: C, 77.79; H, 6.53; N, 4.77; Found: C, 76.33; H, 6.63; N, 4.8; LC-MS (+)-ESI (m/z): calculated 293.14, observed 294.2 [M+1]⁺.

### 8. (E)-2-((1-cyclopropyl-1H-pyrrol-2-yl)methylene)-6,7-dimethoxy-3,4-dihydronaphthalen-1 (2H)-on (8)

Green crystalline solid; yield: 76.13%; 1H-NMR (CDCl3 with 0.05% v/v of TMS, 600 MHz) δ 8.17 (s, 1H, β-olefinic), 7.63 (s, 1H, H-8), 6.88 (dd, J=2.8, 1.4 Hz, 1H, H-5′), 6.69 (s, 1H, H-5), 6.56 (d, J=2.8 Hz, 1H, H-3′), 6.21 (t, J=3.1 Hz, 1H, H-4′), 3.95 (2s, 6H, 2° C. H3), 3.33 (m, 1H, H-1″), 3.13 (td, J=6.7, 1.8 Hz, 2H, H-3), 2.94 (t, J=6.5 Hz, 2H, H-4), 1.12 (td, J=7.2, 5.2 Hz, 2H, H-2a″ and H-3a″), 1.05-0.96 (m, 2H, H-2b″ and H-3b″); ¹³C-NMR (CDCl3, 150 MHZ) δ 186.0, 153.1, 148.1, 137.7, 131.1, 129.5, 127.0, 125.0, 124.9, 113.8, 109.8, 109.5, 108.8, 56.00, 55.96, 28.3, 27.8, 27.4, 7.1; FT-IR (KBr, cm⁻¹): 3132, 3087, 3014 (C═C—H), 2936, 2836 (C═C—H), 1649 (C═O), 1602, 1572, 1506 (C═C); Anal. calcd. For C20H21NO3: C, 74.28; H, 6.55; N, 4.33; Found: C, 74.05; H, 6.64; N, 4.45; LC-MS (+)-ESI (m/z): calculated 323.15, observed 324.2 [M+1]⁺.

### 9. (E)-3-(1-cyclopropyl-1H-pyrrol-2-yl)-1-(3-methoxyphenyl) prop-2-en-1-one (9)

Yellow-brown oil; yield: 37.4%; 1H-NMR (CDCl3 with 0.05% v/v of TMS, 600 MHZ) δ 8.07 (d, J=15.1 Hz, 1H, β-olefinic), 7.57 (dt, J=7.7, 1.3 Hz, 1H, H-6), 7.53 (t, J=2.1 Hz, 1H, H-2), 7.38 (t, J=7.9 Hz, 1H, H-5), 7.31 (d, J=15.8 Hz, 1H, α-olefinic), 7.09 (ddd, J=8.3, 2.8, 1.4 Hz, 1H, H-4), 6.88 (dd, J=2.8, 1.4 Hz, 1H, H-5′), 6.78 (dd, J=3.4, 1.4 Hz, 1H. H-3′), 6.17 (t, J=3.4 Hz, 1H, H-4′), 3.87 (s, 3H, OCH₃-3), 3.39-3.30 (m, 1H, H-1″), 1.10 (td, J=7.2, 5.3 Hz, 2H, H-2a″ and H-3a″), 1.03-0.98 (m, 2H, H-2b″ and H-3b″); ¹³C-NMR (CDCl3, 150 MHz) δ 189.9, 159.8, 140.3, 133.1, 131.6, 129.4, 126.9, 120.7, 118.7, 116.8, 112.8, 109.4, 55.4, 28.3, 7.2; FT-IR (KBr, cm⁻¹): 3096, 3008 (C═C—H), 2934, 2835 (C═C—H), 1652 (C—O), 1567 (C═C); Anal. calcd. for C17H17NO2: C, 76.38; H, 6.41; N, 5.24; Found: C, 75.45; H, 6.767; N, 5.34; LC-MS (+)-ESI (m/z): calculated 267.13, observed 268.1 [M+1]⁺.

### 10. (E)-6,7-dimethoxy-2-(thieno[3,2-b]thiophen-2-ylmethylene)-3,4-dihydronaphthalen-1 (2H)-one (10)

Yellow solid; yield: 71.7%; 1H-NMR (CDCl3, 600 MHZ) δ 8.01 (s, 1H, H-3′), 7.62 (s, 1H, H-8), 7.54 (s, 1H, β-olefinic), 7.48 (d, J=4.8 Hz, 1H, H-5′), 7.28 (d, J=4.8 Hz, 1H, H6′), 6.71 (s, 1H, H-5), 3.97 (s, 3H, OCH₃), 3.96 (s, 3H, OCH₃), 3.24 (td, J=6.5, 1.3 Hz, 2H, H-3), 3.00 (t, J=6.5 Hz, 2H, H-4); ¹³C-NMR (CDCl3, 150 MHZ) δ 185.8, 153.4, 148.3, 142.2, 141.4, 139.4, 137.9, 132.0, 129.6, 126.7, 124.7, 119.5, 109.8, 109.6, 56.3, 28.1, 27.4; FT-IR (KBr, cm⁻¹): 3113, 3081, 3008 (C═C—H), 2930, 2835 (C═C—H), 1646 (C—O), 1597, 1568, 1508 (C═C); Anal. calcd. for C19H1603S2: C, 64.02; H, 4.52; Found: C, 64.16; H, 4.48; N, 0.02; LC-MS (+)-ESI (m/z): calculated 356.05, observed 357.0 [M+1]⁺.

### 11. (E)-6,7-dimethoxy-2-((6-methoxypyridin-2-yl)methylene)-3,4-dihydronaphthalen-1 (2H)-one (11)

Off-white solid; yield: 66.13%; 1H-NMR (CDCl3, 600 MHz) δ 7.63 (s, 1H, H-8), 7.627.57 (m, 2H, β-olefinic and H-4′), 7.06 (d, J=7.6 Hz, 1H, H-3′ or H-5′), 7.10 (s, 1H, overlapped, H-5), 6.69 (d, J=7.6 Hz, 1H, H-3′ or H-5′), 3.98 (s, 3H, OCH₃), 3.96 (2S,6H,2° C. H3), 3.77-3.67 (m, 2H, H-3), 2.97 (t, J=6.2 Hz, 2H, H-4); ¹³C-NMR (CDCl3, 150 MHz) δ 187.1, 163.2, 153.5, 152.9, 148.1, 139.1, 138.8, 138.5, 132.8, 126.5, 120.8, 110.6, 109.9, 109.6, 56.1, 53.5, 28.6, 27.1; FT-IR (KBr, cm⁻¹): 3084 (C═C—H), 2920, 2850 (C═C—H), 1636 (C—O) 1609, 1582, 1509 (C═C); LC-MS (+)-ESI (m/z): calculated 325.13, observed 362.2 [M+1]⁺.

### 12. (E)-6,7-dimethoxy-2-((6-(trifluoromethyl) pyridin-2-yl)methylene)-3,4-dihydronaphthalen-1 (2H)-one (12)

White solid; yield: 67.0%; 1H-NMR (CDCl3, 600 MHz) δ 7.90 (t, J=7.9 Hz, 1H, H-4′), 7.69 (t, J=1.7 Hz, 1H, β-olefinic), 7.62 (s, 1H, H-8), 7.59 (dd, J=7.9, 4.5 Hz, 2H, H-3′ and H-5′), 6.71 (s, 1H, H-5), 3.97 (s, 3H, OCH₃), 3.96 (s, 3H, OCH₃), 3.63 (td, J=6.5, 1.4 Hz, 2H, H-3), 2.97 (t, J=6.5 Hz, 2H, H-4); ¹³C-NMR (CDCl3, 150 MHZ) δ 186.8, 155.9, 153.8, 148.3, 141.5, 139.4, 137.8, 130.7, 129.1, 126.2, 118.9, 109.9, 109.6, 56.13, 56.08, 28.4, 27.0; FT-IR (KBr, cm⁻¹): 3070 (C═C—H), 2928, 2839 (C═C—H), 1657 (C═O), 1588, 1511 (C═C); Anal. calcd. for C19H16F3NO3: C, 62.81; H, 4.44; N, 3.86; Found: C, 62.99; H, 4.60; N, 4.01; LC-MS (+)-ESI (m/z): calculated 363.11, observed 364.1 [M+1]⁺.

### 13. (E)-6,7-dimethoxy-2-((6-(thiophen-3-yl) pyridin-3-yl)methylene)-3,4-dihydronaphthalen-1 (2H)-one (13)

Pale yellow solid; yield: 80.3%; 1H-NMR (CDCl3 with 0.05% v/v of TMS, 600 MHZ) δ 7.94 (dd, J=2.8, 1.4 Hz, 1H, H-2″), 7.81-7.70 (m, 3H, H-4′, β-olefinic and H-4″), 7.65 (s, 1H, H-8), 7.55 (d, J=8.3 Hz, 1H, H-5′), 7.42 (dd, J=4.8, 2.8 Hz, 1H, H-5″), 7.32 (d, J=7.6 Hz, 1H, H-3′), 6.71 (s, 1H, H-5), 3.96 (s, 6H, OCH₃-7 and OCH₃-8), 3.80-3.72 (m, 2H, H-3), 2.99 (t, J=6.2 Hz, 2H, H-4); ¹³C-NMR (CDCl3, 150 MHZ) δ 187.1 (C—O), 155.2 (C-2′), 153.6 (C-7), 152.8 (C-6′), 148.2 (C-6), 142.3 (C-3″), 139.2, 139.1 (C-4a and C-2), 137.1 (C-4′), 132.9 (CB), 126.5 (C-5″), 126.29 (C-4″), 126.26 (C-8a), 125.2 (C-3′), 123.7 (C-2″), 119.0 (C-5′), 109.9 (C-5), 109.6 (C-8), 56.1 (O—CH₃), 56.0 (O—CH₃), 28.6 (C-4), 27.2 (C3); FT-IR (KBr, cm⁻¹): 3092 (C═C—H), 2918, 2851 (C—C—H), 1656 (C═O), 1586, 1508 (C═C), 1262 (C═S); Anal. calcd. for C22H19NO3S: C, 70.01; H, 5.07; N, 3.71; Found: C, 68.54; H, 5.06; N, 3.74; LC-MS (+)-ESI (m/z): calculated 377.11, observed 378.9 [M+1]⁺.

### 14. (E)-6-methoxy-2-((6-(thiophen-3-yl) pyridin-2-yl)methylene)-3,4-dihydronaphthalen-1 (2H)-one (14)

Beige solid; yield: 70.9%; 1H-NMR (CDCl3 with 0.05% v/v of TMS, 600 MHZ) δ 8.14 (d, J=8.3 Hz, 1H, H-8), 7.94 (dd, J=2.8, 1.4 Hz, 1H, H-2″), 7.74 (t, J=7.6, 1H, H-4′), 7.73 (overlapped, 1H, β-olefinic), 7.71 (dd, J=5.5, 1.4 Hz, 1H, H-4″), 7.55 (d, J=7.6 Hz, 1H, H-5′), 7.42 (dd, J=5.2, 3.1 Hz, 1H, H-5″), 7.32 (d, J=7.6 Hz, 1H, H-3′), 6.89 (dd, J=8.6, 2.4 Hz, 1H, H-7), 6.74 (d, J=2.1 Hz, 1H, H-5), 3.88 (s, 3H, OCH3-6), 3.75 (td, J=6.5, 1.8 Hz, 2H, H-3), 3.02 (t, J=6.2 Hz, 2H, H-4); ¹³C-NMR (CDCl3, 150 MHZ) δ 187.2, 163.6, 155.2, 152.9, 146.5, 142.3, 139.4, 137.1, 132.9, 130.8, 127.0, 126.3, 125.3, 123.7, 119.0, 113.3, 112.3, 55.4, 29.2, 27.0; Anal. calcd. for C21H17NO2S: C, 72.60; H, 4.93; N, 4.03; Found: C, 71.33; H, 4.84; N, 4.13; LC-MS (+)-ESI (m/z): calculated 347.10, observed 348.0 [M+1]⁺.

### 15. (E)-7-methoxy-2-((6-(thiophen-3-yl) pyridin-2-yl)methylene)-3,4-dihydronaphthalen-1 (2H)-one (15)

Fluffy off-white crystalline solid; yield: 73.9%; 1H-NMR (CDCl3 with 0.05% v/v of TMS, 600 MHz) δ 7.94 (dd, J=3.4, 1.4 Hz, 1H, H-2″), 7.75 (t, J=7.6 Hz, 1H, H-4′), 7.75 (overlapped, 1H, β-olefinic), 7.72 (dd, J=5.5, 1.4 Hz, 1H, H-4″), 7.64 (d, J=2.8 Hz, 1H, H8), 7.56 (d, J=8.3 Hz, 1H, H-5′), 7.42 (dd, J=5.2, 3.1 Hz, 1H, H-5″), 7.33 (d, J=8.3 Hz, 1H, H-3′), 7.20 (d, J=8.3 Hz, 1H, H-5), 7.10 (dd, J=8.6, 3.1 Hz, 1H, H-6), 3.88 (s, 3H, OCH₃-7), 3.74 (td, J=6.5, 1.8 Hz, 2H, H-3), 2.99 (t, J=6.5 Hz, 2H, H-4); ¹³C-NMR (CDCl3, 150 MHZ) δ 187.2, 163.6, 155.2, 152.9, 146.5, 142.3, 139.4, 137.1, 132.9, 130.8, 127.0, 126.3, 125.3, 123.7, 119.0, 113.3, 112.3, 55.4, 29.2, 27.0; FT-IR (KBr, cm⁻¹): 3107 (C═C—H), 2927, 2833 (C═C—H), 1662 (C═O), 1597, 1579, 1494 (C═C), 1240 (C═S); Anal. calcd. for C21H17NO2S: C, 72.60; H, 4.93; N, 4.03; Found: C, 66.35; H, 4.58; N, 3.91; LC-MS (+)-ESI (m/z): calculated 347.10, observed 348.0 [M+1]⁺.

### 16. (E)-1-(3-methoxyphenyl)-3-(6-(thiophen-3-yl) pyridin-2-yl) prop-2-en-1-one (16)

White solid; yield: 72.7%; 1H-NMR (CDCl3 with 0.05% v/v of TMS, 600 MHZ) δ 8.16 (d, J=15.8 Hz, 1H, β-olefinic), 8.01 (dd, J=2.4, 1.0 Hz, 1H, H-2″), 7.79 (d, J=15.1 Hz, 1H, α-olefinic), 7.77-7.73 (m, 2H, H-4″ and H-4′), 7.70 (d, J=7.6 Hz, 1H, H-6), 7.63 (d, J=8.3 Hz, 1H, H-5′), 7.61 (dd, J=2.8, 1.4 Hz, 1H, H-2), 7.49-7.41 (m, 2H, H-5 and H-5″), 7.36 (d, J=7.6 Hz, 1H, H-3′), 7.16 (dd, J=8.3, 2.8 Hz, 1H, H-4), 3.90 (s, 3H, OCH₃-3); ¹³C-NMR (CDCl3, 150 MHZ) δ 190.6 (C—O), 159.9 (C-3), 153.6 (C-6′), 152.8 (C-2′), 143.1 (C—β), 141.9, (C3″), 139.4 (C-1), 137.5 (C-4′), 129.6 (C-5), 126.4, 126.3 (C-4″ and C-5″), 125.8 (C—α), 124.2 (C-2″), 123.4 (C-3′), 121.4 (C-6), 121.1 (C-5′), 119.6 (C-4), 112.8 (C-2), 55.5 (OCH₃); FT-IR (KBr, cm⁻¹): 3105 (C═C—H), 2999, 2933, 2833 (C═C—H), 1663 (C═O), 1615, 1600, 1575, 1524 (C═C), 1261 (C═S); Anal. calcd. for C19H15NO2S: C, 71.01; H, 4.70; N, 4.36; Found: C, 71.19; H, 4.65; N, 4.43; LC-MS (+)-ESI (m/z): calculated 321.08, observed 322.8 [M+1]⁺.

### 17. (E)-1-(2-methoxyphenyl)-3-(6-(thiophen-3-yl) pyridin-2-yl) prop-2-en-1-one (17)

Pale yellow gummy mass; 1H-NMR (CDCl3 with 0.05% v/v of TMS, 600 MHz) δ 8.00-7.92 (m, 2H, H2″ and β-olefinic), 7.76-7.70 (m, 2H, H-4″ and H-4′), 7.65 (dd, J=7.6, 2.1 Hz, 1H, H-6), 7.63-7.57 (m, 2H, α-olefinic and H-5′), 7.49 (td, J=7.7, 1.8 Hz, 1H, H-4), 7.40 (dd, J=5.2, 3.1 Hz, 1H, H-5″), 7.33 (d, J=7.6 Hz, 1H, H-3′), 7.05 (t, J=7.9 Hz, 1H, H-5), 7.01 (d, J=8.3 Hz, 1H, H-3), 3.93 (s, 3H, OCH₃-2); ¹³C-NMR (CDCl3, 150 MHZ) δ 193.3, 158.3, 153.5, 153.2, 142.0, 141.9, 137.3, 133.0, 130.6, 130.3, 129.1, 126.3, 123.9, 122.7, 120.7, 120.6, 111.7, 55.7; LC-MS (+)-ESI (m/z): calculated 321.08, observed 322.2 [M+1]⁺.

### 18. (E)-1-(4-methoxyphenyl)-3-(6-(thiophen-3-yl) pyridin-2-yl) prop-2-en-1-one (18)

Fluffy white solid; yield: 78.7%; 1H-NMR (CDCl3 with 0.05% v/v of TMS, 600 MHZ) δ 8.21 (d, J=15.1 Hz, 1H, β-olefinic), 8.12 (dt, J=9.4, 2.6 Hz, 2H, H-2 and H-6), 8.01 (dd, J=2.9, 1.2 Hz, 1H, H-2″), 7.80-7.76 (m, 2H, α-olefinic and H-4″), 7.74 (t, J=7.6 Hz, 1H, H-4′), 7.62 (d, J=7.6 Hz, 1H, H-5′), 7.43 (dd, J=4.8, 3.4 Hz, 1H, H-5″), 7.34 (d, J=7.6 Hz, 1H, H-3′), 7.01 (dt, J=9.4, 2.4 Hz, 2H, H-3 and H-5), 3.90 (s, 3H, OCH₃-4); ¹³C-NMR (CDCl3, 150 MHz) δ 189.0, 163.56, 153.5, 153.0, 142.2, 142.0, 137.5, 131.0, 126.4, 126.3, 125.6, 124.1, 123.4, 120.9, 113.9, 55.5; FT-IR (KBr, cm⁻¹): 3109, 3091 (C═C—H), 2961 (C═C—H), 1660 (C═O), 1600, 1575, 1527, 1509 (C═C), 1248 (C═S); Anal. calcd. for C19H15NO2S: C, 71.01; H, 4.70; N, 4.36; Found: C, 70.04; H, 4.77; N, 4.44; LC-MS (+)-ESI (m/z): calculated 321.08, observed 322.1 [M+1]⁺.

### 19. (E)-1-(3,4-dimethoxyphenyl)-3-(6-(thiophen-3-yl) pyridin-2-yl) prop-2-en-1-one (19)

Fluffy white solid; yield: 87.3%; 1H-NMR (CDCl3 with 0.05% v/v of TMS, 600 MHZ) δ 8.22 (d, J=15.1 Hz, 1H, β-olefinic), 8.01 (dd, J=3.4, 1.4 Hz, 1H, H-2″), 7.86-7.70 (m, 4H, H-6, α-olefinic, H-4″, H-4′), 7.68 (d, J=2.1 Hz, 1H, H-2), 7.62 (d, J=6.9 Hz, 1H, H-5′), 7.43 (dd, J=5.2, 3.1 Hz, 1H, H-5″), 7.35 (d, J=7.6 Hz, 1H, H-3′), 6.97 (d, J=8.3 Hz, 1H, H-5), 4.00, 3.97 (2s, 6H, OCH₃-3 and OCH₃-4); ¹³C-NMR (CDCl3, 150 MHz) δ 188.9, 153.5, 153.4, 153.0, 149.3, 142.2, 142.0, 137.5, 131.2, 126.4, 126.3, 125.4, 124.1, 123.5, 123.4, 120.9, 110.7, 110.0, 56.1, 56.0; FT-IR (KBr, cm⁻¹): 3103 (C═C—H), 2961, 2839 (C═C—H), 1660 (C—O), 1610, 1578, 1512 (C═C), 1270 (C═S); Anal. calcd. for C20H17NO3S: C, 68.36; H, 4.88; N, 3.99; Found: C, 67.04; H, 4.84; N, 4.13; LC-MS (+)-ESI (m/z): calculated 351.09, observed 352.1 [M+1]⁺.

### 20. (E)-3-(6-(thiophen-3-yl) pyridin-2-yl)-1-(2,4,6-trimethoxyphenyl) prop-2-en-1-one

(20) Off-white solid; yield: 49.8%; 1H-NMR (CDCl3, 600 MHz) § 8.04 (d, J=15.8 Hz, 1H, β-olefinic), 7.97 (dd, J=3.4, 1.4 Hz, 1H, H-2″), 7.80-7.69 (m, 2H, H-4″ and H-4″), 7.66 (d, J=15.8 Hz, 1H, α-olefinic), 7.60 (d, J=7.6 Hz, 1H, H-5′), 7.53 (d, J=9.0 Hz, 1H, H-6), 7.40 (dd, J=4.8, 3.4 Hz, 1H, H-5″), 7.35 (d, J=6.9 Hz, 1H, H-3′), 6.77 (d, J=8.3 Hz, 1H, H-5), 3.99 (s, 3H, OCH₃-2), 3.93 (s, 3H, OCH₃), 3.92 (s, 3H, OCH₃); ¹³C-NMR (CDCl3, 150 MHz) δ 191.1, 157.2, 154.0, 153.5, 153.3, 142.1, 142.0, 141.6, 137.3, 130.3, 126.5, 126.28, 126.25, 125.9 123.9, 122.7, 120.6, 107.2, 62.0, 61.0, 56.1; FT-IR (KBr, cm⁻¹): 3123 (C═C—H), 2964 (C═C—H), 1652 (C═O), 1601, 1582, 1526 (C═C); Anal. calcd. for C21H19NO4S: C, 66.12; H, 5.02; N, 3.67; Found: C, 65.61; H, 5.11; N, 3.66; LC-MS (+)-ESI (m/z): calculated 381.10, observed 382.0 [M+1]⁺.

### 21. (E)-3-(6-(thiophen-3-yl) pyridin-2-yl)-1-(2,4,6-trimethoxyphenyl) prop-2-en-1-one (21)

White solid; 1H-NMR (CDCl3, 600 MHz) δ 7.94 (dd, J=3.4, 1.4 Hz, 1H, H-2″), 7.73-7.67 (m, 2H, H-4′ and H-4″), 7.56 (d, J=7.6 Hz, 1H, H-5′), 7.47 (d, J=15.8 Hz, 1H, β-olefinic), 7.42-7.34 (m, 2H, α-olefinic and H-5″), 7.31 (d, J=7.6 Hz, 1H, H-3′), 6.17 (s, 2H, H-3 and H-5), 3.86 (s, 3H, OCH₃-4), 3.77 (s, 6H, OCH₃-2 and OCH₃-6); ¹³C-NMR (CDCl3, 150 MHZ) & 194.6, 162.5, 158.9, 153.5, 153.4, 142.9, 141.9, 137.2, 132.4, 126.3, 124.0, 122.3, 120.5, 111.7, 90.7, 55.9, 55.4; FT-IR (KBr, cm⁻¹): 3104,3052 (C═C—H), 2939, 2839 (C═C—H), 1635 (C—O), 1607, 1584 (C═C); Anal. calcd. for C21H19NO4S: C, 66.12; H, 5.02; N, 3.67; Found: C, 65.64; H, 5.15; N, 3.68; LC-MS (+)-ESI (m/z): calculated 381.10, observed 382.0 [M+1]′.

### 22. (E)-3-(6-(thiophen-3-yl) pyridin-2-yl)-1-(3,4,5-trimethoxyphenyl) prop-2-en-1-one (22)

Pale yellow solid; yield: 58.0%; 1H-NMR (CDCl3 with 0.05% v/v of TMS, 600 MHZ) δ 8.13 (d, J=15.1 Hz, 1H, β-olefinic), 7.99 (dd, J=2.6, 1.2 Hz, 1H, H-2″), 7.85-7.72 (m, 3H, α-olefinic, H-4 and H-4″), 7.64 (d, J=7.6 Hz, 1H, H-5′), 7.43 (dd, J=4.8, 3.4 Hz, 1H, H5″), 7.37 (d, J=7.6 Hz, 1H, H-3′), 7.34 (s, 2H, H-2 and H-6), 3.97 (s, 6H, OCH₃-3 and OCH₃-5), 3.95 (s, 3H, OCH₃-4); ¹³C-NMR (CDCl3, 150 MHz) δ 189.6, 153.6, 153.1, 152.8, 143.0, 142.7, 141.9, 137.6, 133.3, 126.4, 126.2, 125.5, 124.1, 123.4, 121.1, 106.3, 61.0, 56.4; LC-MS (+)-ESI (m/z): calculated 381.10, observed 382.1 [M+1]⁺.

### 23. (E)-1-(4-(methylsulfonyl)phenyl)-3-(6-(thiophen-3-yl) pyridin-2-yl) prop-2-en-1-one (23)

Fluffy pale yellow solid; yield: 79.1%; 1H-NMR (CDCl3 with 0.05% v/v of TMS, 600 MHz) δ 8.24 (d, J=8.3 Hz, 2H, H-2 and H-6), 8.15 (d, J=15.1 Hz, 1H, β-olefinic), 8.12 (d, J=8.3 Hz, 2H, H-3 and H-5), 8.01 (dd, J=3.4, 1.4 Hz, 1H, H-2″), 7.86-7.75 (m, 3H, α-olefinic, H-4′ and H-4″), 7.67 (d, J=8.3 Hz, 1H, H-5′), 7.45 (dd, J=5.2, 3.1 Hz, 1H, H-5″), 7.38 (d, J=7.6 Hz, 1H, H-3′), 3.12 (s, 3H, CH₃); ¹³C-NMR (CDCl3, 150 MHz) δ 189.8, 153.8, 152.2, 144.7, 143.8, 142.2, 141.7, 137.7, 129.4, 127.8, 126.6, 126.3, 125.0, 124.3, 123.9, 121.6, 44.4; FT-IR (KBr, cm⁻¹): 3075, 3010 (C═C—H), 2925, 2852 (C═C—H), 1660 (C═O), 1603, 1563, 1524 (C═C), 1304, 1287 (S═O), 1260 (C═S); Anal. calcd. for C19H15NO3S2: C, 61.77; H, 4.09; N, 3.79; Found: C, 62.42; H, 5.00; N, 3.25; LC-MS (+)-ESI (m/z): calculated 369.05, observed 370.0 [M+1]⁺.

### 24. (E)-1-(2-hydroxyphenyl)-3-(6-(thiophen-3-yl) pyridin-2-yl) prop-2-en-1-one (24)

Fluffy yellow solid; yield: 80.7%; 1H-NMR (CDCl3 with 0.05% v/v of TMS, 600 MHz) § 12.80 (s, 1H, OH), 8.34 (d, J=14.5 Hz, 1H, β-olefinic), 8.04 (dd, J=8.3, 1.4 Hz, 1H, H-6), 8.02 (dd, J=3.4, 1.4 Hz, 1H, H-2″), 7.88 (d, J=15.1 Hz, 1H, α-olefinic), 7.81-7.74 (m, 2H, H-4′ and H-4″), 7.65 (d, J=6.9 Hz, 1H, H-5′), 7.59-7.50 (m, 1H, H-4), 7.45 (dd, J=5.2, 3.1 Hz, 1H, H-5″), 7.37 (d, J=7.6 Hz, 1H, H-3′), 7.05 (dd, J=8.3, 1.4 Hz, 1H, H-3), 6.97 (td, J=7.6, 1.4 Hz, 1H, H-5); 13C-NMR (CDCl3, 150 MHz) δ 194.2, 163.6, 153.7, 152.4, 143.5, 141.8, 137.6, 136.6, 130.2, 126.5, 126.3, 124.3, 124.1, 123.9, 121.4, 120.1, 119.0, 118.5; FT-IR (KBr, cm⁻¹): 3104 (O—H), 3061 (C═C—H), 1660 (C═O), 1603, 1563, 1524 (C═C), 1304, 1287 (S═O), 1260 (C═S); Anal. calcd. for C18H13NO2S: C, 70.34; H, 4.26; N, 4.56; Found: C, 69.12; H, 4.14; N, 4.81; LC-MS (+)-ESI (m/z): calculated 307.07, observed 308.0 [M+1]⁺.

### 25. (E)-3-(6-(thiophen-3-yl) pyridin-2-yl)-1-(2-(trifluoromethyl)phenyl) prop-2-en-1-one (25)

Pale yellow solid; yield: 61.2%; 1H-NMR (CDCl3, 600 MHz) δ 7.96 (dd, J=2.8, 1.4 Hz, 1H, H-2″), 7.79 (d, J=8.3 Hz, 1H, H-3), 7.73 (t, J=7.9 Hz, 1H, H-4′), 7.71 (dd, J=5.5, 1.4 Hz, 1H, H-4″), 7.68-7.60 (m, 4H, H-4, β-olefinic, H-5′ and H-5), 7.52 (d, J=7.6 Hz, 1H, H-6), 7.40 (dd, J=4.8, 2.8 Hz, 1H, H-5″), 7.37-7.28 (m, 2H, α-olefinic, H-3′); ¹³C-NMR (CDCl3, 150 MHz) δ 195.3, 153.8, 152.2, 146.2, 141.6, 138.7, 137.5, 131.6, 130.1, 130.0, 128.1, 127.9 (q, J=32.3 Hz, C-2), 126.8 (q, J=4.8 Hz, C-3 or C-1), 126.5, 126.2, 124.3, 123.6 (q, J=274.5, CF₃) 123.0, 121.3; FT-IR (KBr, cm⁻¹): 3040 (C═C—H), 2984 (C═C—H), 1677 (C═O), 1614, 1578, 1522 (C═C), 1308 (C—F), 1270 (C═S); Anal. calcd. for C19H12F3NOS: C, 63.50; H, 3.37; N, 3.90; Found: C, 62.97; H, 3.53; N, 4.09; LC-MS (+)-ESI (m/z): calculated 359.06, observed 359.9 [M+1]⁺.

### 26. (E)-1-(4-fluorophenyl)-3-(6-(thiophen-3-yl) pyridin-2-yl) prop-2-en-1-one (27)

Off-white Solid; yield: 64.7%; 1H-NMR (CDCl3, 600 MHz) δ 8.17 (d, J=15.1 Hz, 1H, β-olefinic), 8.14 (m, 2H, H-2 and H-6), 8.01 (dd, J=2.8, 1.4 Hz, 1H, H-2″), 7.86-7.73 (m, 3H, α-olefinic, H-4′ and H-4″), 7.64 (d, J=7.6 Hz, 1H, H-5′), 7.44 (dd, J=5.2, 3.1 Hz, 1H, H-5″), 7.36 (d, J=7.6 Hz, 1H, H-3′), 7.20 (tt, J=9.0, 2.4 Hz, 2H, H-3 and H-5).

### In Silico ADMET Screening

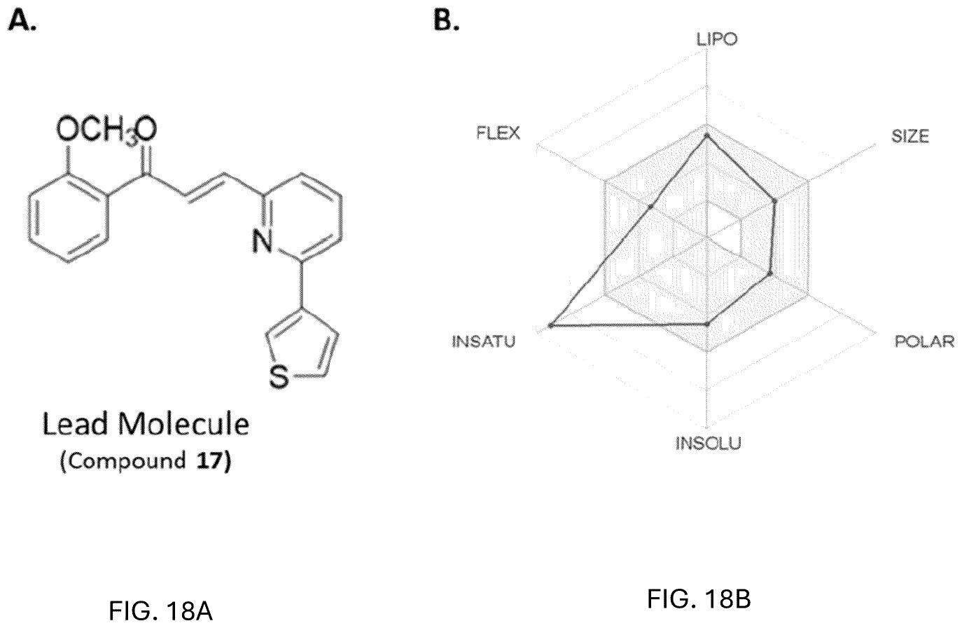

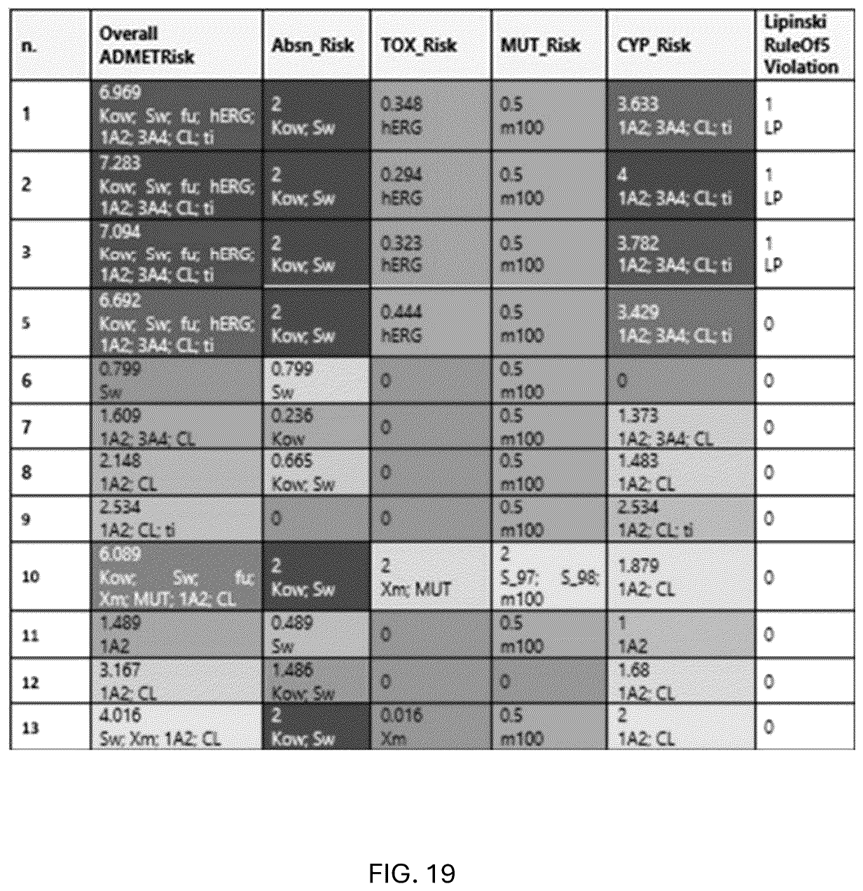

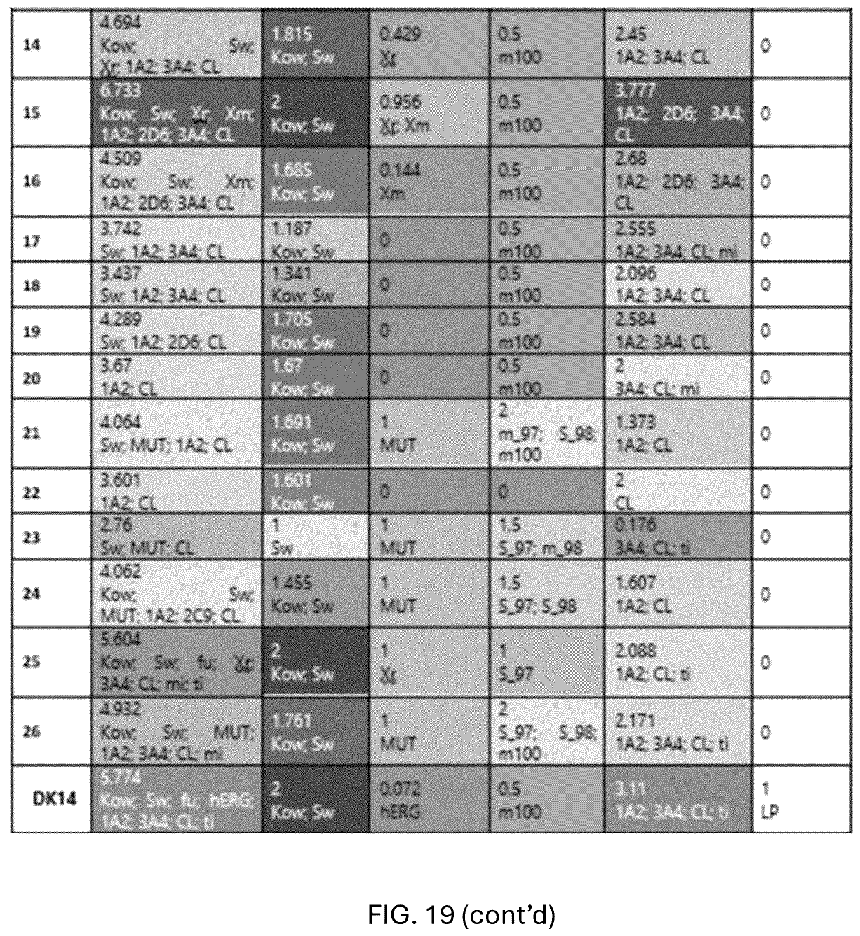

In this study, Applicant incorporated absorption, distribution, metabolism, excretion and toxicity (ADMET) in silico prediction in the design process to screen all the proposed analogs and prioritize hits with favorable profiles. Two ADMET predicting software were used, ADMET Predictor and SwissADME. FIGS. 18A-18B shows the predicted drug-likeness properties for compound 17 (generated by the SwissADME web tool). The lead compound showed favorable properties in all tested aspects except for insaturation. Nevertheless, this is not a major limitation as this property is related to the feasibility of synthesis rather than the pharmacokinetic profile. The expected ADMET risk scores for all analogs as predicted by ADMET Predictor are summarized in FIGS. 18A and 18B. Generally, all thienyl-based chalcones (13-26), the most active series in the study, had favorable ADMET profiles with overall risk below the ADMET threshold of 6.5, except for compound 15.

General Screening (Compounds 1-16) Against Prostate Cancer Cell lines

Initially, sixteen novel chalcone analogs were synthesized and screened for their antiproliferative activity and effect on cell morphology against two of the most aggressive AR negative human prostate cancer cell lines (PC3 and DU145), which represent castration-resistant prostate cancer (CRPC). Compounds were screened at 10 M concentrations in comparison to the vehicle (0.1% DMSO) as the negative control, docetaxel as positive control, and the parent compound (DK14) as reference. Compounds showing >50% reduction in cell viability at 10 μM were selected for dose-response evaluation and further anticancer mechanistic study.

### Cell Viability

The in vitro cytotoxicity of synthesized chalcones against PCa and TNBC cell lines was evaluated using AlamarBlue viability assay. AlamarBlue is a reliable and highly sensitive method that can quantitively reflect cell viability. AlamarBlue (resazurin) is a cell-permeable, weakly fluorescent blue dye that is reduced by mitochondrial enzymes in viable cells to resorufin, a highly fluorescent pink molecule. Unlike the widely used MTT (3-(4,5-dimethyl thiazolyl-2)-2,5-diphenyltetrazolium bromide) colorimetric assay, AlamarBlue has the advantage of being water-soluble; thus, it does not need crystal solubilization and can be directly quantified after reduction by fluorescence or UV reader.

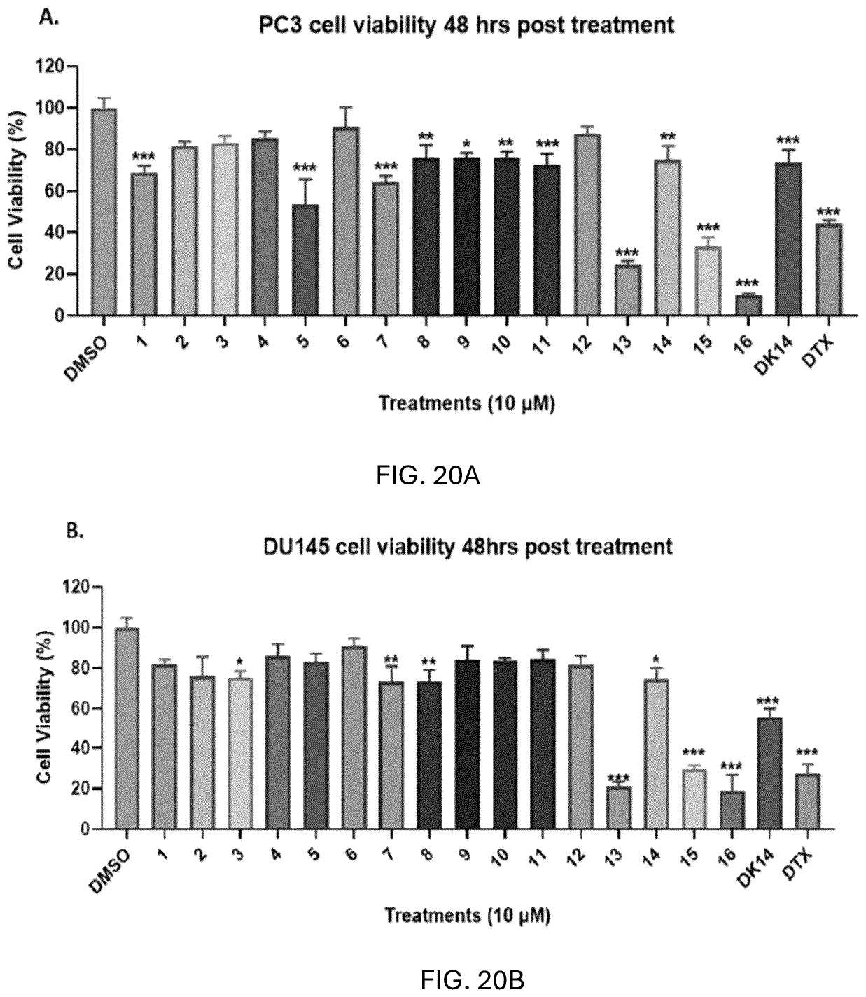

The cytotoxic activities of compounds (1-16) were primarily screened at 10 μM concentration against AR negative PCa cell lines (PC3 and DU145) using docetaxel as a positive control. After 48 hours of treatment, the cell viability of treated cells was calculated relative to DMSO-treated controls (FIGS. 20A-20B). Overall, out of the sixteen tested compounds, thienyl pyridine containing chalcones (13 and 16) induced the most significant reduction in cell viability in both cell lines, which was more prominent than the reduction with the reference drug (docetaxel). Interestingly, thienyl pyridine was not previously linked to in any chalcone, and Applicantare the first group to report this molecular hybrid.

### Effect of Series a (Compounds 1-5)

Based on the excellent activity of DK14 chalcone on TNBC (34), closed ring (tetralone-based) chalcone analogs bearing the same substituents (OCH₃ and nitrogen mustard) were synthesized and evaluated against prostate cancer cell lines. The cell viability results indicated that both DK14 and its related tetralone based chalcone analogs (compounds 1-5) displayed moderate to no cytotoxic effect at 10 μM (<50% reduction in viability) against PCa cell lines (PC3 and DU145) as compared to TNBC cell lines (IC50 6.3-9.22 μM). Compounds with a methoxy substituent at position-5 (compound 1 and 5) displayed preferential cytotoxicity to PC3 cell line, whereas analogs bearing methoxy at position 7 (compound 3) or its equivalent in open-chain chalcone (DK14) induced higher cytotoxicity in DU145. Although DK14 was slightly more cytotoxic than its corresponding tetralone analog in both tested cell lines, the difference between them was not statistically significant (P-value 0.44 and 0.99).

### Effect of Series B (Compounds 6-9)

Next, a second attempt was made to improve the activity and pharmacokinetic profile of DK 14. In this series, the nitrogen mustard group in DK14 was replaced with cyclopropyl pyrrole, a potential bio-isostere with a highly improved pharmacokinetic profile compared to the parent compound. Similar to series A, pyrrole-based chalcones showed marginal cytotoxicity. Comparing the cytotoxicity of DK14 with its matched cyclopropyl pyrrole-based non-cyclic chalcone (9) revealed a statistically significant difference in DU145 cell line in-favor DK14 (P-value 0.02). On the other hand, cyclopropyl pyrrole cyclic (tetralone-based) chalcones (7 and 8) bearing a different methoxy substituents pattern showed comparable cytotoxicity to DK14. Notably, H-pyrrole tetralone-based chalcone (6) did not significantly reduce cell proliferation in any of the cell lines, suggesting that cyclopropyl moiety is involved for anticancer activity.

### Effect of Series C (Compounds 10-16)

Due to unsatisfactory results with DK14 related analogs, a new line of chalcone derivatives was synthesized and evaluated. Among different heteroaromatic rings, a focus was given to the pyridine ring due to its promising anticancer activity and repeated success in the clinically approved drugs. Different 3′-pyridine substituted chalcones were developed, among which thienyl pyridine-containing analogs (13, 15, and 16) showed excellent cytotoxicity. Compound 16 induced the most significant reduction in cell viability of PC3 and DU145 (89.94% and 81.05%, respectively). Interestingly, compound 16 performed better than the first-line drug (docetaxel), especially in PC3 cell line (89.94% vs. 55.89%, P-value <0.01). Although docetaxel started killing cancer cells at a much lower concentration and displayed a lower IC50, its cytotoxicity reached a plateau and was less effective than the synthesized analogs at 10 μM concentration, suggesting the presence of a docetaxel resistant subpopulation. Based on the highly promising activity, compounds 13, 15 and 16, were selected for further evaluation to assess their antitumor potential and explore their mechanism of action against prostate cancer.

### Morphological Examination

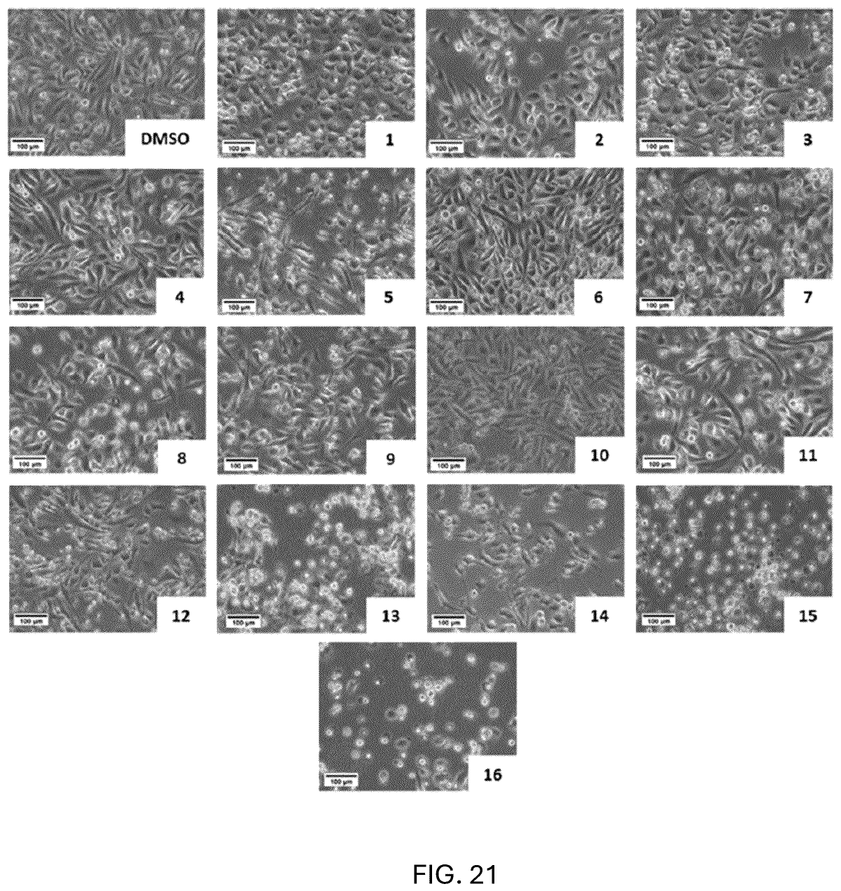

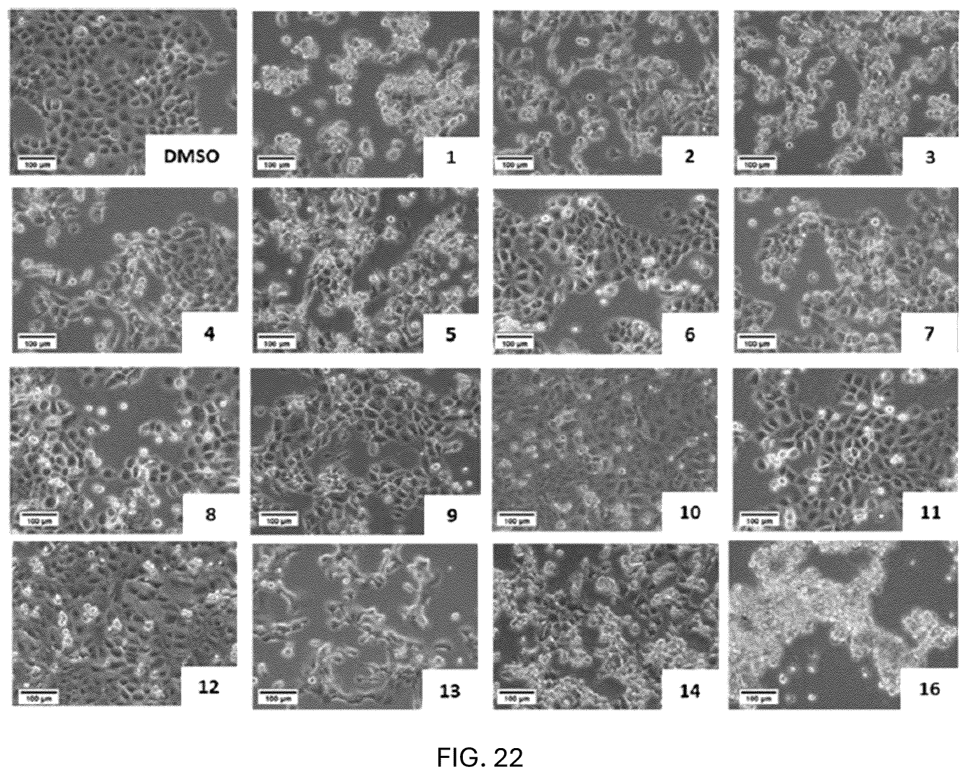

The induction of apoptosis in cancer cells has been widely considered an important criterion in developing anticancer drugs. To confirm whether the synthesized compounds could reduce cell viability and induce apoptosis in PC3 and DU145, prostate cancer cells were treated with compounds 1-16 at 10 μM concentrations for 48 hours. Cells were examined for the formation of apoptotic bodies or death by phase-contrast microscopy (FIGS. 21 and 22). More importantly, cells were monitored for characteristic changes in cell morphology as they can provide insight into cell differentiation, invasiveness, survival, and genetic alterations.

Consistent with the cell viability results, compounds 13, 15 and 16 induced the most significant morphological changes. Treatment with these compounds for 48 hours caused cell detachment, shrinkage, and wall deformation, indicating cell death in PC3 and DU145. Notably, nitrogen mustard-containing compounds (1 and 3) caused the elongated spindle-like PC3 cells to become more round and larger in size. Since small cell size and elongated shape are correlated with the metastatic potential in human cancer cells, these compounds might have a role in blocking metastasis (42). These findings are consistent with observation reported in Khalifa et al. study on related non-cyclic nitrogen mustard containing chalcone (DK14), which shows a similar pattern of morphological changes in addition to a reduction in cell migration and invasion. Likewise, the cyclopropyl-containing compound (7) caused PC3 cells to be rounder, but to a lower extent due to the formation of apoptotic bodies, suggesting that this moiety can act as a potential bio isostere for nitrogen mustard. For the scope of this study, thienyl pyridine-based chalcones compounds 13-16 were tested.

### Molecular Pathway Study of Compounds 13, 15, and 16

Based on the cell viability and morphological examination screening of the first sixteen synthesized analogs, thienyl pyridine-based chalcones (13, 15 and 16) showed the most promising activity against both cell lines (PC3 and DU145). Therefore, these compounds were selected for further mechanistic studies to evaluate their antitumor potential on castration-resistant prostate cancer (CRPC).

Since the ultimate goal of designing a new drug is to improve the efficacy and reduce non-specific toxicities, the IC50 against PCa cell lines and LD50 against normal cells for the selected analogs were evaluated to assess the potency and selectivity. Next, additional assays were conducted to explore the mechanism behind the observed anticancer activity. The effect of the compounds on the main carcinogenesis-related cellular processes, namely colony formation, cell cycle, apoptosis, migration, EMT, and angiogenesis, was explored. Besides, their effect on major cancer-related molecular pathways was studied by western blot. The compounds were studied in vitro against AR-negative PCa cell lines (PC3 and DU145) and in ovo using the CAM of the chicken embryos. Docetaxel, first-line chemotherapy for CRPC, was used as a reference drug and positive control.

### IC50 and Selectivity Evaluation

To assess whether the new analogs can serve as potential lead anticancer agents with improved efficacy and safety profile, the IC50 and LD50 were calculated for compounds showing >50% reduction in the initial cell viability screening. Prostate cancer (PC3 and DU145) and normal primary dental bulb cells were treated with increasing concentrations of compounds (13, 15 and 16) for 48 hours, and their viability was assessed by Alamar Blue.

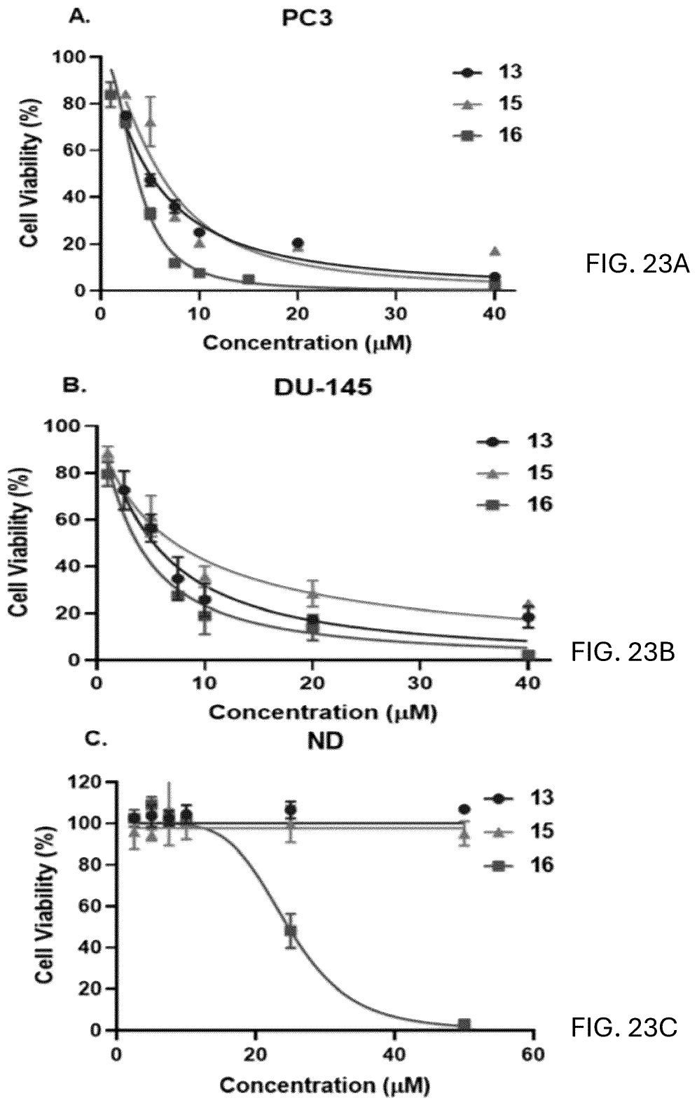

As seen in FIGS. 23A-23C, the three compounds showed a dose-dependent reduction in cell viability against both PC3 and DU145 cell lines. Compound 16 showed the most potent cytotoxicity with an IC50 of 4.3 and 5.2 M against PC3 and DU145, respectively (Table 4). All analogs were slightly more potent in PC3 than in DU145. Interestingly, the compounds exhibited preferential cytotoxicity towards prostate cancer cell line as compared to normal cells. Compounds were at least 4.7-8.8 times more toxic to cancer cells than normal cells.

TABLE 4
IC50 and LD50 of compounds 13, 15 and 16 on
AR-negative PCa and normal dental cells.

| | 13 | 15 | 16 |
|---|---|---|---|
| PC3 IC50 (μM) | 5.60 ± 0.38 | 6.60 ± 0.49 | 4.32 ± 0.37 |
| DU145 IC50 (μM) | 5.63 ± 1.07 | 6.47 ± 0.57 | 5.20 ± 0.46 |
| ND LD50 (μM) | >50.0 | >50.0 | 24.67 ± 1.03 |

ND = Normal primary dental cells; Values are expressed as Mean ± SEM of three independent experiments. IC50 and LD50 for each experiment were computed by non-linear regression using GraphPad Prism.

### Effect on Cell Morphology

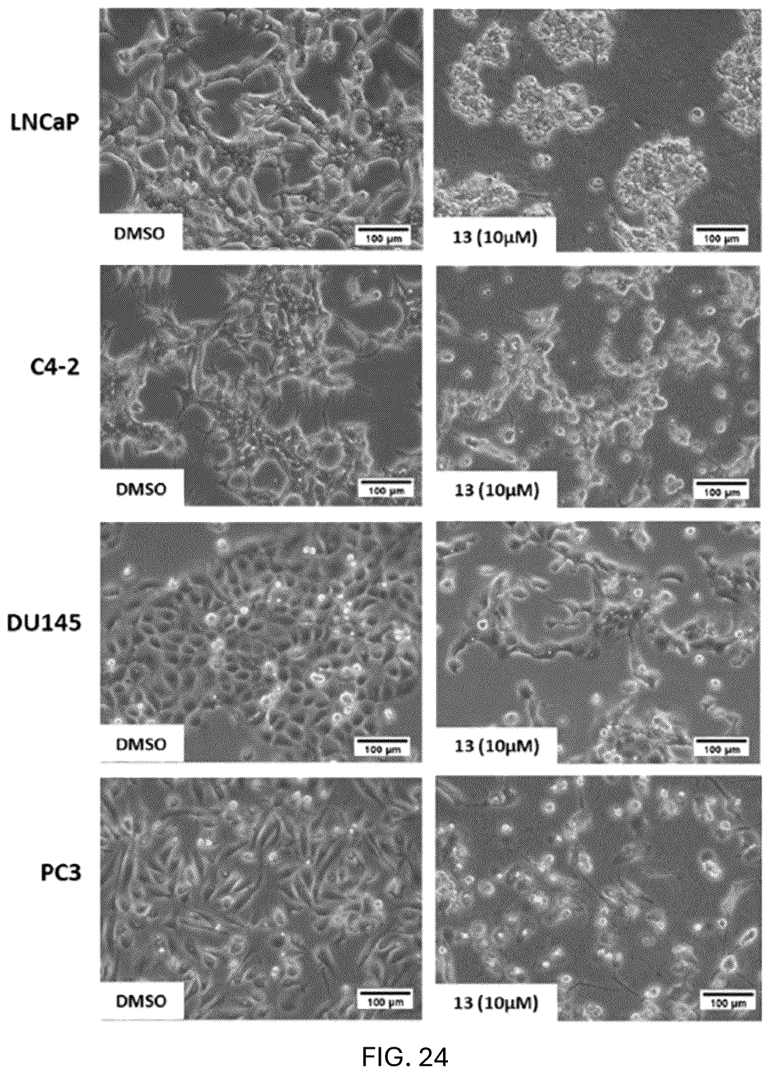

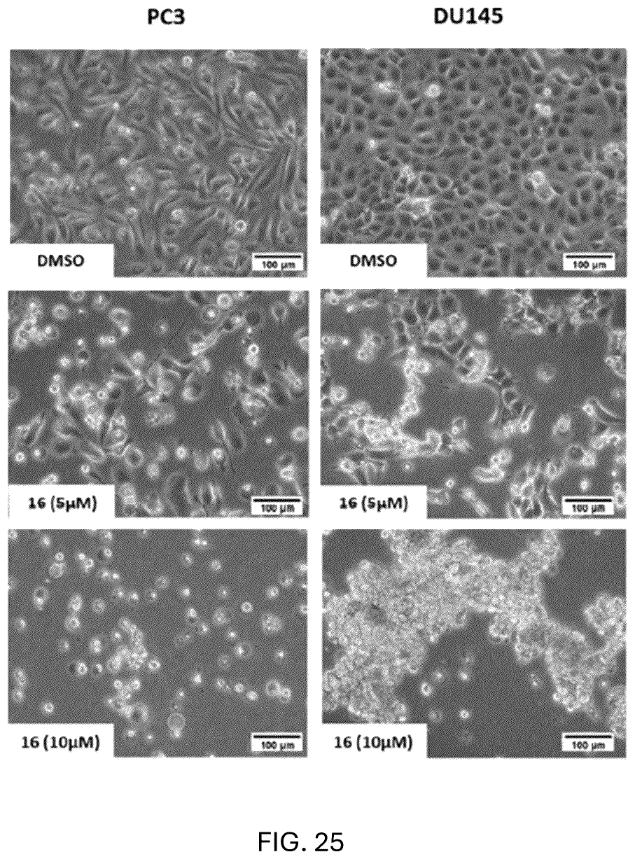

Based on the promising findings and the potent cytotoxic activity observed in the general screening with compounds (14-16) against AR-negative prostate cancer cell lines, Applicant decided to investigate further their effect on the morphology of two AR-positive cell lines (C42 and LNCaP) in addition to the AR-negative PCa (PC3 and DU145) cell lines. As shown in FIG. 24, compound 13 caused C4-2 and LNCaP to lose their shape, shrink in size, and increased cell death. Notably, the compound was profoundly more cytotoxic to the more aggressive and highly invasive prostate cancer cell lines (PC3) as compared to the less aggressive AR-positive cells. Moreover, Applicant examined the effect of compound 16 at a lower concentration (5 μM) since the cells were almost completely dead at 10 μM. Treatment of PC3 and DU145 cells with 5 μM of compound 16 showed increased cell detachment, the formation of apoptotic bodies, and cell shrinkage, without other characteristic changes on cell adhesion (FIG. 25).

### Effect on Apoptosis Pathway

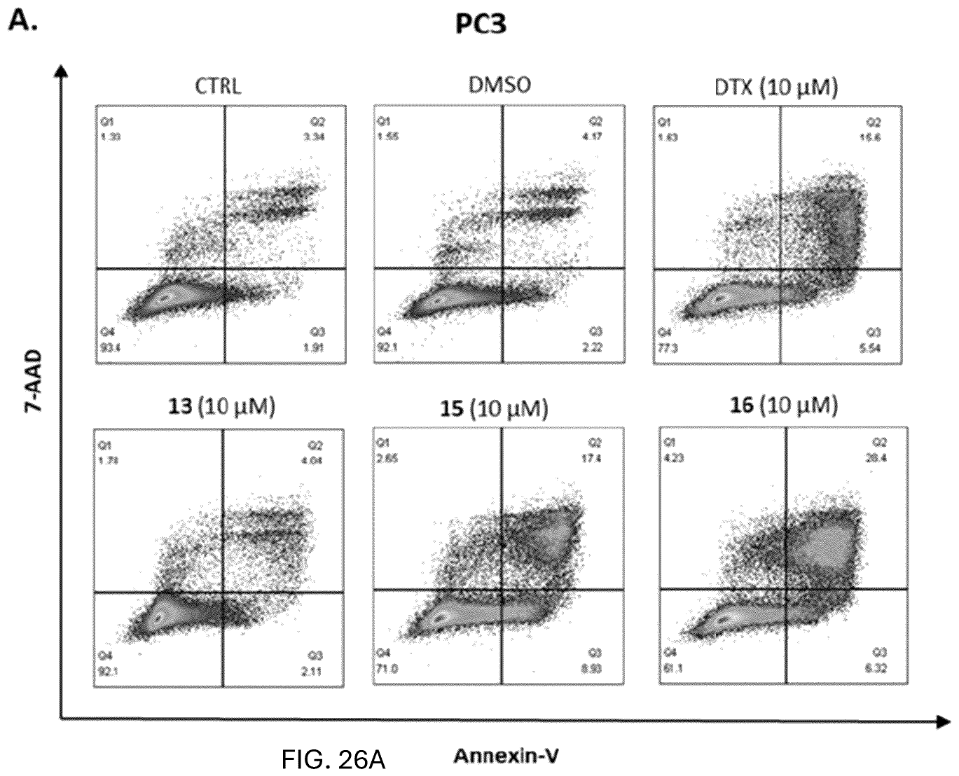

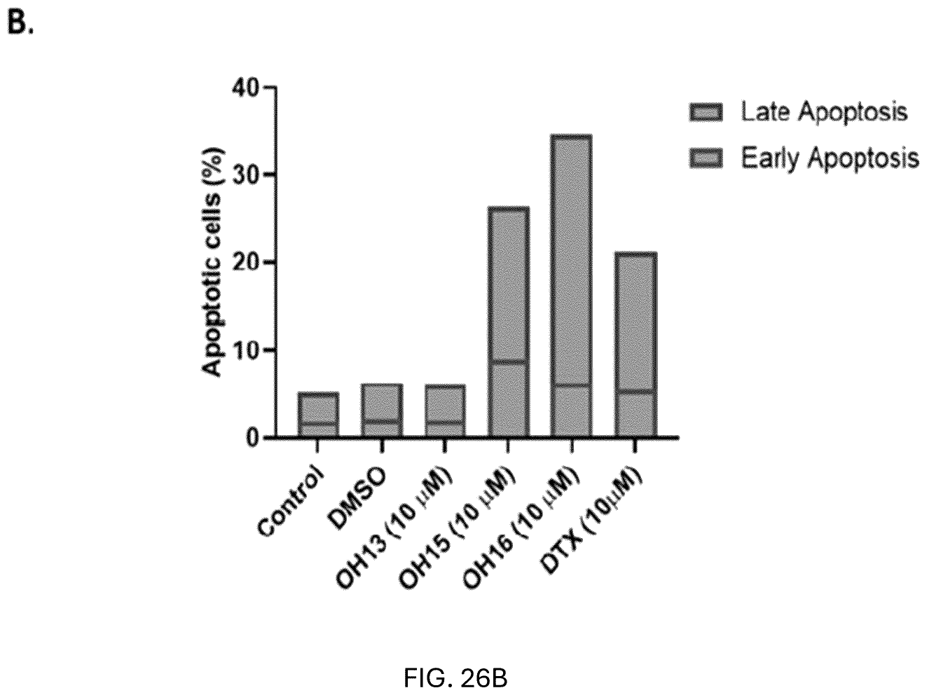

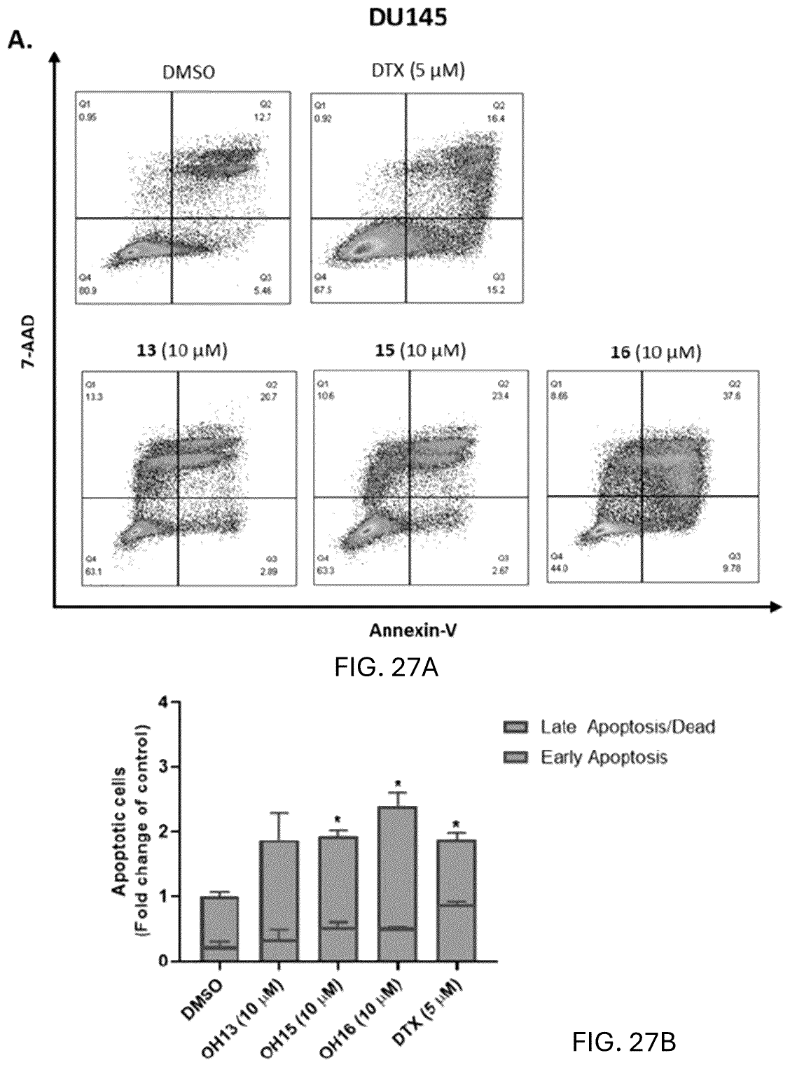

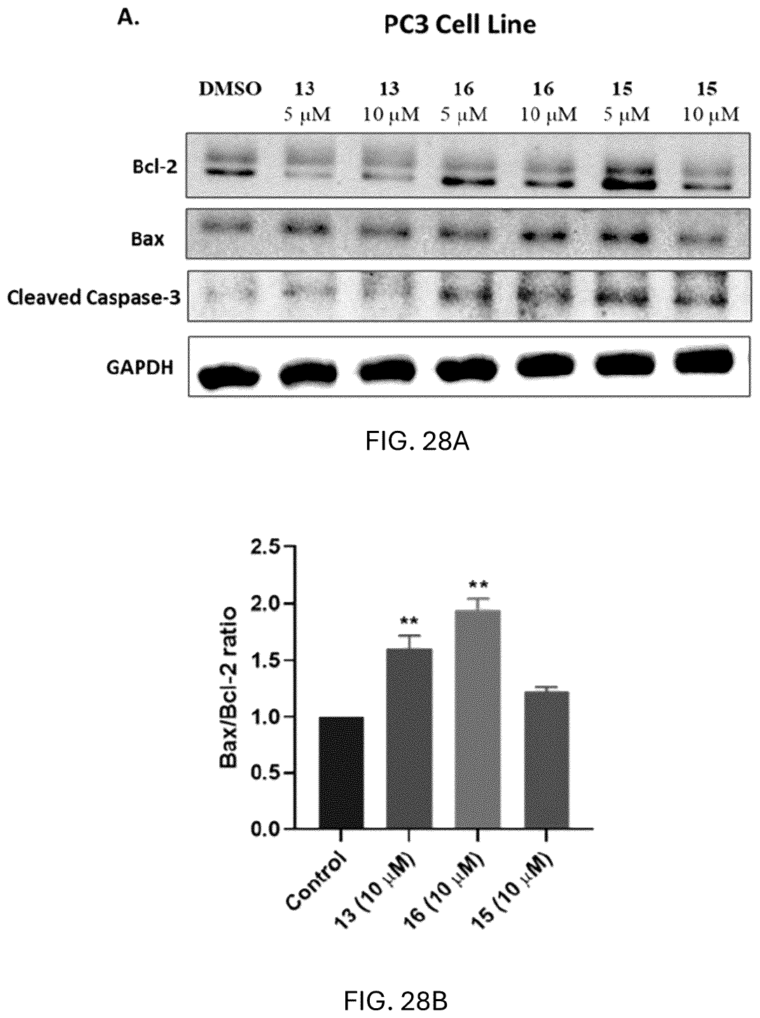

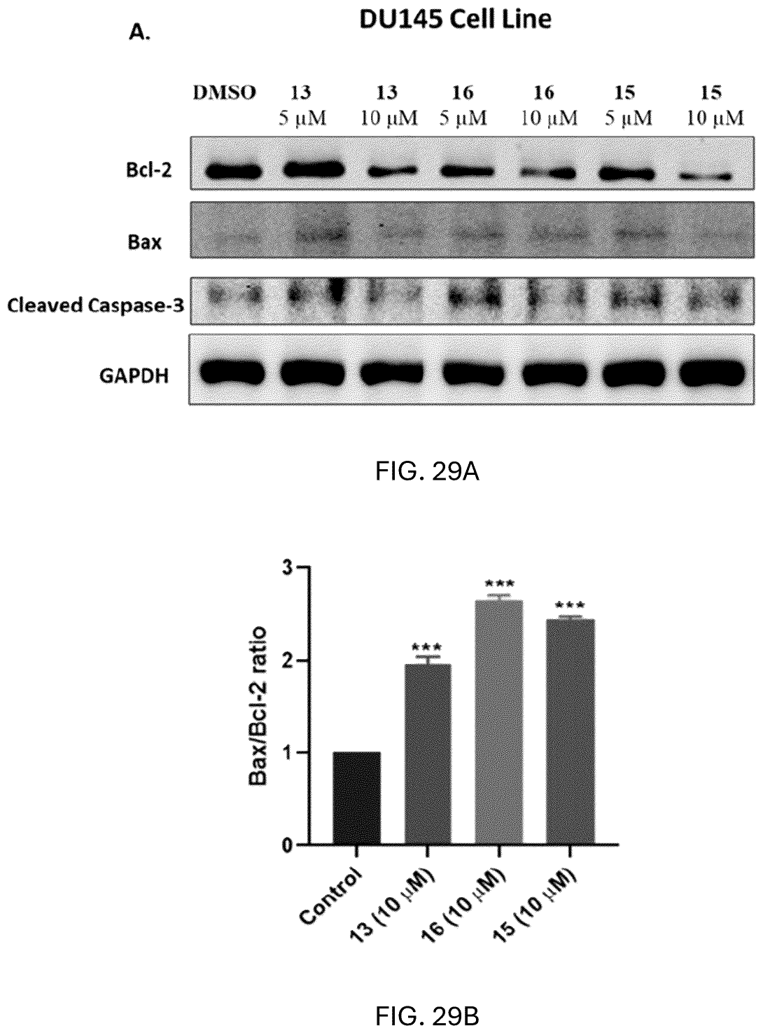

Based on the morphological observations, Applicant set out to test that the cytotoxic activity of the molecules (13, 15 and 16) is at least partially mediated by apoptosis induction. To confirm these findings, Applicant decided to directly measure the percentage of apoptotic cells by Annexin V-FITC/7-AAD assay using flow cytometry. As shown in FIGS. 26A-26B and 27A-27B, compound 16 resulted in profound induction of apoptosis in both PC3 and DU145 by 34.7% and 47.8%, respectively. Interestingly, the apoptosis induced by compound 16 was higher than apoptosis with docetaxel, first-line chemotherapy in mCRPC. Further, Applicant examined the effect of the compounds on the expression of three apoptosis-related biomarkers (pro-apoptotic proteins: Bax and caspase-3, and anti-apoptotic protein: Bcl-2) in PC3 and DU145 cell lines (FIGS. 28A-28B and 29A-29B). The findings showed that following 48 hours of treatment, the three compounds can enhance the expression of Bax and downregulate Bcl-2, which led to a significant increase in Bax/Bcl-2 ratio in both investigated cell lines as compared to the control. Of note, compound 16 was the most effective among the tested compounds.

### Effect on Cell Cycle

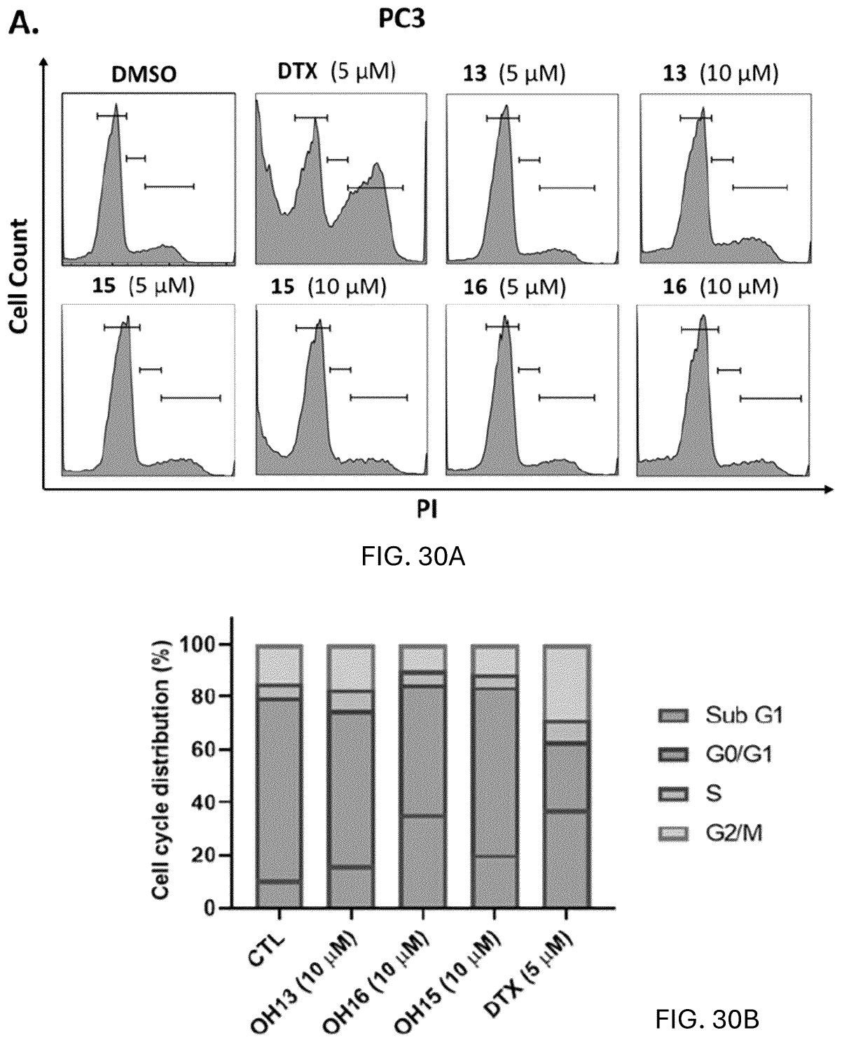

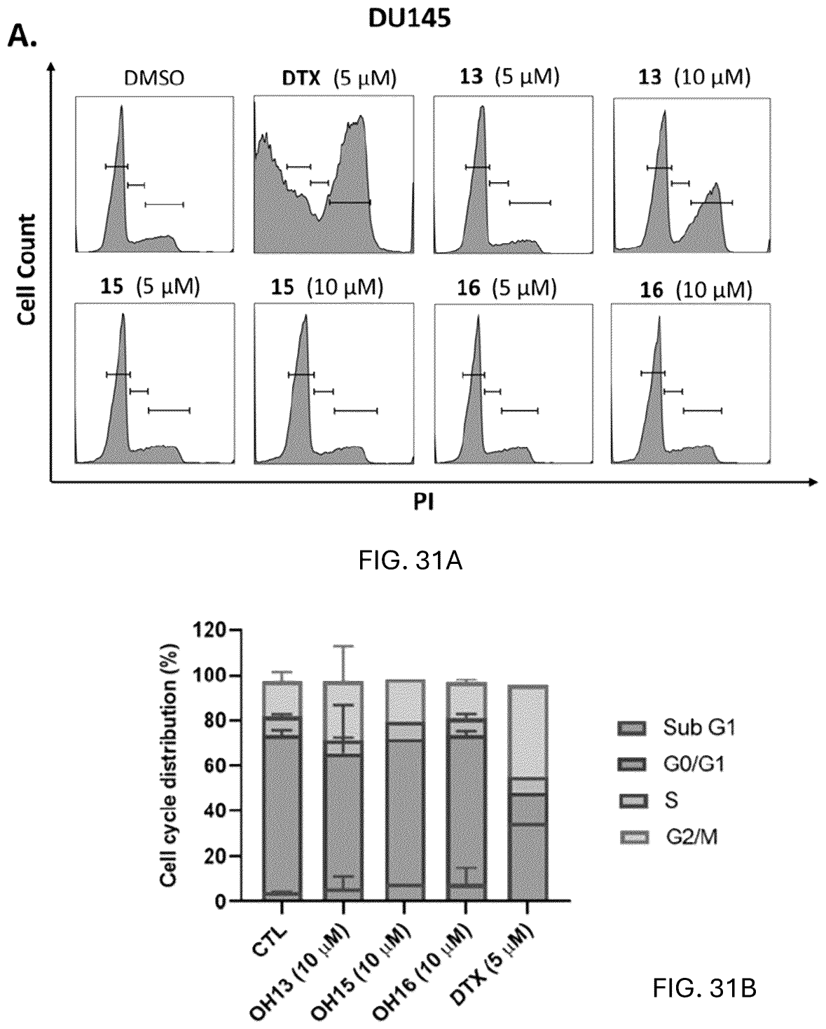

To examine whether the antiproliferative effect induced by compounds (13, 15 and 16) on PC3 and DU45 cell lines is associated with cell cycle arrest, treated cells were stained with propidium iodide (PI) and subjected to flow cytometric analysis. The distribution of cells across different cell cycle phases was assigned based on the intensity of PI stain, which correlates with the quantity of DNA content in each phase. A representation of different cell cycle phases can be seen in FIGS. 30A and 31A, which represent in order cells in G0/G1, S, and G2/M phases. The results showed that compounds 13, 15 and 16 induced a slight accumulation in G2/M and sub G1 phases, which was accompanied by a reduction in G1/G0 and S phases in DU145 cell line; however, the effect was not statistically significant (P>0.05). On the other hand, the G2/M phase was markedly increased with docetaxel by 1.9-2.6 folds as compared to control. These findings suggest that the compounds act through a distinct mechanism of action.

### Effect on Colony Formation

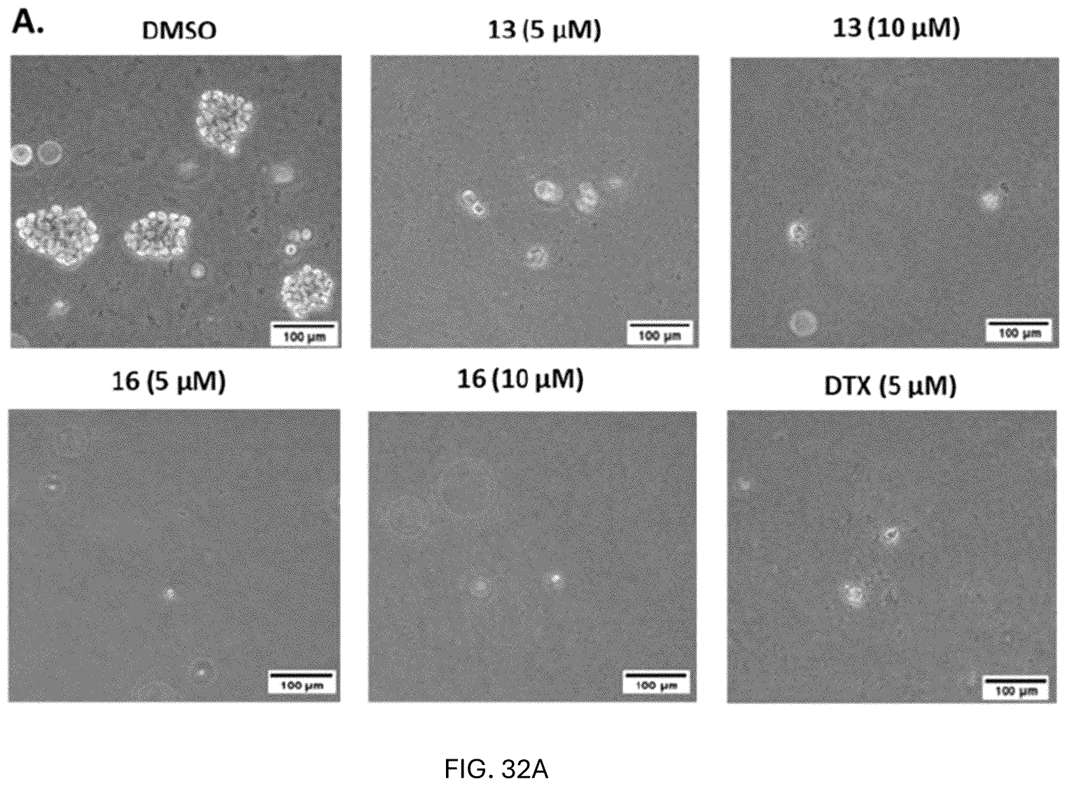

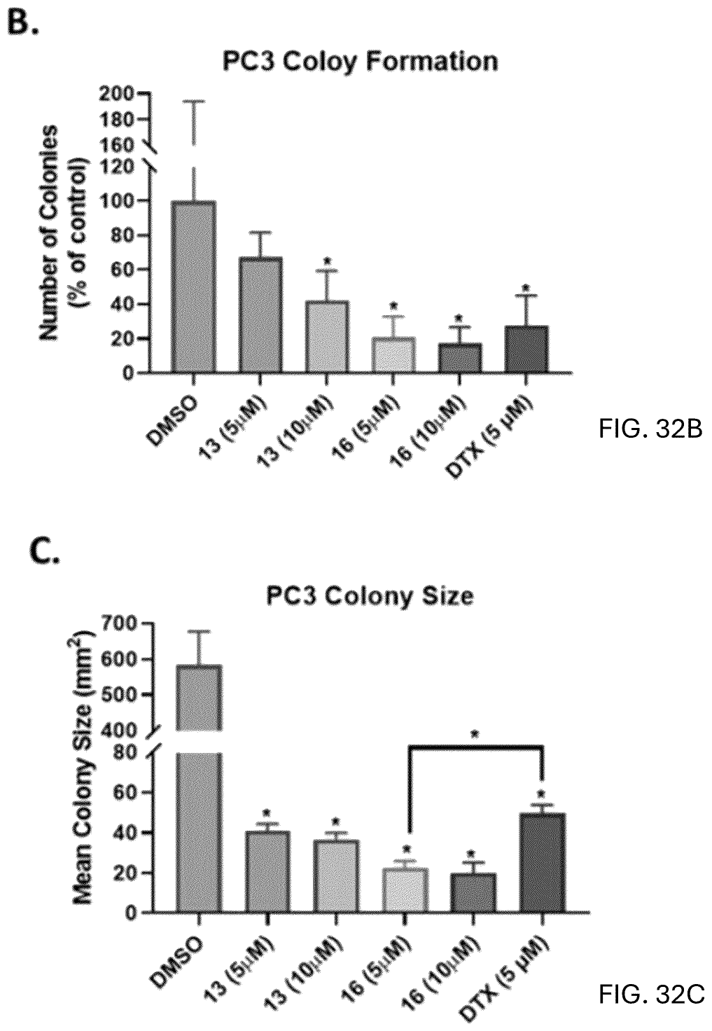

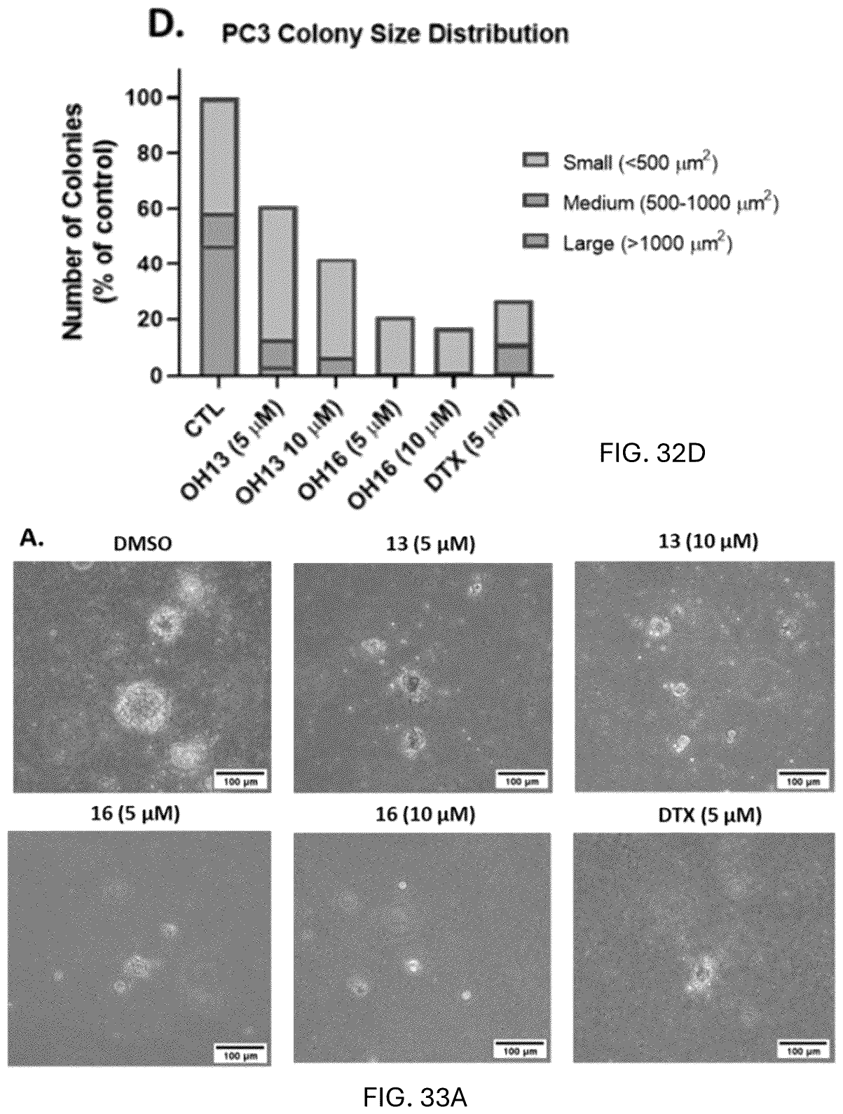

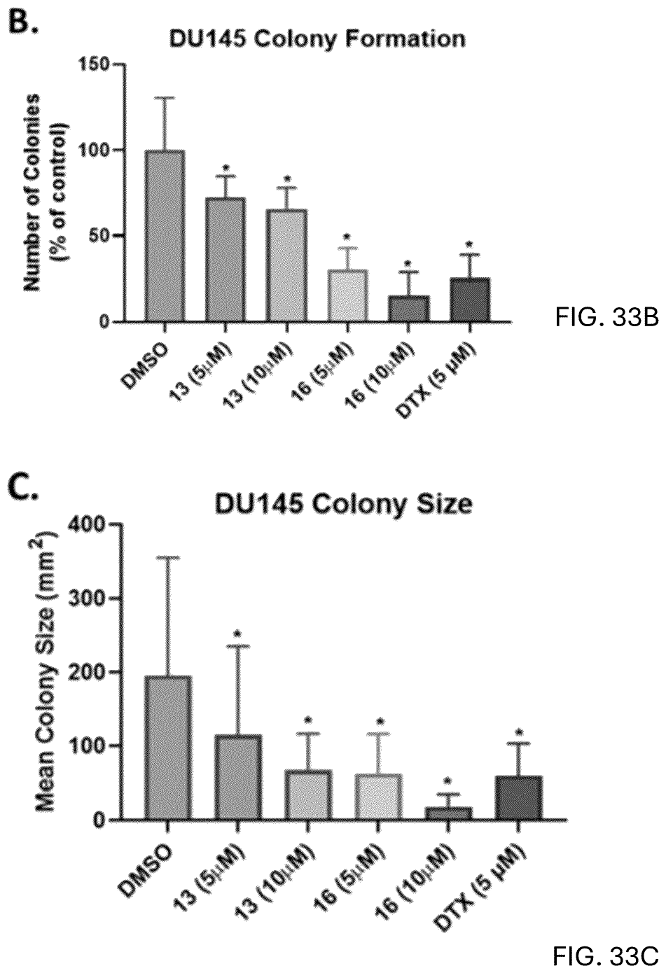

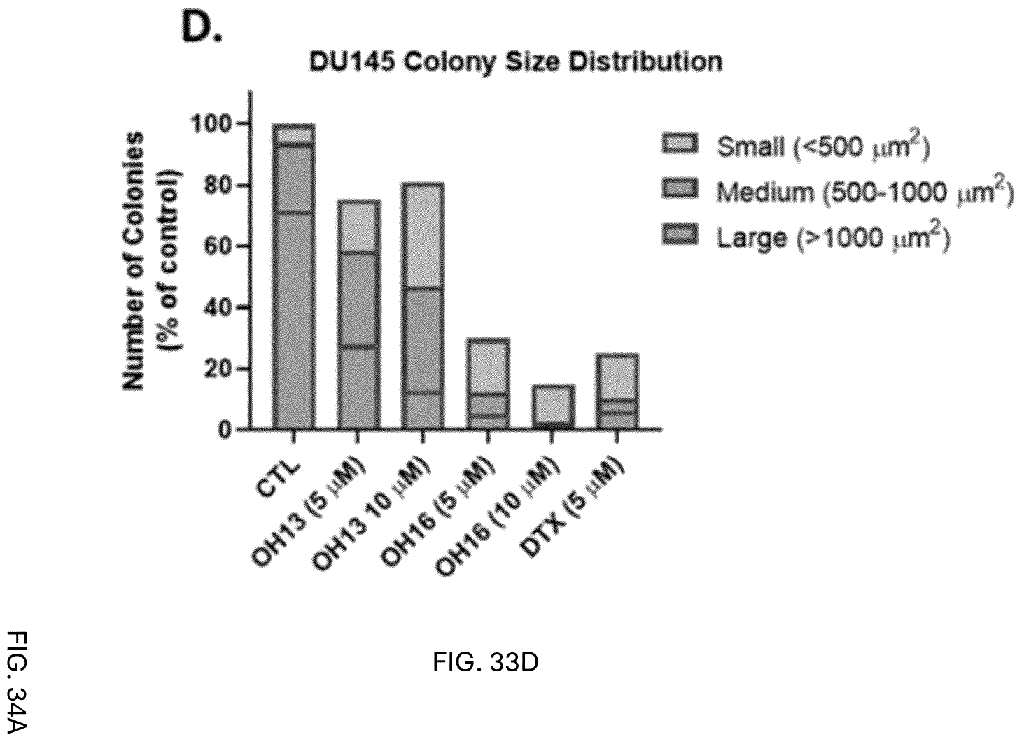

Anchorage-independent growth is a well-known characteristic of transformed cells that correlate with in vivo tumorigenic potential. Soft agar colony formation assay is a robust in vitro method used to estimate the ability of cells to grow and form tumors in-vivo. Therefore, Applicant investigated colony formation of PC3 and DU145, in soft agar, under the effect of compounds 13 and 16 at 5 and 10 μM, for 14 days. FIGS. 32A-32D and 33A-33D show that compounds 13 and 16 resulted in a significant reduction in both the total number of colonies and average colony size in both cell lines. Specifically, compound 16 at 10 μM significantly reduced the number of colonies by 82.8% and 84.9% in PC3 and DU145 cell lines, in comparison to control, respectively. Similarly, the average size of detected colonies dramatically reduced from 583.2 to 20.0 mm² and from 196.0 to 18.7 mm² in PC3 and DU14, respectively, when exposed to 10 μM of compound 16 as compared to control. Interestingly, the reduction in average colony size induced by compound 16 on PC3 cells was stronger than the positive control, docetaxel, when compared at the same concentration (P<0.001).

### Effect on Cells Migration

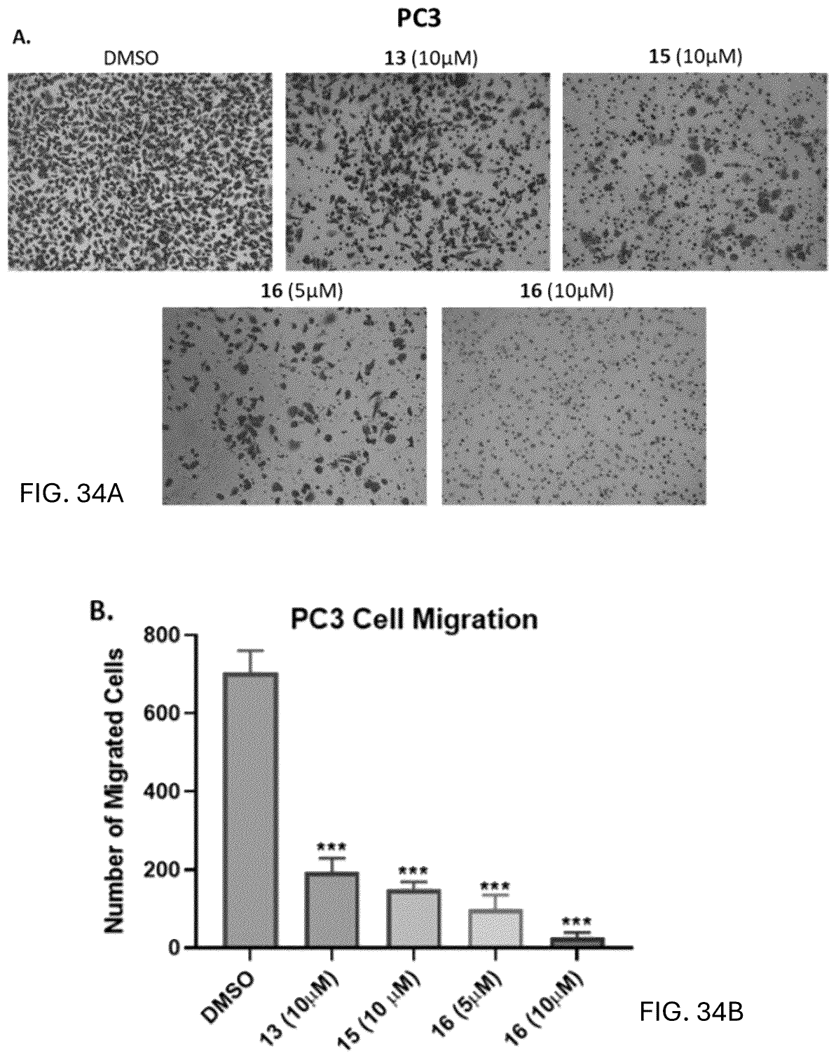

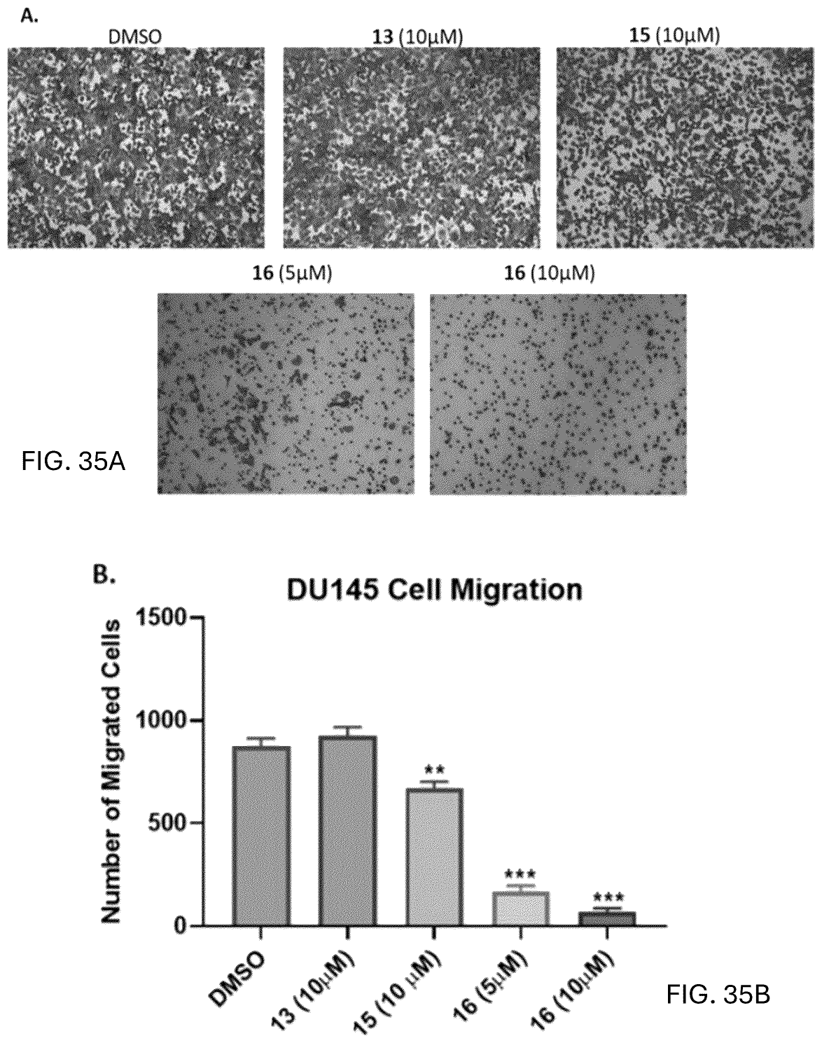

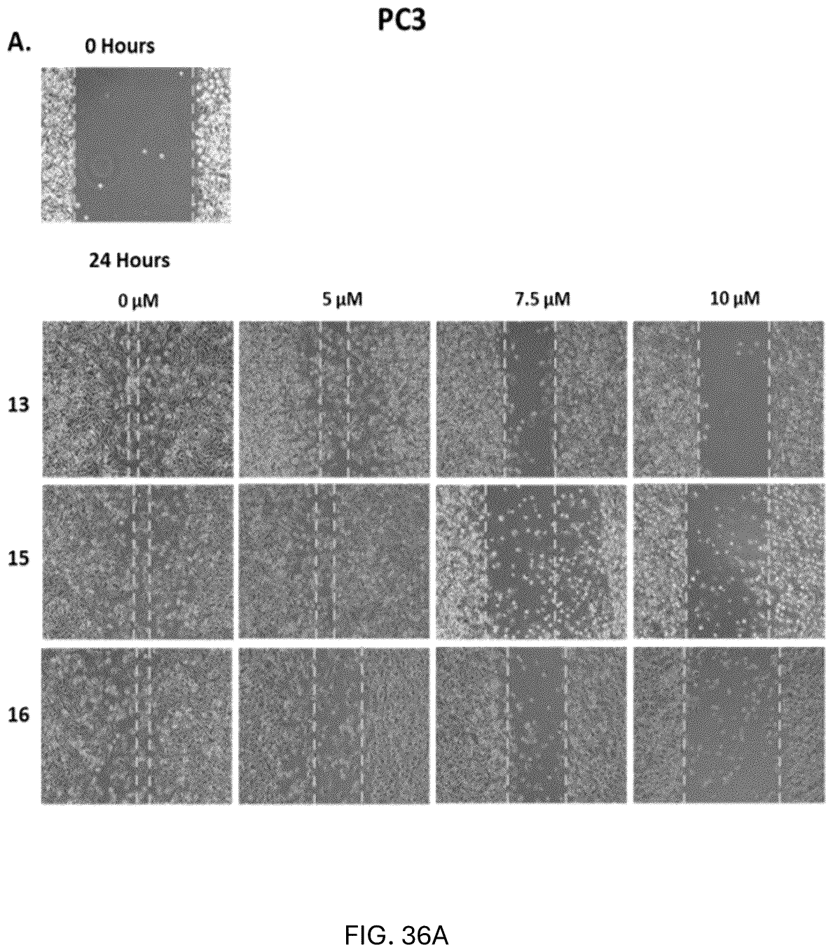

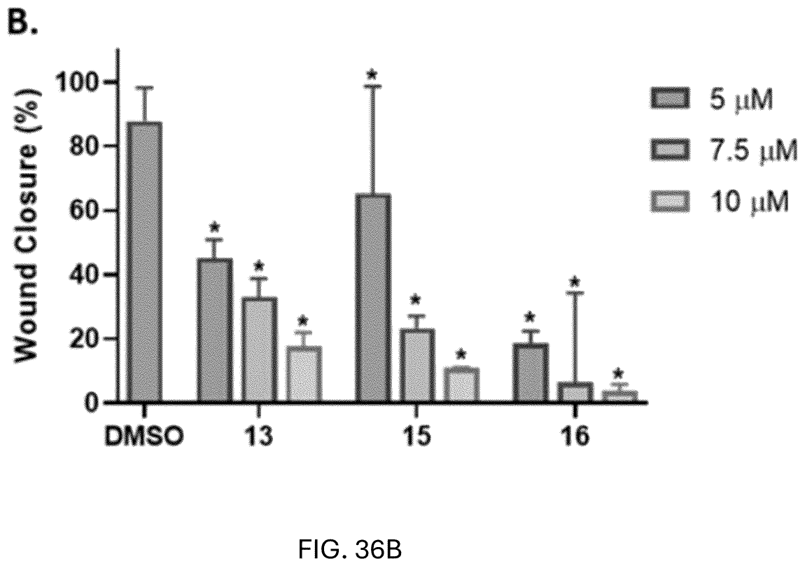

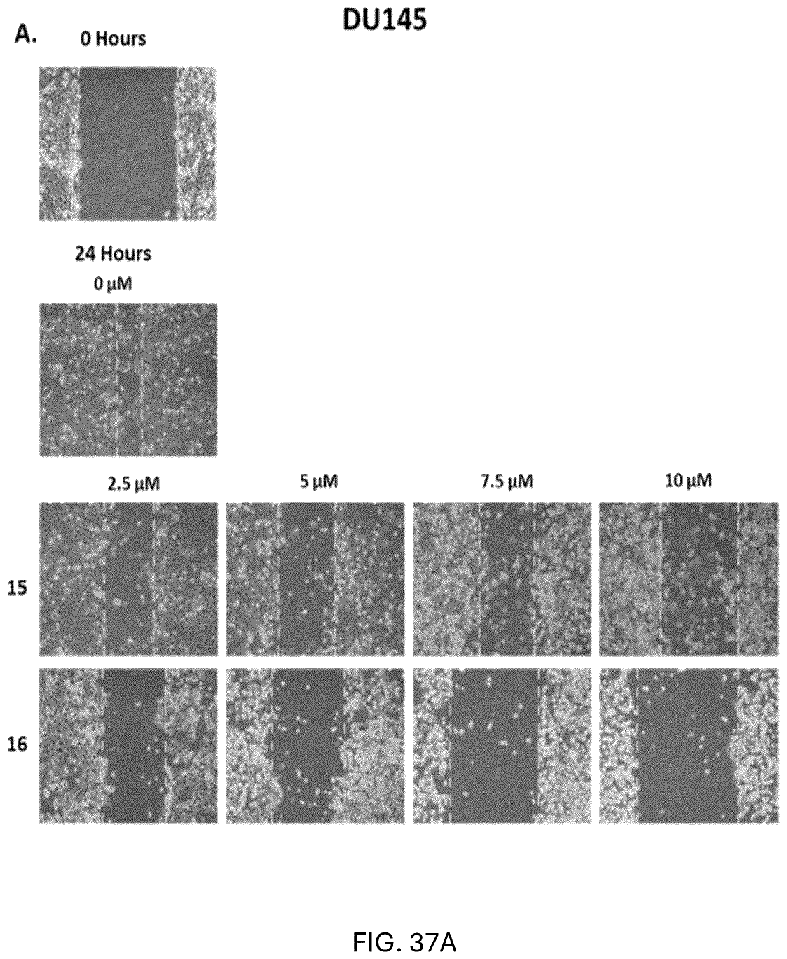

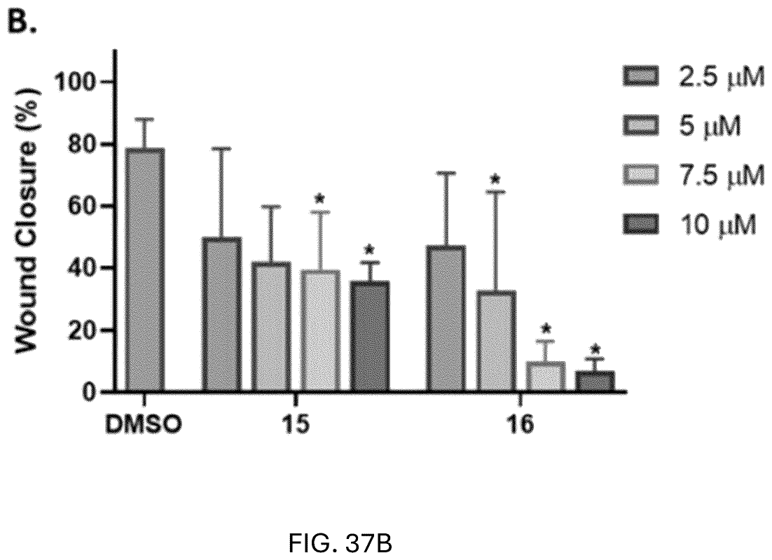

While localized prostate cancer has a good prognosis, progression to metastatic PCa dramatically reduces the 5-year survival rate to 30.2%. Therefore, Applicant investigated the effect of compounds 13, 15 and 16 on inhibiting the migratory potential of the invasive prostate cancer cell lines (PC3 and DU145). Two assays were used for this purpose; the first evaluated the effect on trans-well migration across pores and the other investigated migration over the plate surface. The data revealed that compounds 13, 15 and 16 significantly reduced trans-well migration of PC3 cells by 72.4%, 78.8%, and 96.2% (P<0.0001), respectively, as compared to control when tested at 10 μM concentration. Moreover, the same compounds showed a dose-dependent reduction in wound closure against both cell lines, which was significant starting from 5 μM concentration in PC3 cells. The results are shown in FIGS. 34A-34B, 35A-35B, 36A-36B, and 37A-37B.

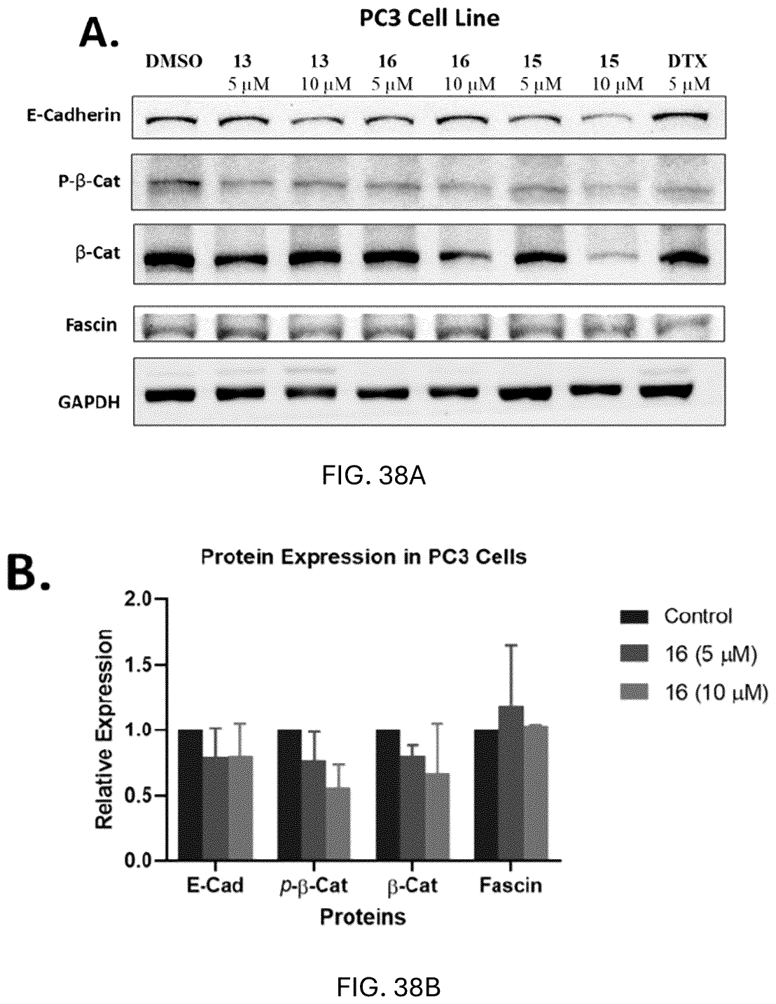

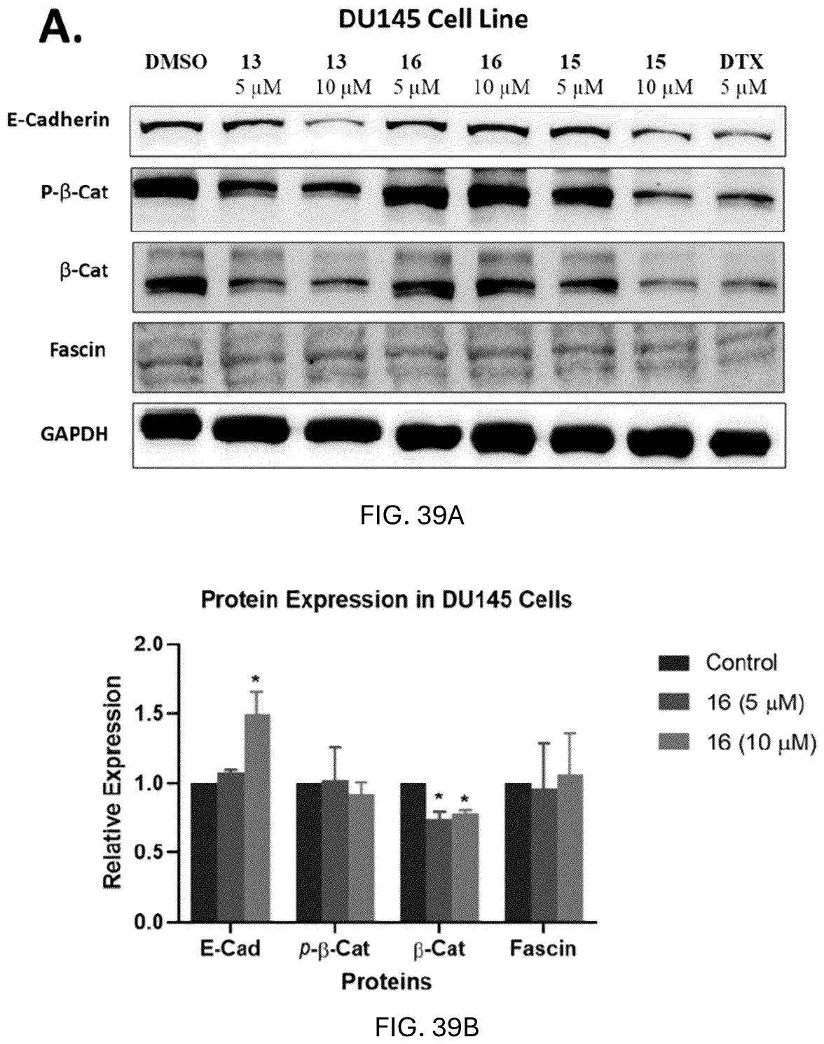

Based on findings from cell migration assays, Applicant set out to test that compounds 13, 15, and 16 might play a role in modulating the EMT process, which is linked to cancer invasion and metastasis. Therefore, Applicant explored the effect of the compounds on key proteins involved in EMT using two cell lines (PC3 and DU145). The explored proteins included E-cadherin, total and phosphorylated β-catenin, and fascin. As shown in FIGS. 38A-38B and 39A-39B, there was a trend towards modest reduction in total and phospho-β-catenin in both cell lines, significant only for total β-catenin in DU145. Additionally, compound 16 was able to significantly upregulate the expression of E-cadherin in DU145 cells (P-value 0.05). However, no significant changes were noted for the compounds on the expression of fascin in both cell lines. These findings indicate that compounds 13, 15 and 16 may target EMT in PCa; however, the observed effect was not strong enough to explain the anticancer potential of the compounds.

Effect on other Molecular Pathways

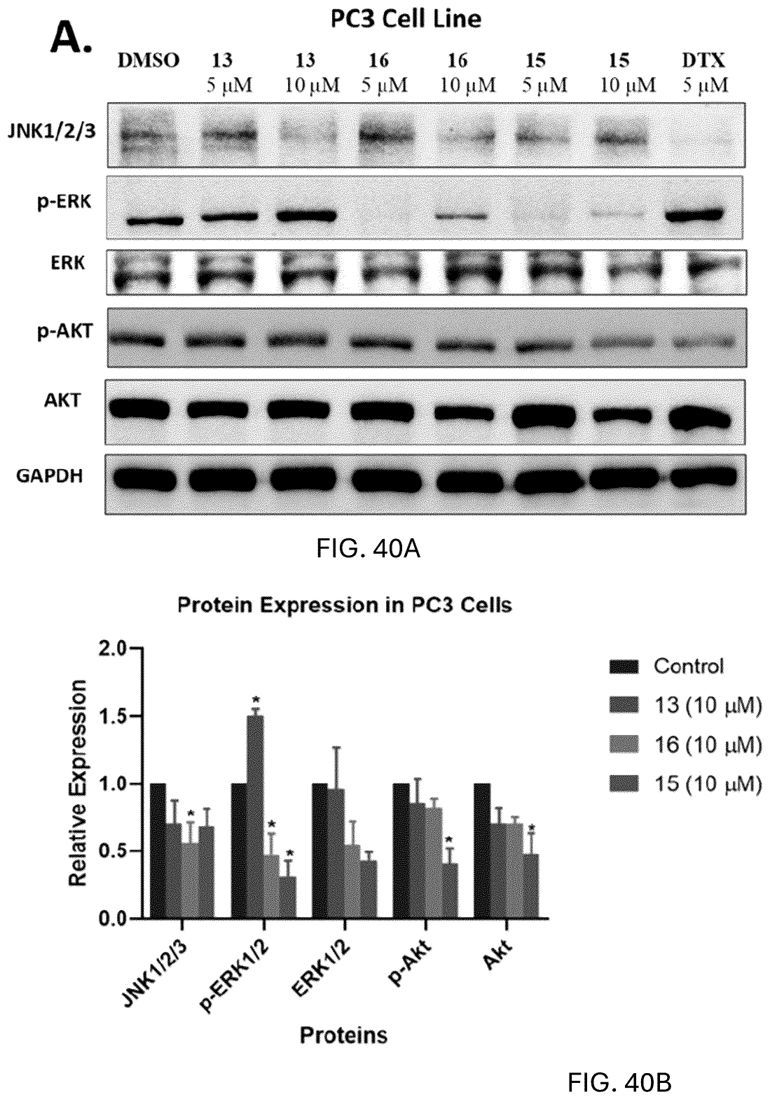

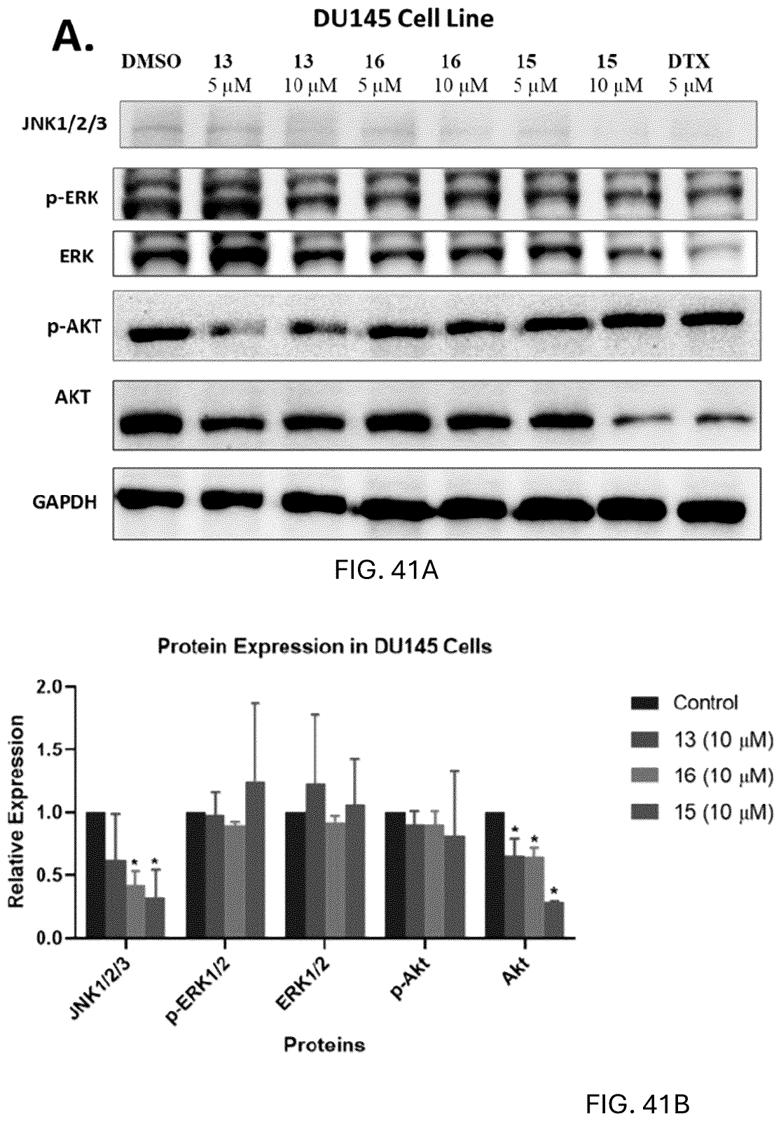

Applicant then explored the direct effect of the most active compounds on JNK1/2/3, total and phosphorylated ERK, and total and phosphorylated AKT. The results revealed that in PC3 cell line, molecule 16 had significantly reduced the expression of JNK1/2/3 and phosphorylated ERK1/2, without significantly altering ERK1/2, p-Akt, and Akt (FIGS. 40A-40B). In effect comparable to that of compound 16, compound 15 had reduced the expression of JNK1/2/3, p-ERK1/2, Akt and p-Akt. Both compounds maintained the same effect on JNK1/2/3 in DU145 cell line (FIGS. 41A-41B). All compounds were also able to maintain the downregulation of AKT in DU145 cell line without altering the expression of p-Akt.

### Effect on Angiogenesis

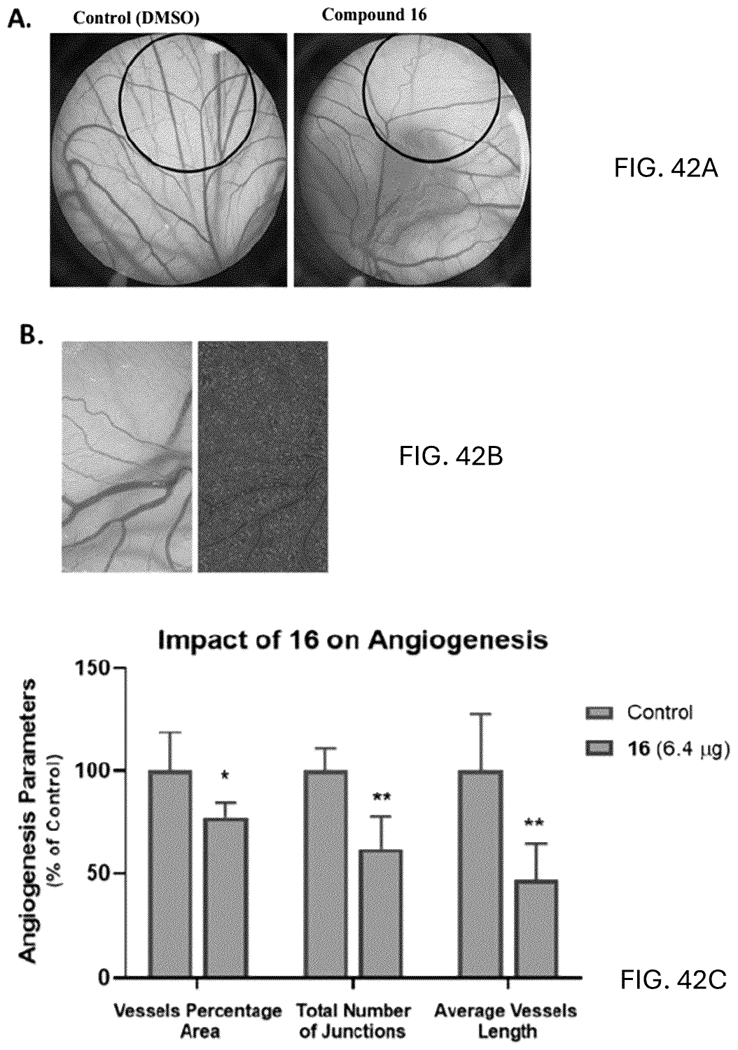

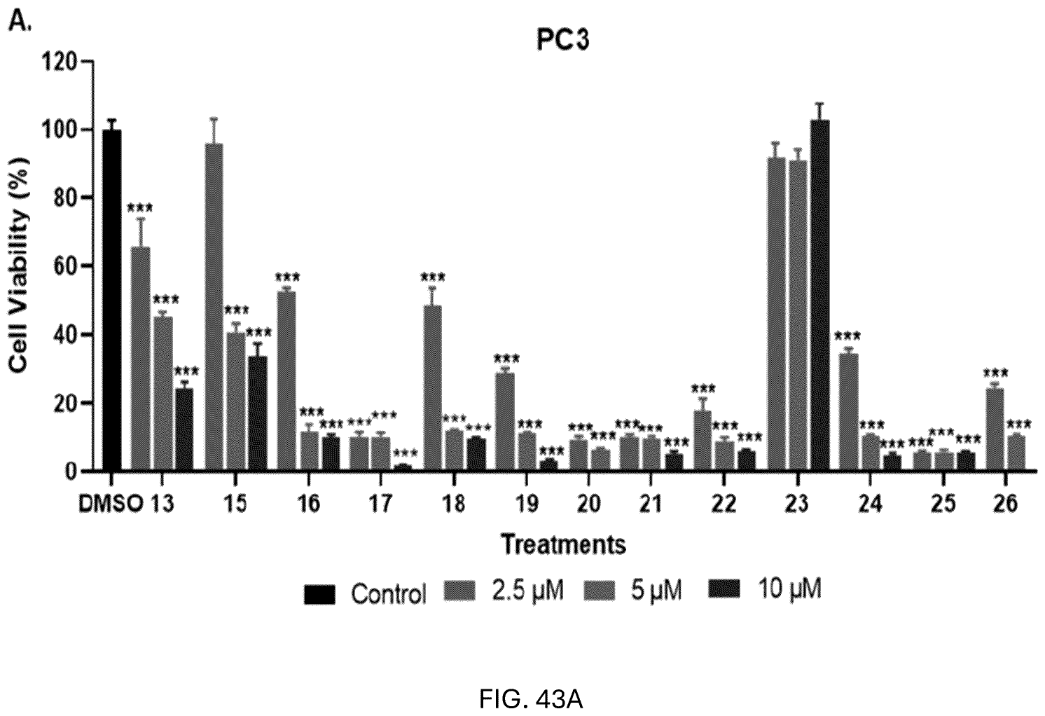

Uncontrolled angiogenesis is a key factor in cancer progression, invasion, and metastasis. Therefore, Applicant sought to investigate the effect of the compounds on angiogenesis using the CAM of chicken embryos. Treating embryos with compound 16 led to a significant reduction in average vessel length, a total number of junctions, and vessel area, suggesting that it acted as an angiogenesis inhibitor (FIGS. 42A-42B).

### Expanded SAR Study (Compounds 13-26)

Out of the sixteen synthesized and evaluated compounds in the first phase of the study, thienyl pyridine chalcone hybrids (13, 15 and 16) exhibited the most promising anticancer potential against two of the most aggressive prostate cancer cell lines (PC3 and DU145). This was clear from their highly potent cytotoxicity and attractive effect on modulating cancer-related pathways. Ten additional thienyl pyridine chalcone hybrids were synthesized to explore structural attributes that may enhance or diminish the activity. The structural diversity in the new analogs was introduced by varying substitutions at ring A of the chalcone while keeping the thienyl pyridine and the enone moieties intact.

Three main aspects were explored in the SAR study: the impact of α-conformational restriction (cyclic analogs), particularly with tetralone ring; the effect of varying the methoxylation pattern; and the effect of replacing methoxy with other electron-donating groups (EDG) or electron-withdrawing groups (EWG).

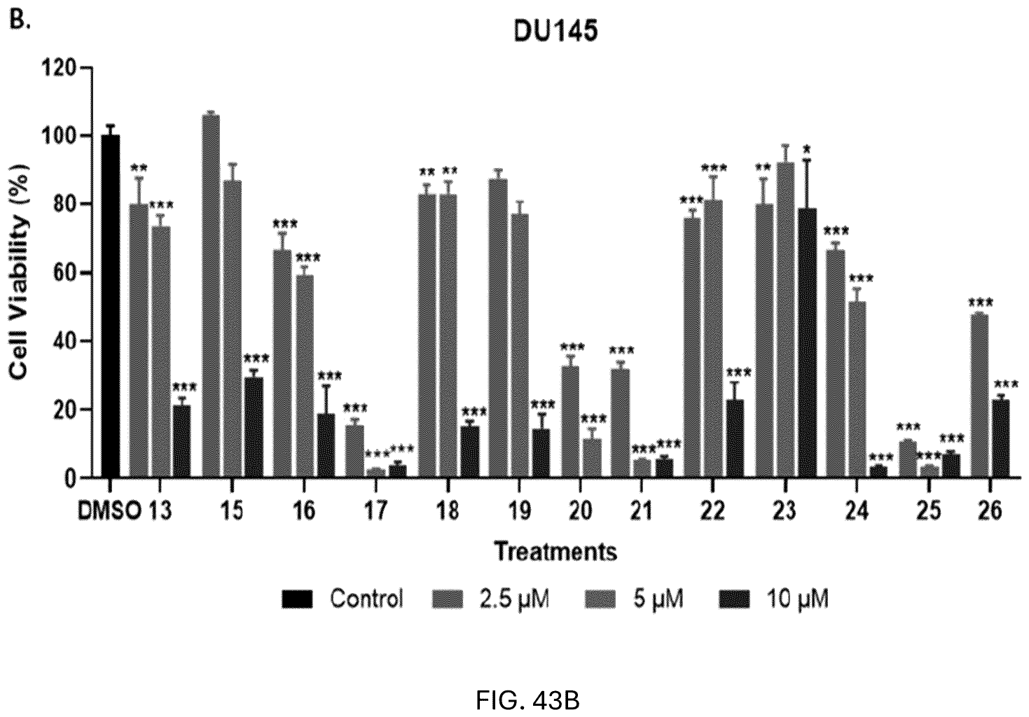

As shown in FIGS. 43A-43B, all the newly synthesized analogs (17-26) possessed excellent cytotoxicity, especially against PC3 cell line (>90% reduction in cell viability at 10 μM concentration) except for compound 23. To distinguish between the activity of different analogs, compounds were tested at lower concentrations (2.5 and 5 μM).

First, Applicant evaluated the cytotoxicity of tetralone-based chalcones (13, 14 and 15) in comparison with their matched non-cyclic chalcones (16, 18 and 19). The data clearly shows that the non-cyclic chalcones displayed higher cytotoxicity, suggesting that the tetralone ring lowered the antiproliferative activity of the thienyl pyridine chalcone hybrids.

Next, Applicant examined various mono, di, and tri-methoxy substituted analogs. Interestingly, changing the position of the methoxy group from C₃ to C₂ resulted in a profound improvement in the cytotoxicity where the reduction in cell viability of PC3 induced by 2.5 μM treatment increased from 47.5% to 84.7% for compound 16 and 17, respectively. Investigation of various methoxy substituted analogs revealed that 2-methoxy induced the most favorable antiproliferative activity, followed by 2,3,4- and 2,4,6-tri-methoxy substituents. On the other hand, analogs with 4-methoxy substituents, including 3,4-dimethoxy and 3,4,5-trimethoxy, showed reduced activity, especially against DU145 cell line.

Then, Applicant evaluated the effect of incorporating other EDG or EWG at position-2 and −4. Replacing 4-methoxy in (18) with 4-fluoro (26) improved the cytotoxic activity. In contrast, substituting it with methyl sulfonyl (23) was detrimental for the activity. For position 2, replacing the methoxy in (17) with trifluoromethyl group maintained relatively similar cytotoxicity, whereas replacing it with less bulky EDG (hydroxy) decreased the cytotoxicity.

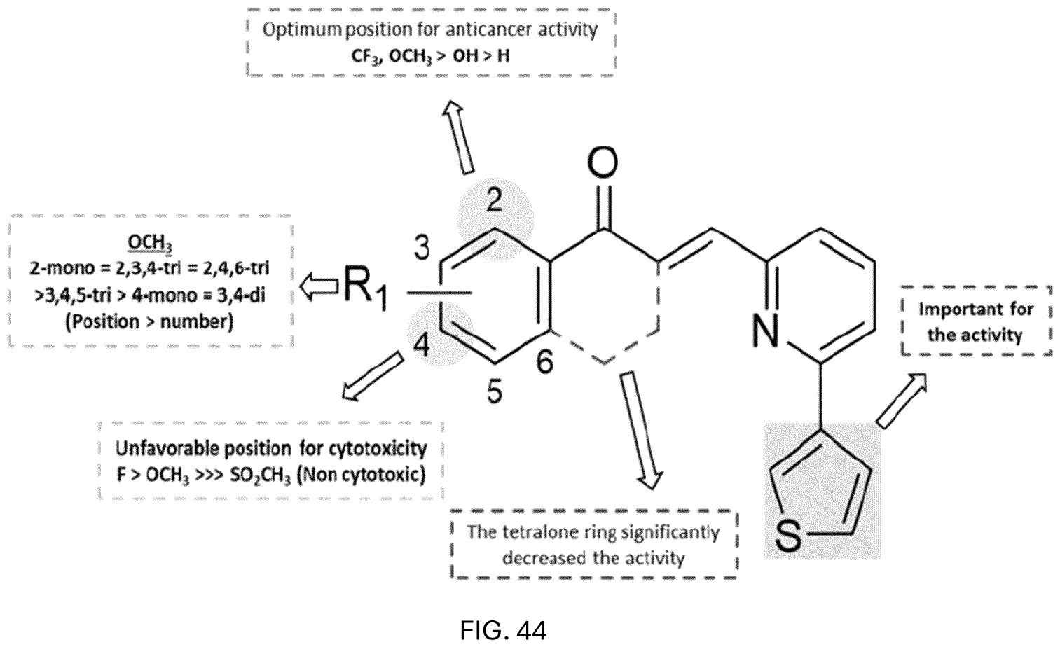

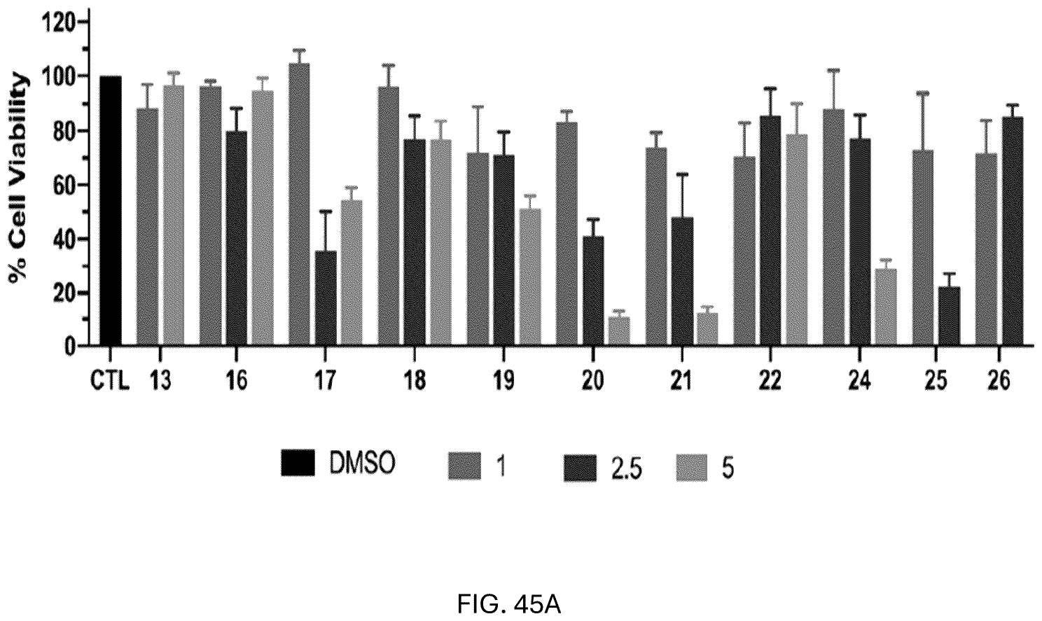

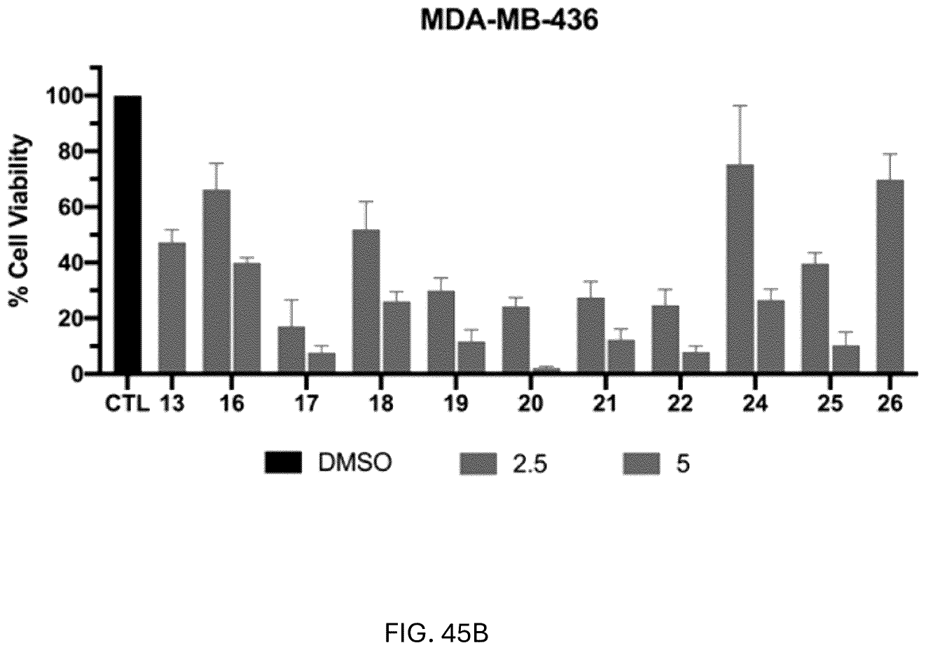

Overall, 2-methoxy and 2-trifluoromethyl substitutions on ring A are optimum for the antiproliferative potential of thienyl-pyridine chalcone hybrids (FIG. 44).

### General Screening (Compounds 13-26) Against TNBC Cell Lines

Out of the three synthesized series, series C (compounds 13-26) showed profound potent activity as compared to the first two series. More importantly, this series includes thienyl pyridine moiety that was incorporated for the first time within a chalcone scaffold. Besides, several analogs of series C showed favorable physiochemical properties as predicted by ADMET and Swiss ADME databases. Therefore, further work and screening against TNBC focused on series C.

Although screening against prostate cancer showed that compounds 17 and 25 showed the most potent activity. All compounds of series C were examined against TNBC since various tumor types and cell lines might respond differently to the tested compounds/treatments.

In agreement with findings against AR negative PCa cell lines, compounds 17 and 25 showed promising potent activity against TNBC cell lines at a concentration of 2.5 and 5 μM. On the other hand, other analogs (e.g., 21 and 20) showed a comparable effect on cell viability when tested against TNBC cell lines at 2.5 and 5 μM. Therefore, the four analogs were further tested and their IC50s were assessed to select the two most potent analogs. Compounds 17 and 25 showed a slightly higher potency than compounds 21 and 20. Although the difference was not statistically significant, compounds 17 and 25 were selected for further experimentation due to their superiority against prostate cancer as well.

### IC50 Evaluation

To assess whether the new analogs can serve as potential lead anticancer agents with improved efficacy, the IC50 was calculated for the two most potent analogs. TNBC cell lines (MDA-MB-231 and MDA-MB-436) cells were treated with increasing concentrations of compounds OH17 and OH25 for 24 and 48 hours, and their viability was assessed by Alamar blue. Besides, the effect of these analogs was compared to docetaxel, a first-line chemotherapy, as a positive control.

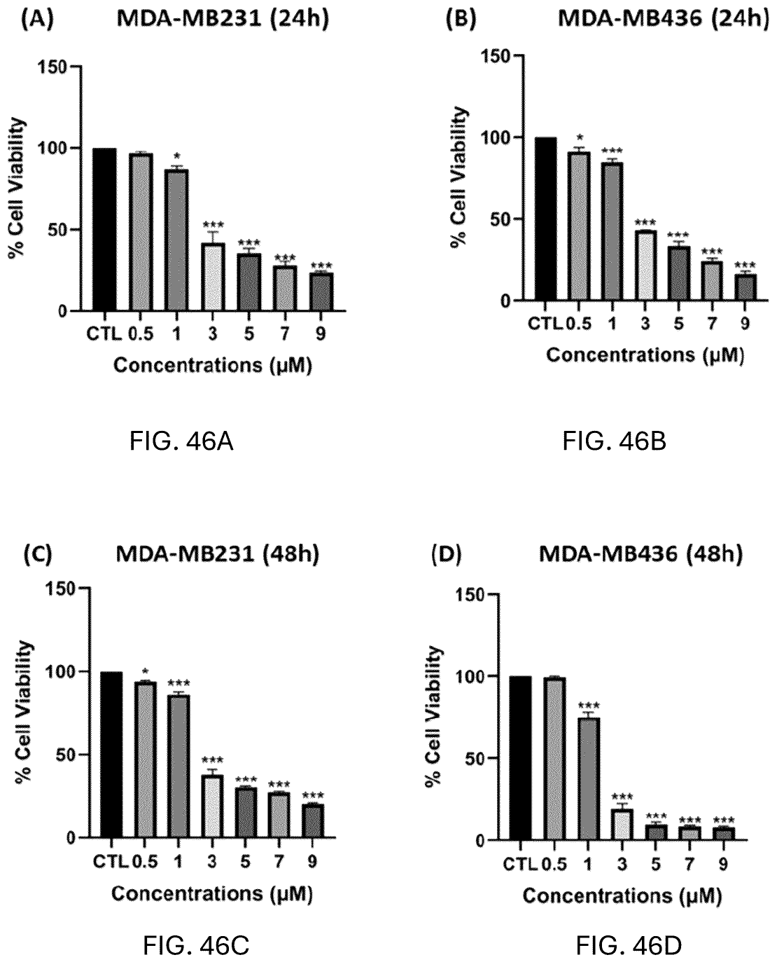

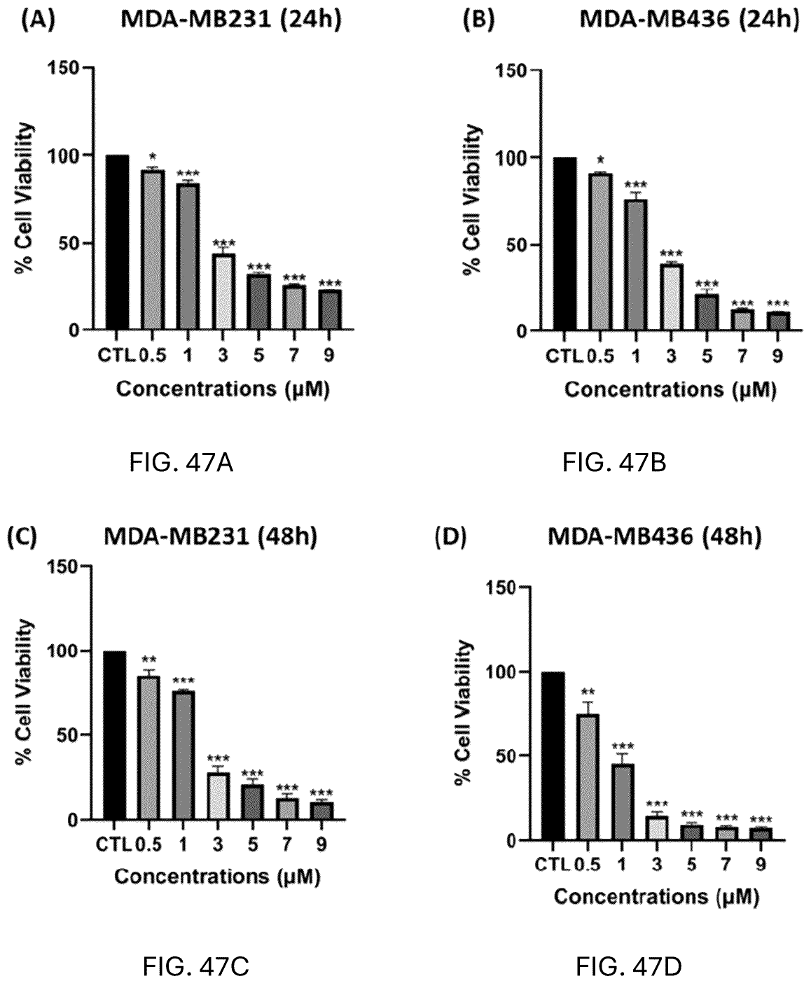

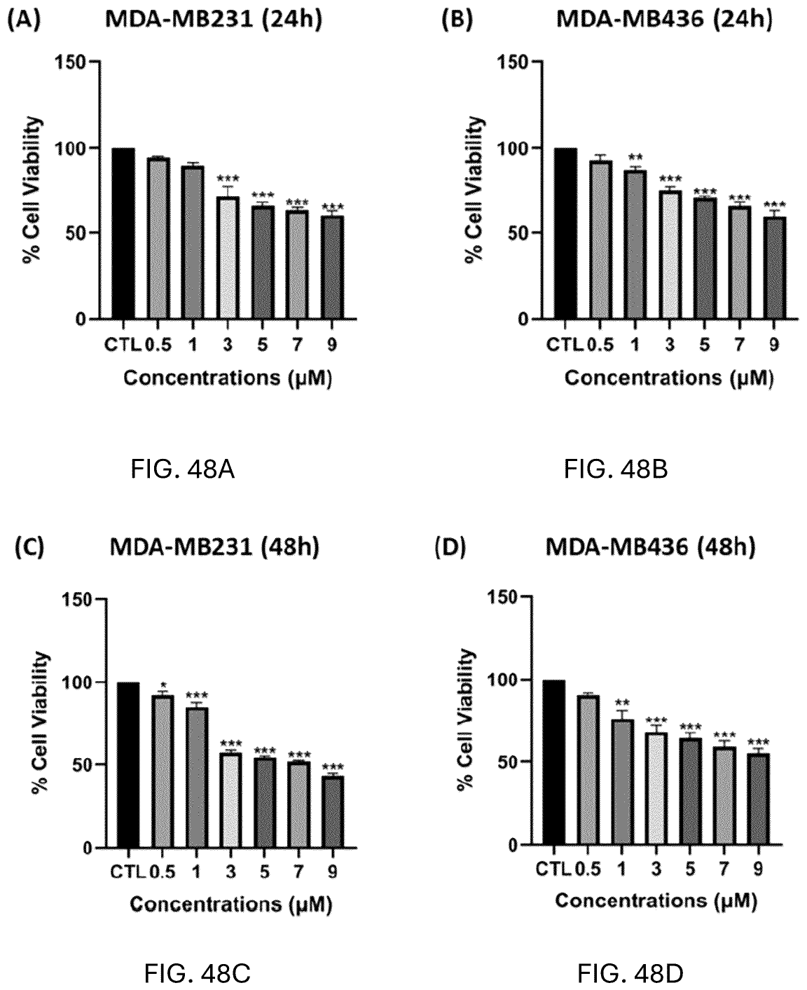

As seen in FIGS. 46A-46D, 47A-47D, and 48A-48D, both compounds showed a dose-dependent reduction in cell viability against TNBC cell lines in a time-dependent manner. Compound 25 showed the most potent cytotoxicity with an IC50 of 0.87 and 2.1 μM against MDA-MB-436 and MDA-MB-231, respectively, when treated for 48 hours (Table 5). Both analogs were slightly more potent in MDA-MB-436 than in MDA-231. Interestingly, the compounds exhibited higher cytotoxicity towards TNBC cell lines as compared to docetaxel.

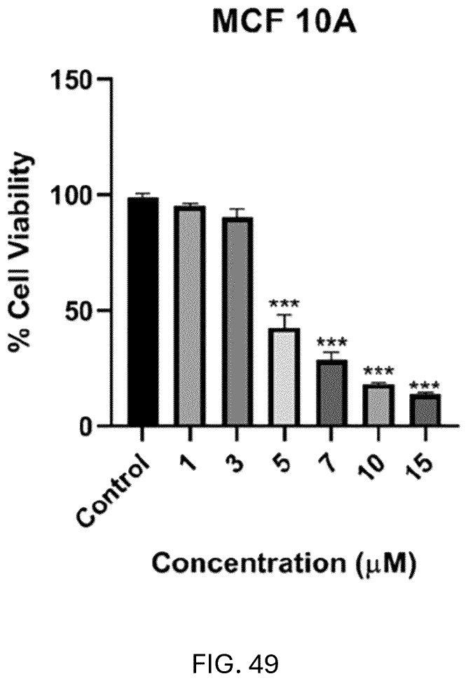

Moreover, both compounds 17 and 25 showed greater selectivity towards cancerous cell lines (MDA-231 and MDA-436) as compared to normal cells (MCF10a) (FIG. 49 and Table 6).

TABLE 5
IC50 of compounds OH17, OH25 and Docetaxel
(DTX) against TNBC cell lines

| | | |
|---|---|---|
| OH17 (μM) | OH25 (μM) | DTX (μM) |

24 hrs. post treatment

| | | | |
|---|---|---|---|
| MDA-MB-231 | 3.6 ± 0.5 | 3.5 ± 1.47 | 11.02 ± 1.7 |
| MDA-MB-436 | 3.2 ± 0.2 | 2.4 ± 0.2 | 11.9 ± 2.7 |

48 hrs. post treatment

| | | | |
|---|---|---|---|
| MDA-MB-231 | 3.2 ± 0.08 | 2.1 ± 0.4 | 6.43 ± 0.6 |
| MDA-MB-436 | 1.6 ± 0.2 | 0.87 ± 0.2 | 9.8 ± 2.0 |

Values are expressed as Mean ± SEM of three independent experiments. IC50 for each experiment were computed by non-linear regression using GraphPad Prism.

TABLE 6
Effect of compounds OH33 on non-tumorigenic epithelial cell
line after 48 hrs. and selectivity index of compound OH33

| | OH25 (μM) |
|---|---|
| MCF 10A | 3.44 ± 0.4 |
| SI of MCF 10A/MDA-MB231 | 1.8 |
| SI of MCF 10A/MDA-MB436 | 3.9 |

### Effect on Cell Morphology

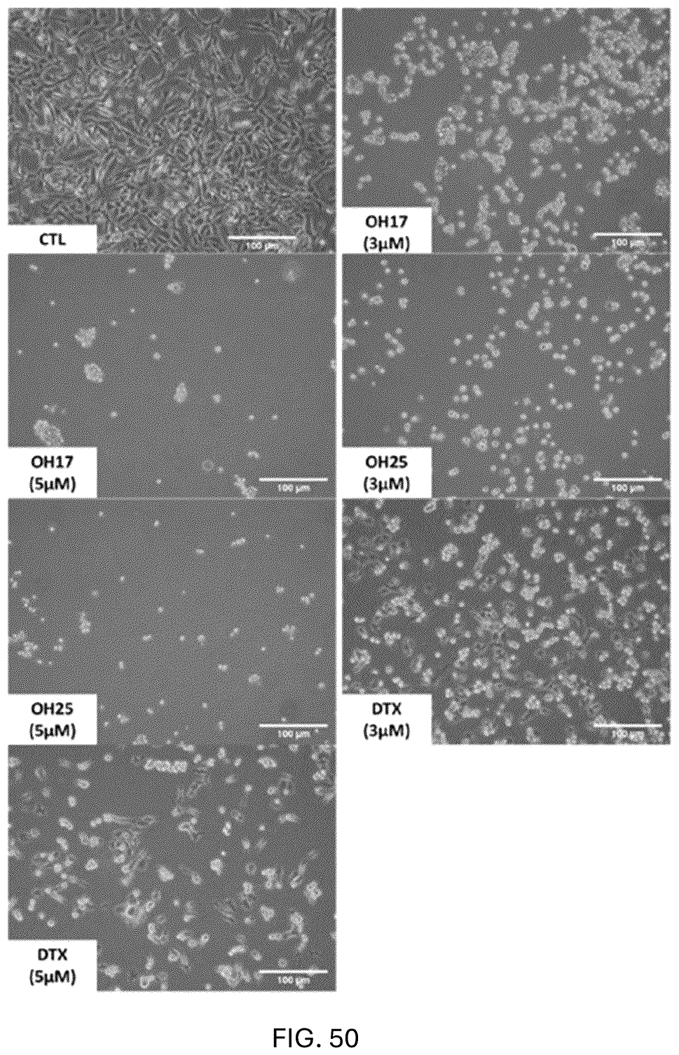

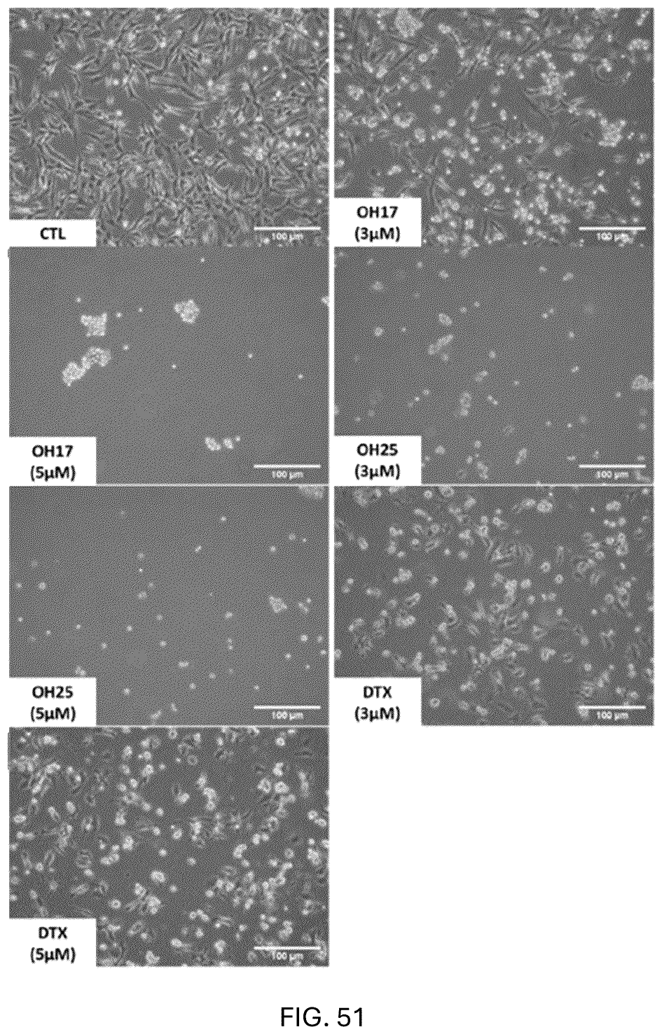

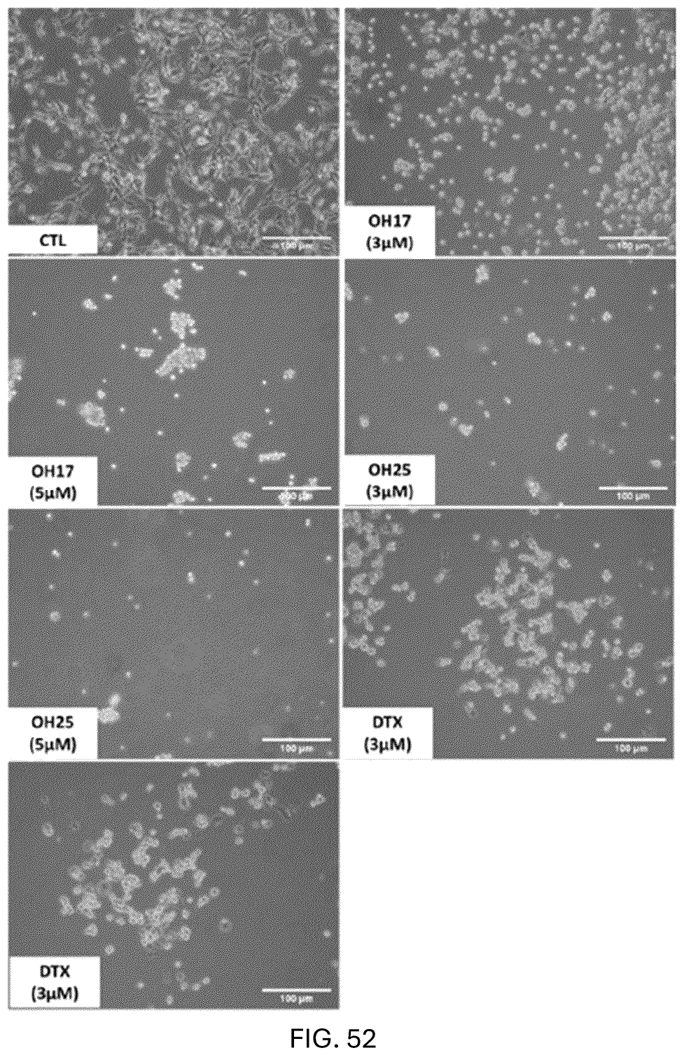

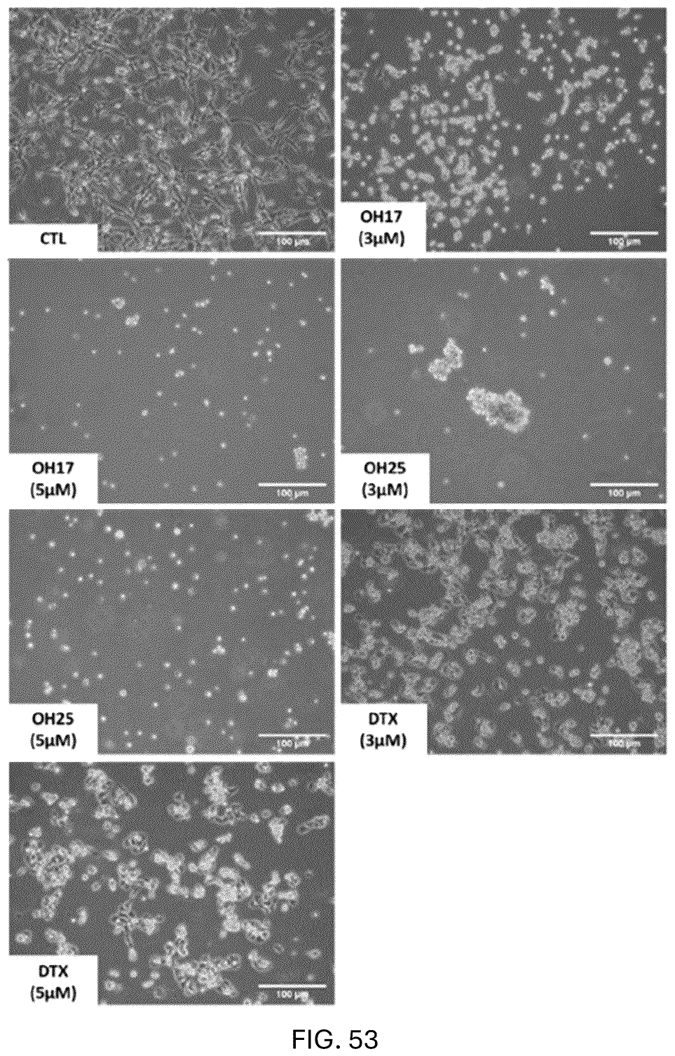

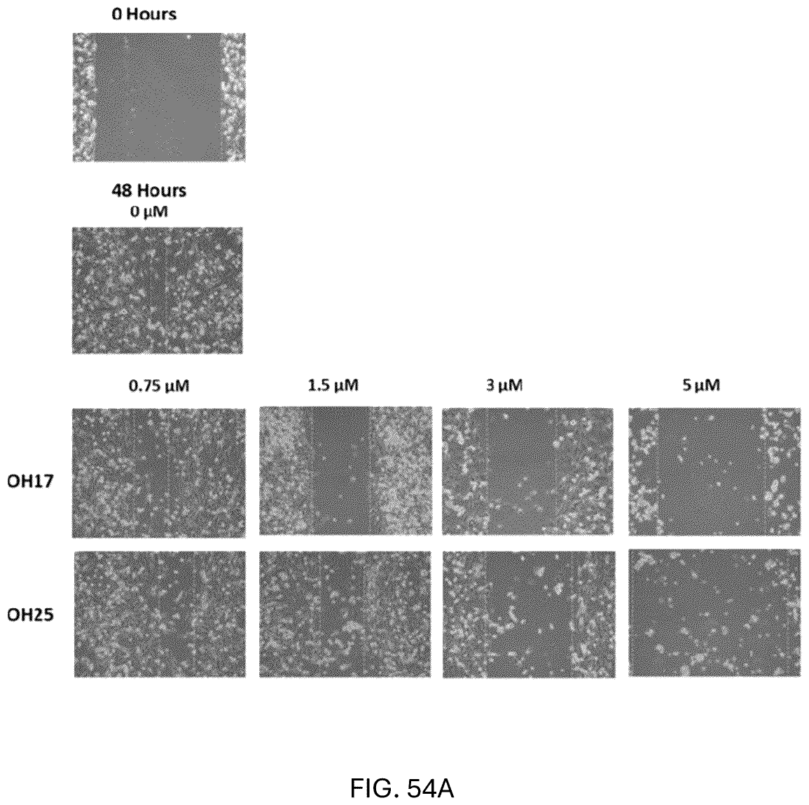

Based on the promising findings and the potent cytotoxic activity observed in the cell viability assessment against TNBC cell lines, Applicant investigated further effect on cell morphology and cancer related pathways. As shown in FIGS. 50-53, compounds OH17 and OH25 caused MDA-231 and MDA-436 to lose their shape, shrink in size, form apoptotic bodies, and increased cell death. Notably, the new compounds (17 and 25) were examined at a lower concentration (3 μM) since the cells were almost completely dead at 5 μM which indicate their superiority to compounds developed at the first phase of the study. Additionally, more cell death was observed in wells treated with compounds 17 and 25 as compared to wells treated with docetaxel when tested at the same concentration which confirms the cell viability data.

### Effect on Cell Migration

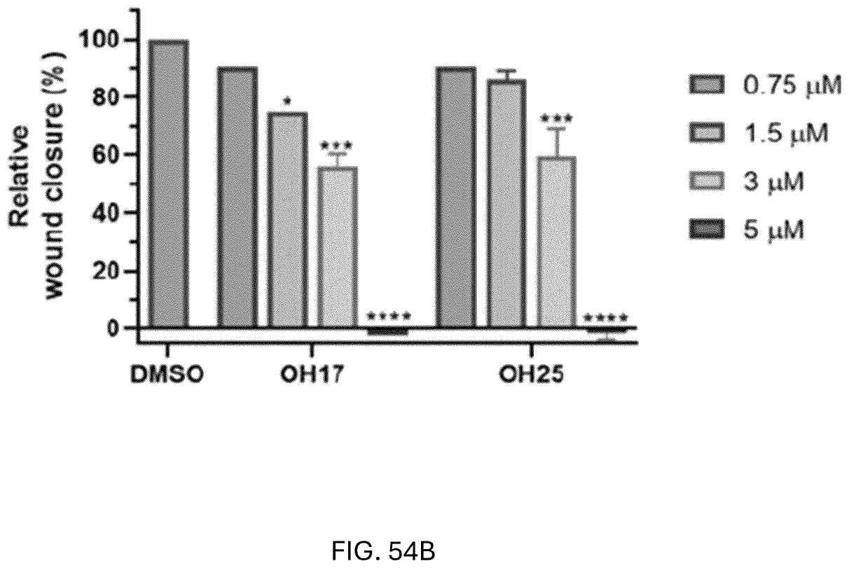

While localized breast cancer has a good prognosis, progression to metastatic breast cancer dramatically reduces the 5-year survival. Therefore, Applicant investigated the effect of compounds 17 and 25 on inhibiting the migratory potential of the TNBC cell lines (MDA-MB-231). The data revealed that compounds 17 and 25 significantly reduced cell migration of MDA-MB-231 in a dose-dependent manner (FIGS. 54A-54B).

### Effect on Colony Formation

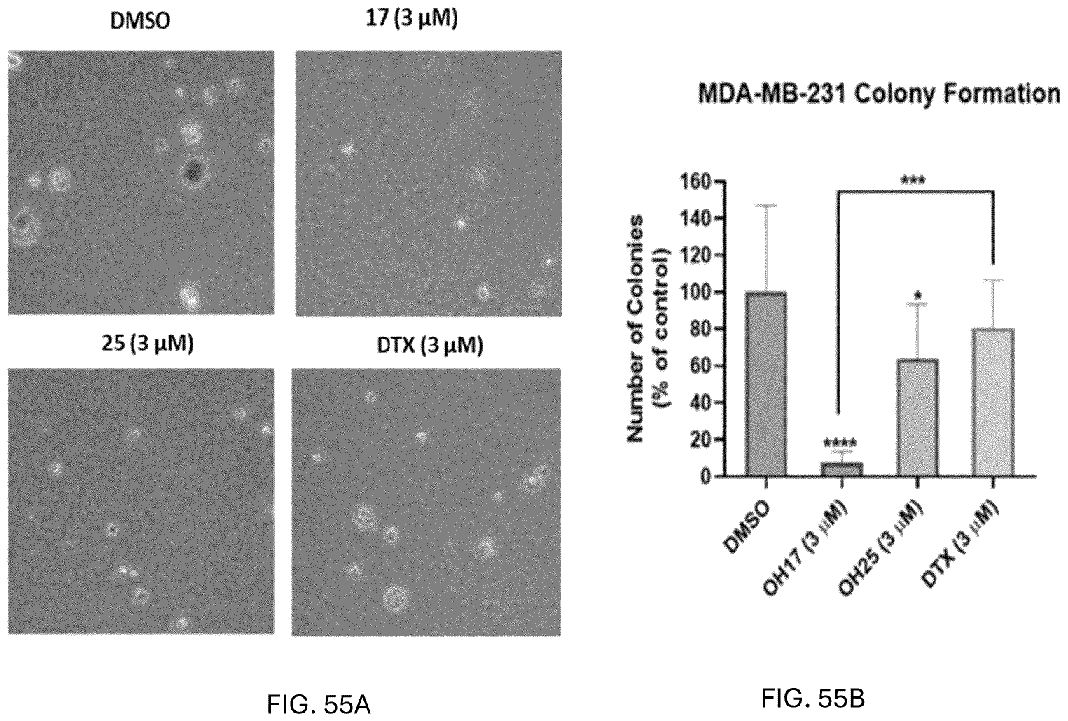

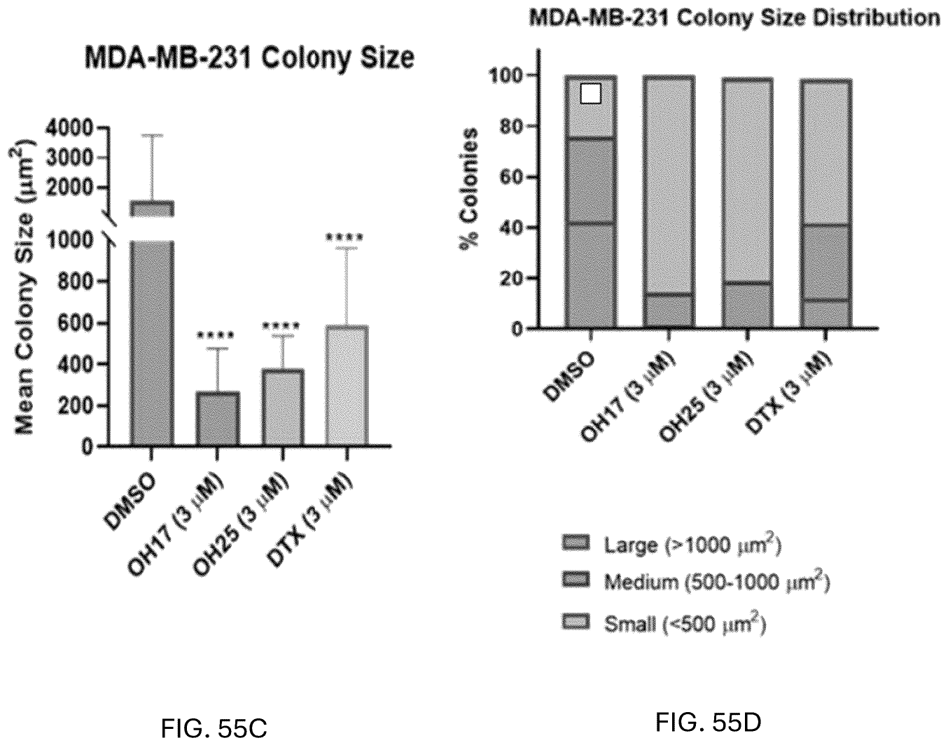

Applicant investigated colony formation of MDA-MB-231 and MDA-MB-436, in soft agar, under the effect of compounds 17 and 25, for 28 days. FIGS. 55A-55D show that compounds 17 and 23 resulted in a significant reduction in both the total number of colonies and average colony size in both cell lines. Interestingly, the reduction in number of colonies induced by compound 17 on MDA-231 cells was stronger than the positive control, docetaxel, when compared at the same concentration (P<0.001).

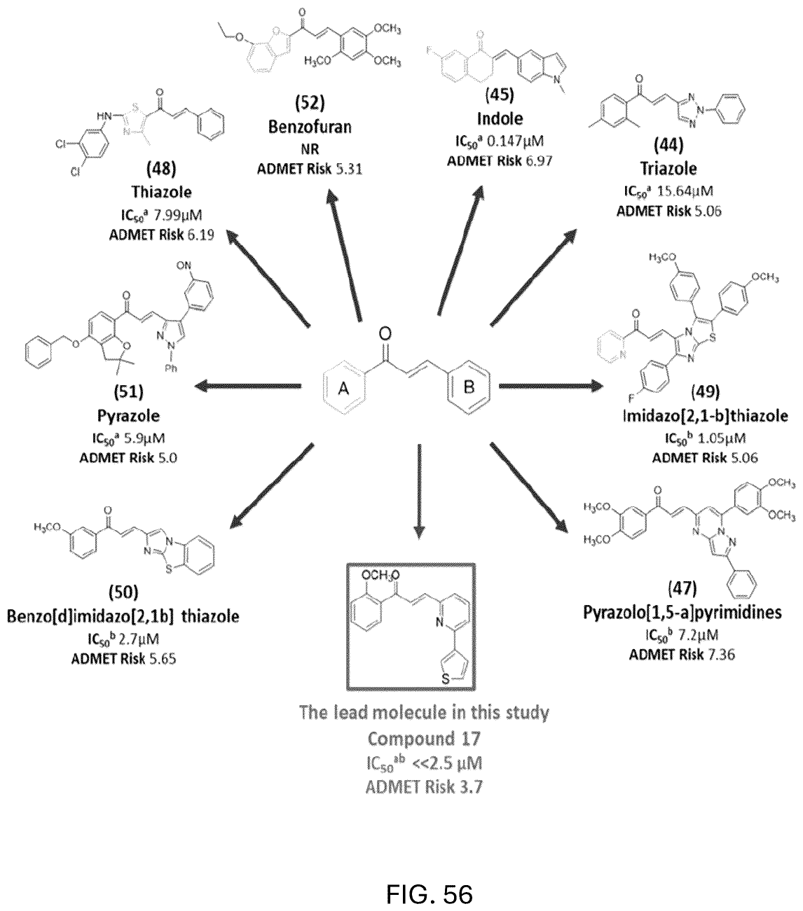

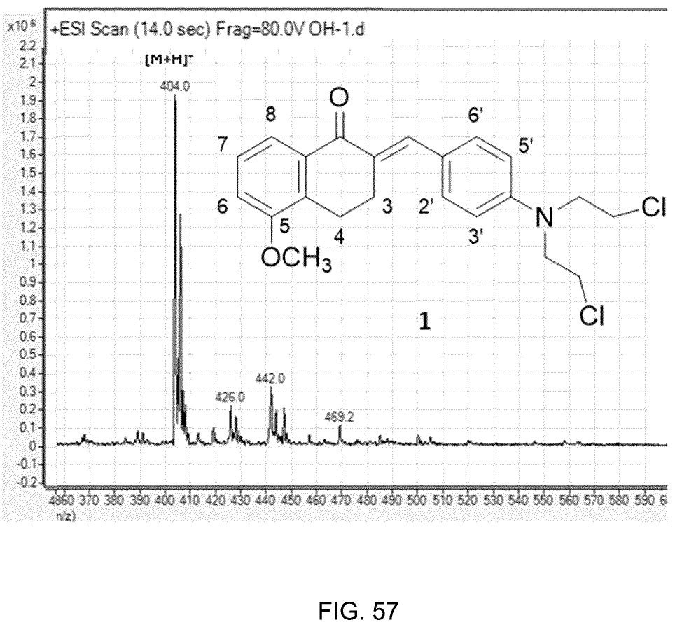

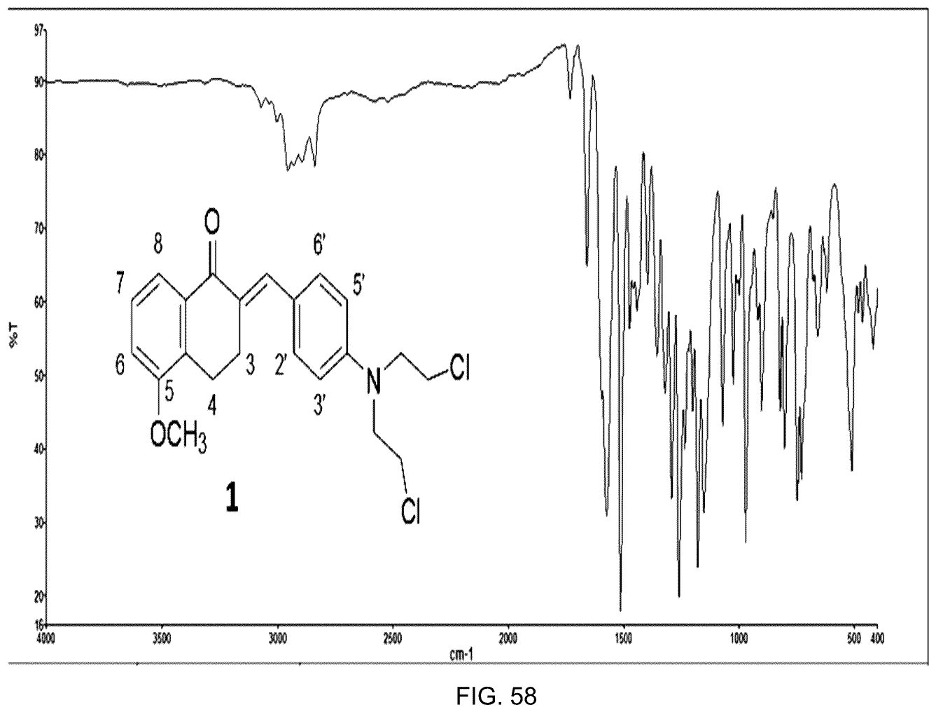

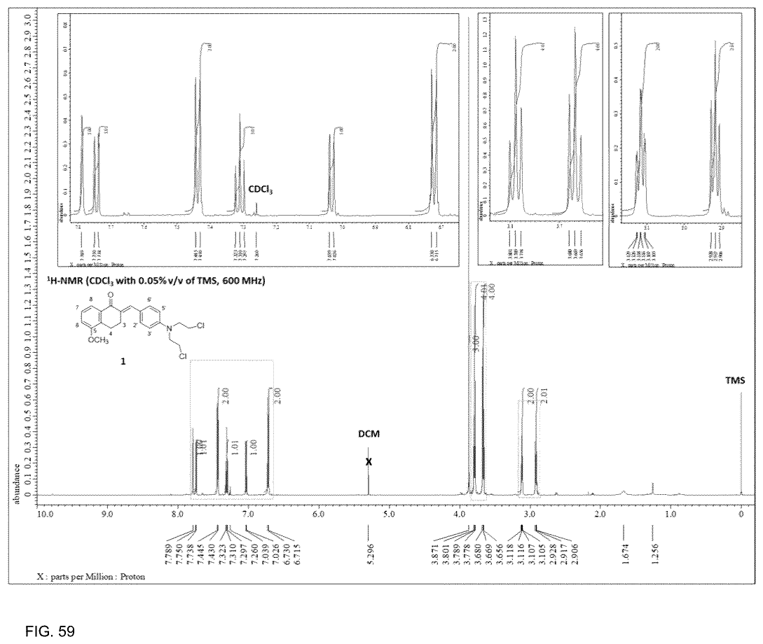

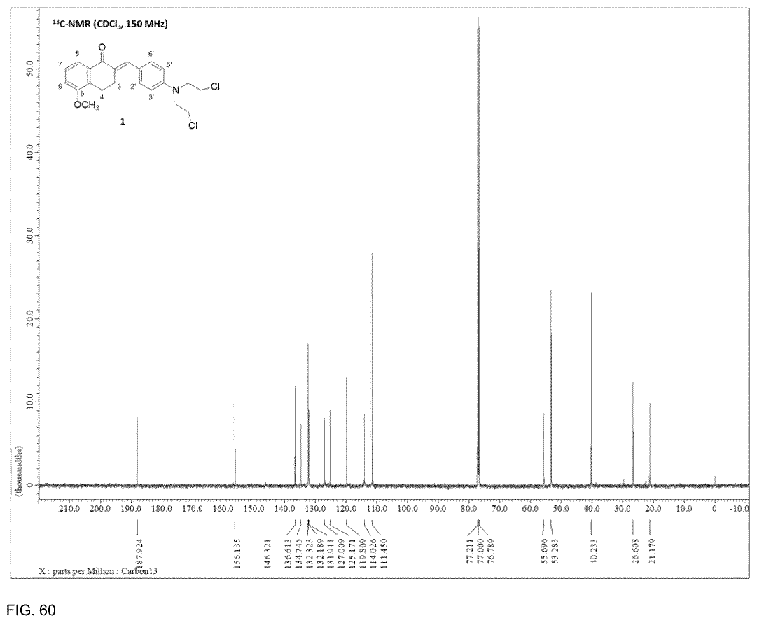

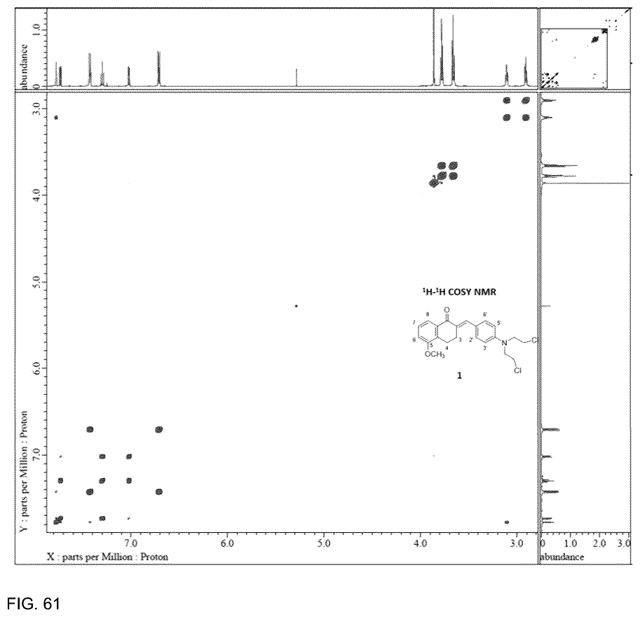

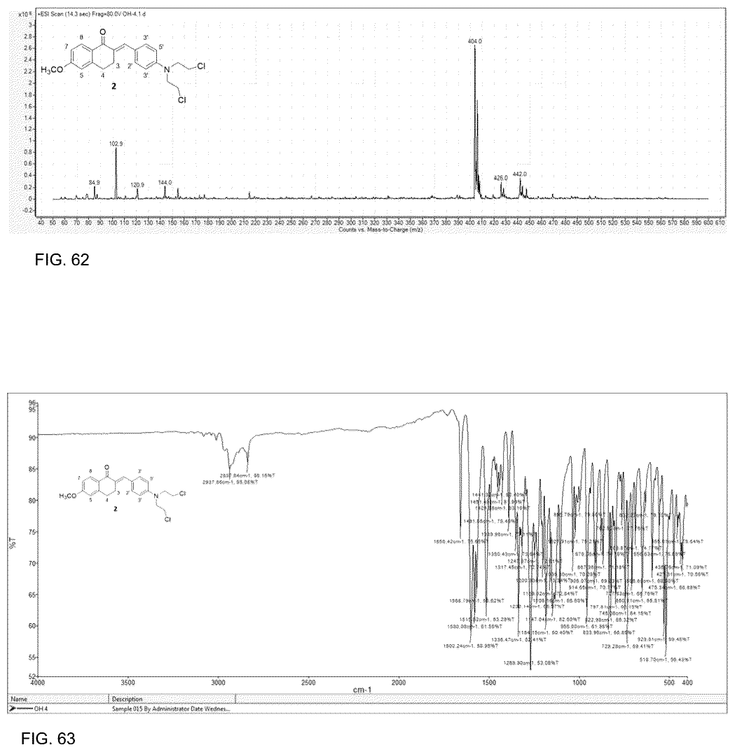

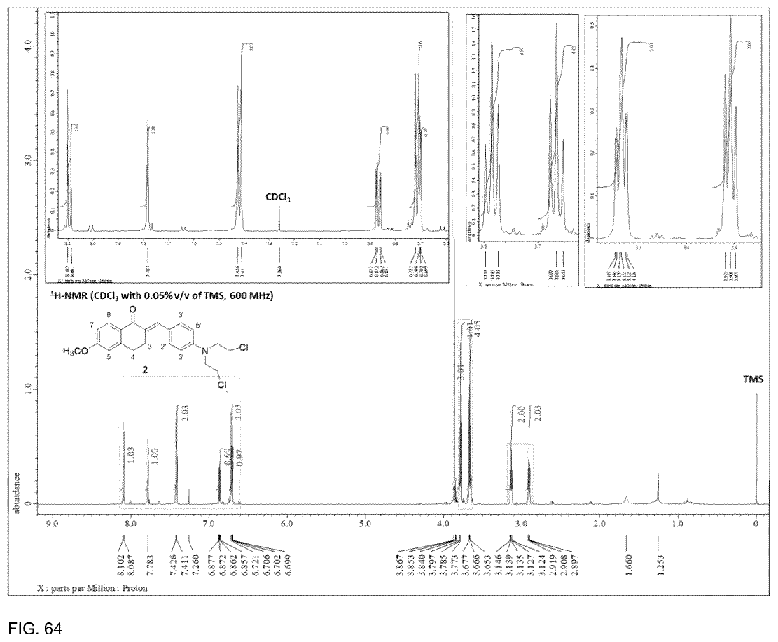

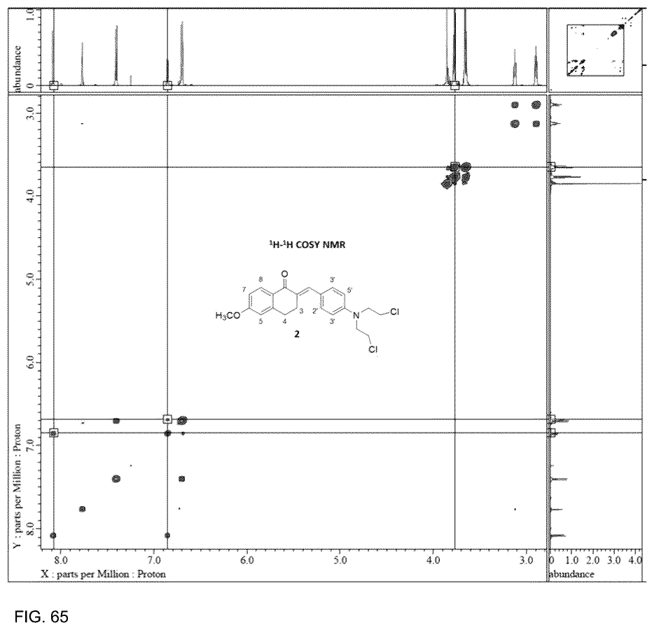

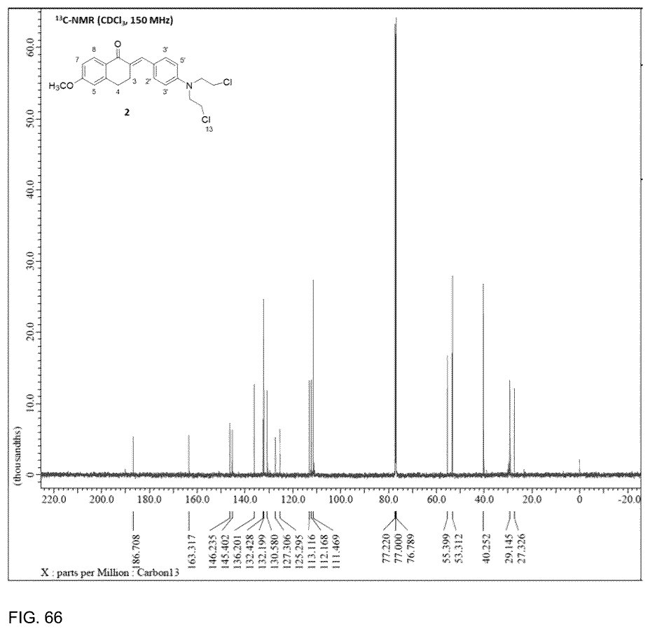

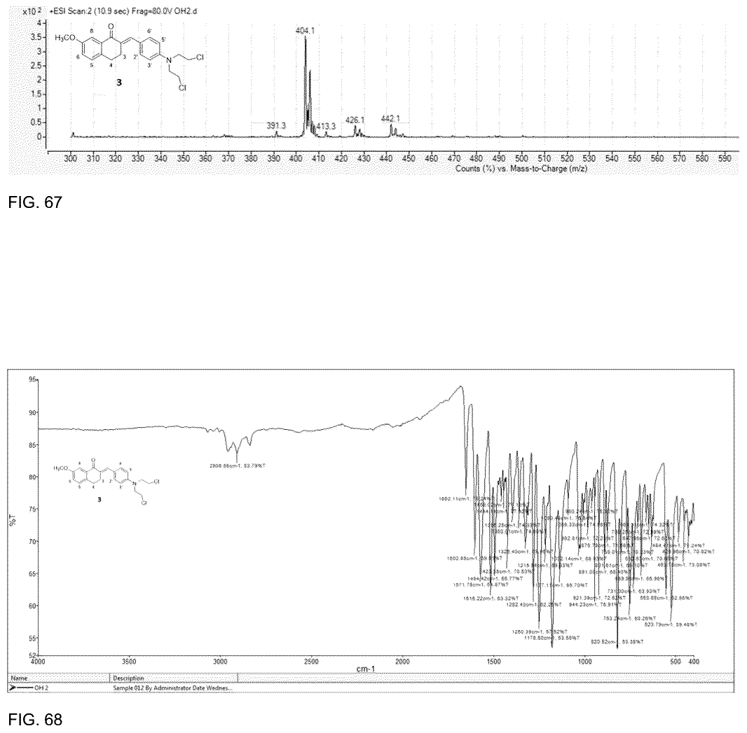

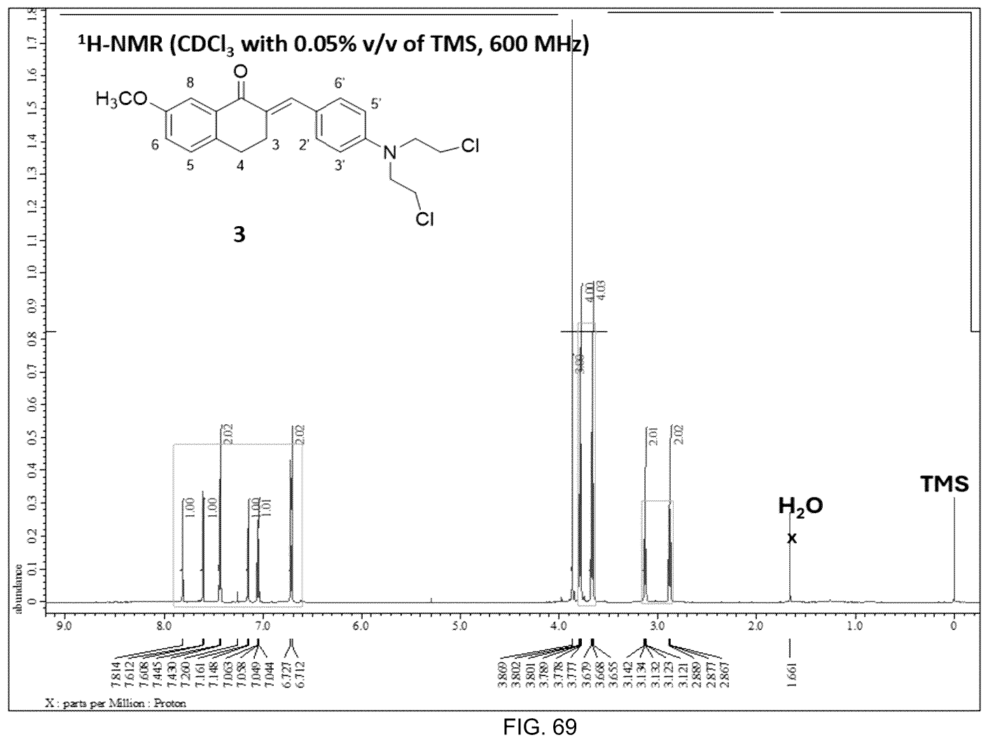

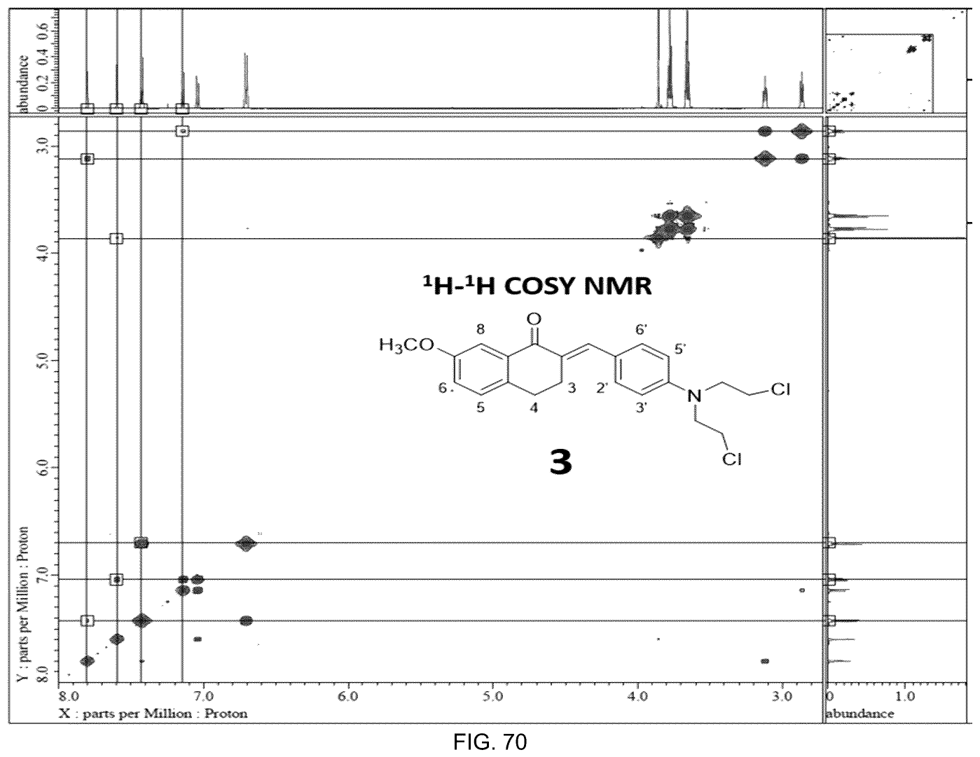

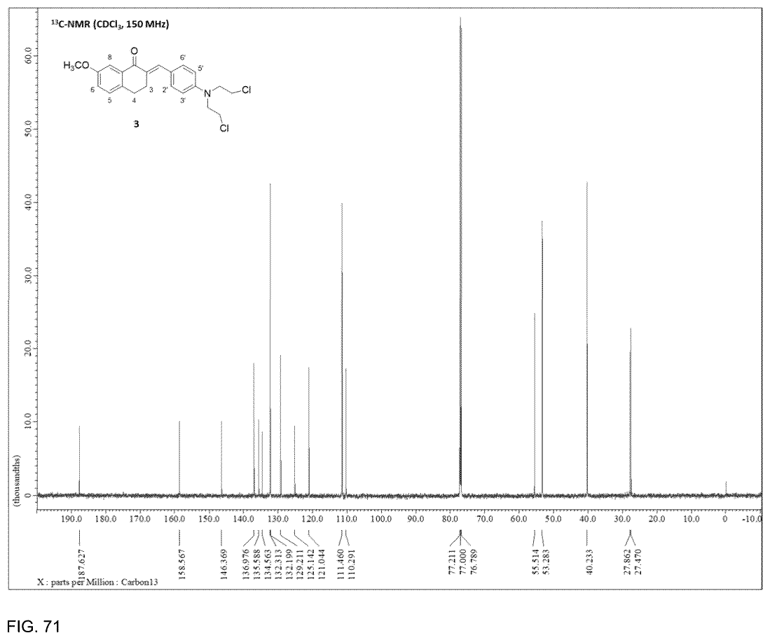

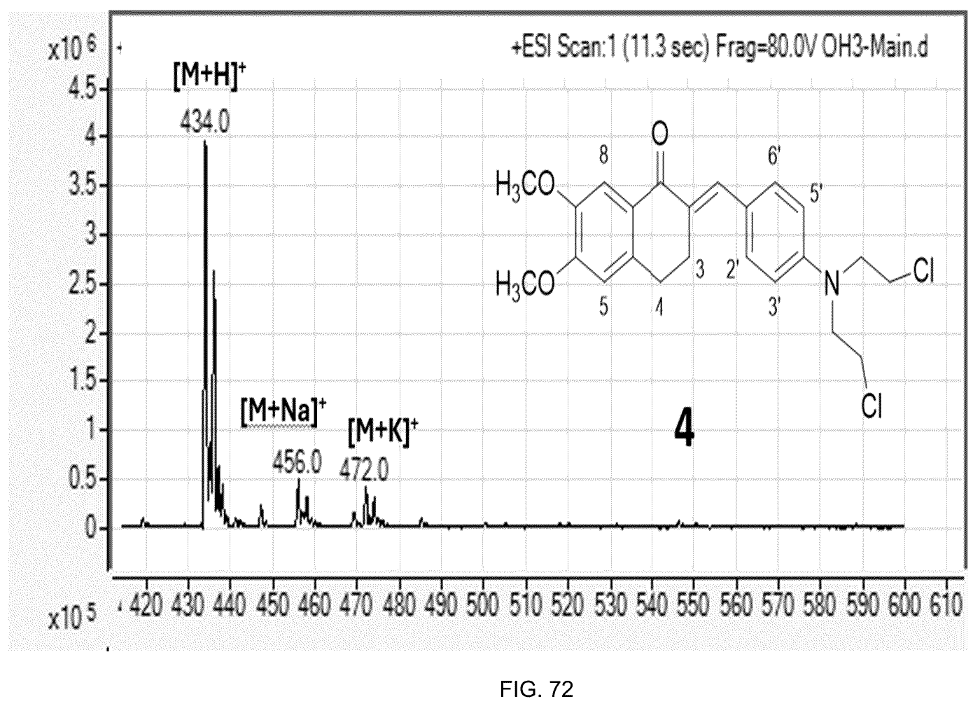

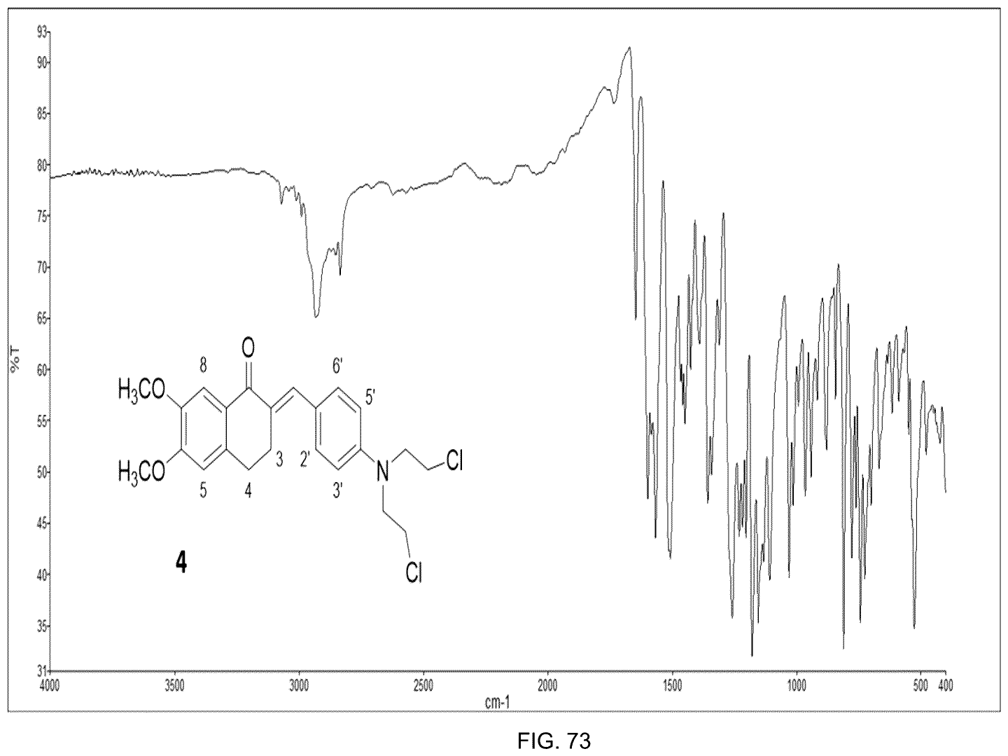

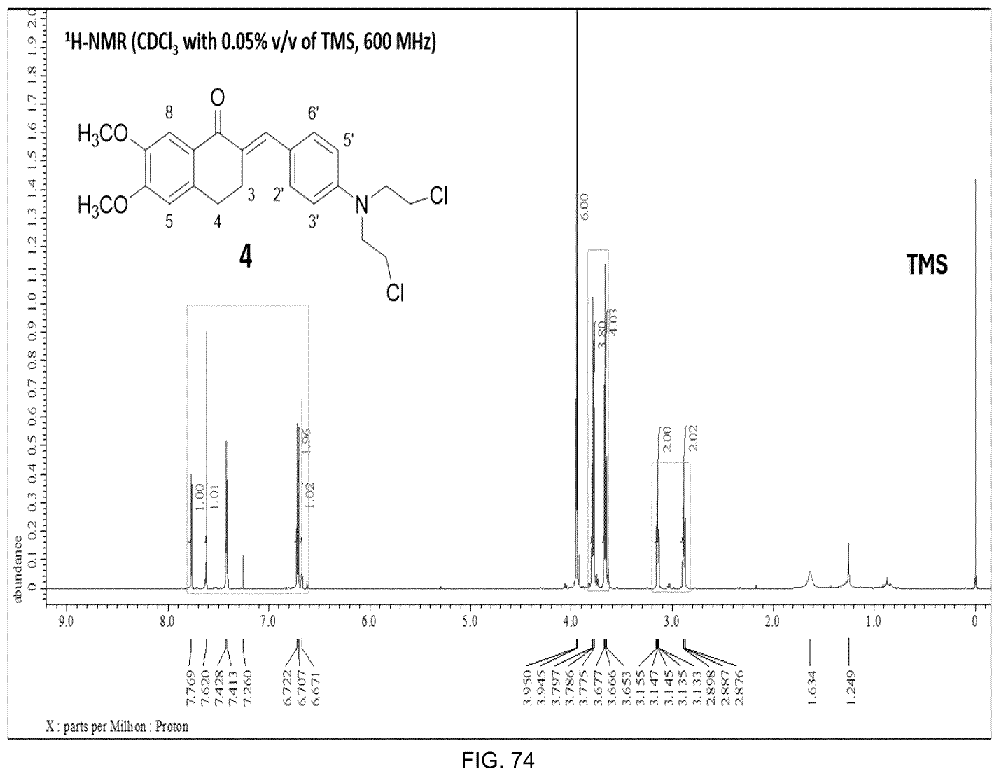

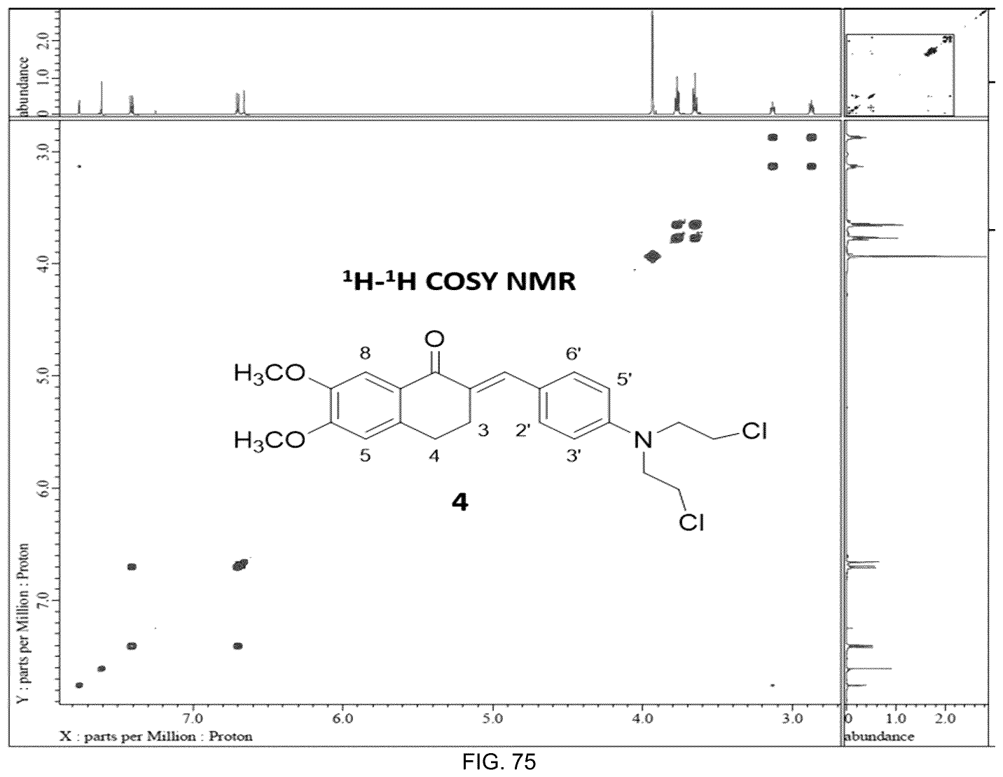

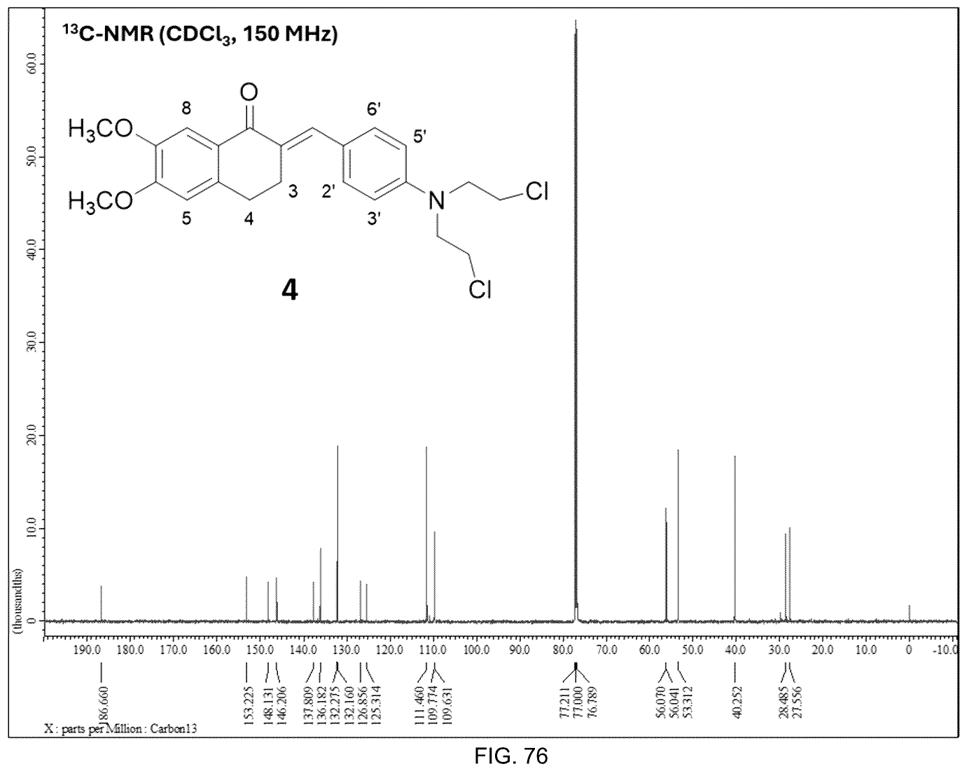

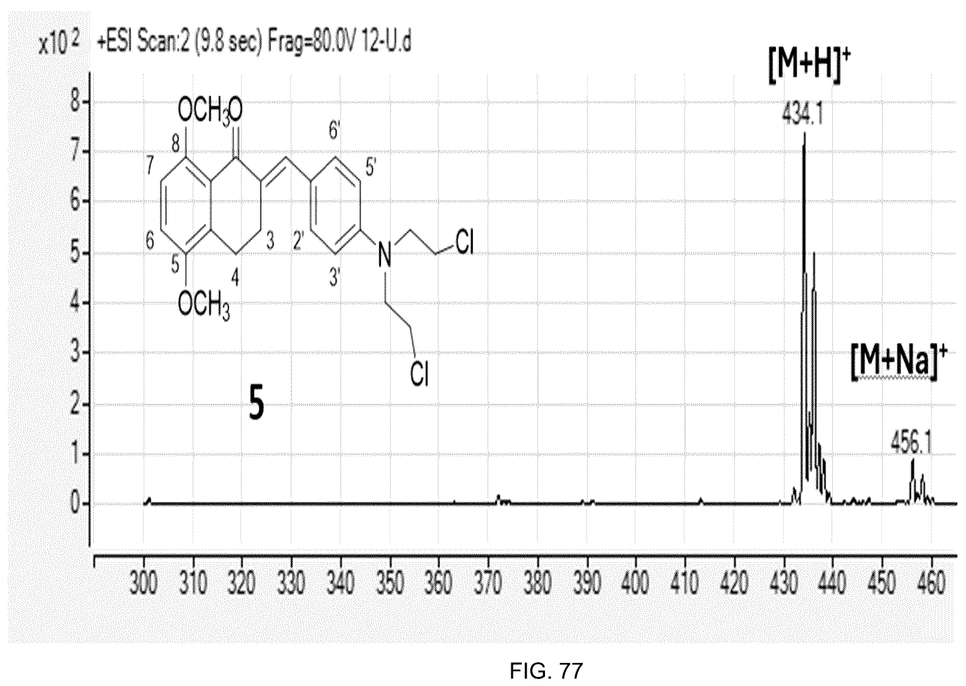

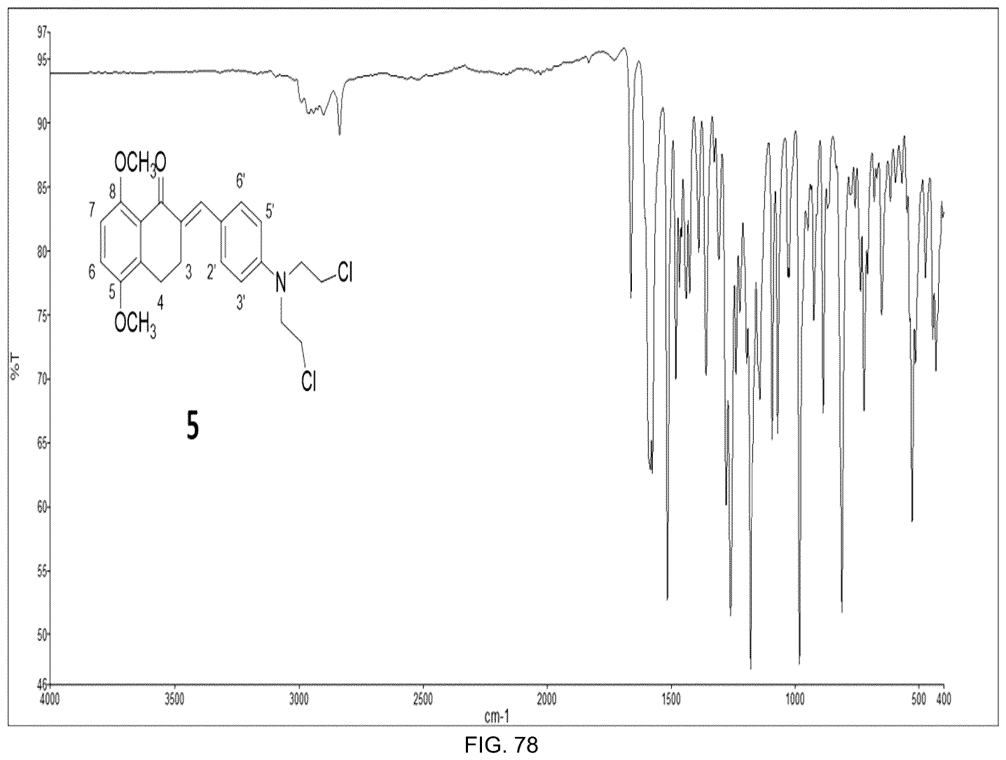

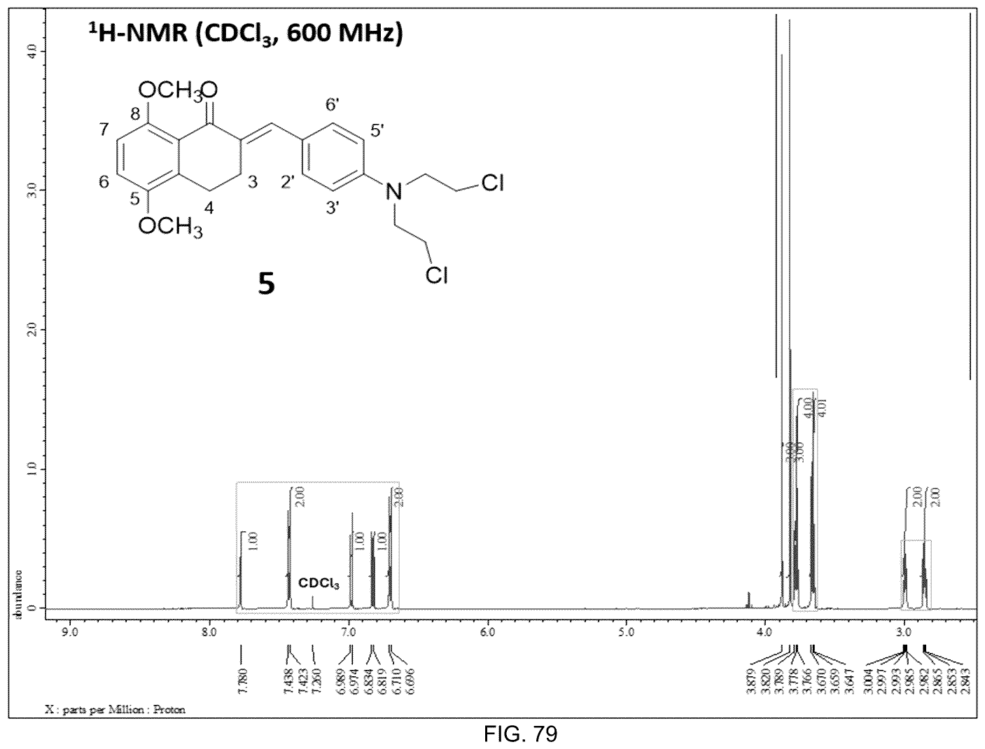

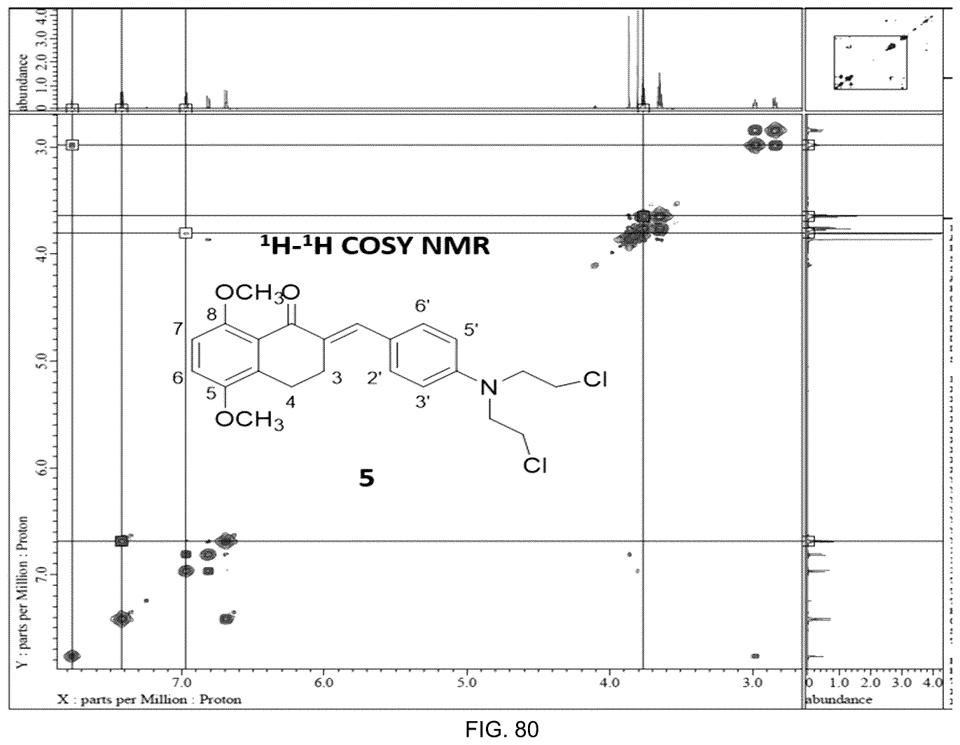

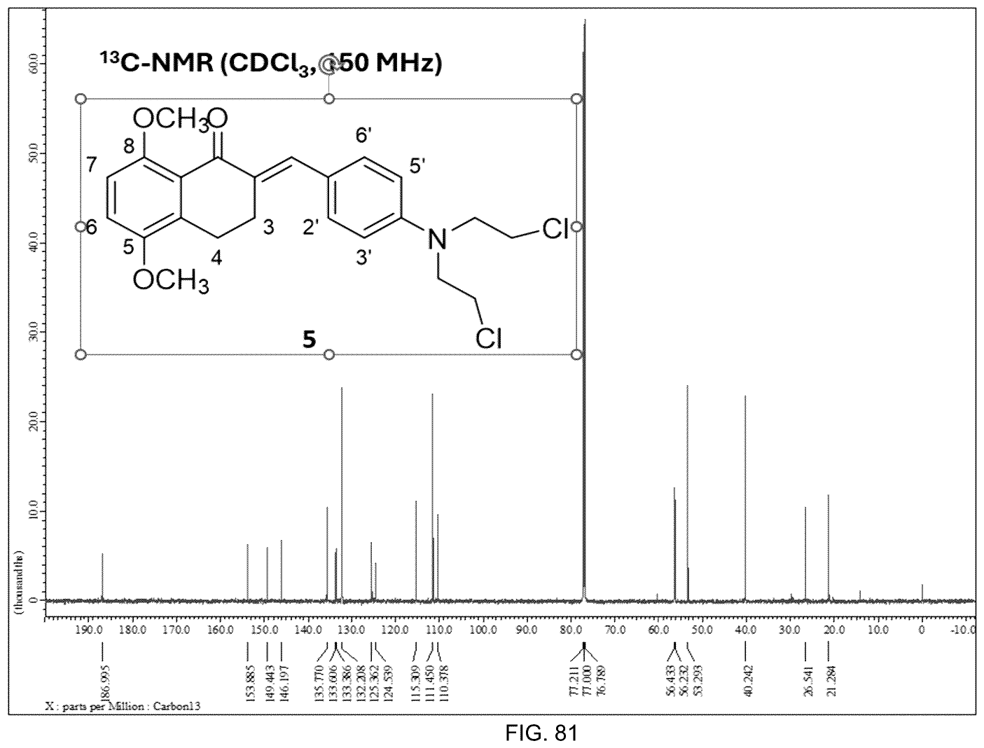

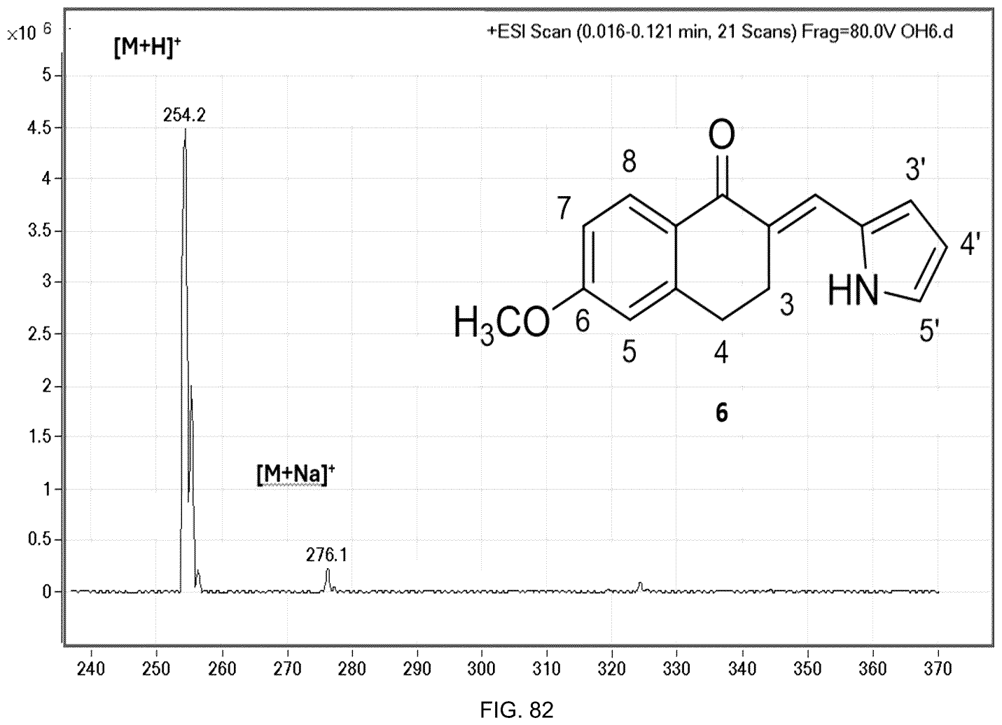

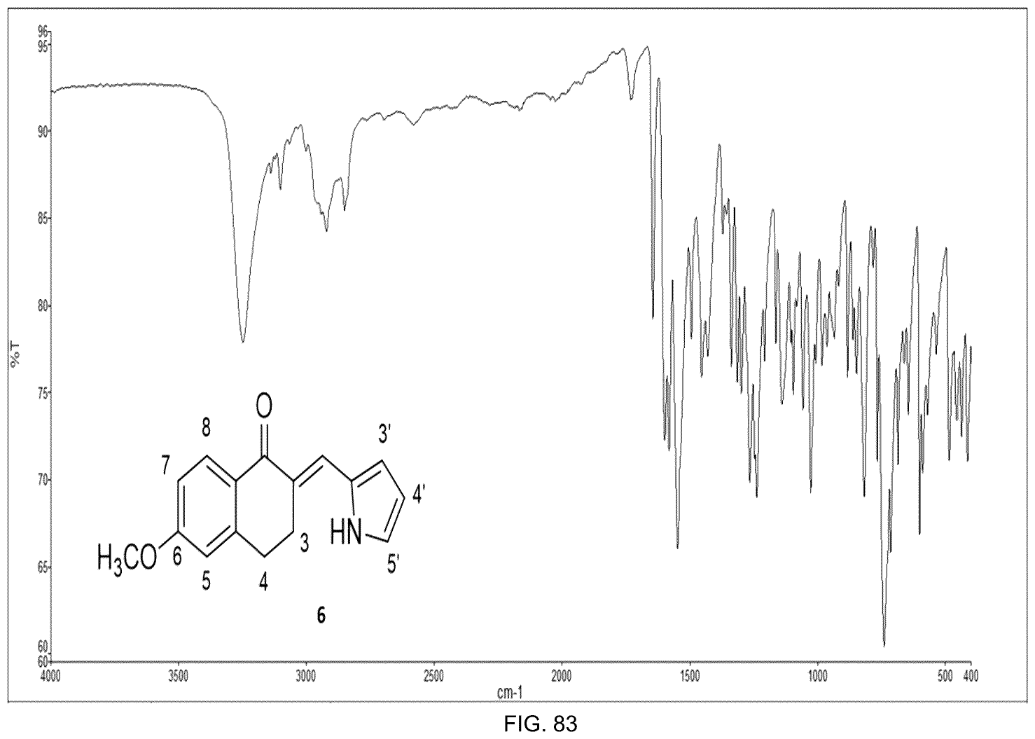

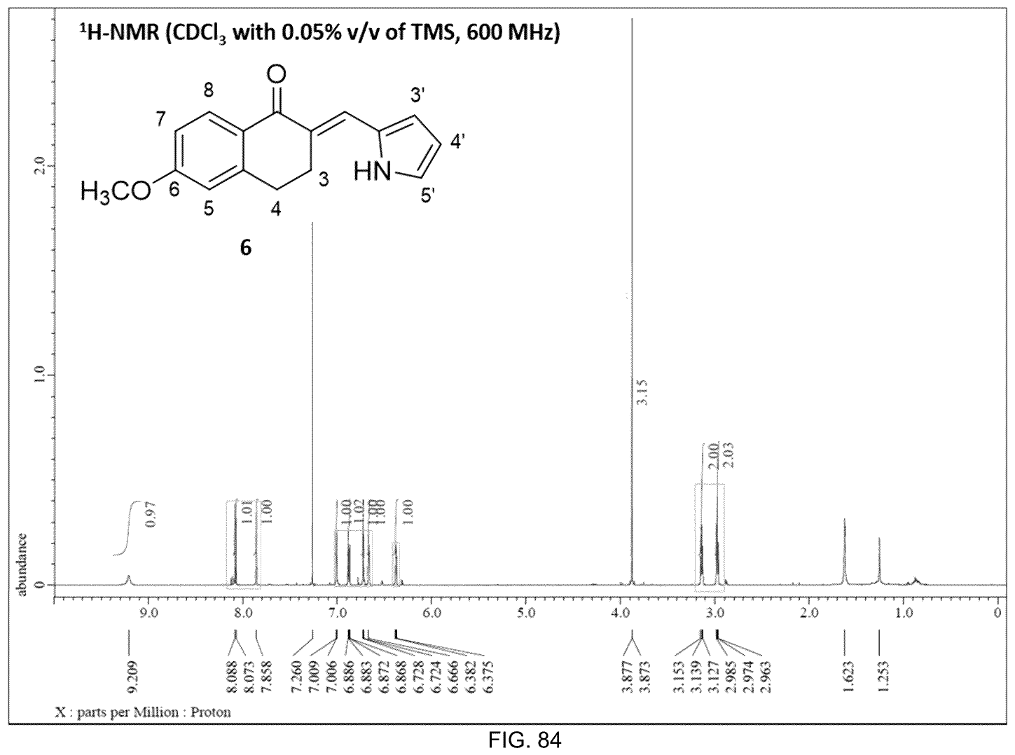

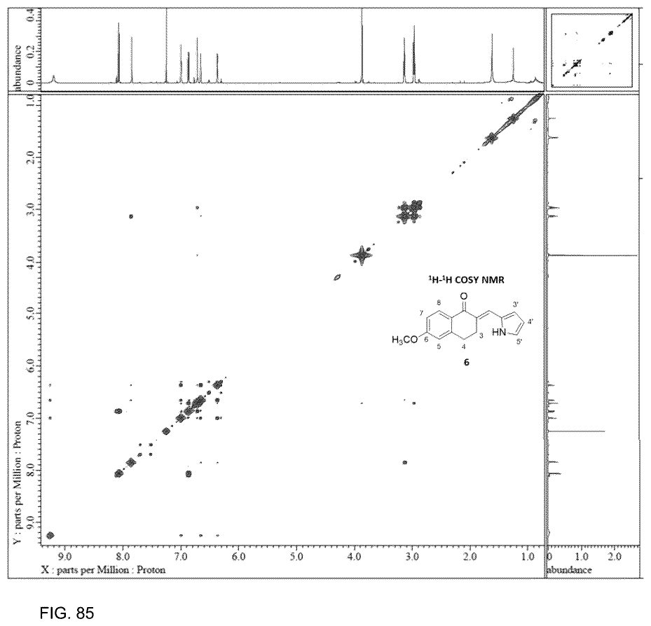

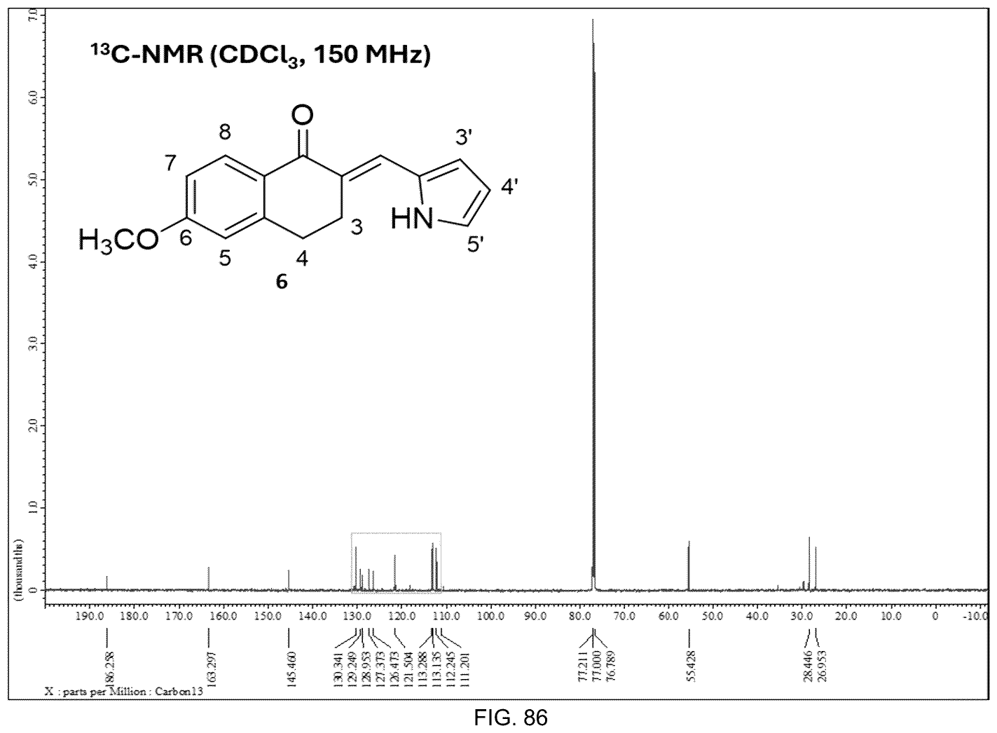

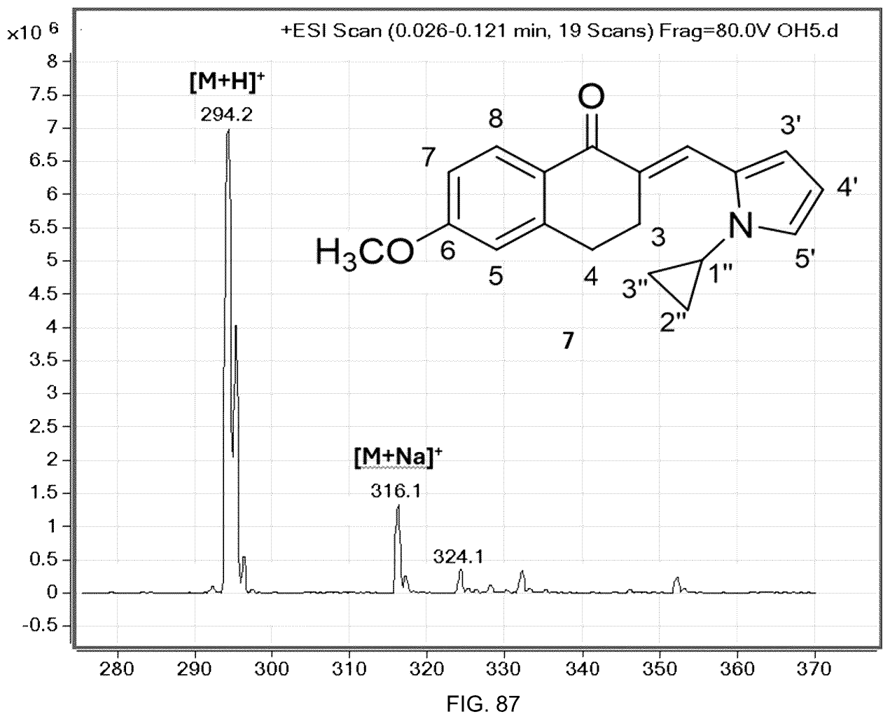

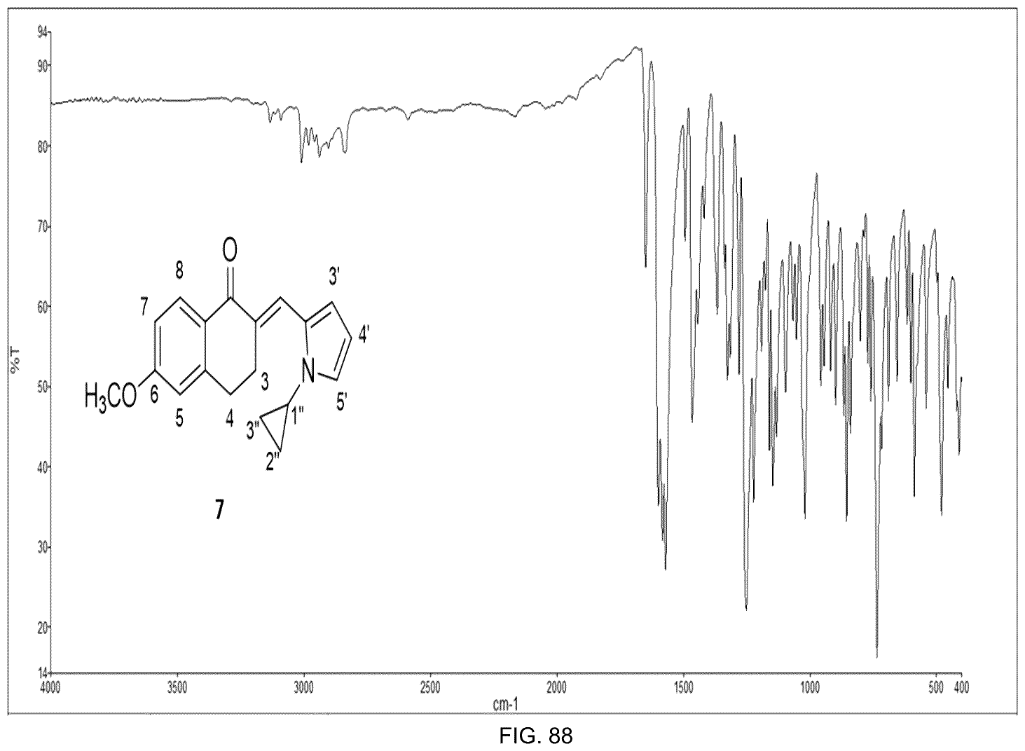

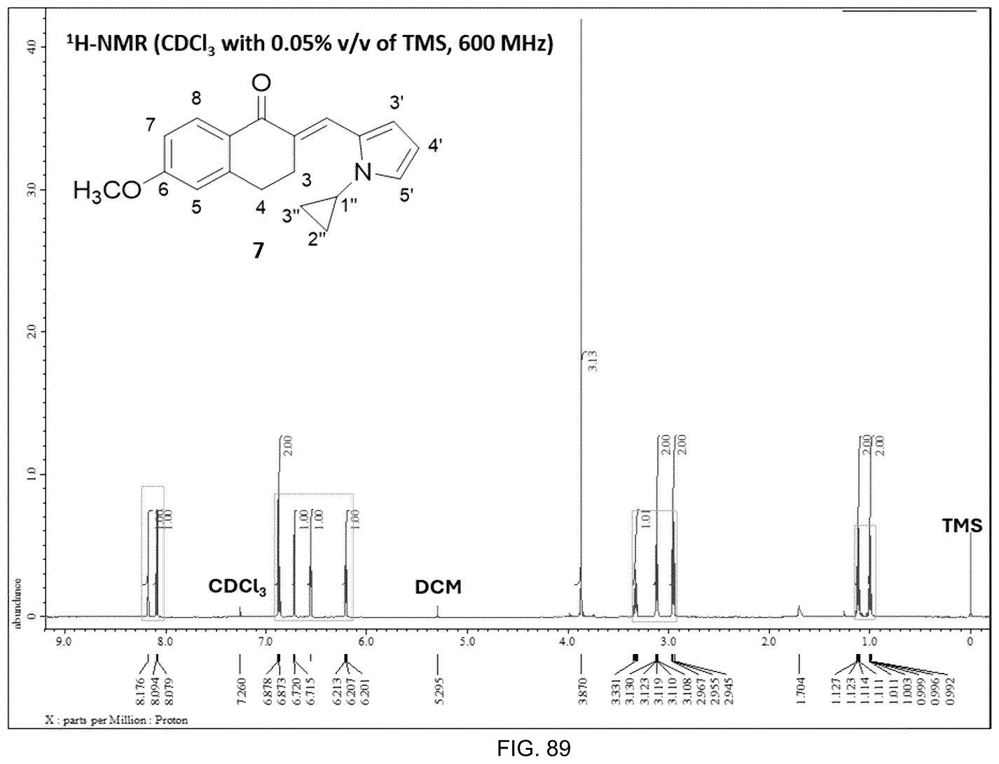

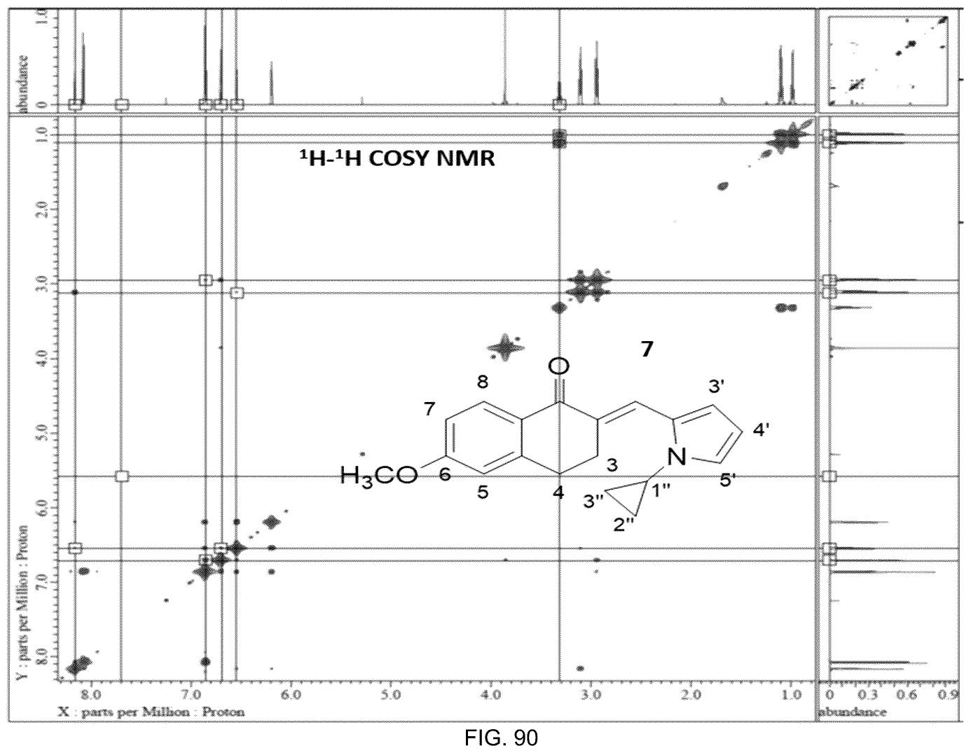

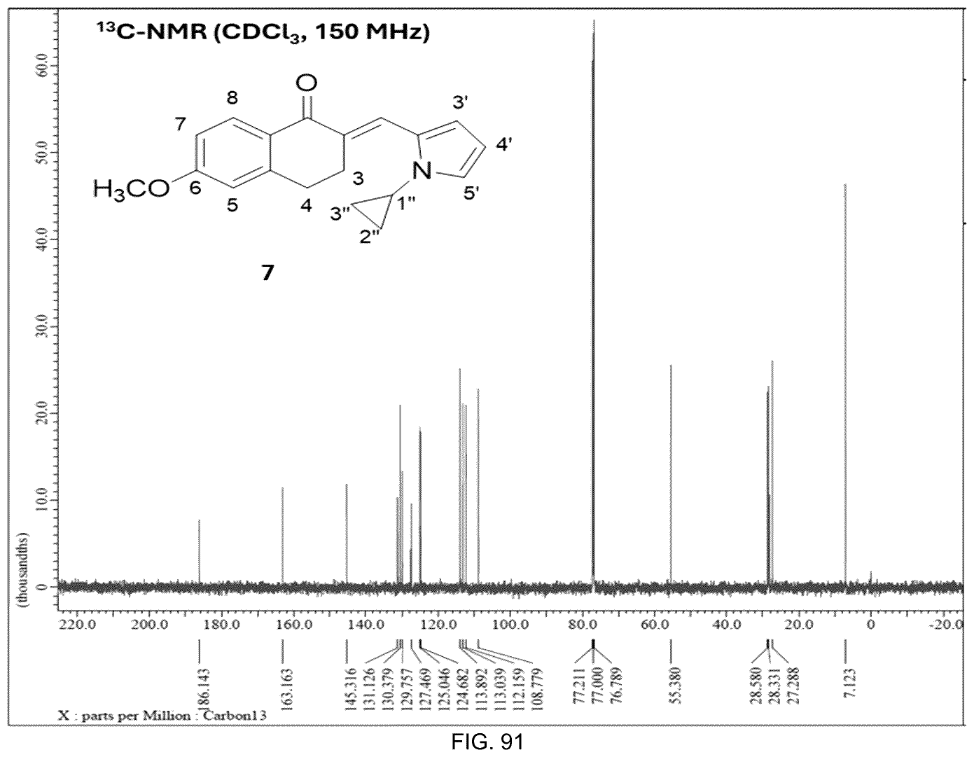

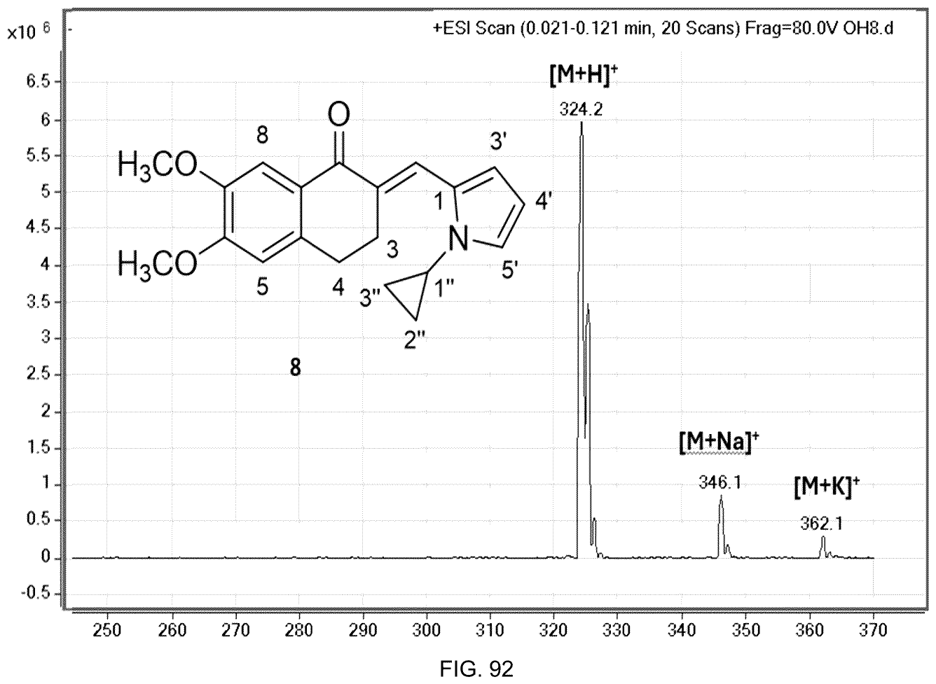

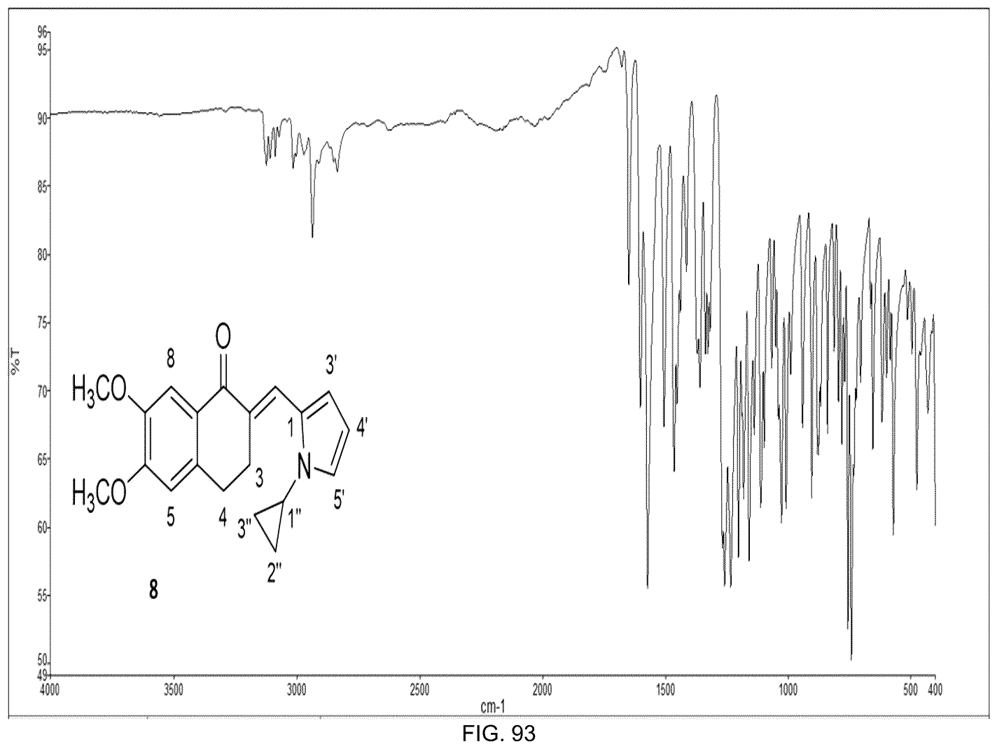

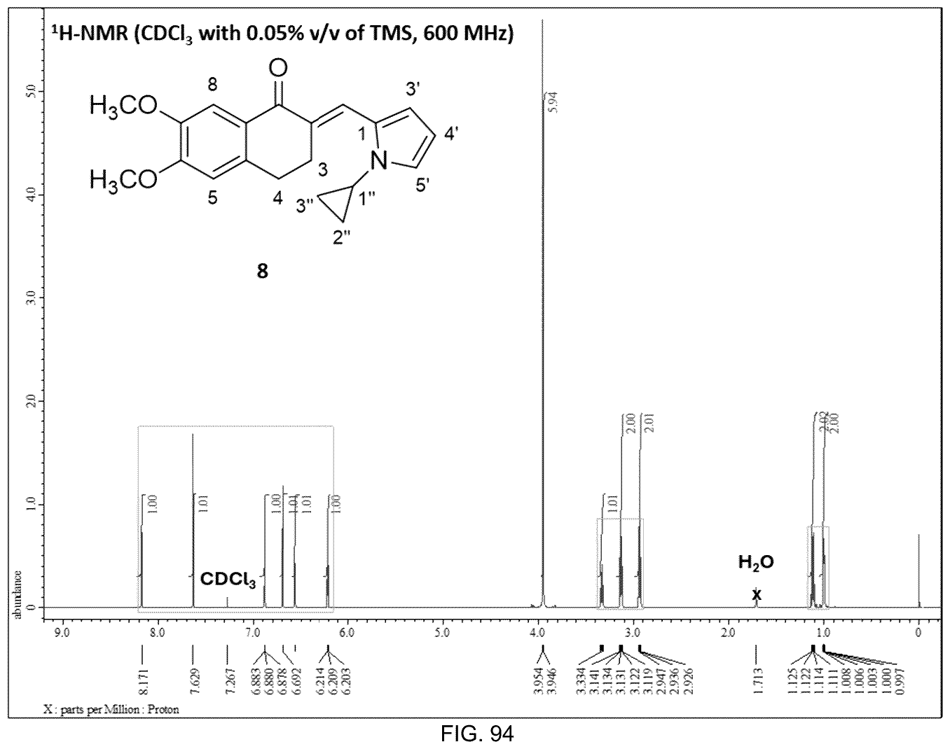

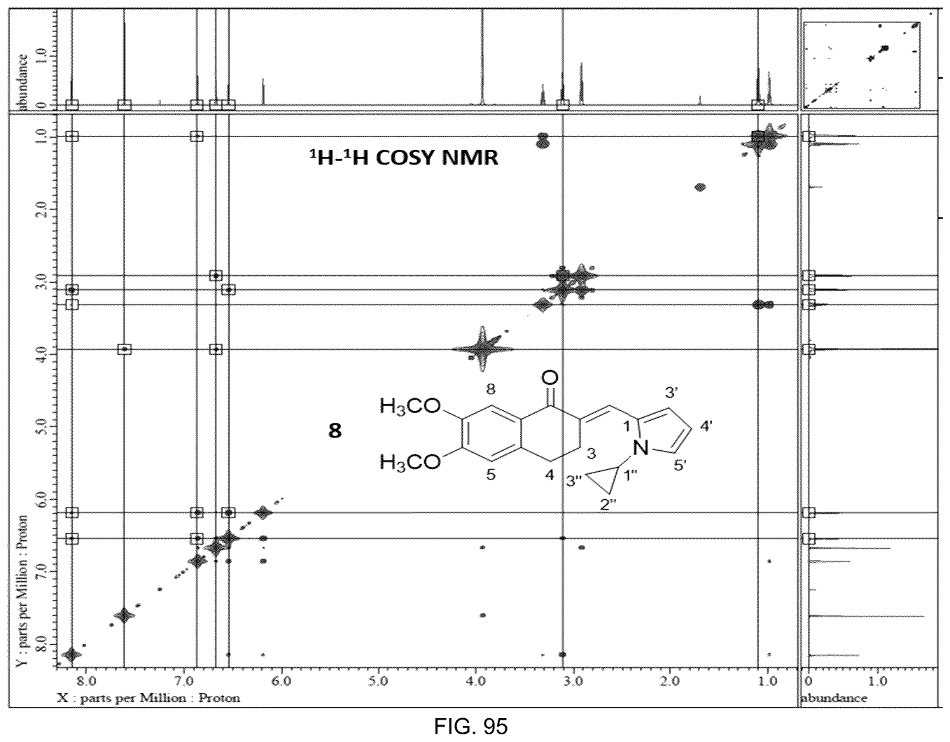

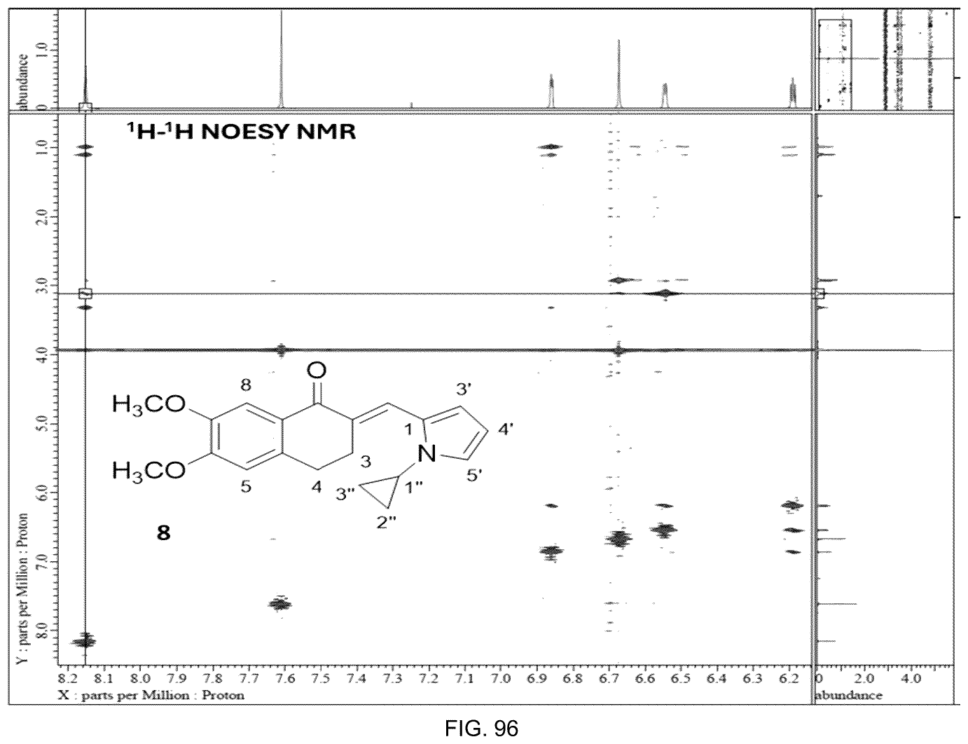

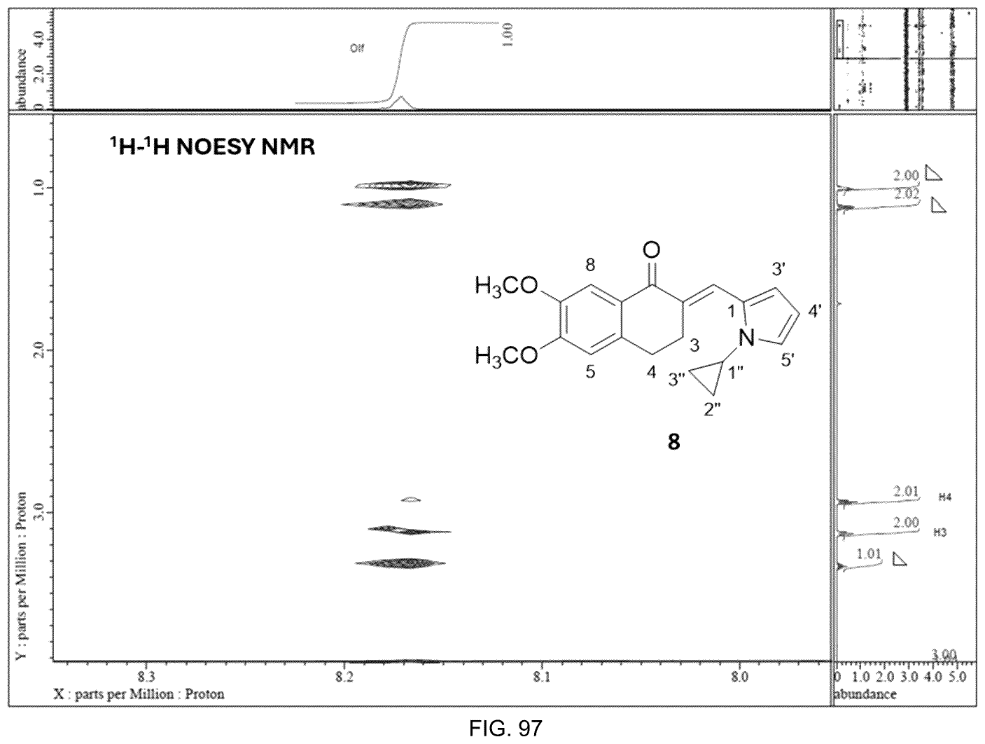

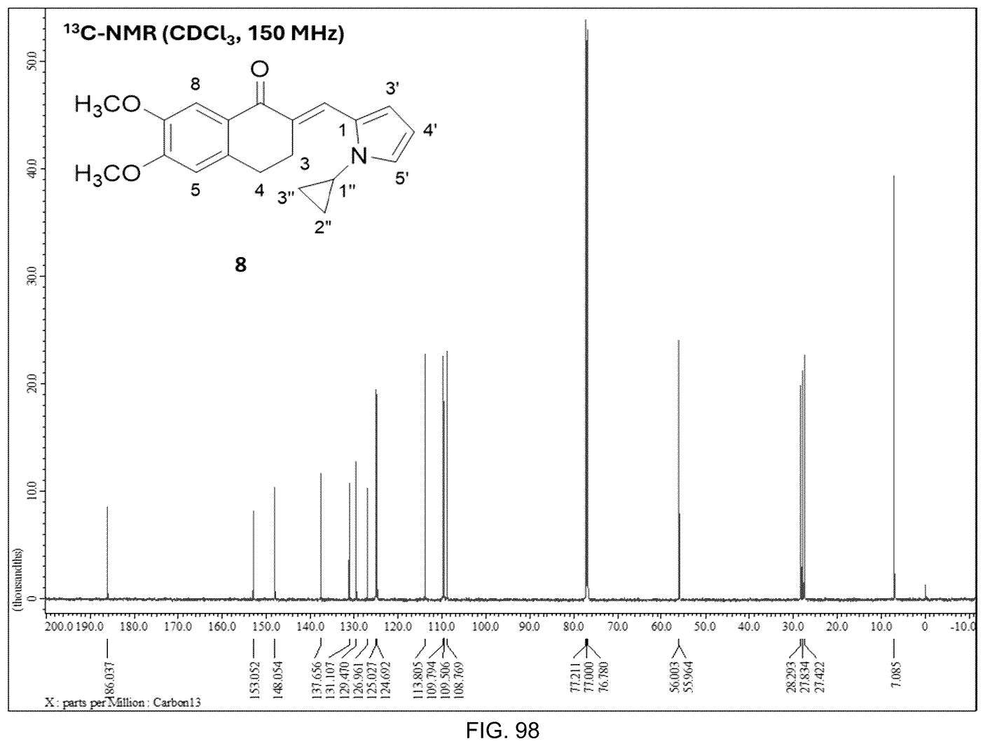

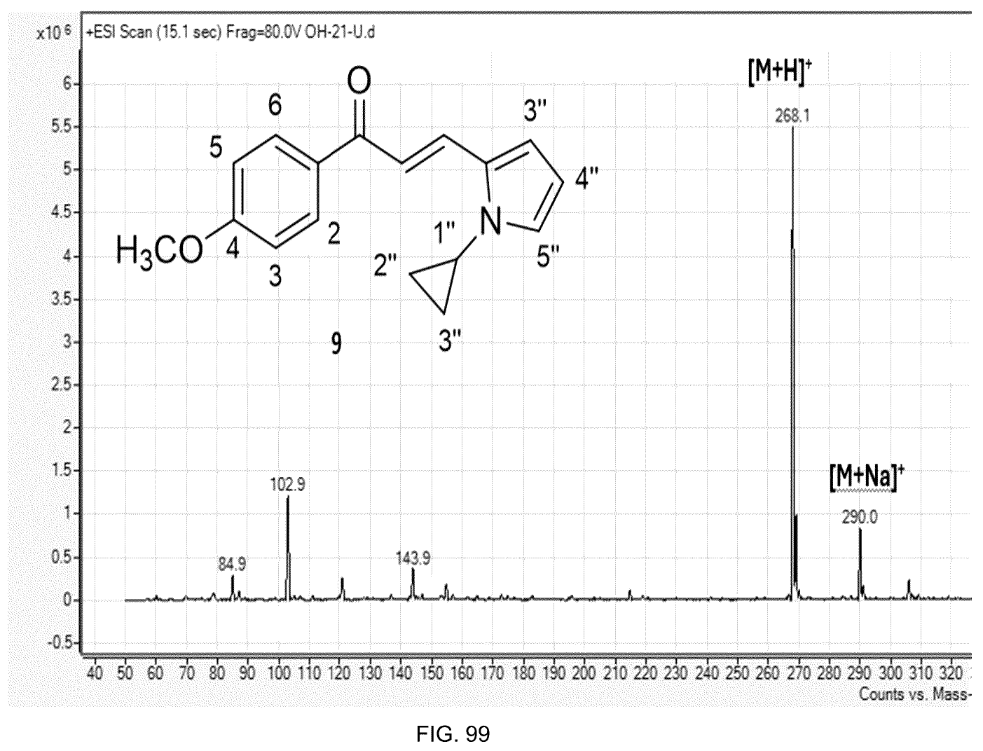

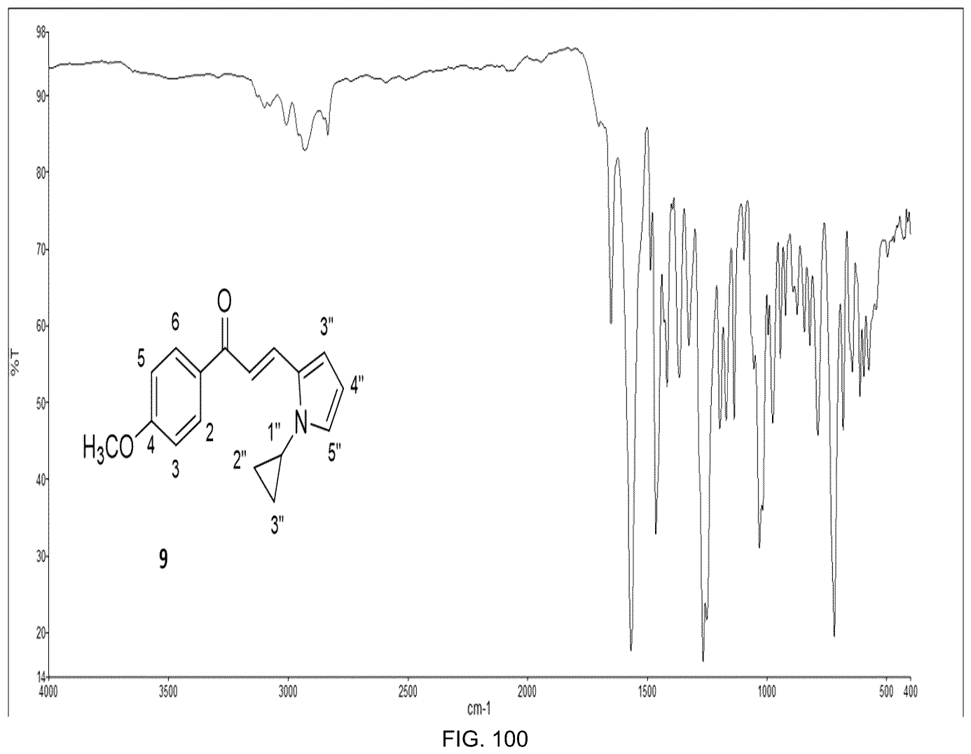

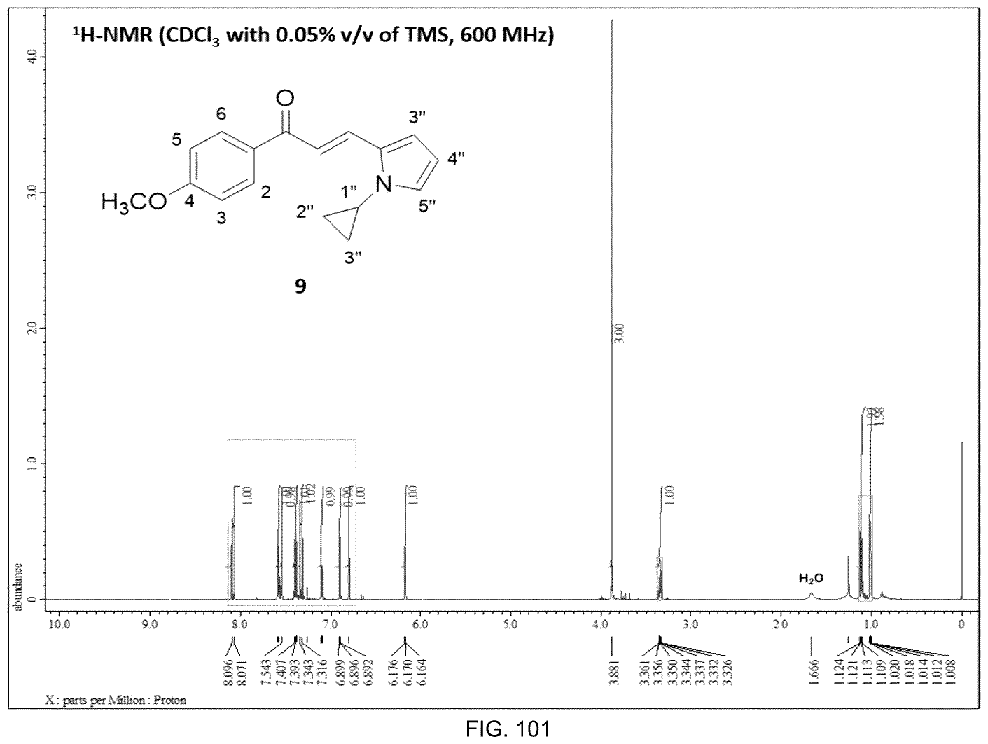

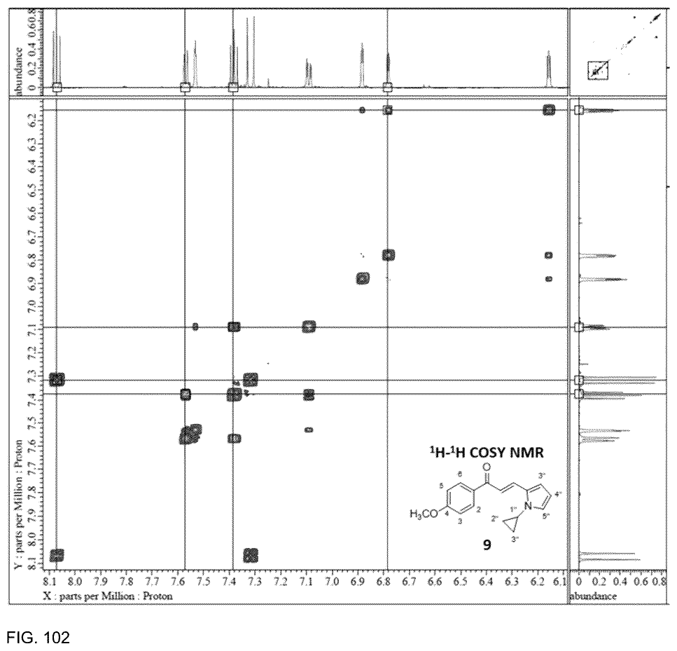

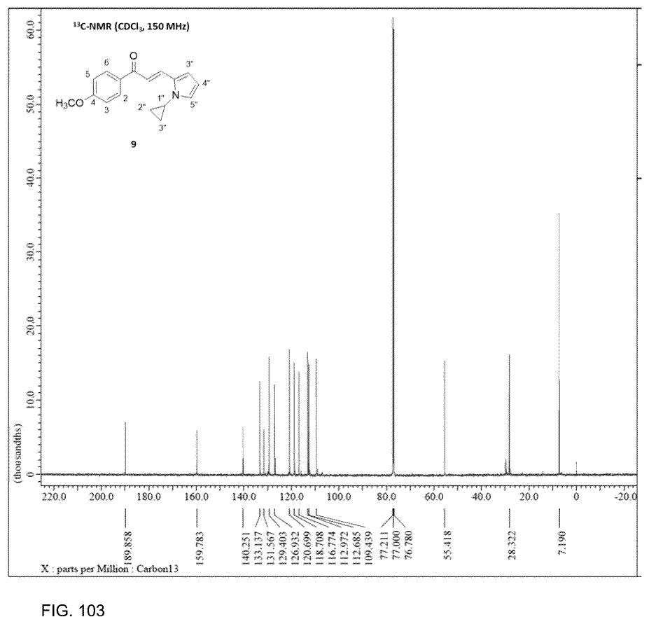

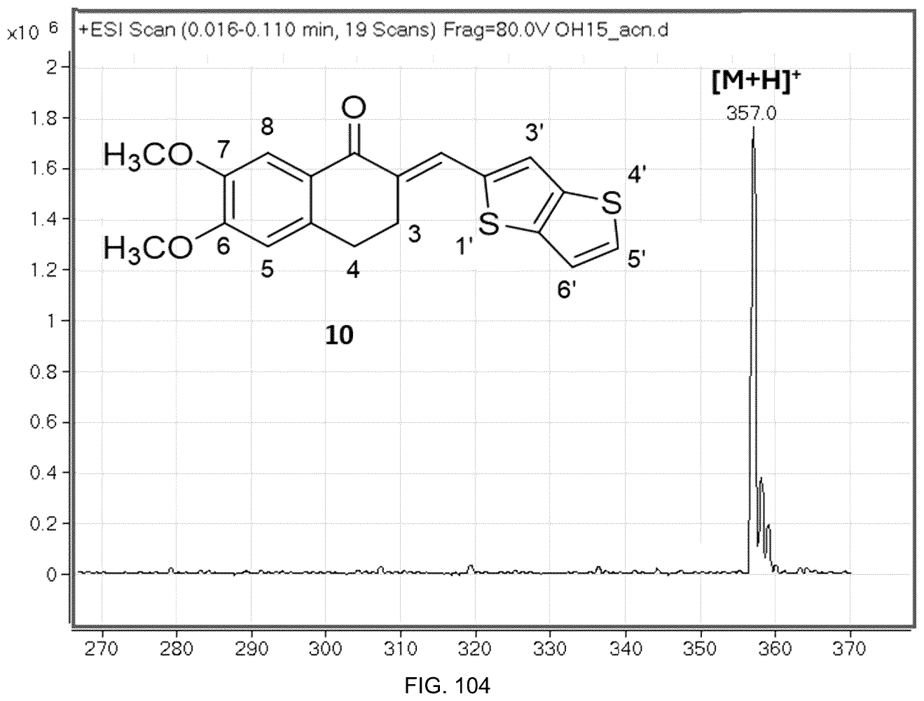

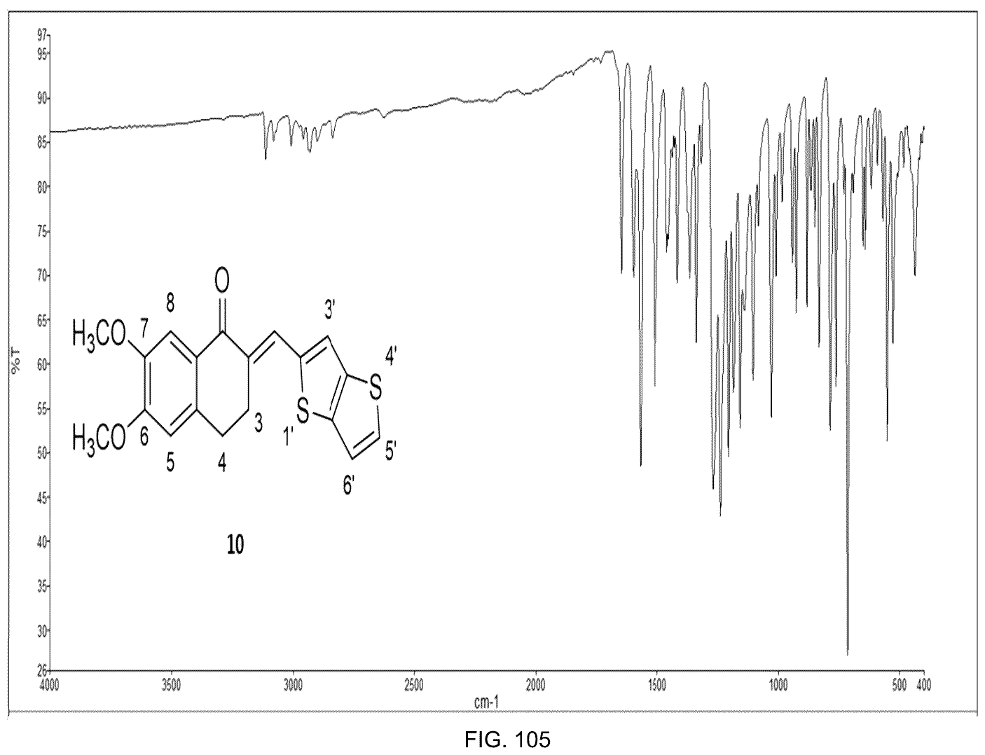

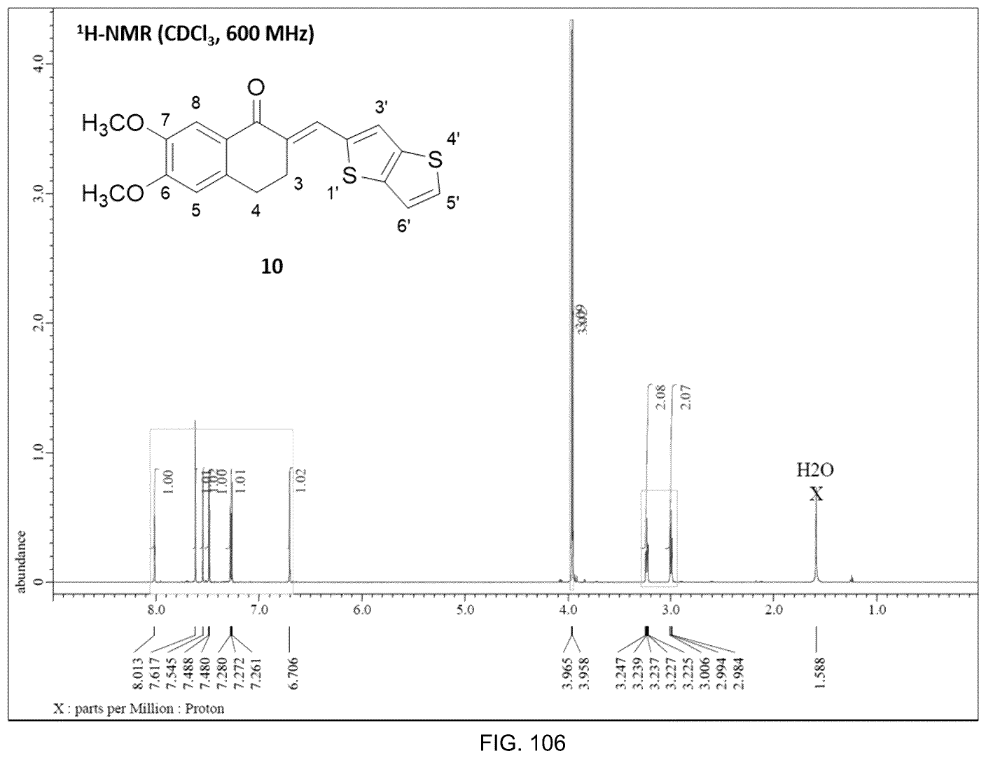

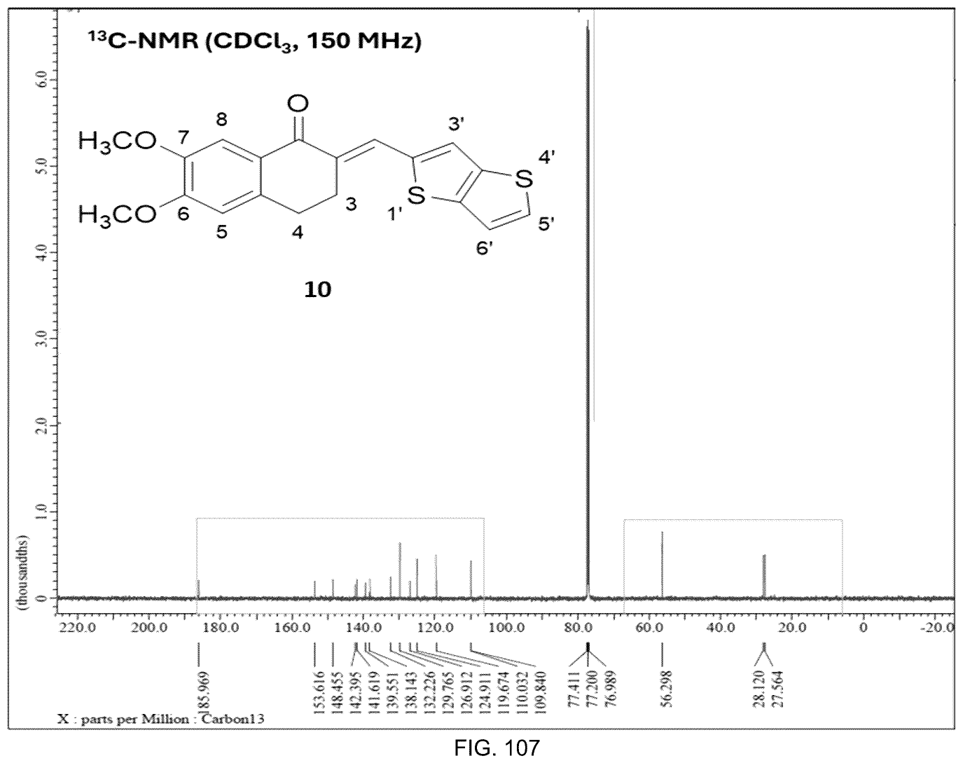

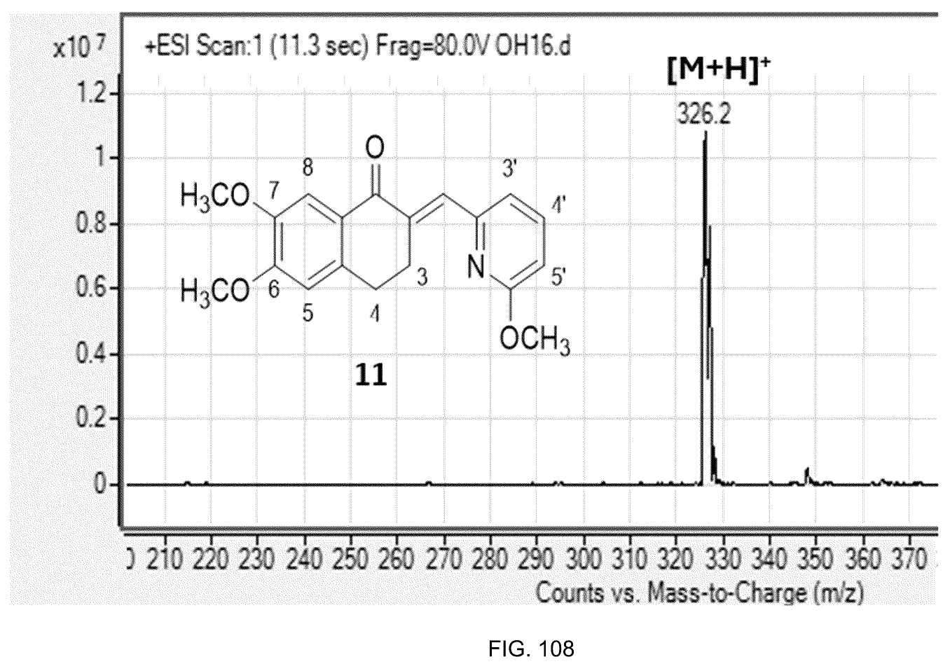

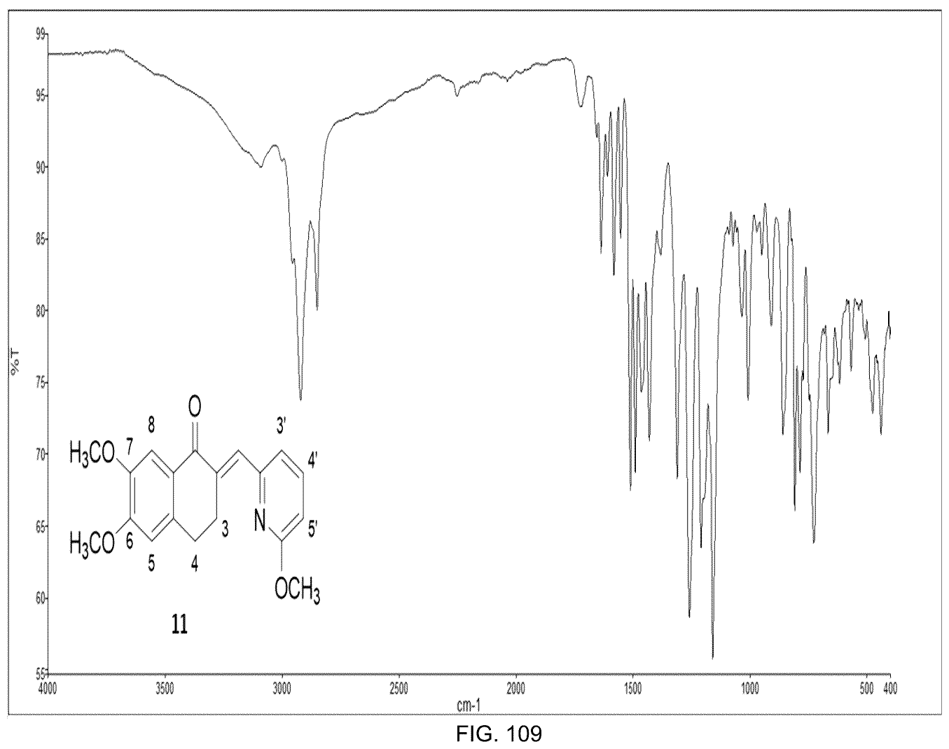

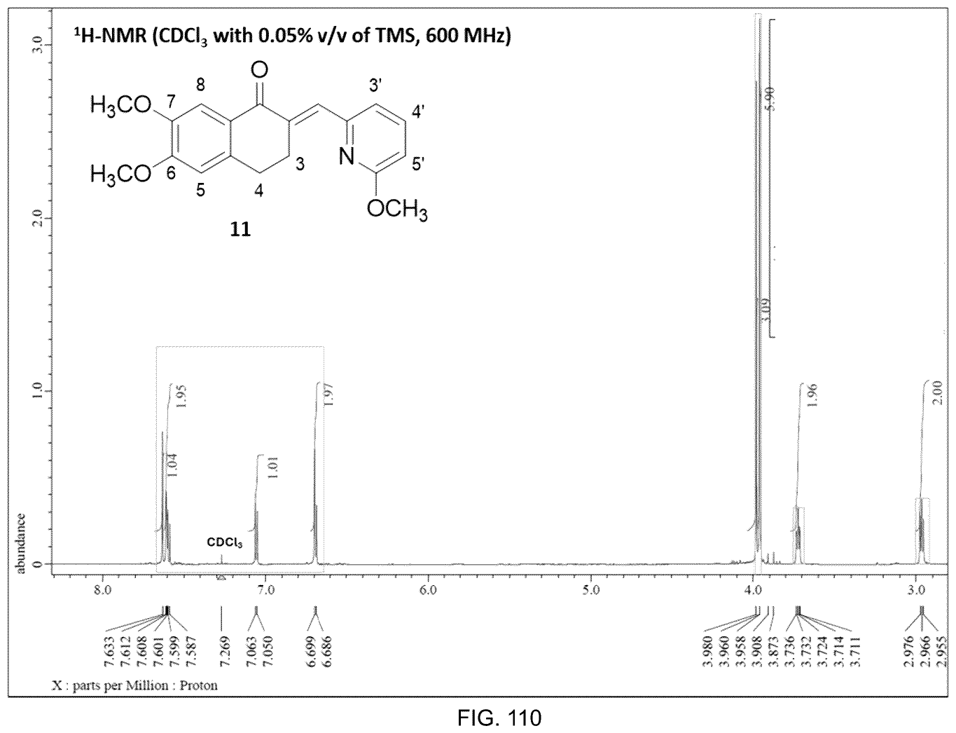

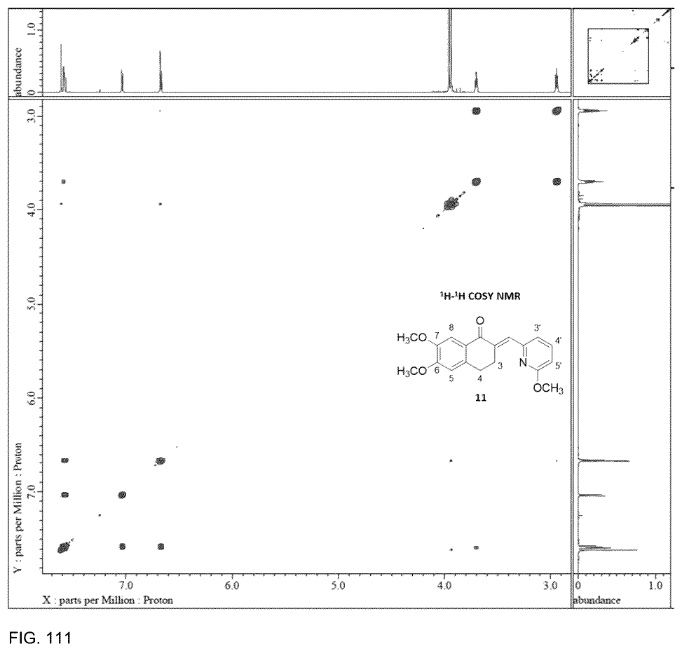

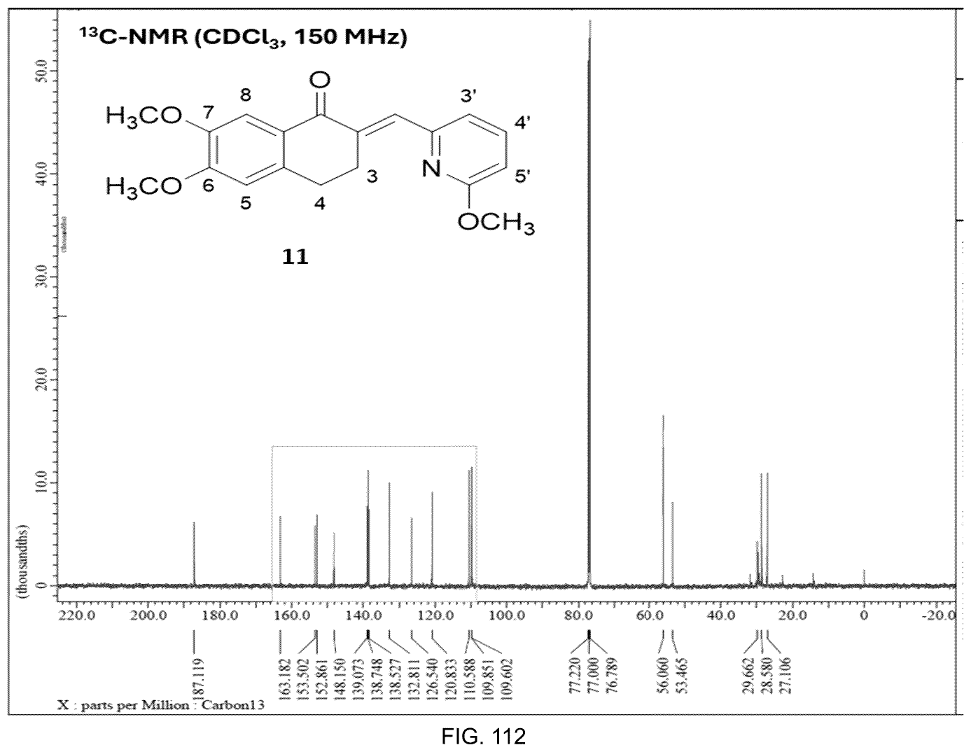

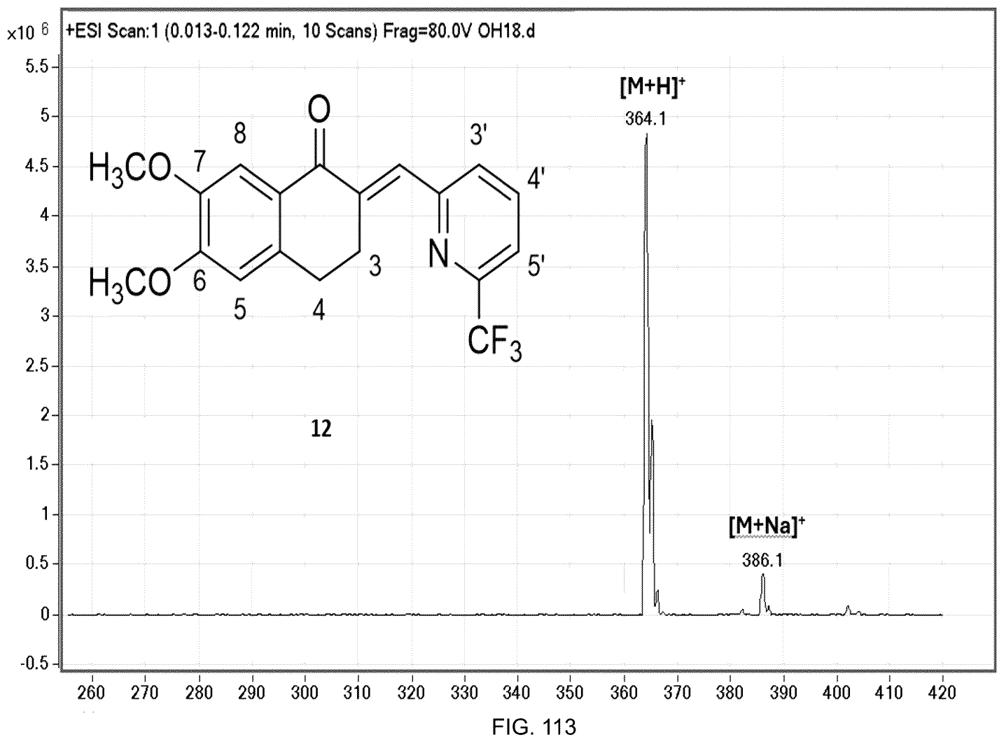

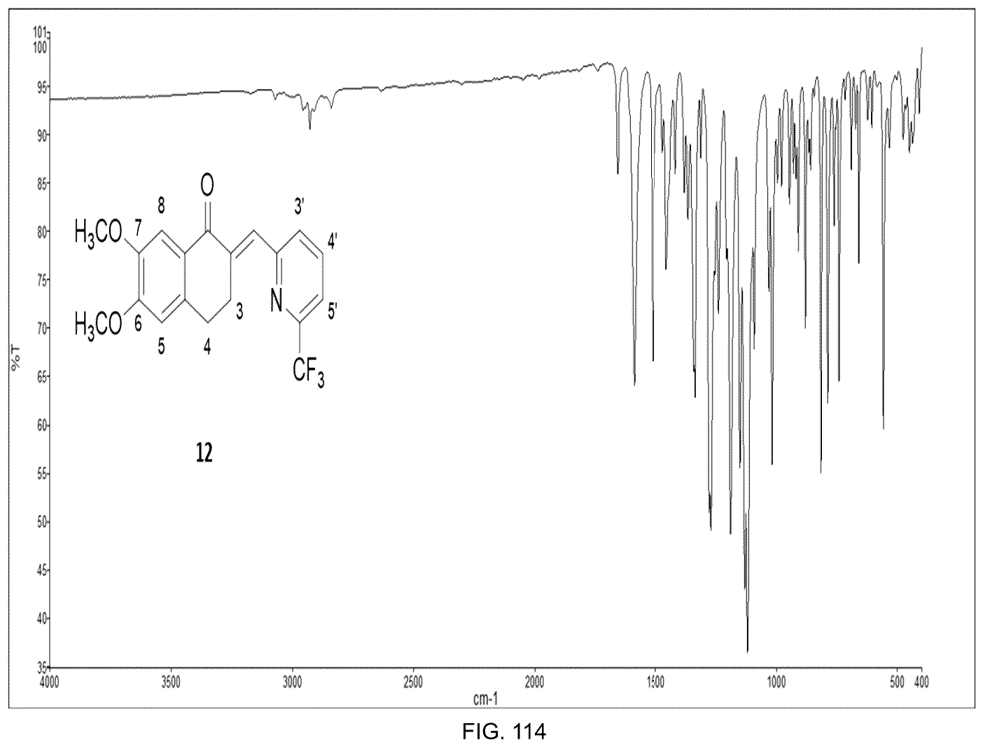

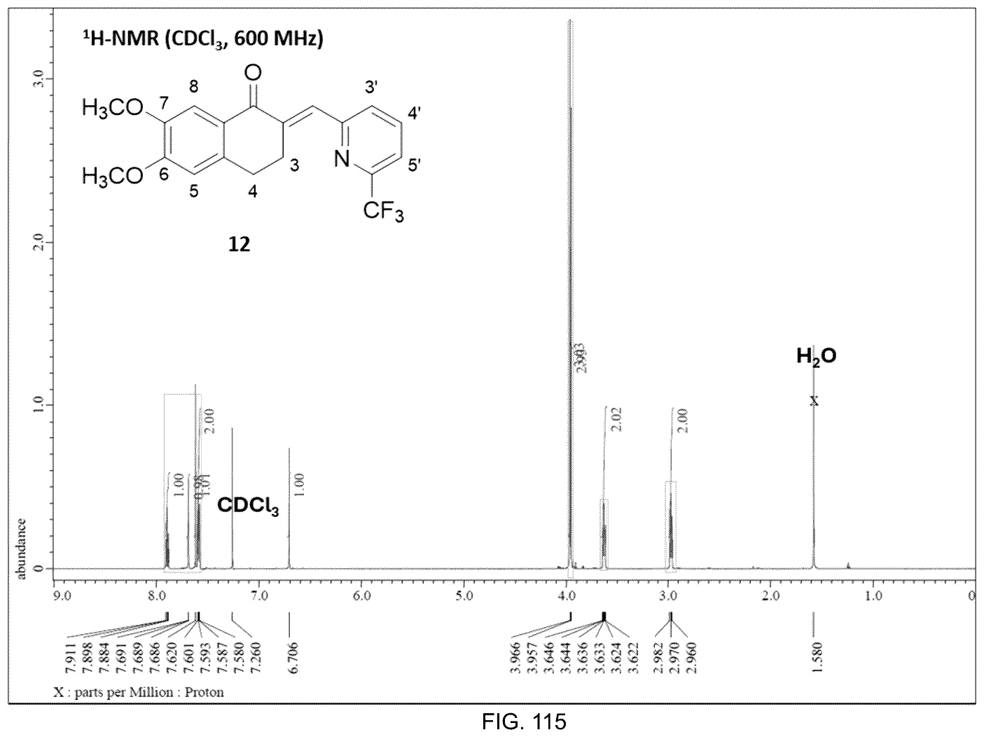

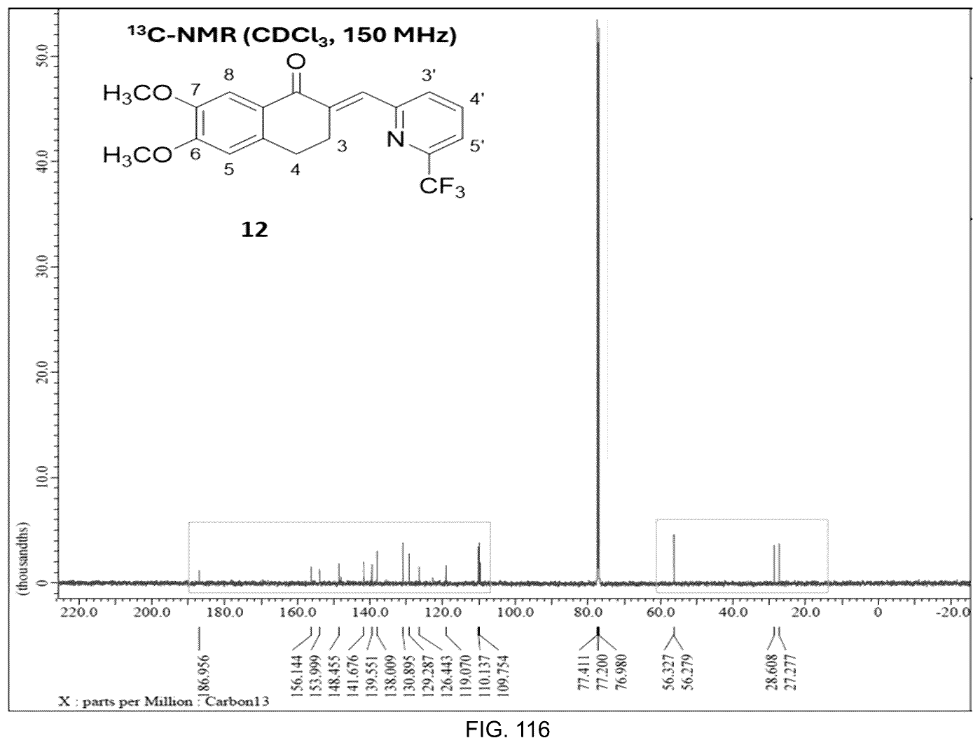

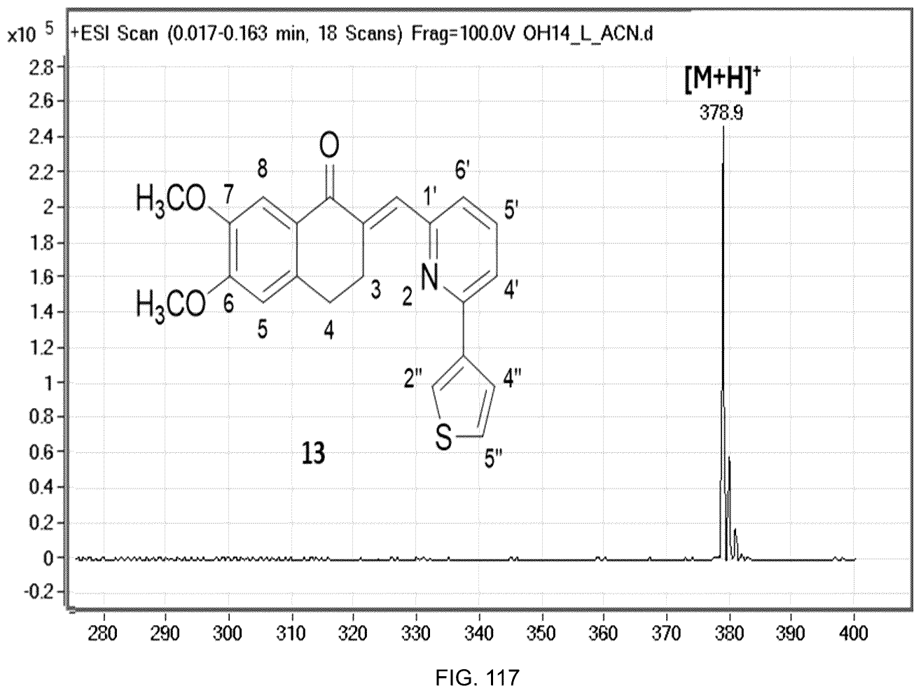

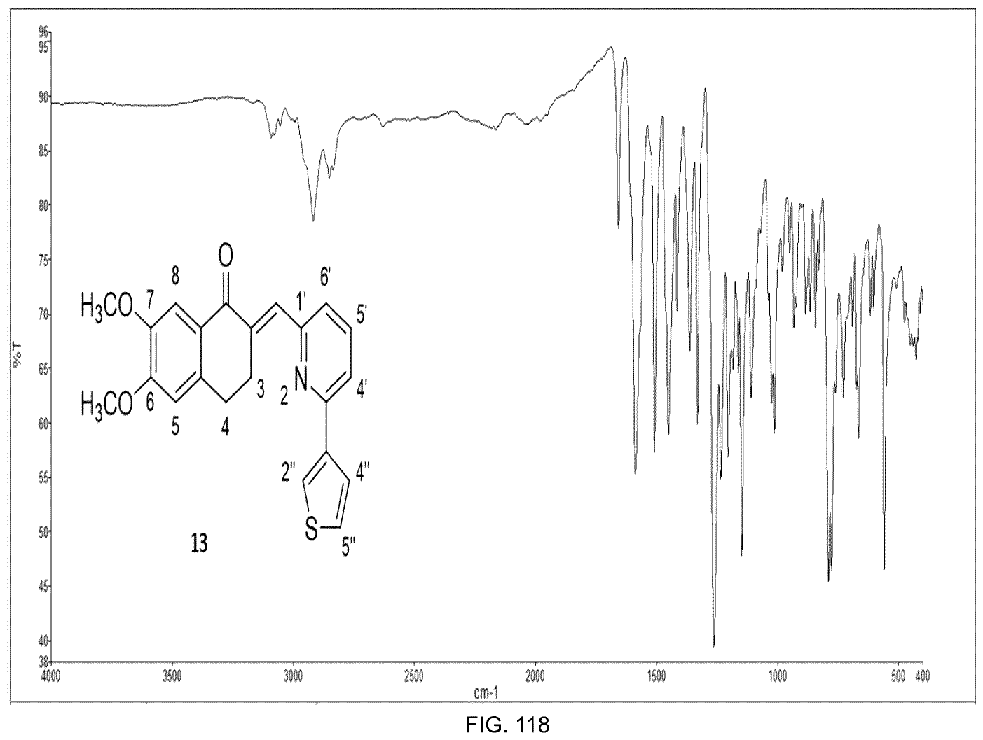

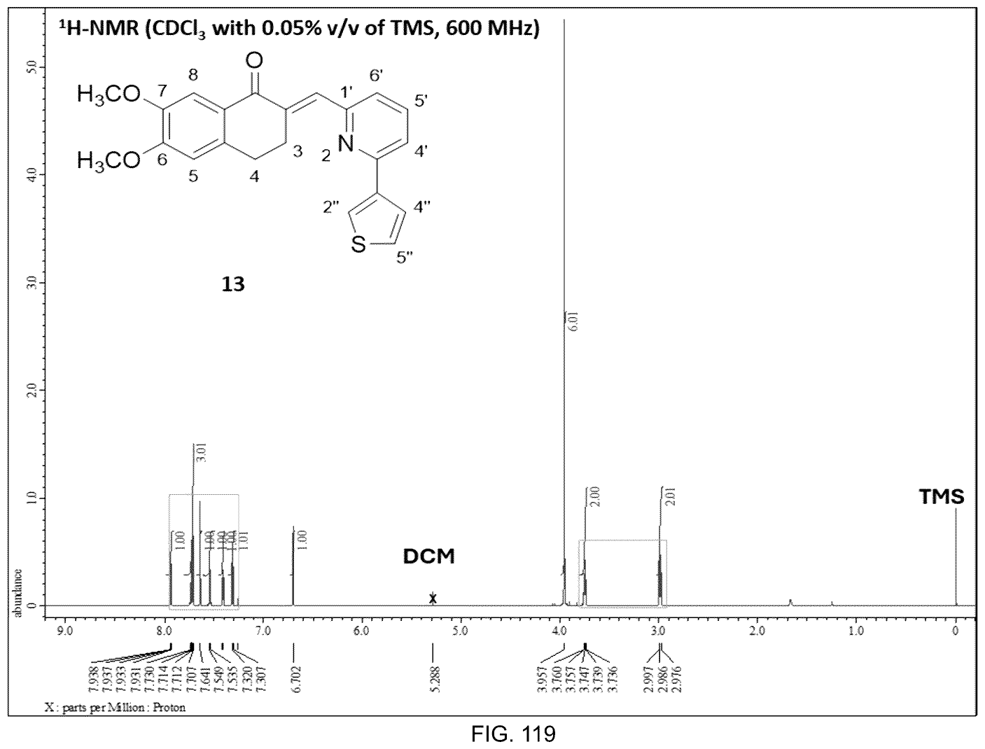

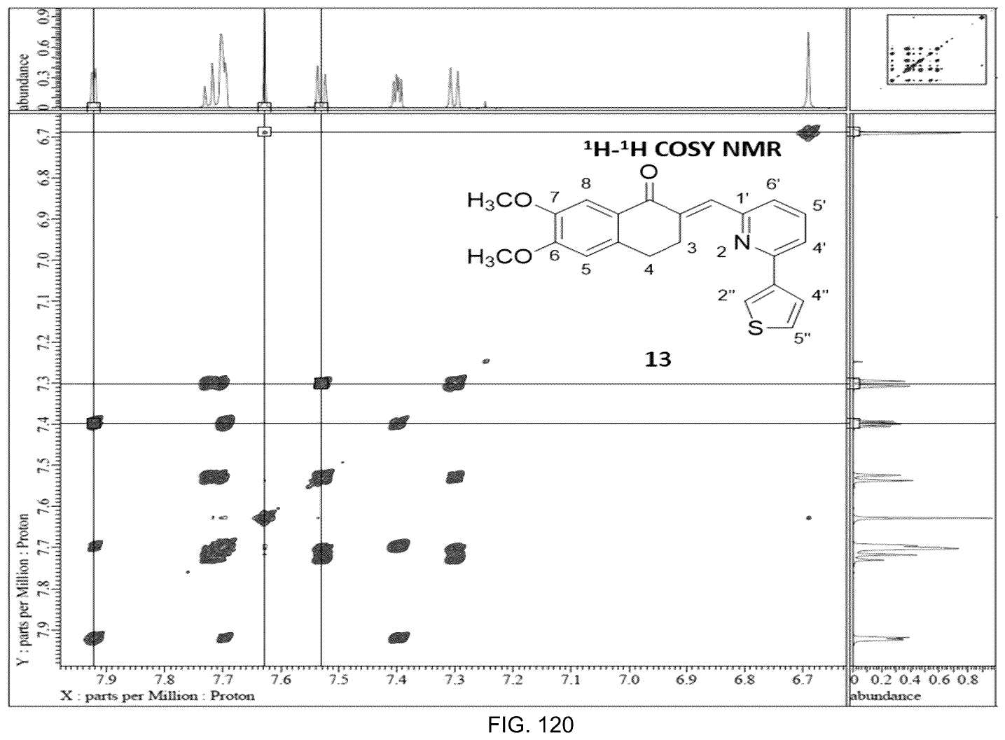

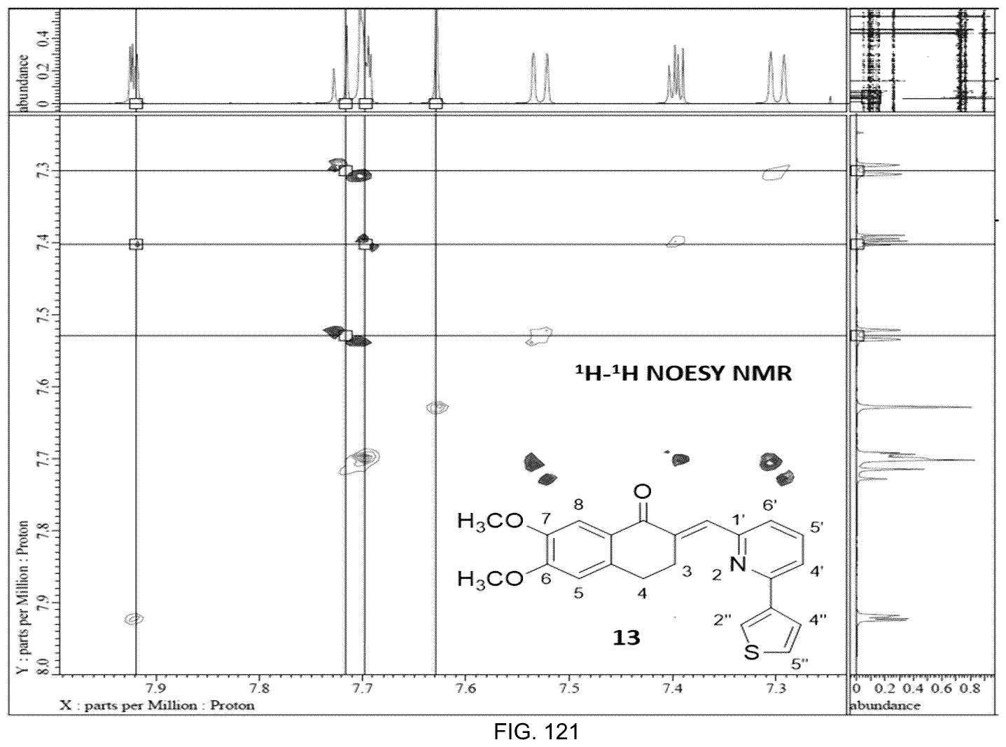

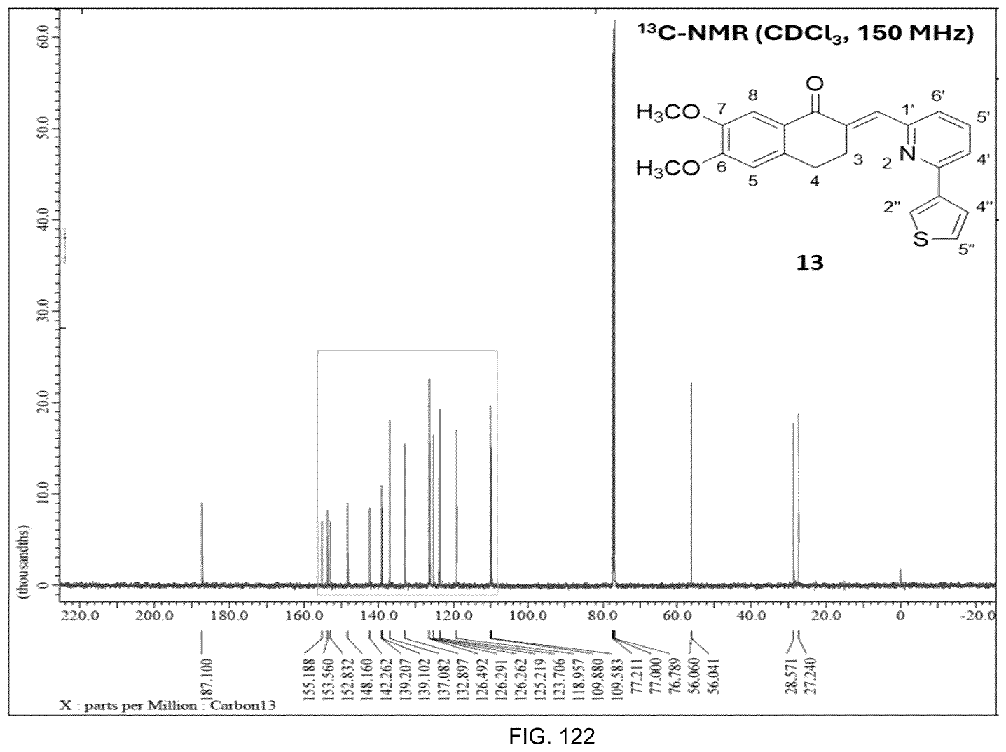

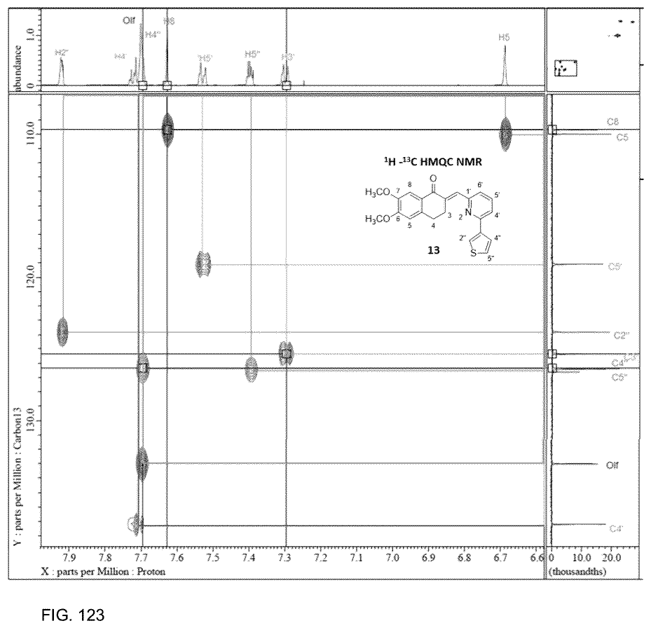

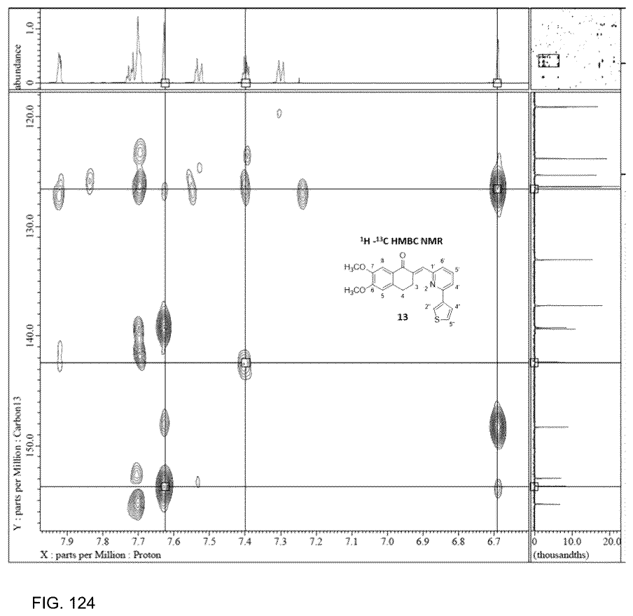

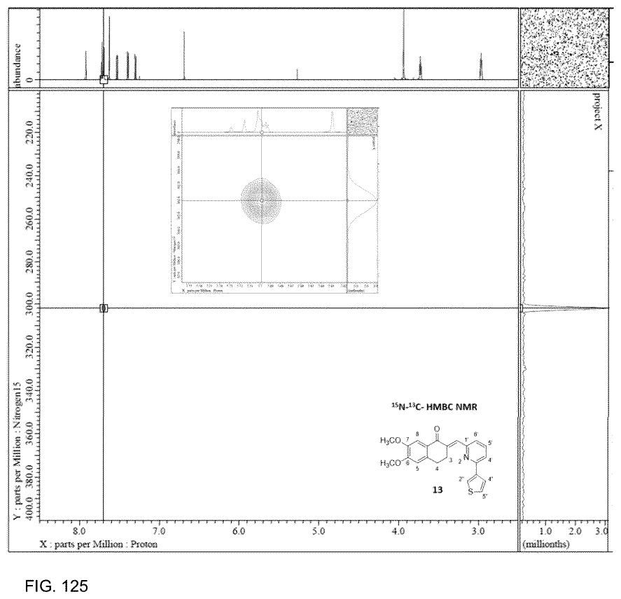

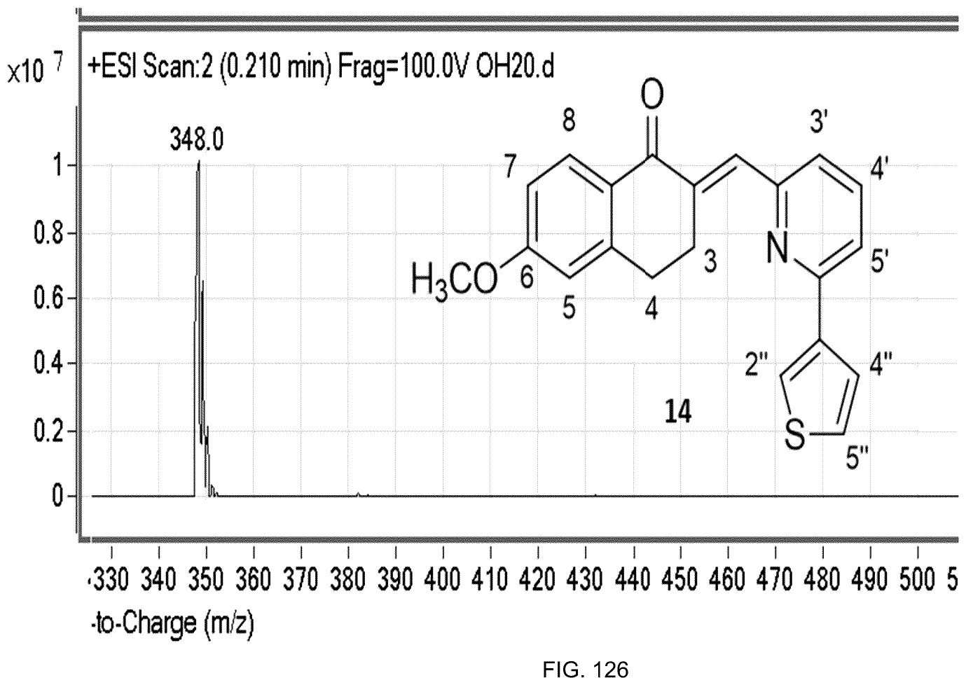

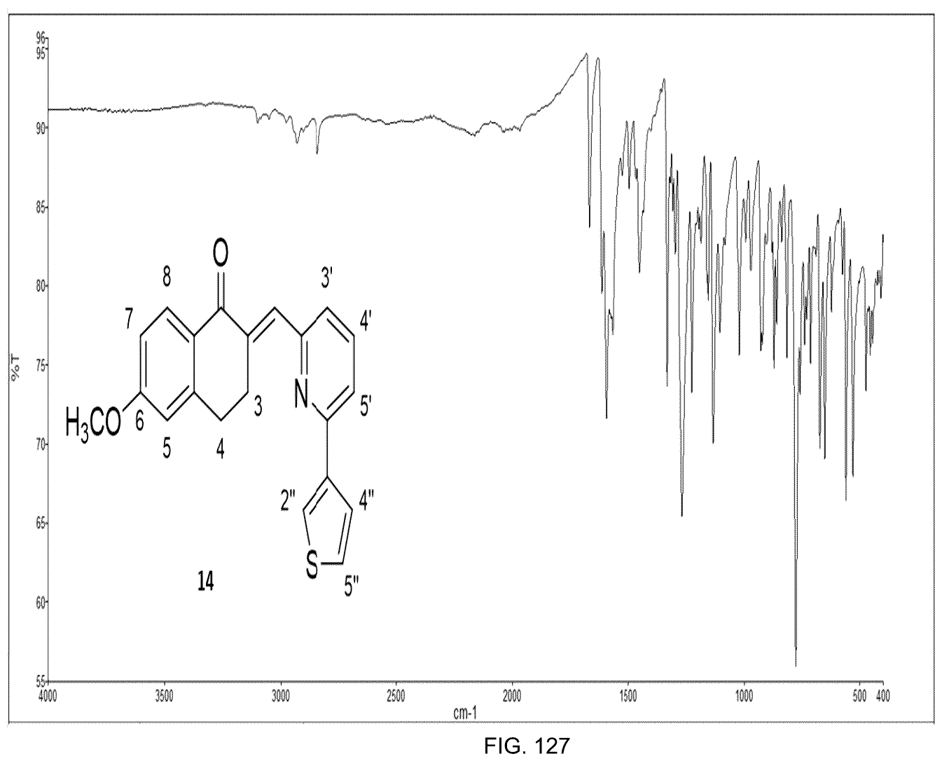

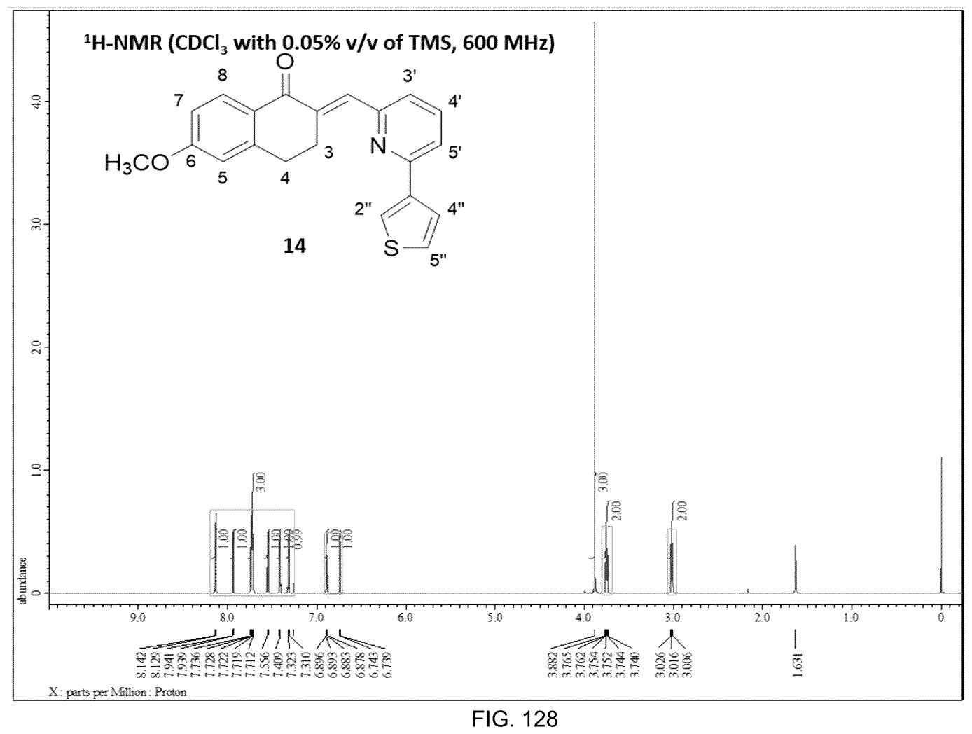

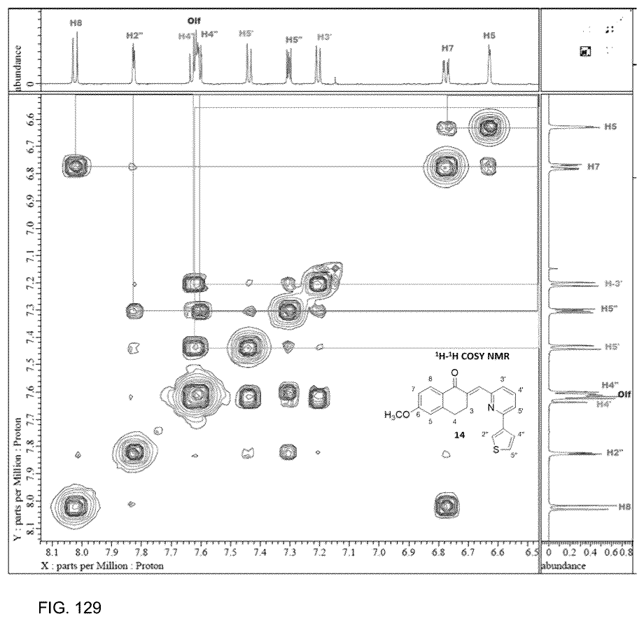

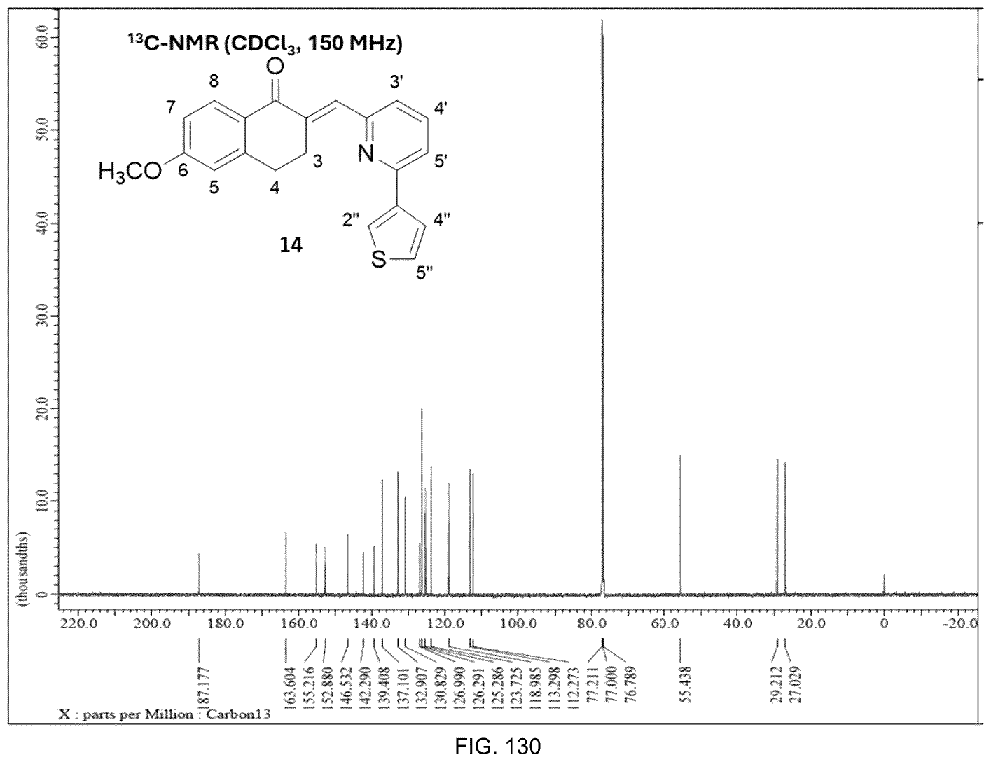

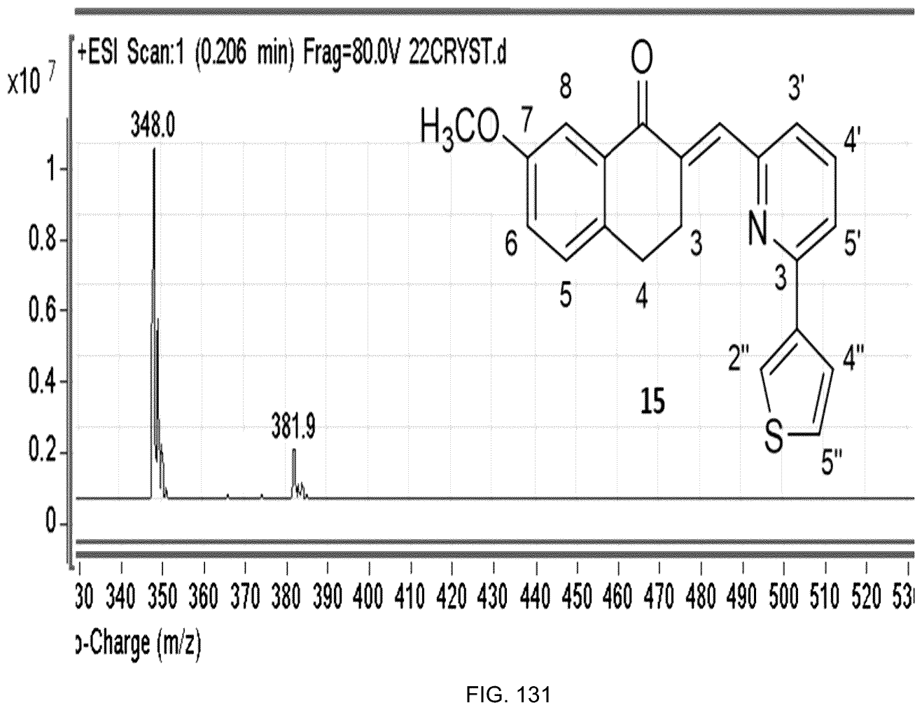

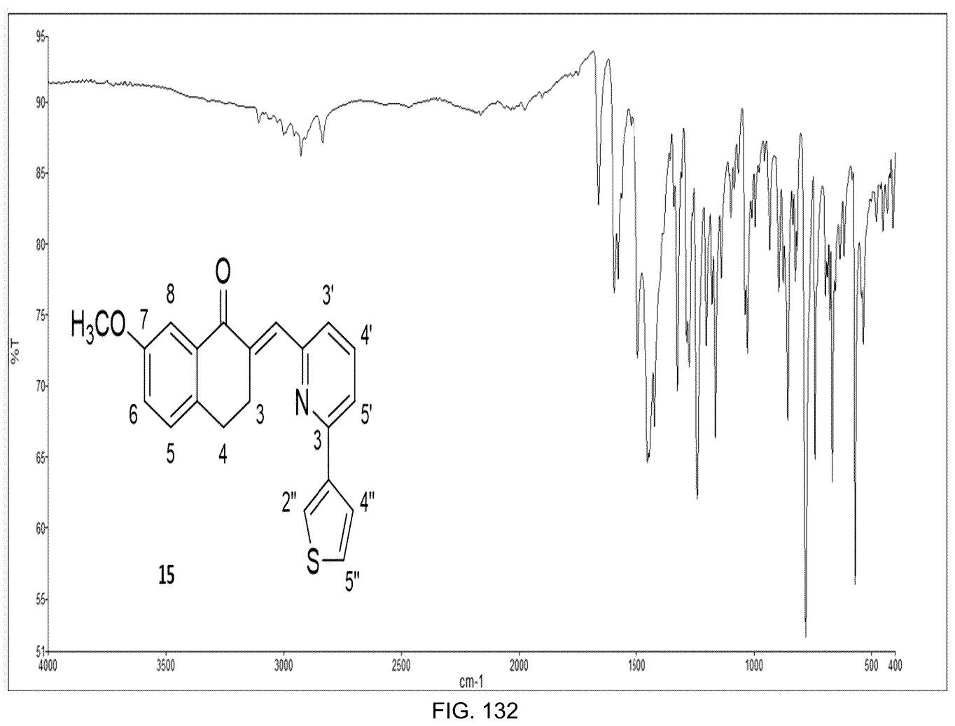

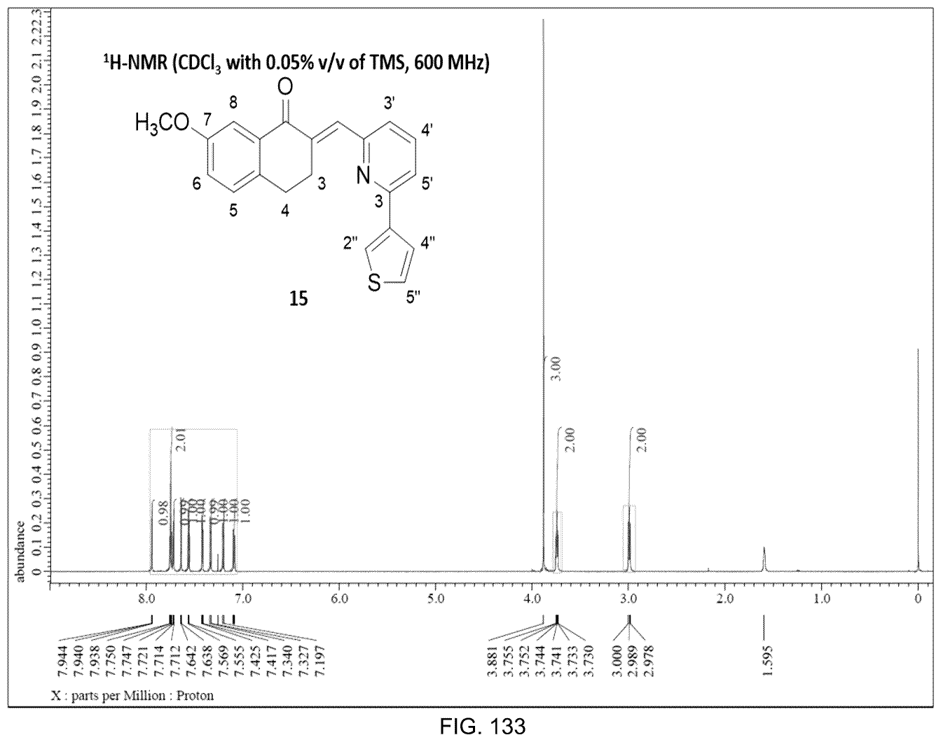

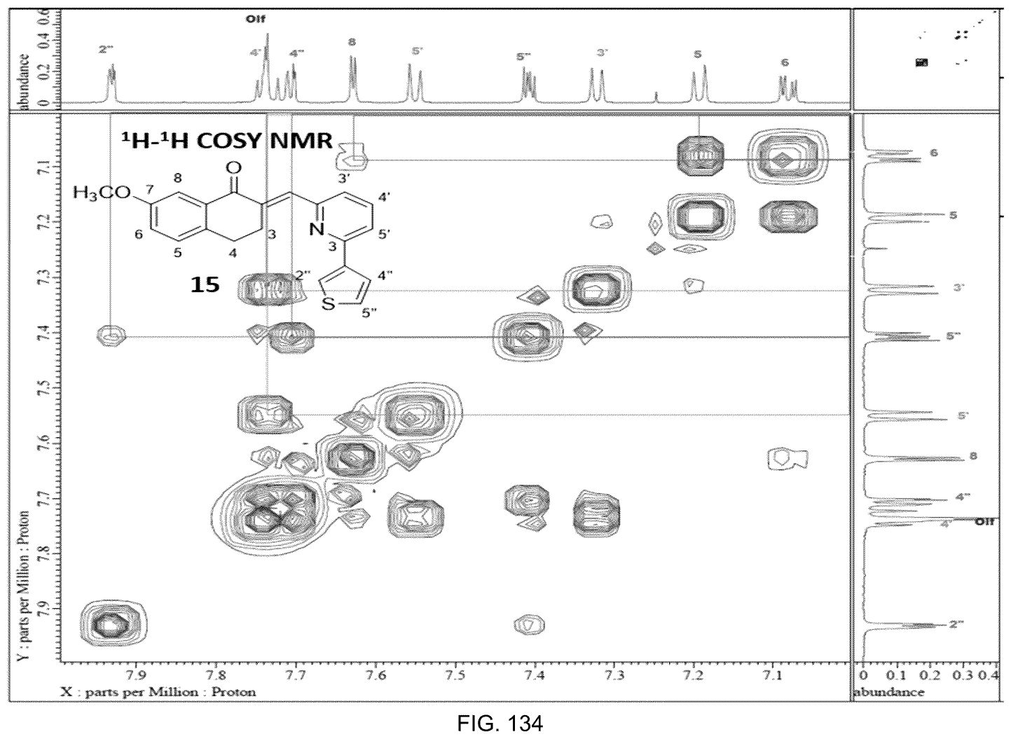

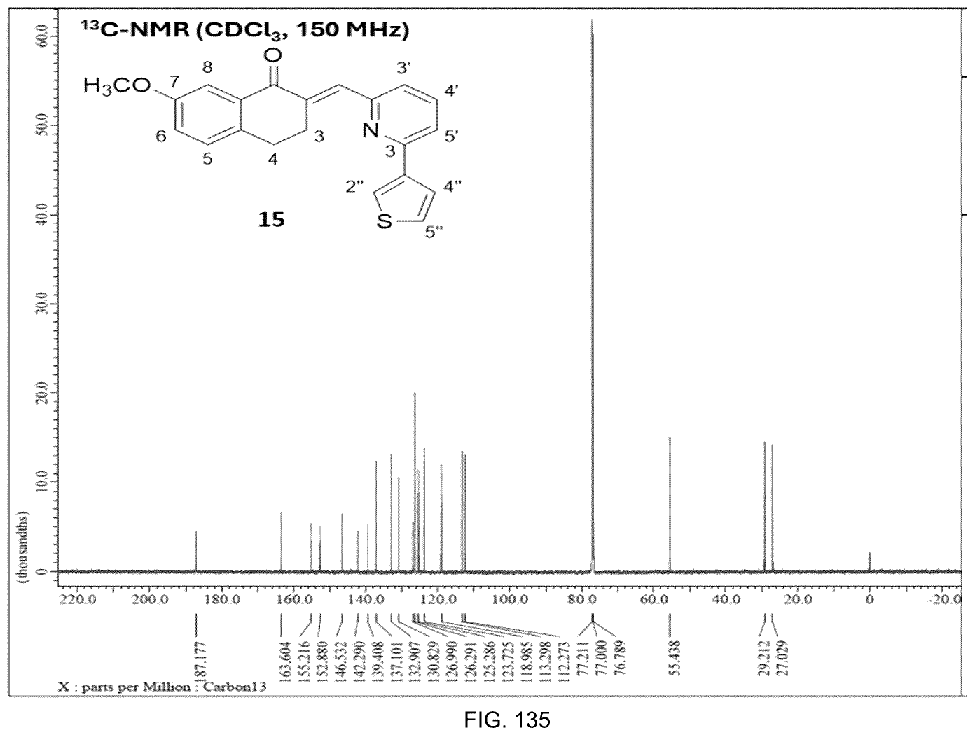

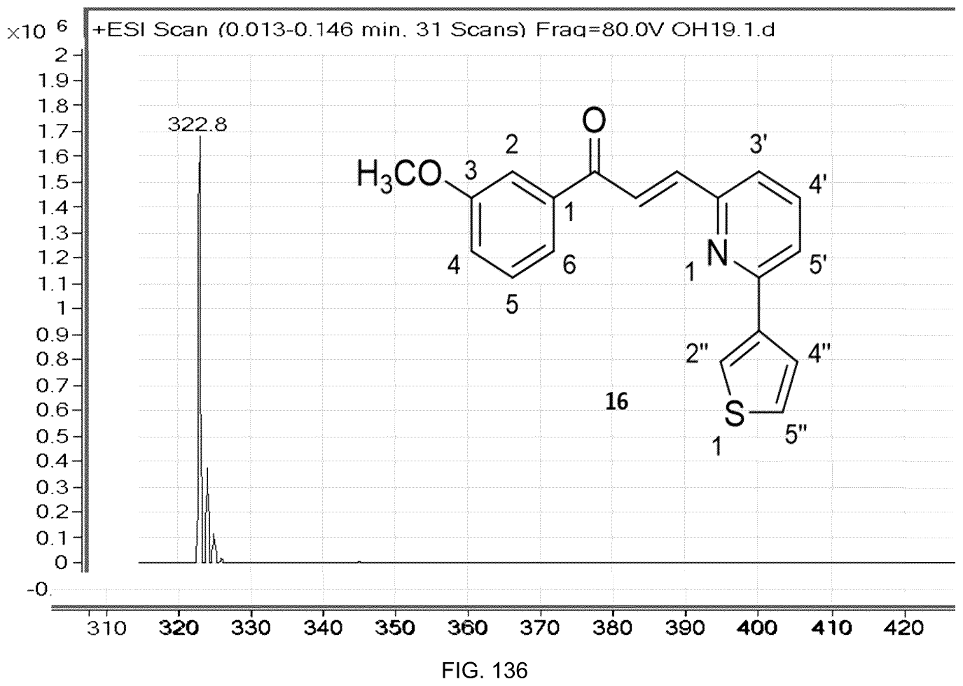

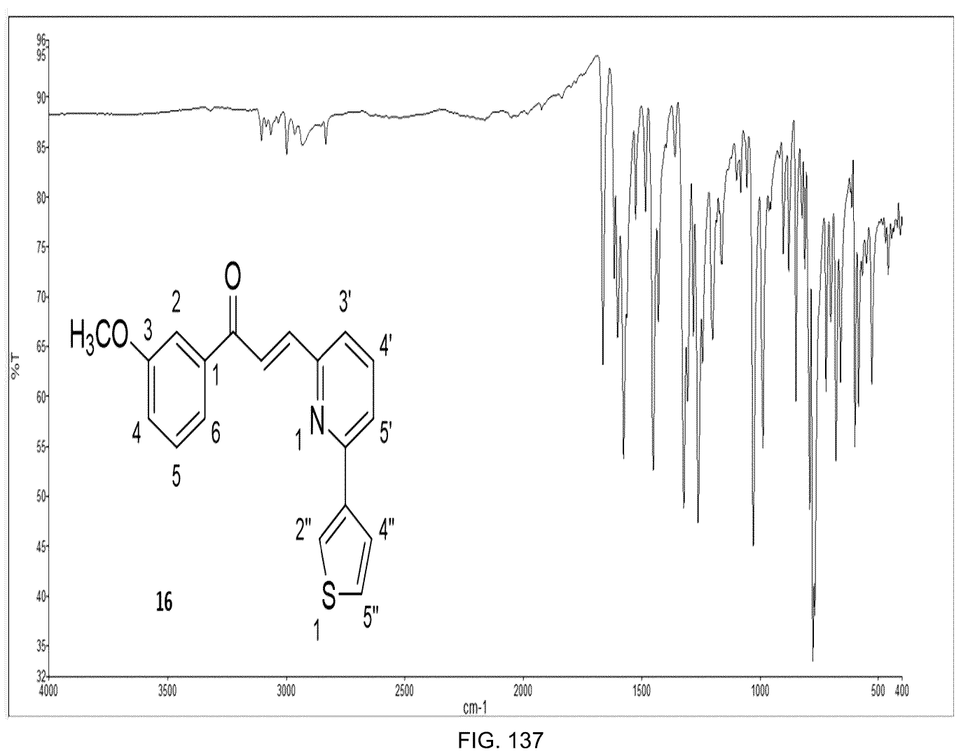

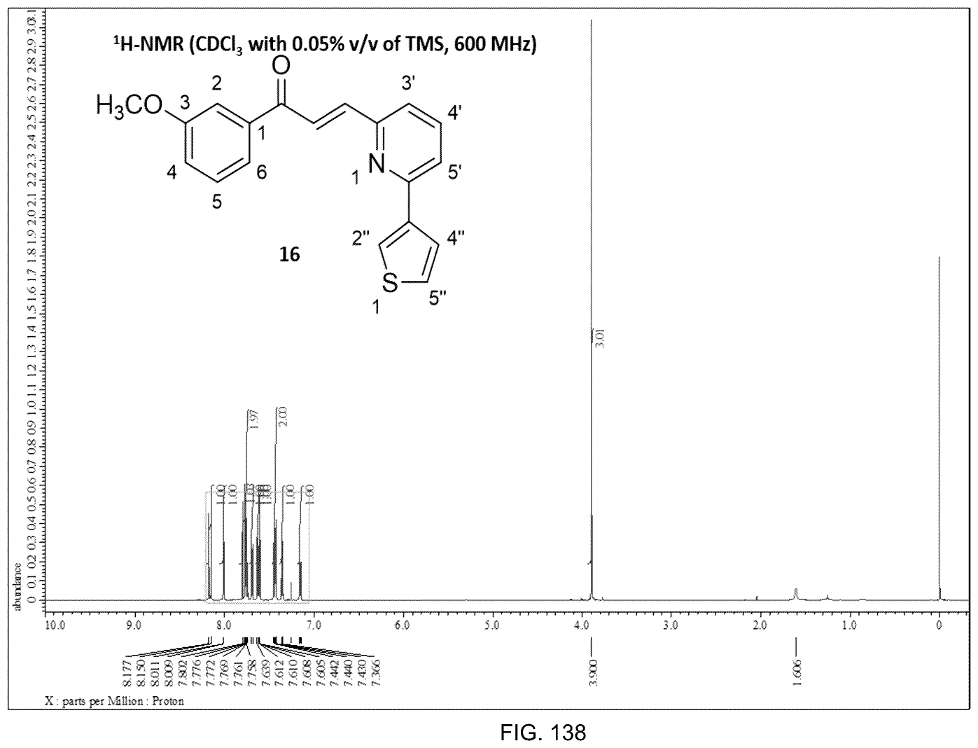

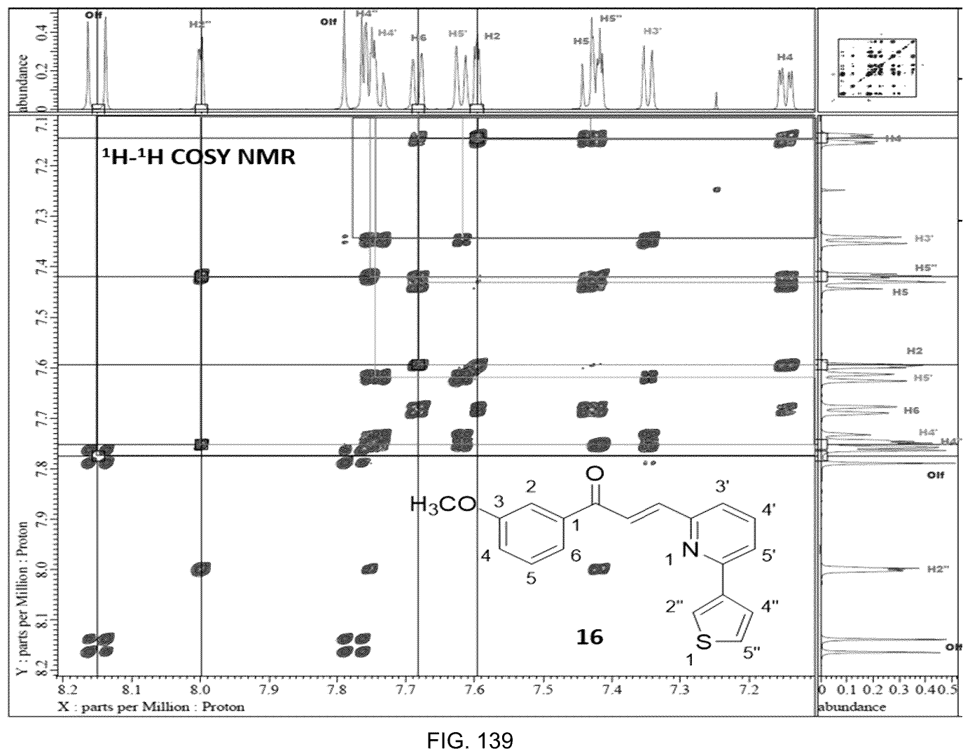

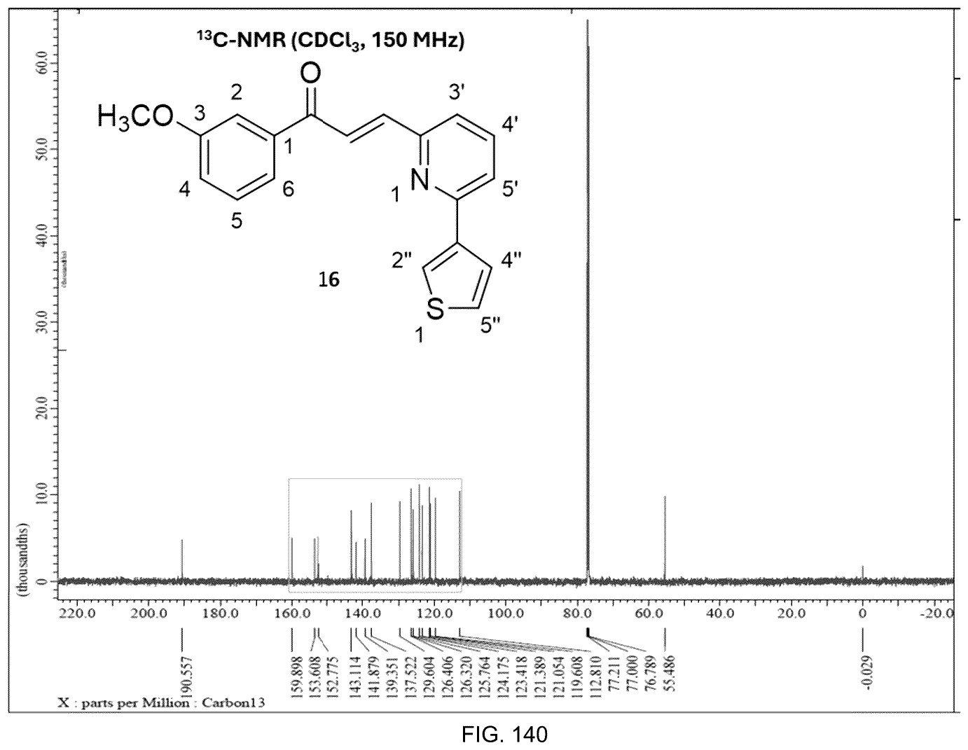

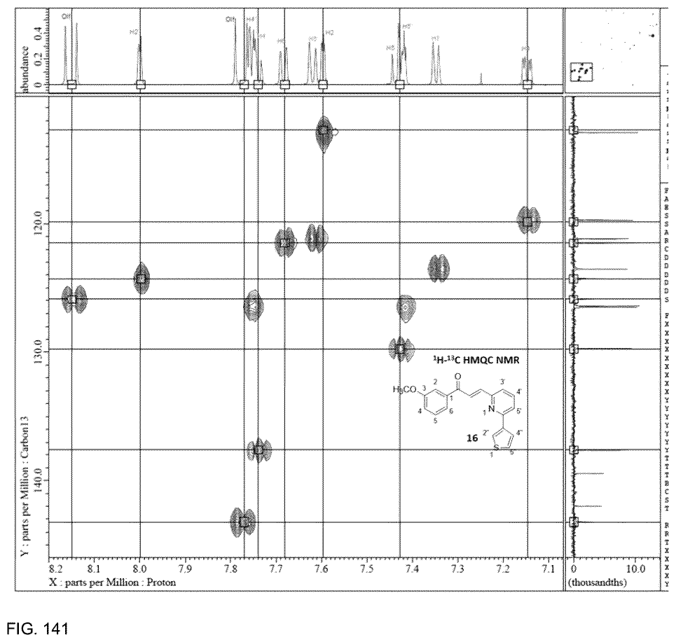

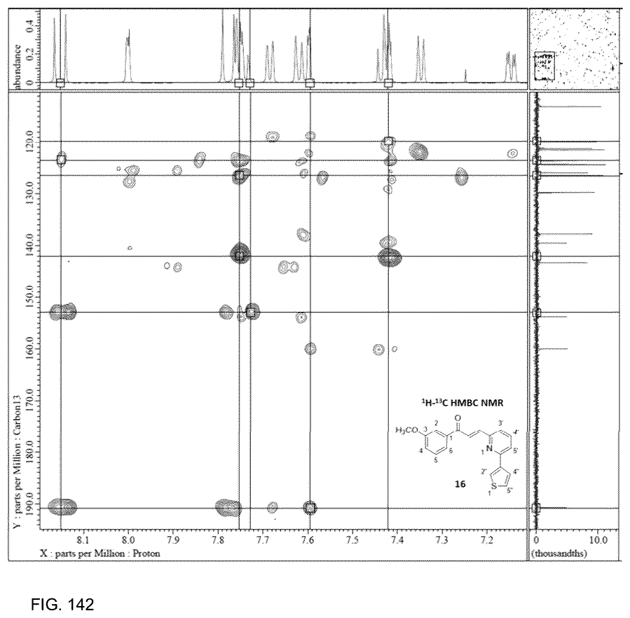

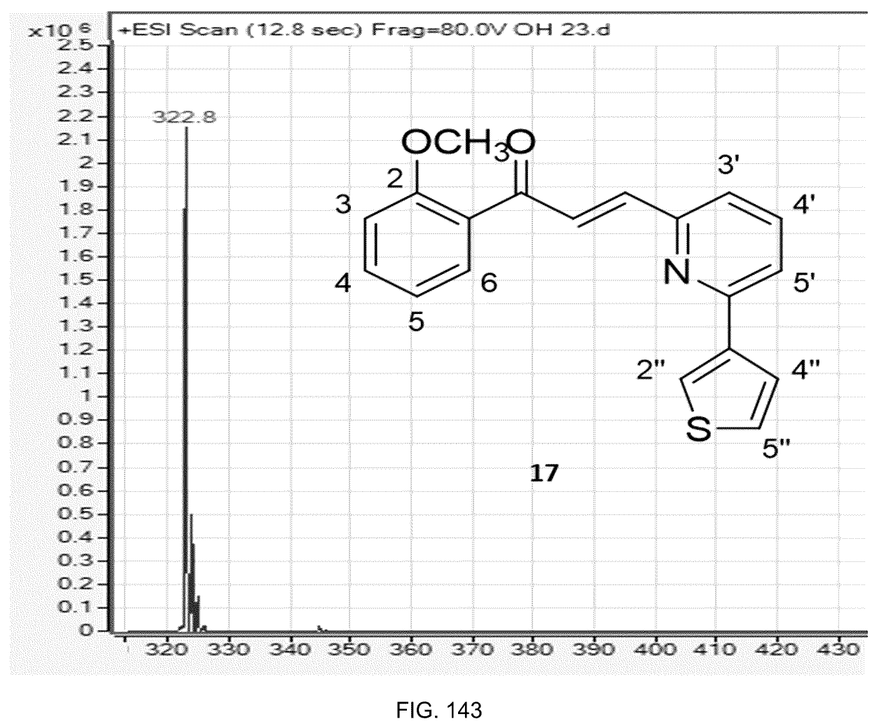

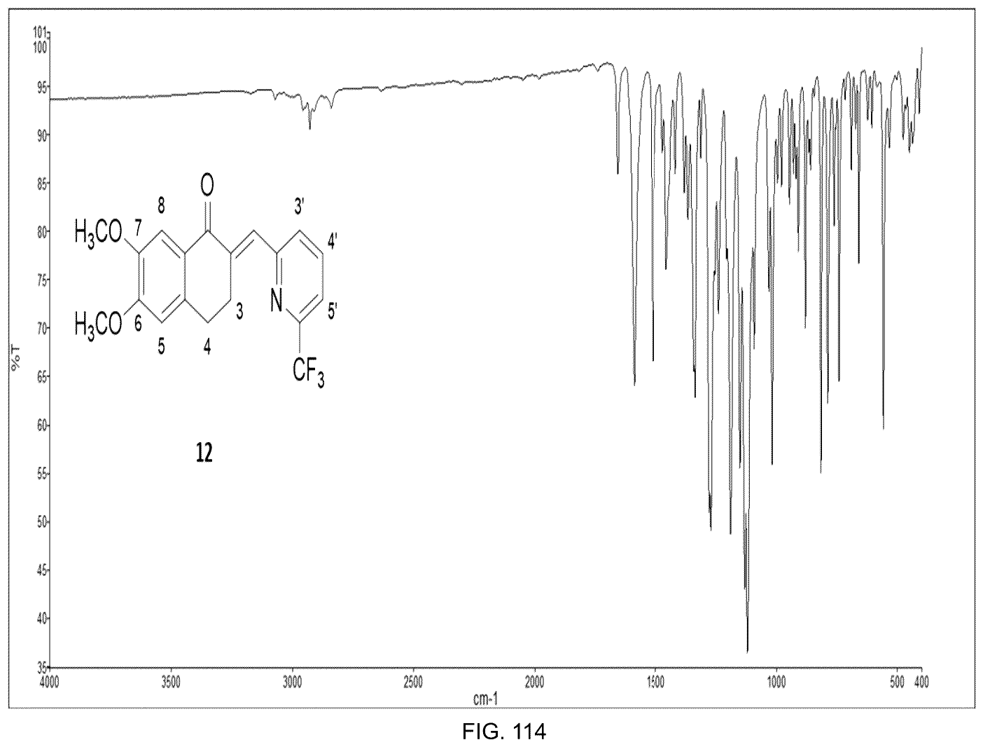

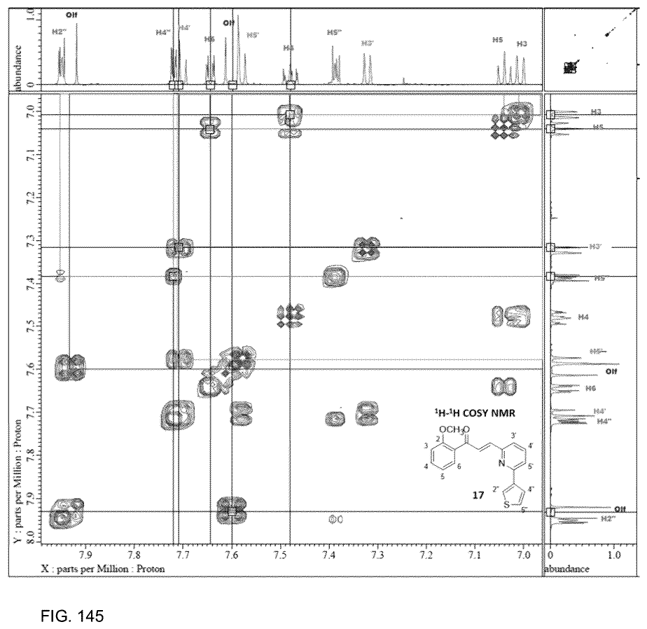

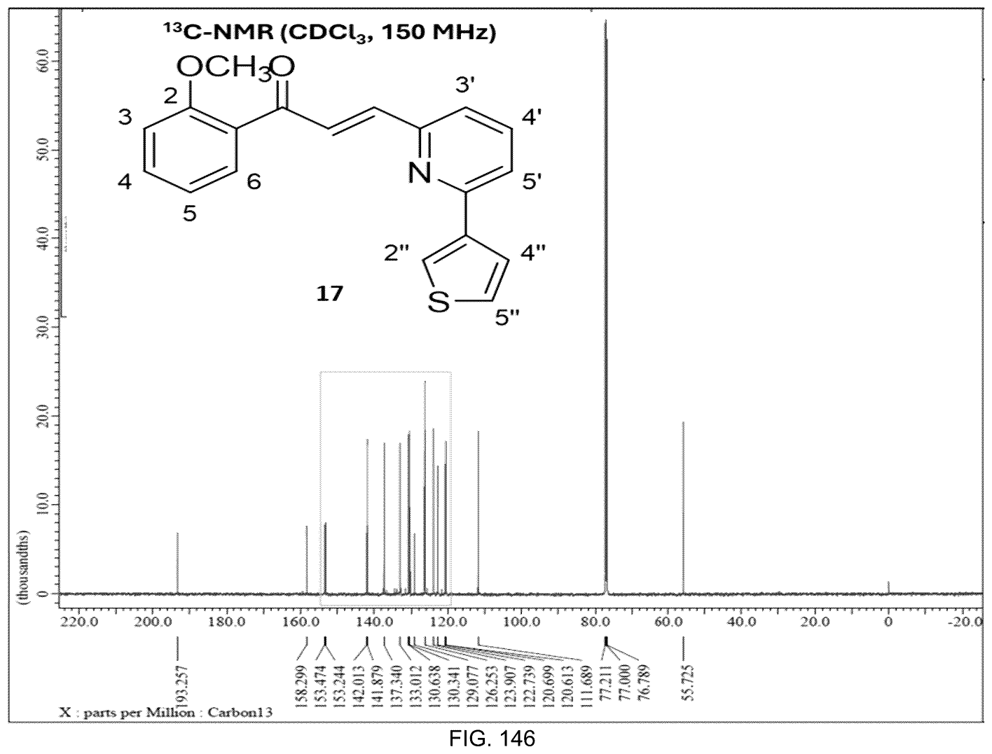

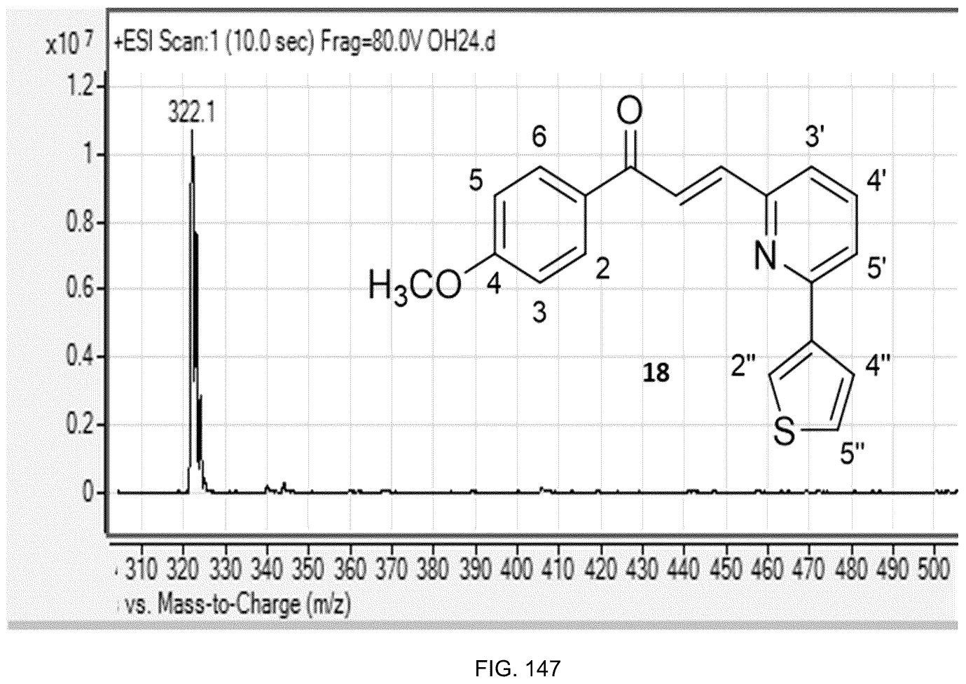

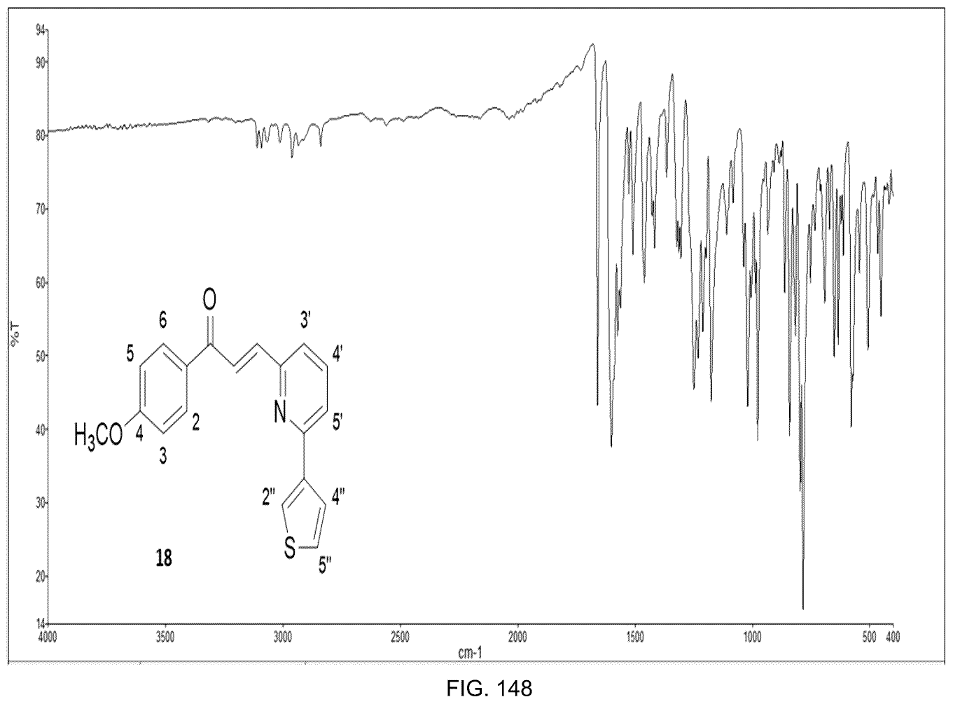

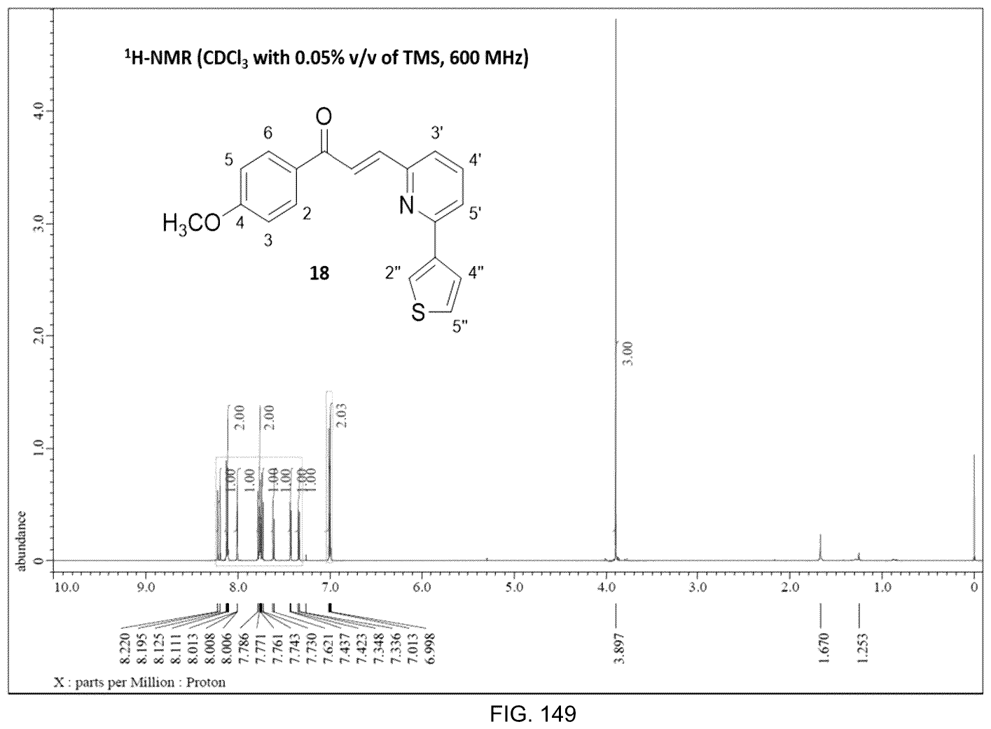

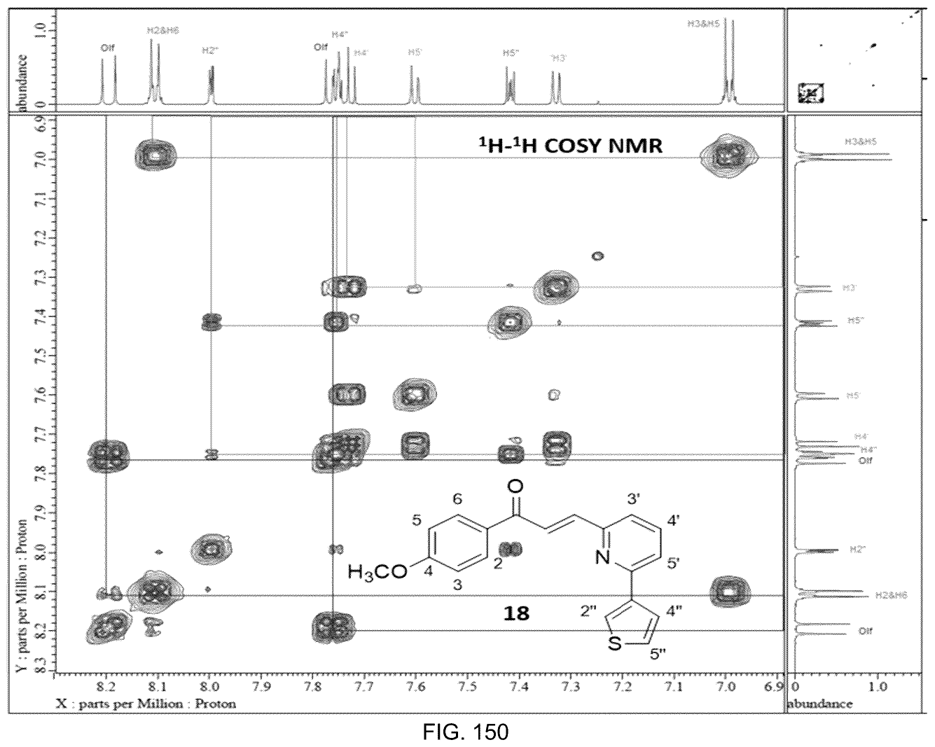

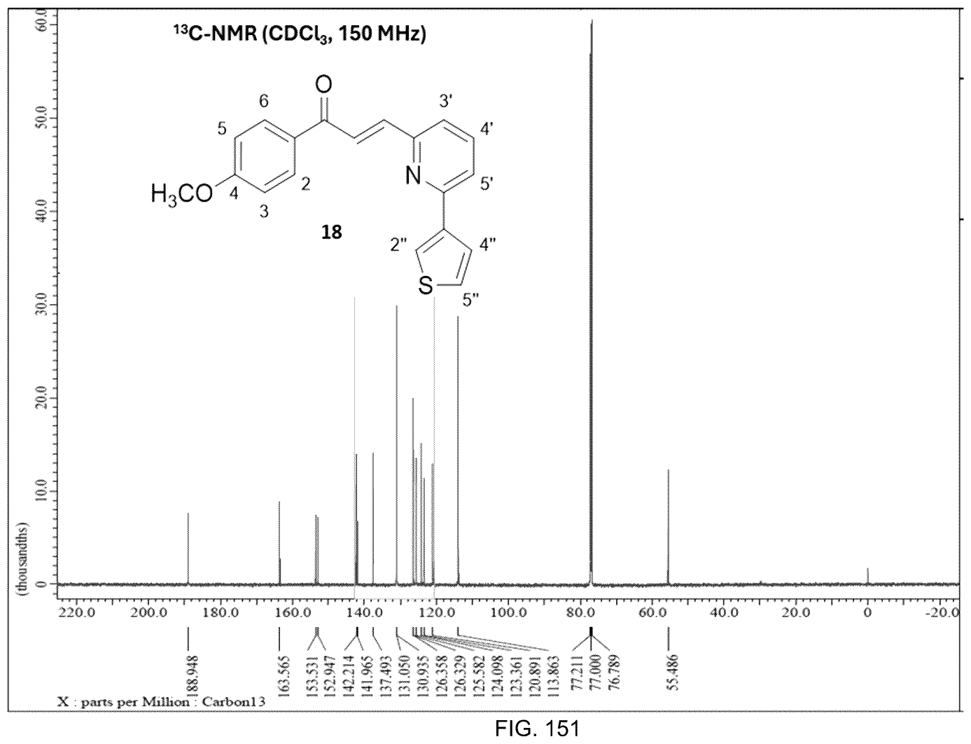

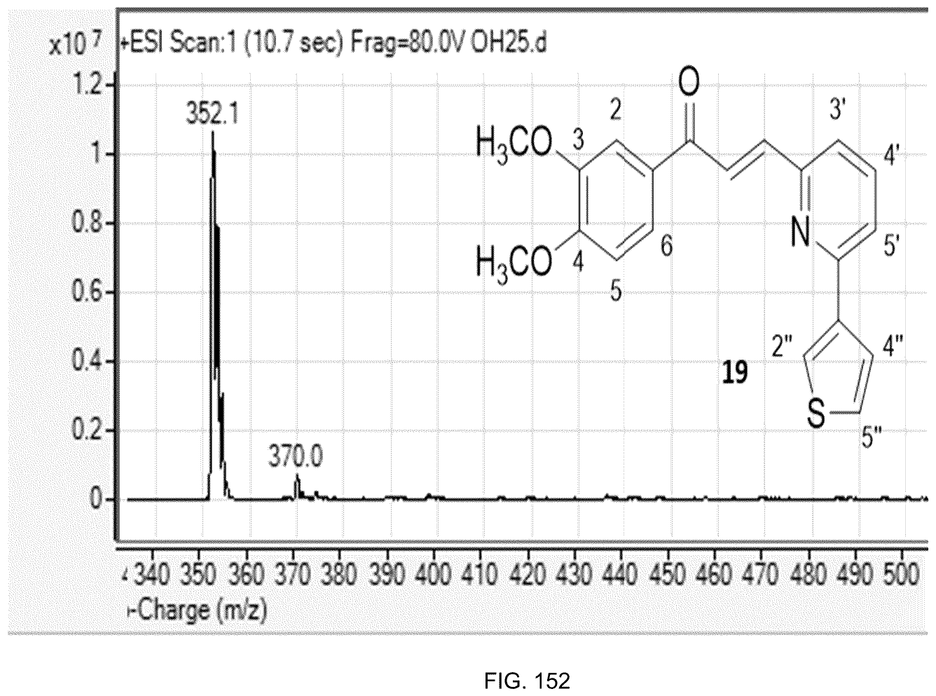

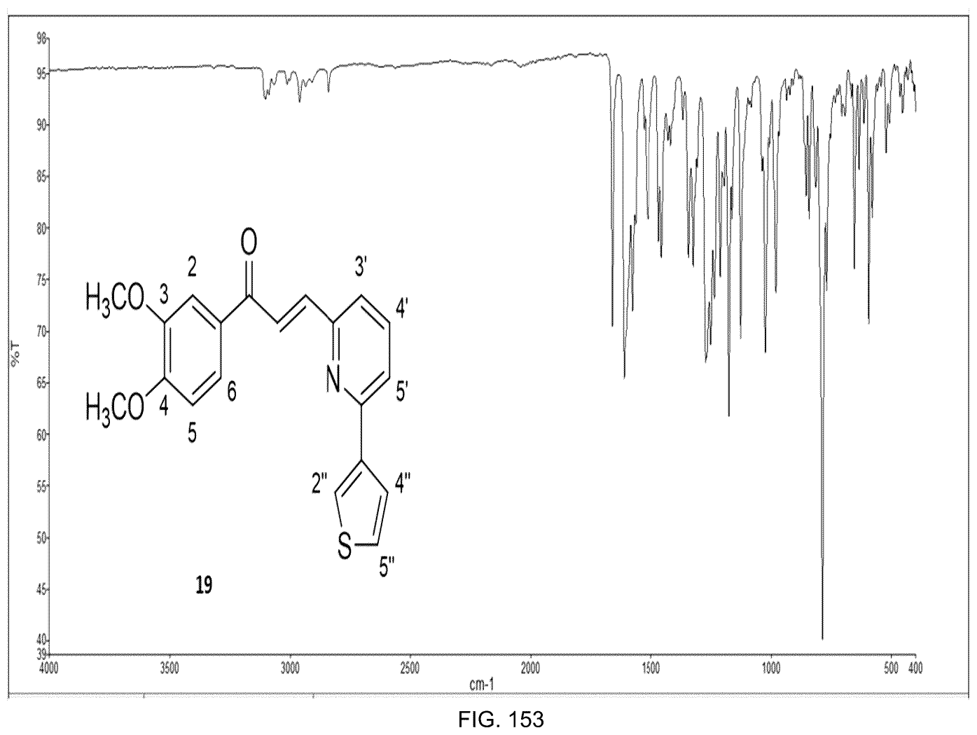

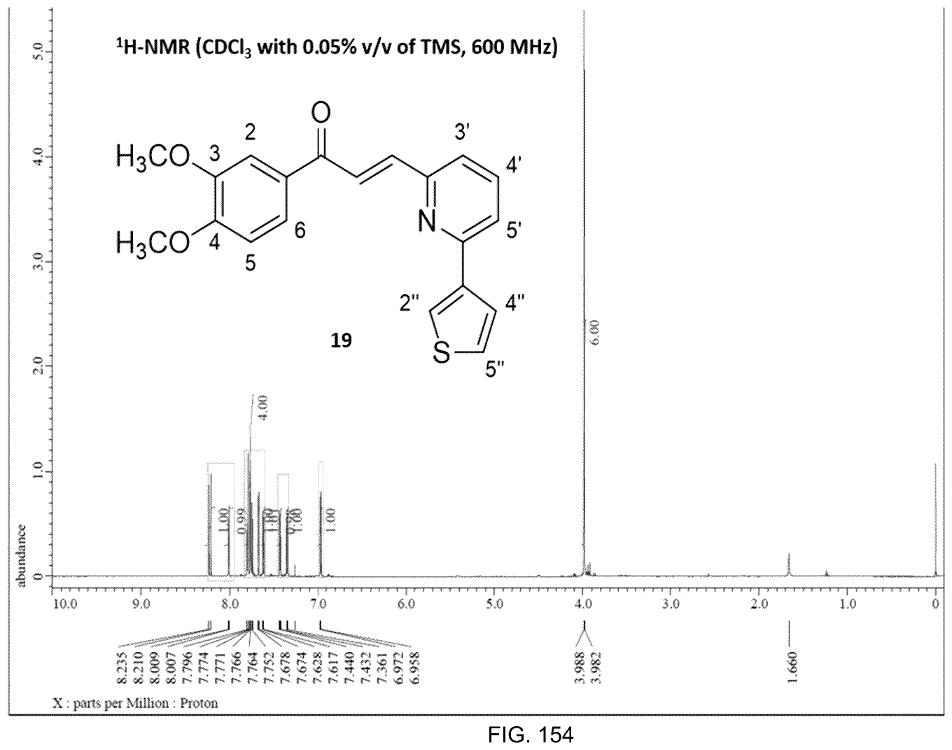

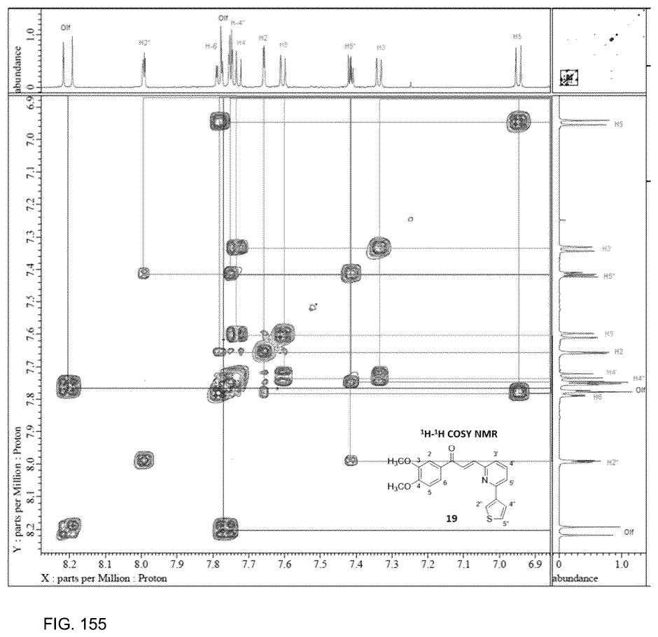

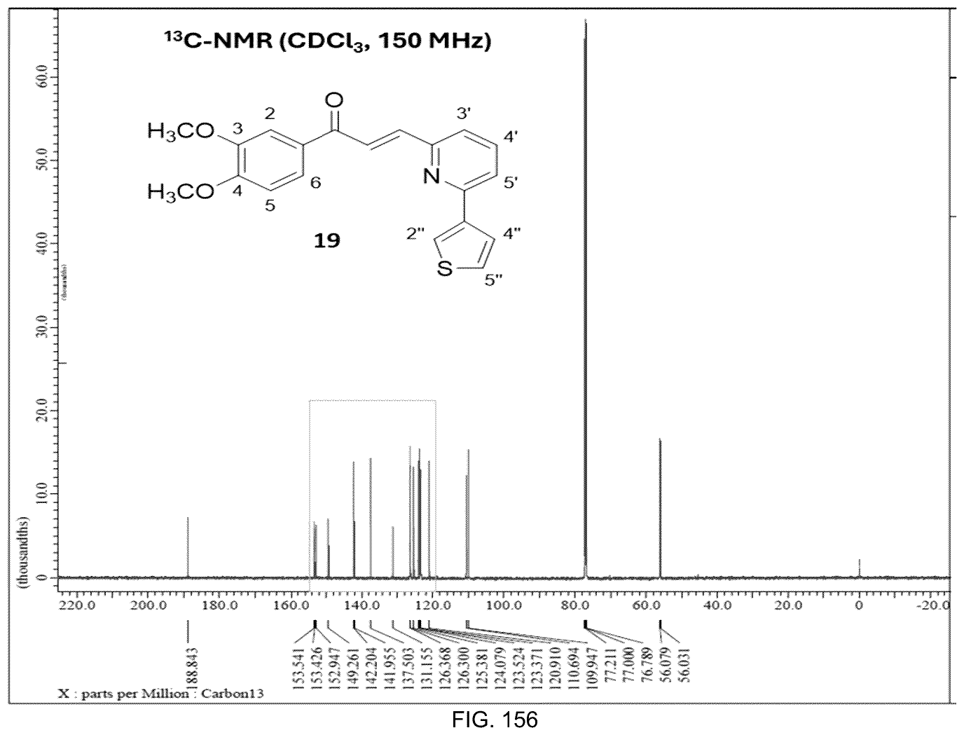

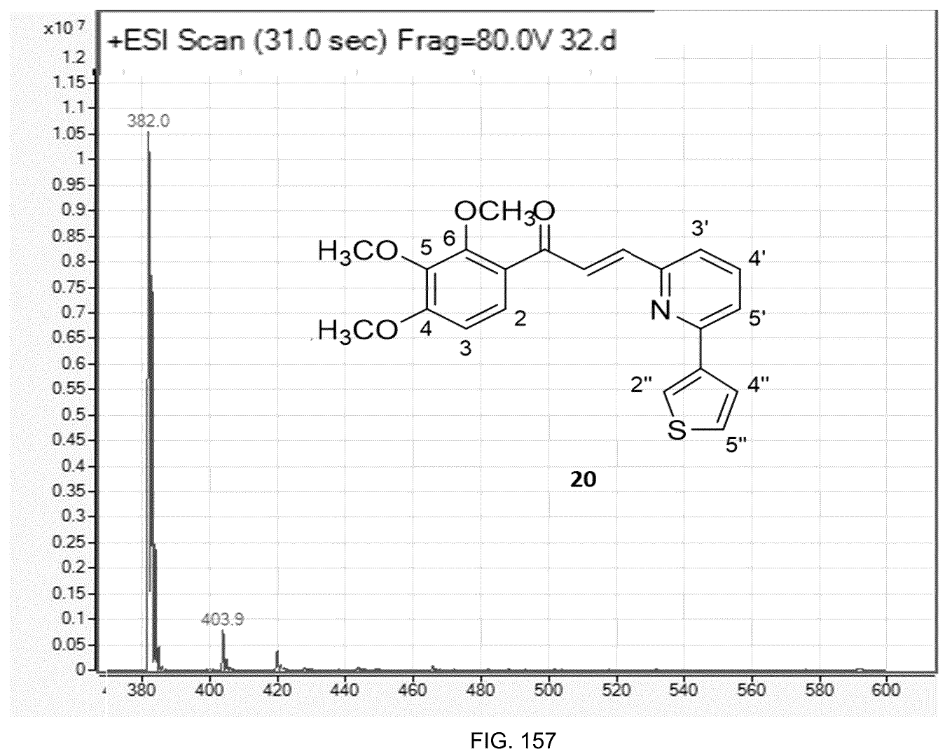

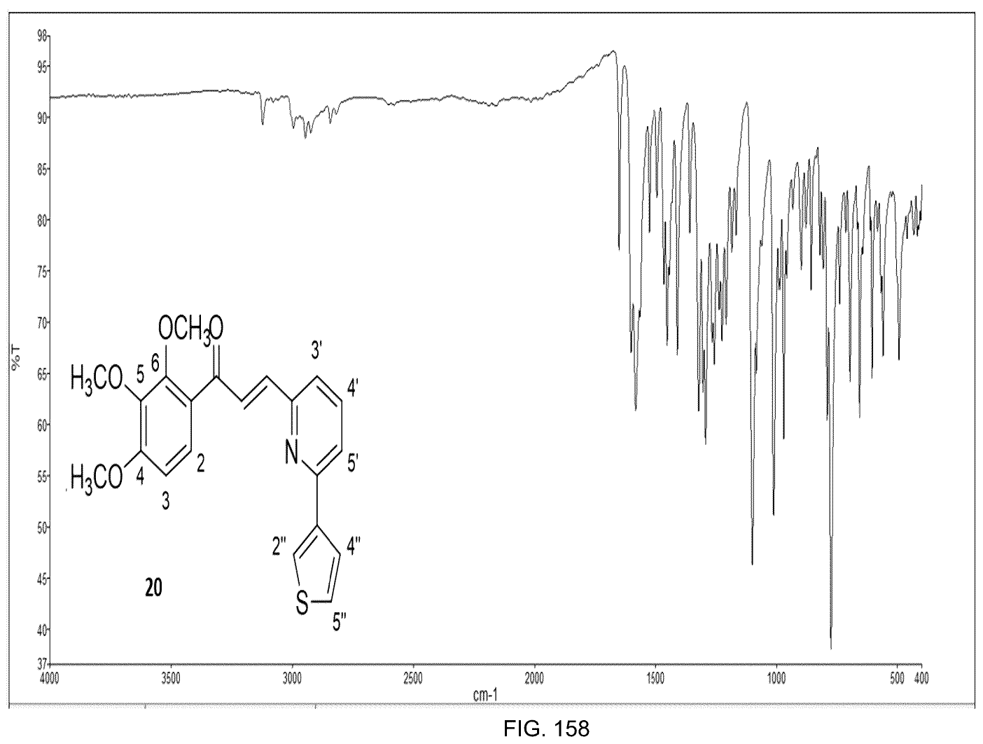

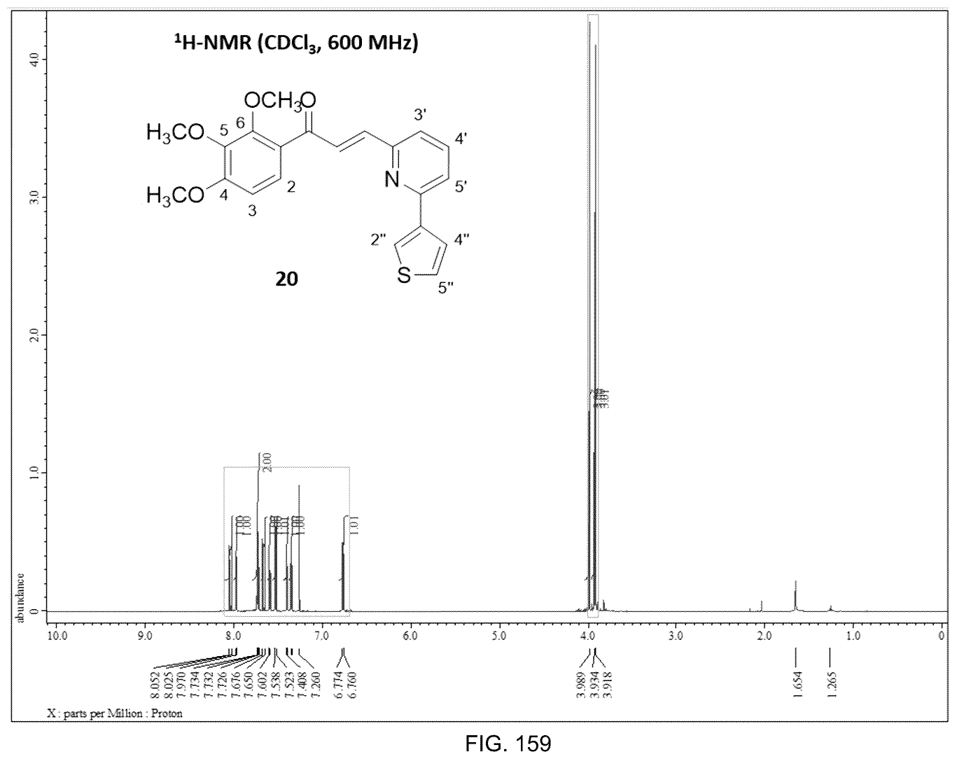

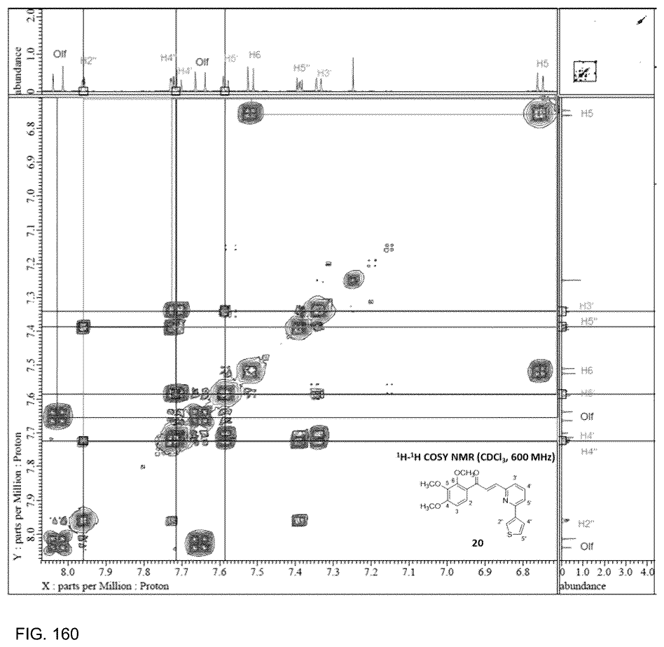

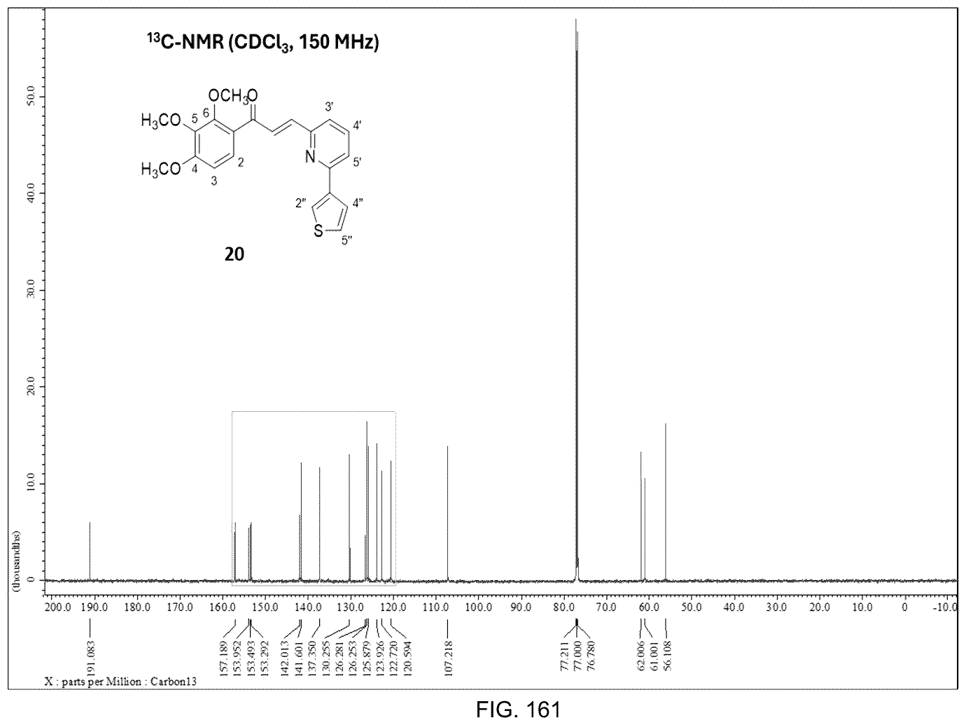

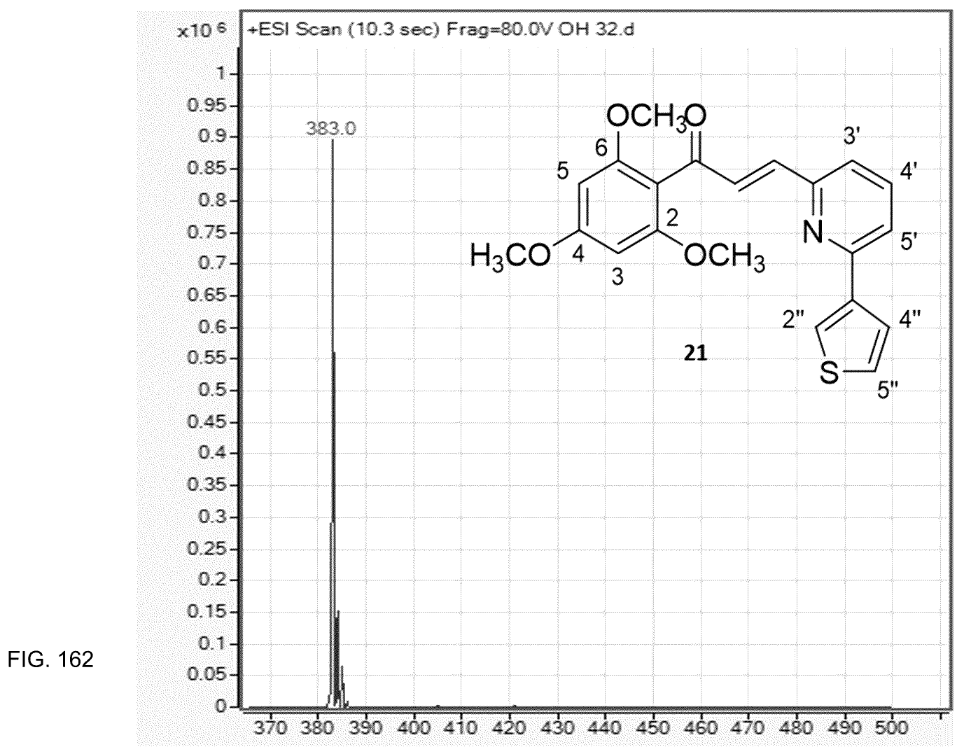

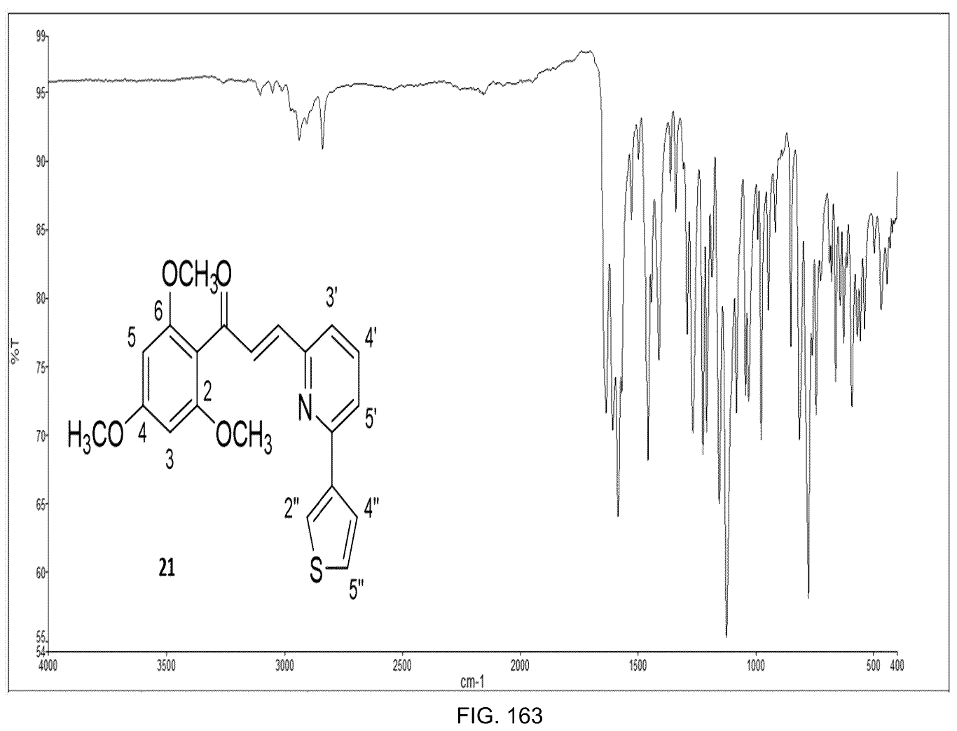

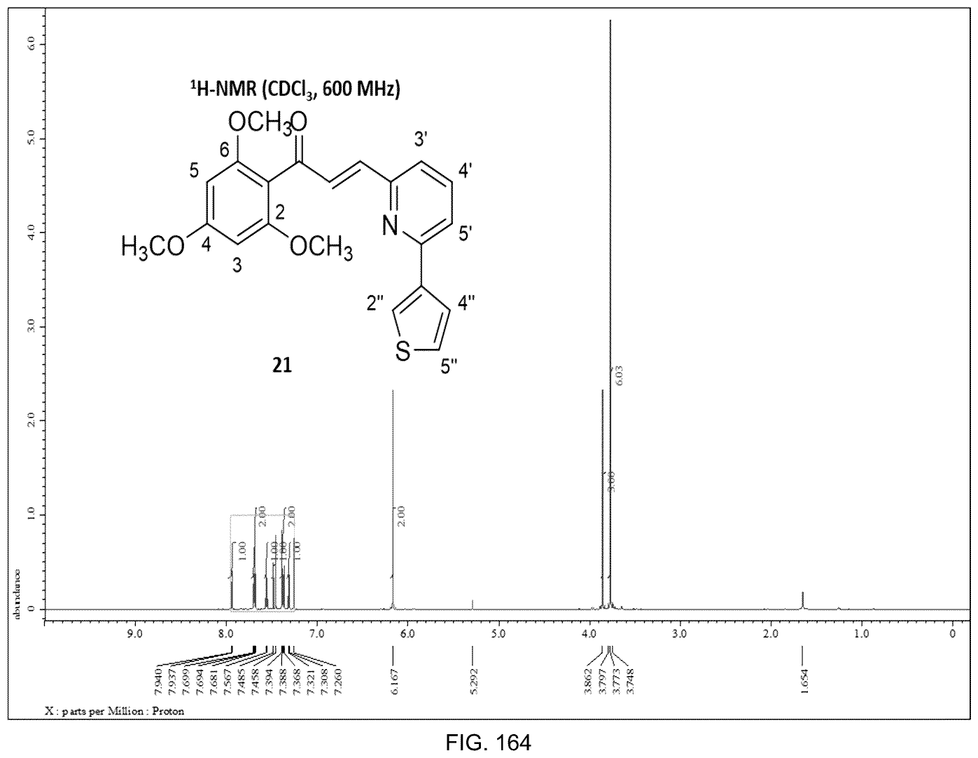

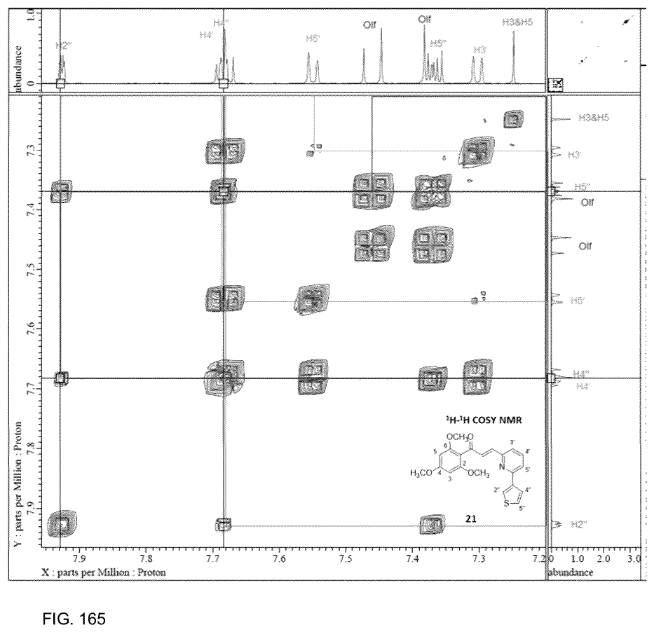

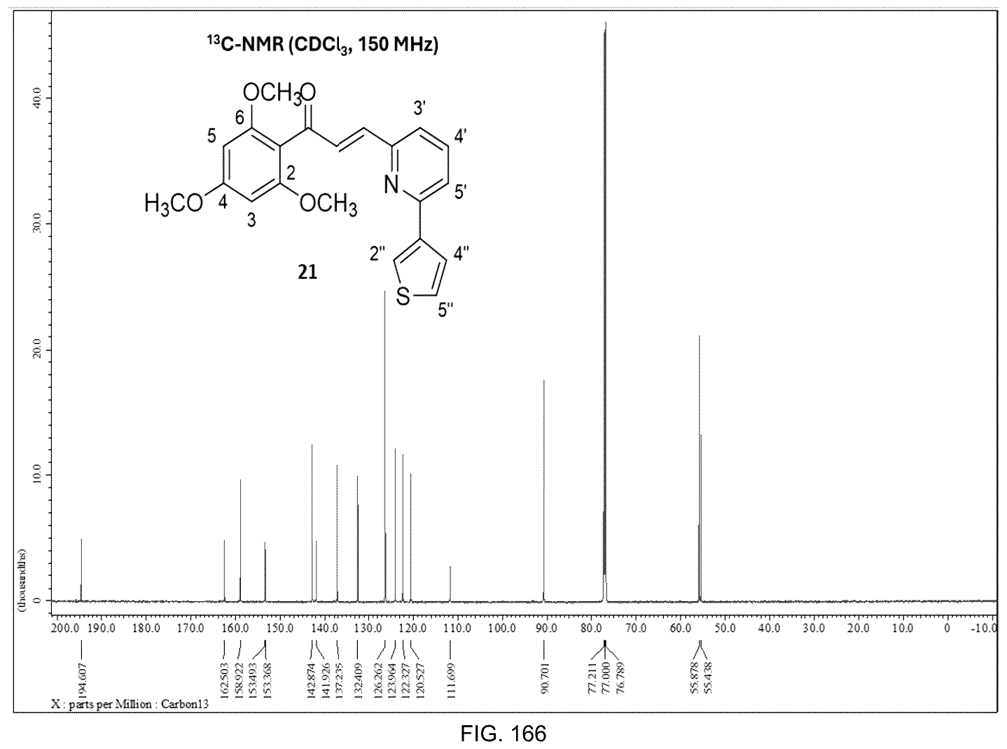

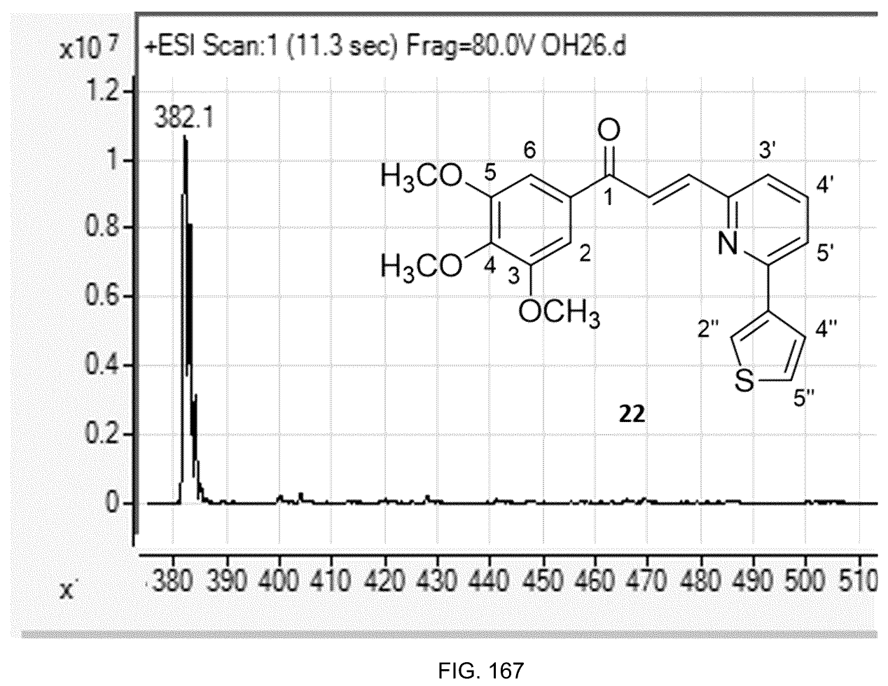

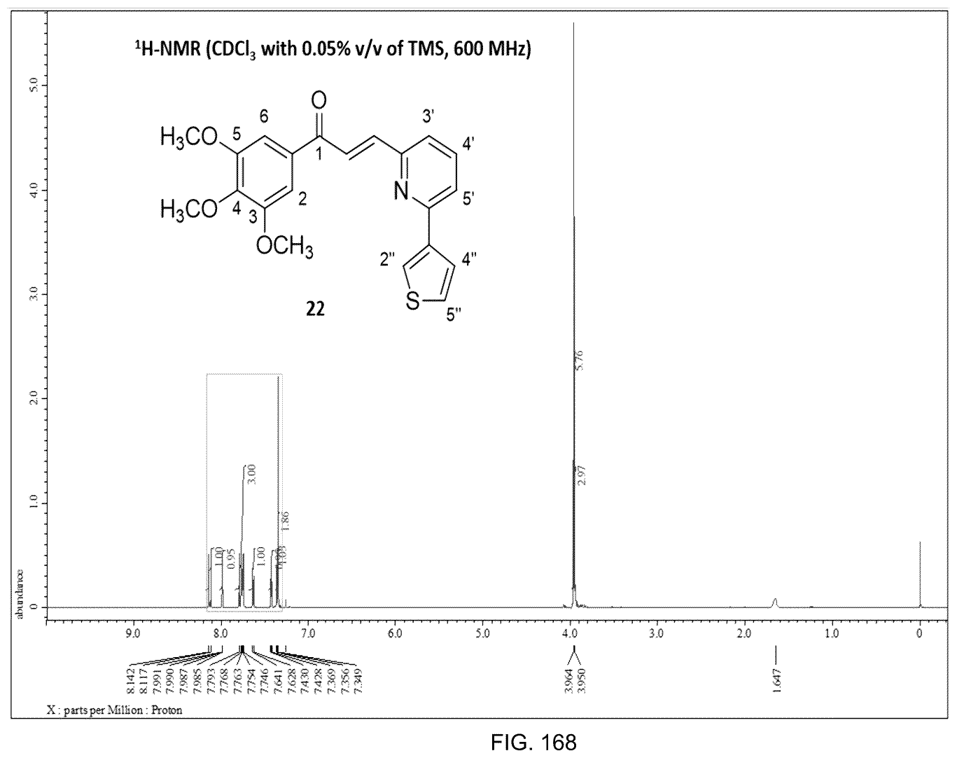

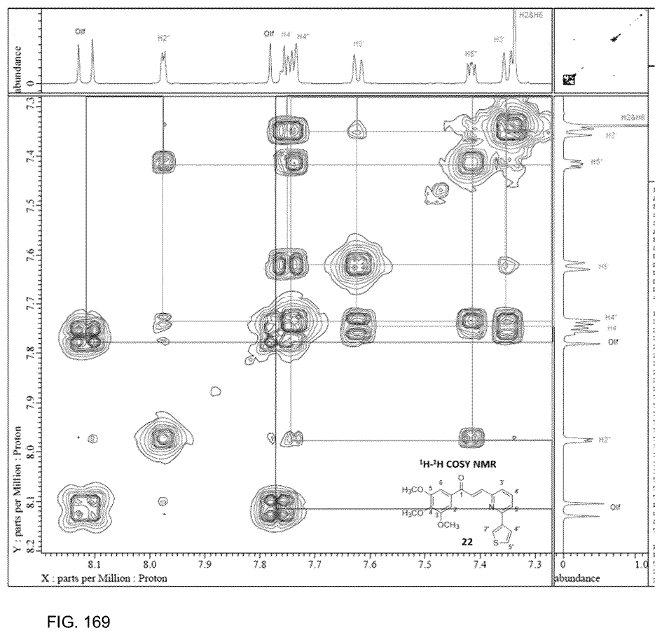

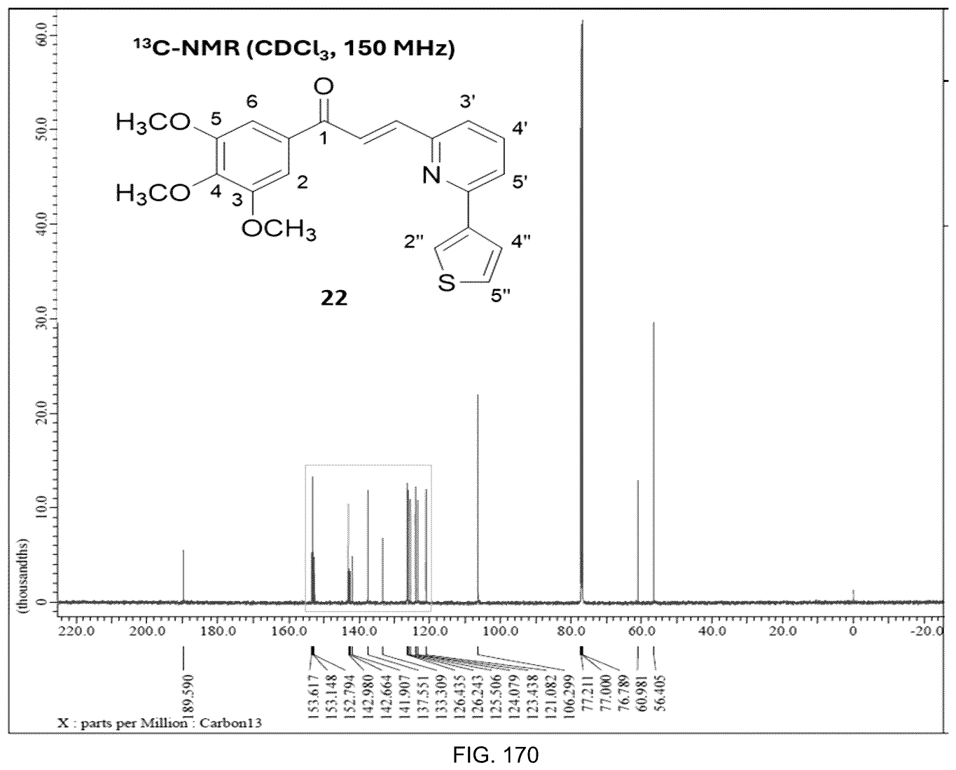

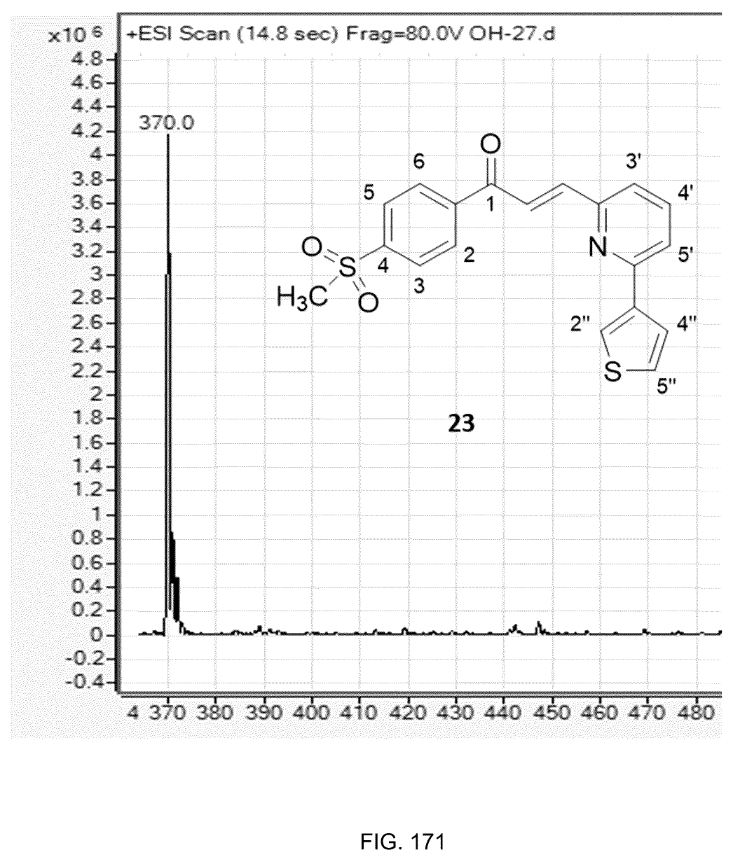

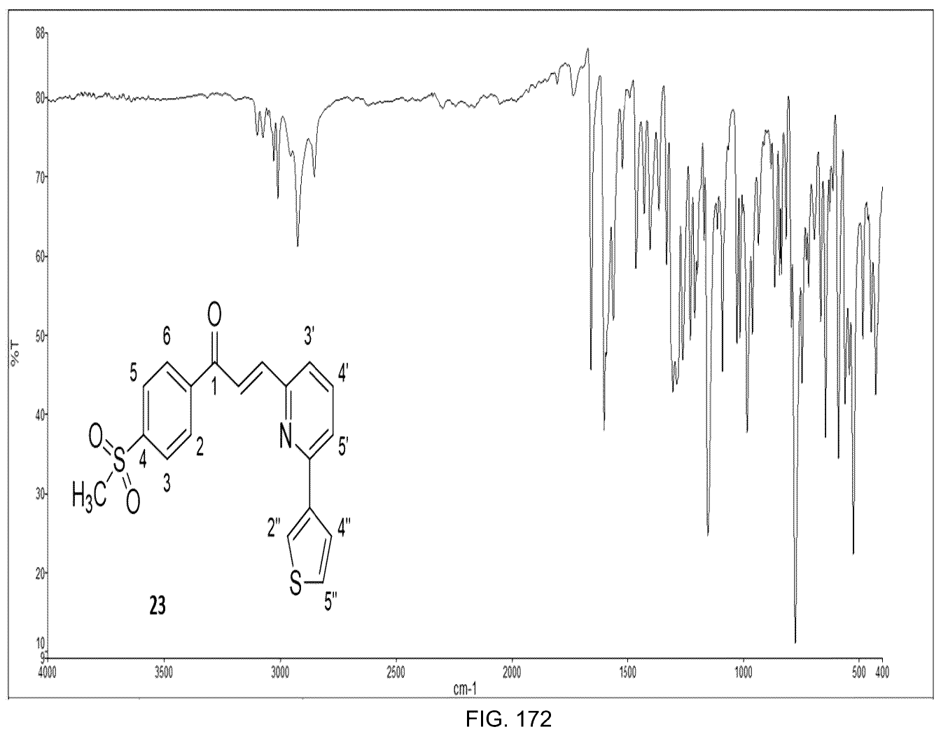

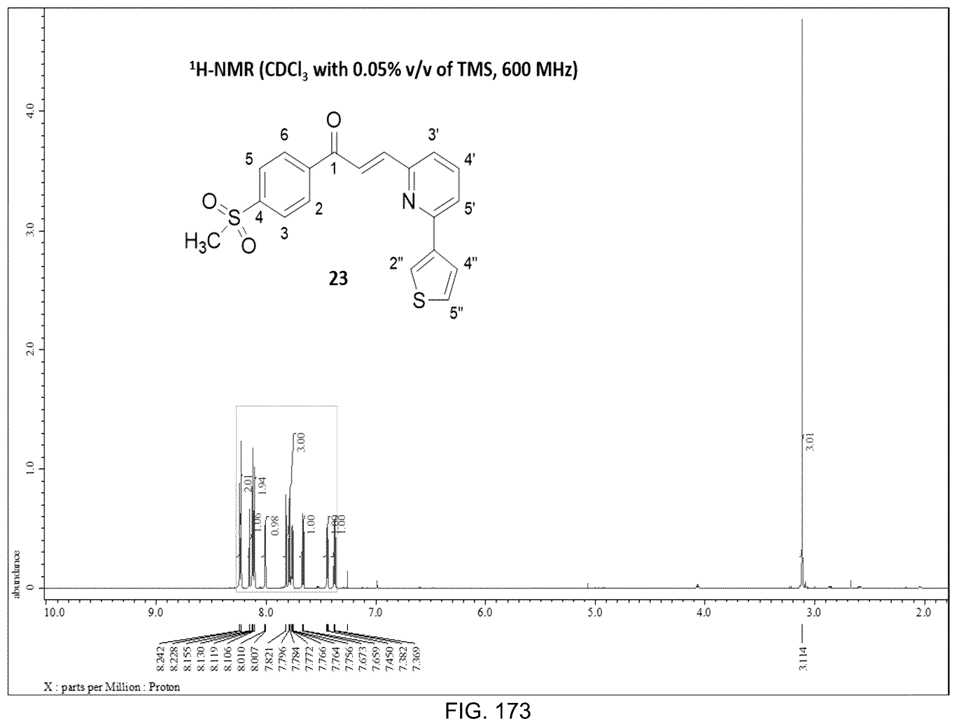

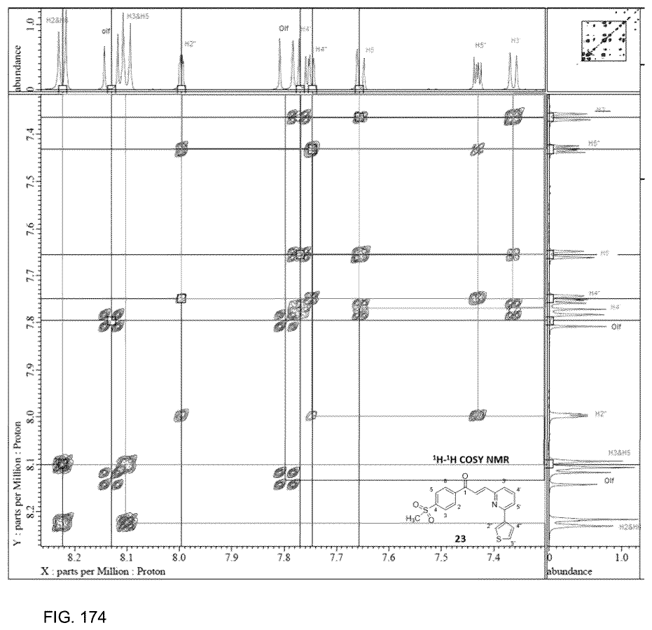

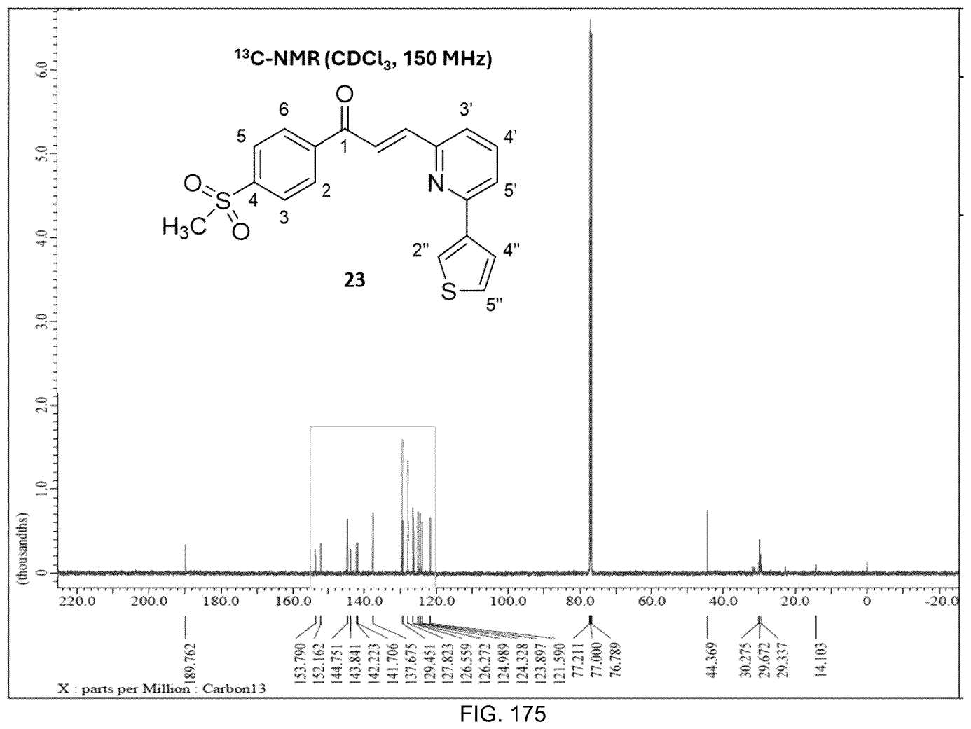

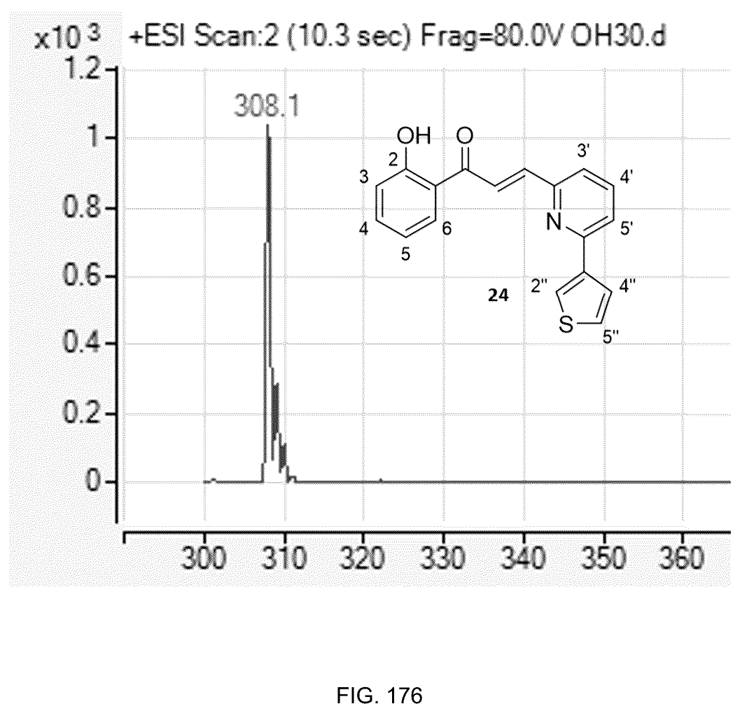

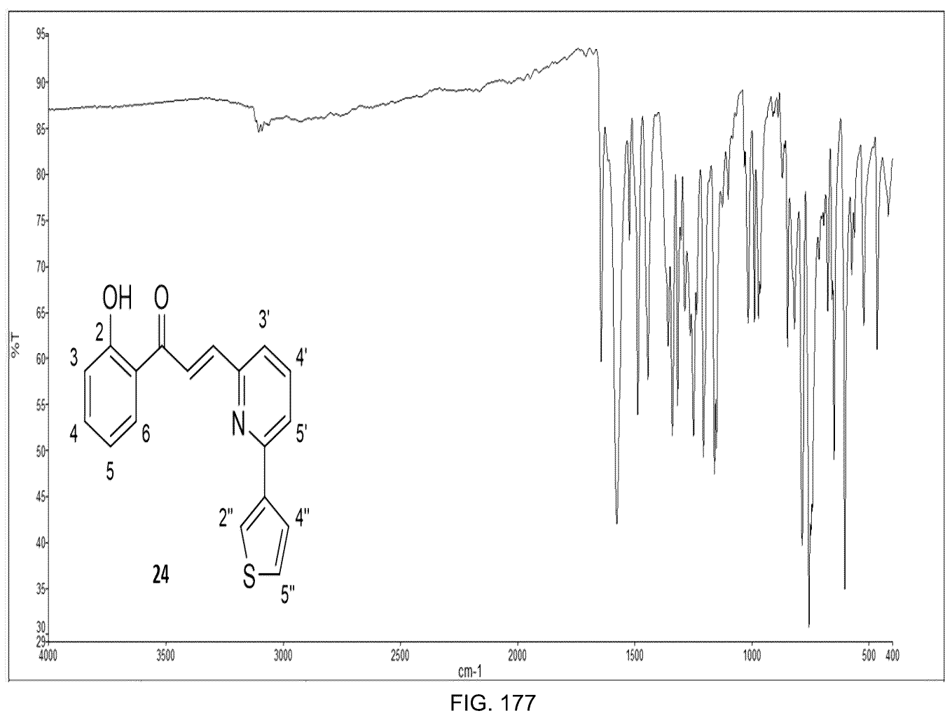

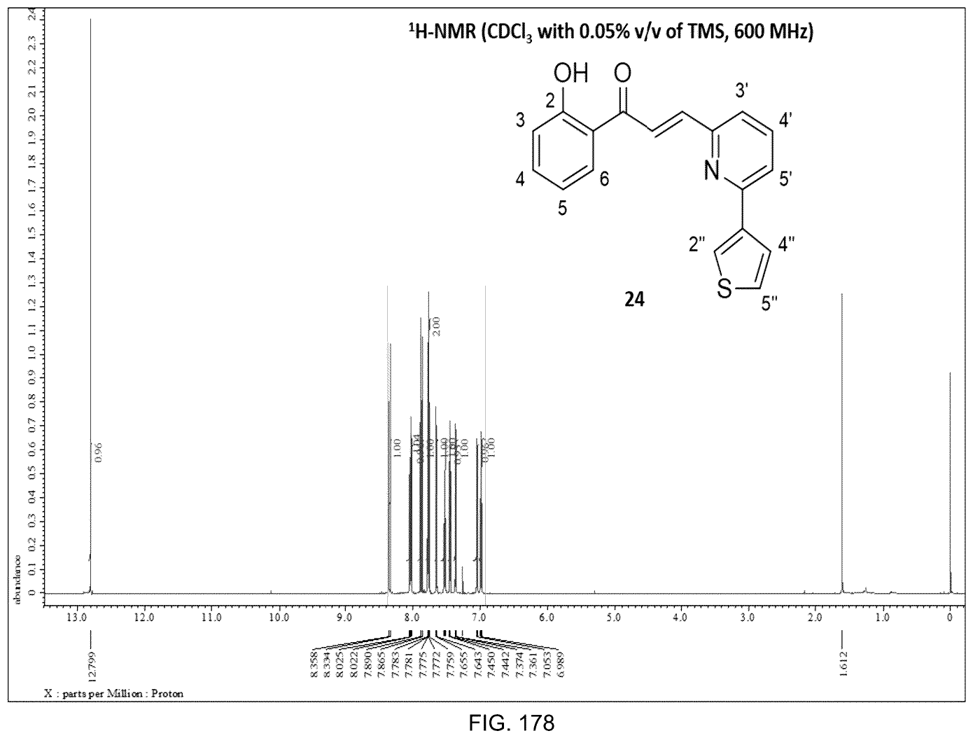

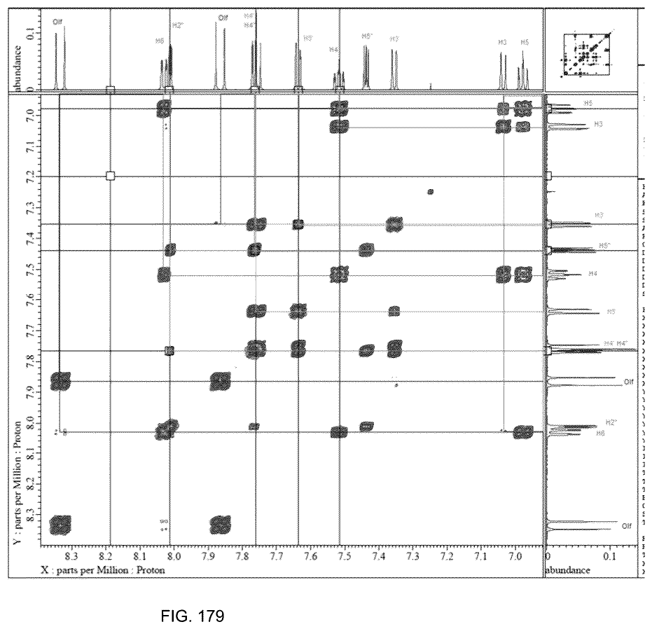

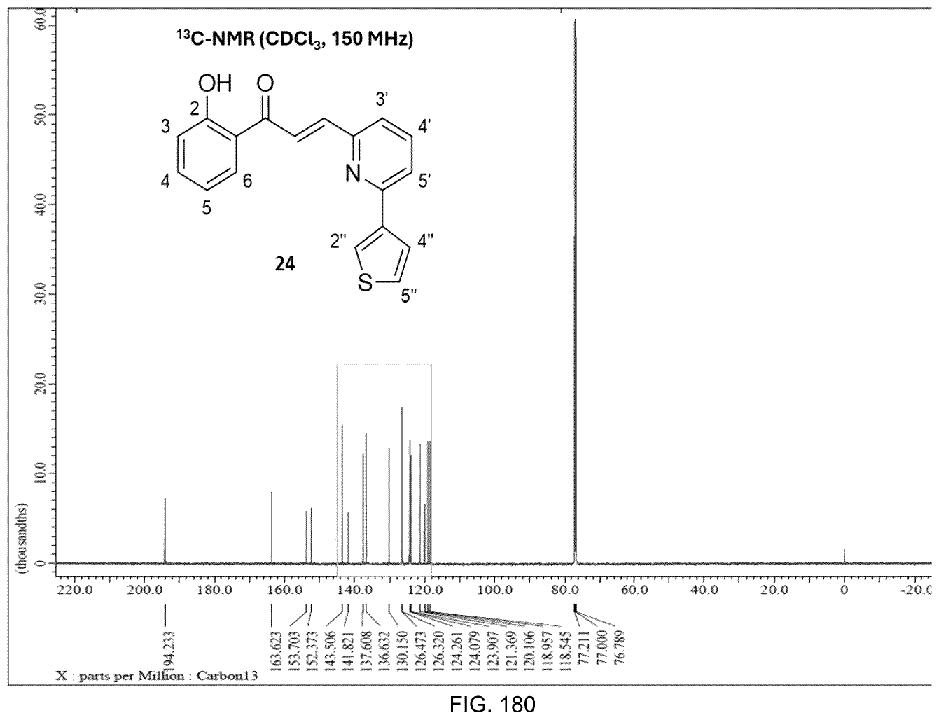

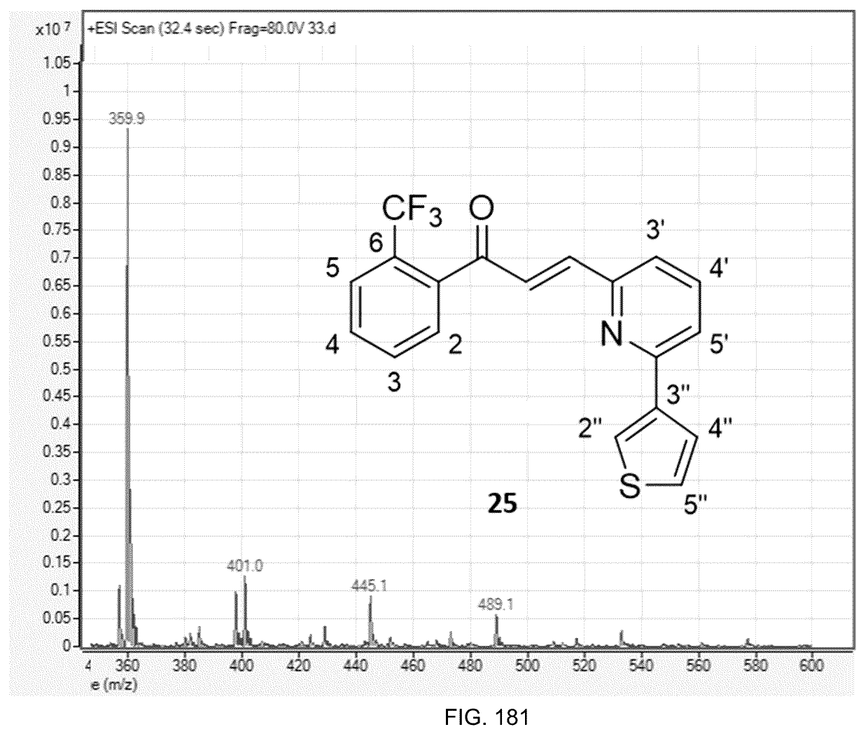

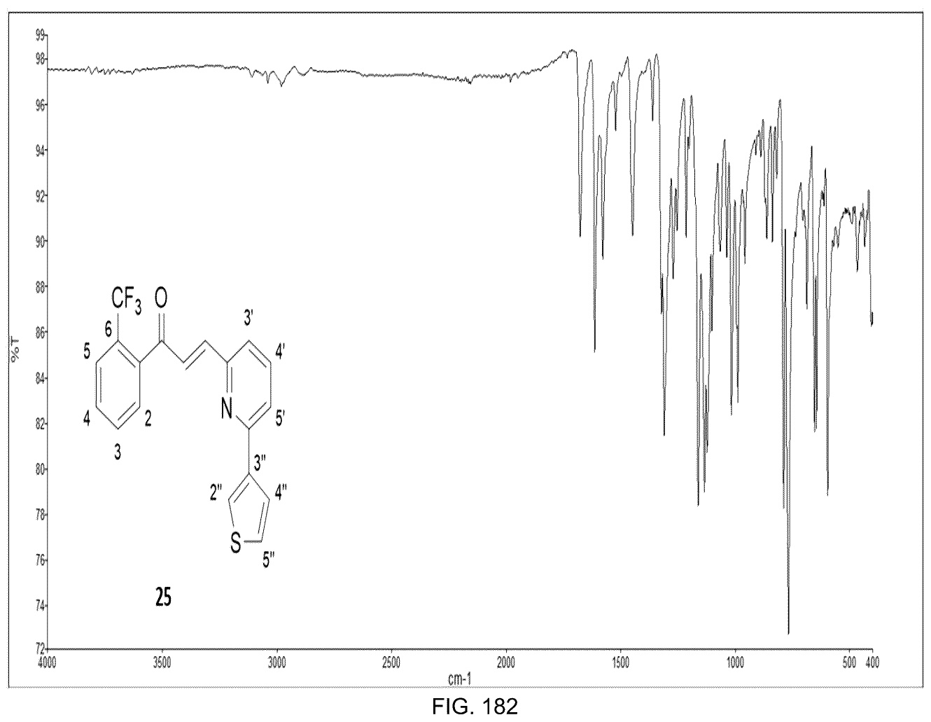

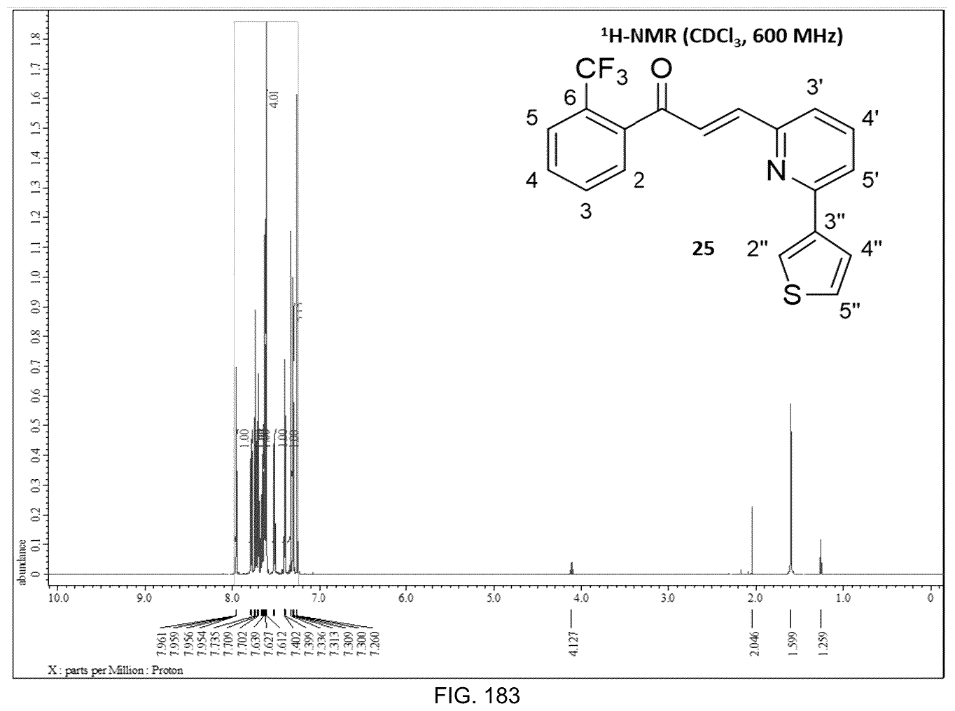

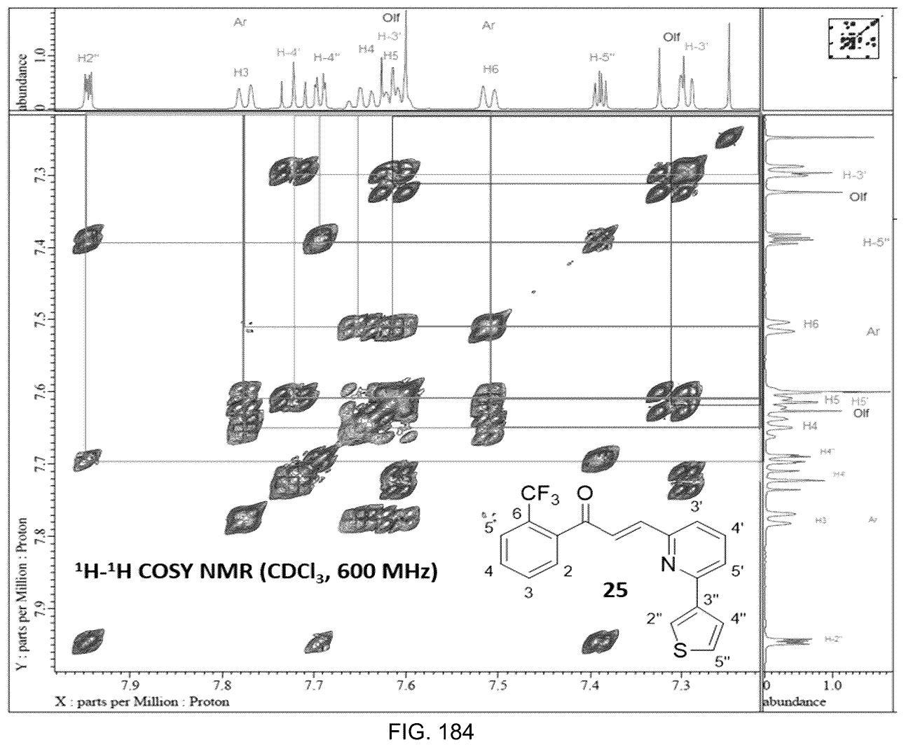

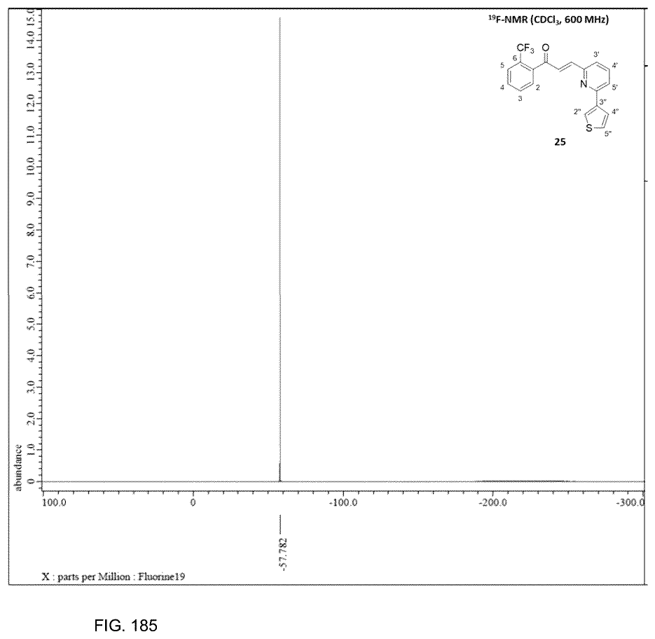

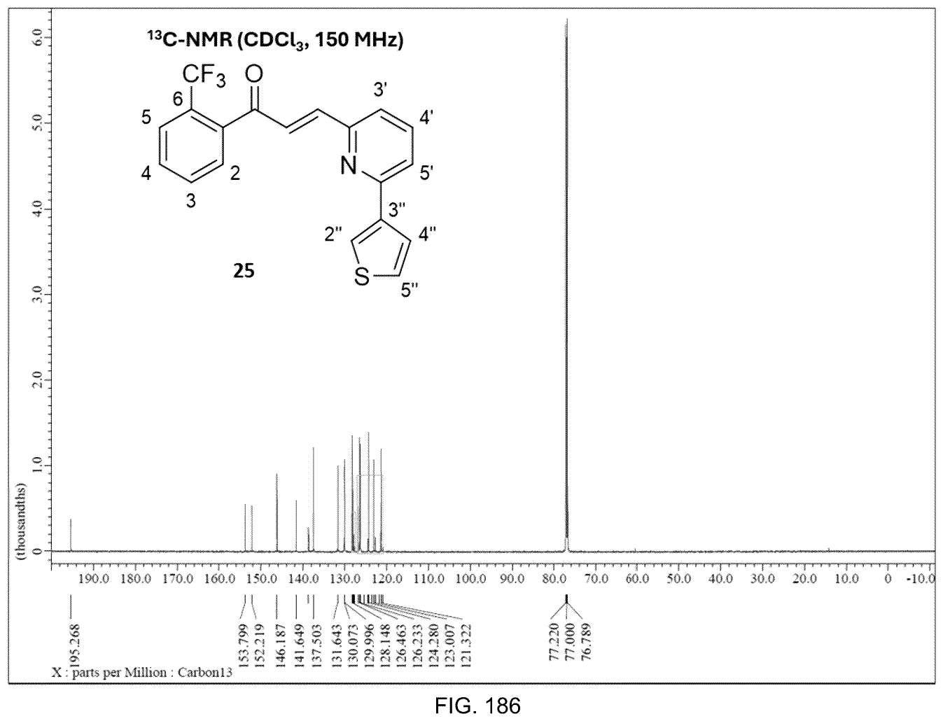

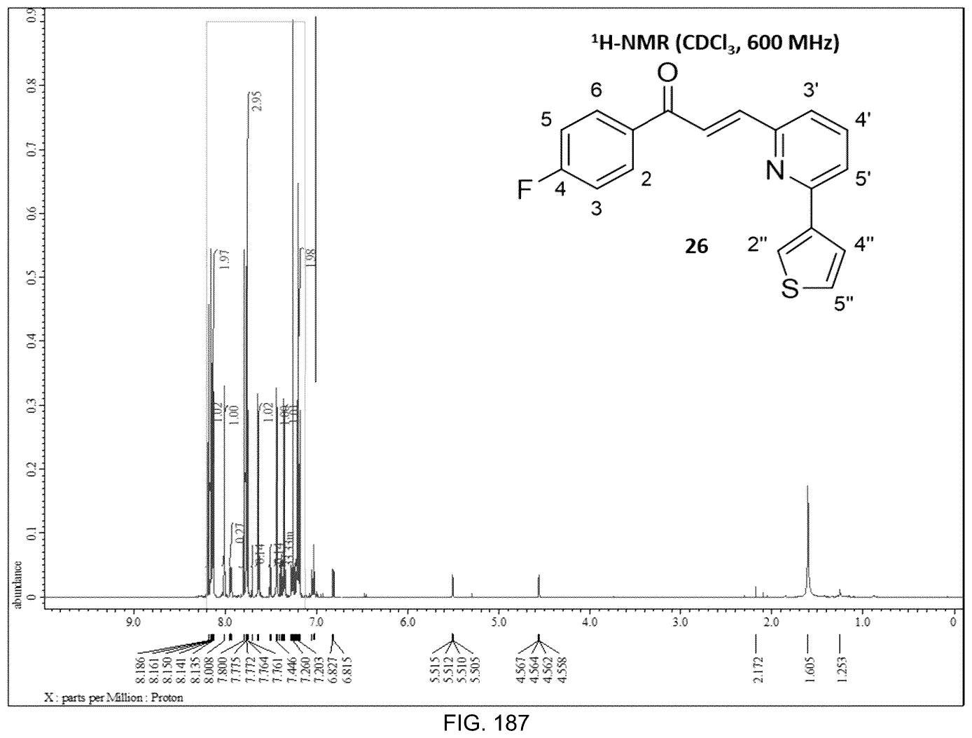

The findings from the first phase of the study (screening of compounds 116) indicate that thienyl pyridine-based chalcone (16) is a highly promising lead compound for the treatment of CRPC. Ten additional analogs (17-26) were synthesized and evaluated against CRPC and TNBC cell lines. The SAR study showed that non-cyclic chalcones were significantly more potent than their matched cyclic (tetralone-based) analogs. Changing the methoxy group's position in compound 16 from C3 to C2 appears to have dramatically improved the cytotoxicity. Treatment of PC3 cells with compound 17 at 2.5 μM concentration reduced cell viability by 84.7% as compared to 47.5% reduction with compound 16. Similarly, compound 17 showed a highly potent cytotoxic activity against TNBC cell lines with IC50 of 1.6 and 3.2 μM against MDA-MB-436 and MDA-MB-231, respectively. Moreover, replacing the methoxy with trifluoromethyl functional group in compound 25 resulted in a similar improvement in cytotoxicity (IC50 0.87-2.1). While compounds 17 and 25 showed relatively similar efficacy, compound 17 showed a more favorable ADMET profile. FIG. 56 summarizes the IC50 and ADMET risk for all heteroaromatic-based chalcones reported in the literature with activity against prostate cancer. Taken both the efficacy and the pharmacokinetic profile into consideration, the lead molecule (17) is among the most promising chalcone hybrids privileged with both high potency and relatively favorable ADMET profile.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A compound according to Formula (I): 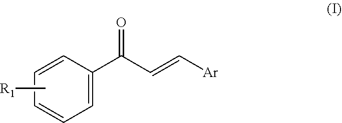
or a pharmaceutically acceptable salt thereof,
wherein
Ar is 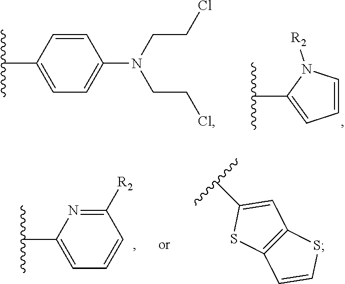
R₁ is —OCH₃, —OH, —CF₃, —SO₂CH₃, or —F; and
R₂ is absent, —H, cyclopropyl, —OCH₃, —CF₃, or thienyl.

2. The compound of claim 1, which has the structure of Formula (II): 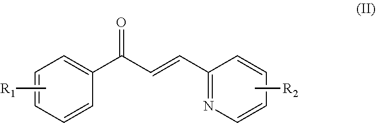
wherein R₂ is OCH₃, CF₃, or thienyl.

3. The compound of claim 2, wherein the R1 is —OCH₃.

4. The compound of claim 2, wherein the R1 is-CF₃.

5. The compound of claim 2, wherein the R₂ is thienyl.

6. The compound of claim 5, wherein the R₂ is 3-thienyl.

7. The compound of claim 2, which has the structure of Formula (III)

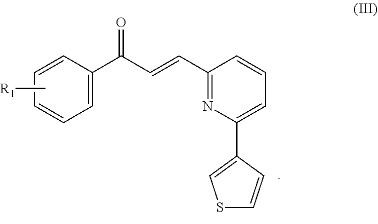

8. The compound of claim 2, which is any one of compounds 13-26 in FIG. 15.

9. The compound of claim 2, which has the structure of the following formula:

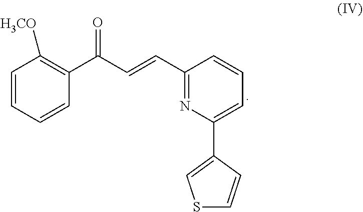 [SMILES: COc1ccccc1C(=O)/C=C/c1cccc(-c2ccsc2)n1]

10. The compound of claim 2, which has the structure of the following formula:

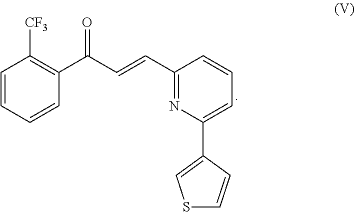 [SMILES: O=C(/C=C/c1cccc(-c2ccsc2)n1)c1ccccc1C(F)(F)F]

11. A pharmaceutical composition comprising the compound of claim 1 and a pharmaceutically acceptable carrier.

12. A method of treating a disease, the method comprising administering the compound of claim 1 or the pharmaceutical composition comprising the compound to a subject in need thereof.

13. The method of claim 12, wherein the disease is a cancer.

14. The method of claim 12, wherein the cancer is a breast cancer.

15. The method of claim 14, wherein the breast cancer is TNBC.

16. The method of claim 15, wherein the cancer is a prostate cancer.
